SEQUENCE LISTING (6 sequences)

SEQ ID NO: 1 - RNA, 1925 nt, synthetic construct
  source 1..1925: mol_type = other RNA
  modified_base 1..1925: mod_base = m1f
gggagagtcccgcagtcggcgtccagcggctctgcttgttcgtgtgtgtgtcgttgcaggccttattcaagcttaccatggaggacgccaagaacatcaagaagggccccgccccgttctaccccctggaggacgggaccgcgggcgagcagctccacaaggccatgaagcggtacgccctggtgcccgggaccatcgccttcacggacgcccacatcgaggtcgacatcacctacgcggagtacttcgagatgagcgtgcgcctggccgaggccatgaagcggtacggcctcaacaccaaccaccgcatcgtggtctgctccgagaacagcctgcagttcttcatgcccgtgctgggggccctcttcatcggcgtggcggtcgccccggccaacgacatctacaacgagcgggagctgctgaactccatgggcatcagccagcccaccgtggtgttcgtctccaagaaggggctccagaagatcctgaacgtgcagaagaagctgccgatcatccagaagatcatcatcatggacagcaagacggactaccagggcttccagtccatgtataccttcgtgaccagccacctccccccggggttcaacgagtacgacttcgtccccgagtccttcgaccgcgacaagaccatcgccctgatcatgaacagctccggcagcacggggctgcccaagggcgtggccctcccccaccggaccgcgtgcgtgcgcttctcccacgcccgggacccgatcttcggcaaccagatcatccccgacaccgccatcctgagcgtcgtgcccttccaccacgggttcggcatgttcaccacgctggggtacctcatctgcggcttccgcgtggtcctgatgtaccggttcgaggaggagctgttcctccgctccctgcaggactacaagatccagagcgccctgctcgtgcccaccctgttctccttcttcgccaagagcaccctgatcgacaagtacgacctctccaacctgcacgagatcgcgagcggcggggccccgctgagcaaggaggtgggcgaggccgtcgccaagcggttccacctccccgggatccgccagggctacgggctgaccgagacgacctccgccatcctgatcacccccgagggcgacgacaagcccggcgcggtggggaaggtggtcccgttcttcgaggccaaggtggtcgacctcgacaccggcaagacgctgggggtgaaccagcggggcgagctgtgcgtgcgcgggcccatgatcatgagcggctacgtcaacaaccccgaggccaccaacgccctcatcgacaaggacggctggctgcactccggggacatcgcctactgggacgaggacgagcacttcttcatcgtggaccggctcaagagcctcatcaagtacaagggctaccaggtggcgcccgccgagctggagtccatcctgctccagcacccgaacatcttcgacgccggggtcgccggcctgcccgacgacgacgcgggggagctgcccgccgccgtggtggtcctcgagcacggcaagaccatgaccgagaaggagatcgtggactacgtggccagccaggtcacgaccgccaagaagctgcgcggcggggtggtgttcgtcgacgaggtgcccaagggcctgaccgggaagctggacgcgcggaagatccgcgagatcctcatcaaggccaagaagggcggcaagatcgccgtctgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - RNA, 1925 nt, synthetic construct; identical to SEQ ID NO: 1
  source 1..1925: mol_type = other RNA

SEQ ID NO: 3 - RNA, 1925 nt, synthetic construct
  source 1..1925: mol_type = other RNA
  modified_base 1: mod_base = OTHER; note = methylated at 2'O of ribose sugar
  modified_base 1..1925: mod_base = m1f
aggagagtcccgcagtcggcgtccagcggctctgcttgttcgtgtgtgtgtcgttgcaggccttattcaagcttaccatggaggacgccaagaacatcaagaagggccccgccccgttctaccccctggaggacgggaccgcgggcgagcagctccacaaggccatgaagcggtacgccctggtgcccgggaccatcgccttcacggacgcccacatcgaggtcgacatcacctacgcggagtacttcgagatgagcgtgcgcctggccgaggccatgaagcggtacggcctcaacaccaaccaccgcatcgtggtctgctccgagaacagcctgcagttcttcatgcccgtgctgggggccctcttcatcggcgtggcggtcgccccggccaacgacatctacaacgagcgggagctgctgaactccatgggcatcagccagcccaccgtggtgttcgtctccaagaaggggctccagaagatcctgaacgtgcagaagaagctgccgatcatccagaagatcatcatcatggacagcaagacggactaccagggcttccagtccatgtataccttcgtgaccagccacctccccccggggttcaacgagtacgacttcgtccccgagtccttcgaccgcgacaagaccatcgccctgatcatgaacagctccggcagcacggggctgcccaagggcgtggccctcccccaccggaccgcgtgcgtgcgcttctcccacgcccgggacccgatcttcggcaaccagatcatccccgacaccgccatcctgagcgtcgtgcccttccaccacgggttcggcatgttcaccacgctggggtacctcatctgcggcttccgcgtggtcctgatgtaccggttcgaggaggagctgttcctccgctccctgcaggactacaagatccagagcgccctgctcgtgcccaccctgttctccttcttcgccaagagcaccctgatcgacaagtacgacctctccaacctgcacgagatcgcgagcggcggggccccgctgagcaaggaggtgggcgaggccgtcgccaagcggttccacctccccgggatccgccagggctacgggctgaccgagacgacctccgccatcctgatcacccccgagggcgacgacaagcccggcgcggtggggaaggtggtcccgttcttcgaggccaaggtggtcgacctcgacaccggcaagacgctgggggtgaaccagcggggcgagctgtgcgtgcgcgggcccatgatcatgagcggctacgtcaacaaccccgaggccaccaacgccctcatcgacaaggacggctggctgcactccggggacatcgcctactgggacgaggacgagcacttcttcatcgtggaccggctcaagagcctcatcaagtacaagggctaccaggtggcgcccgccgagctggagtccatcctgctccagcacccgaacatcttcgacgccggggtcgccggcctgcccgacgacgacgcgggggagctgcccgccgccgtggtggtcctcgagcacggcaagaccatgaccgagaaggagatcgtggactacgtggccagccaggtcacgaccgccaagaagctgcgcggcggggtggtgttcgtcgacgaggtgcccaagggcctgaccgggaagctggacgcgcggaagatccgcgagatcctcatcaaggccaagaagggcggcaagatcgccgtctgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - RNA, 1925 nt, synthetic construct; identical to SEQ ID NO: 3
  source 1..1925: mol_type = other RNA
  modified_base 1: mod_base = OTHER; note = methylated at 2'O of ribose sugar

SEQ ID NO: 5 - RNA, 1925 nt, synthetic construct; identical to SEQ ID NO: 3
  source 1..1925: mol_type = other RNA
  modified_base 1..1925: mod_base = m1f
  modified_base 1: mod_base = OTHER; note = methylated at 2'O of ribose sugar
  modified_base 2: mod_base = OTHER; note = methylated at 2'O of ribose sugar

SEQ ID NO: 6 - RNA, 1925 nt, synthetic construct; identical to SEQ ID NO: 3
  source 1..1925: mol_type = other RNA
  modified_base 1: mod_base = OTHER; note = methylated at 2'O of ribose sugar
  modified_base 2: mod_base = OTHER; note = methylated at 2'O of ribose sugar

(19) United States
(12) Patent Application Publication
(10) Pub. No.: US 2026/0226091 A1
(43) Pub. Date: Aug. 6, 2026

(54) CAP ANALOGS WITH 5'-TERMINAL ACYCLIC GUANOSINE DERIVATIVE

(71) Applicant: CureVac SE, Tübingen (DE)

(72) Inventors: Rainer Joachim SCHWARZ, Tübingen (DE); Sarah MORENO, Tübingen (DE)

(73) Assignee: CureVac SE, Tübingen (DE)

(21) Appl. No.: 19/151,490

(22) PCT Filed: Jan. 31, 2024
(86) PCT No.: PCT/EP2024/052372

§ 371 Date: Jul. 28, 2025

(30) Foreign Application Priority Data
Jan. 31, 2023 (WO) PCT/EP2023/052352

(51) Int. Cl. C07H 21/02; C07H 1/00

(52) U.S. Cl. CPC C07H 21/02; C07H 1/00

(57) ABSTRACT

The present invention is inter alia concerned with (A) a compound of formula (I) as defined herein or a salt, stereoisomer, tautomer or deuterated version thereof, (B) a cap analog comprising a 5′ terminal acyclonucleoside, wherein the acyclonucleoside comprises a linear unbranched or linear single-branched structure of formula (II) instead of a ribose, wherein the 5′ terminal acyclonucleoside is optionally deuterated (C) an RNA molecule comprising at least three nucleotides and comprising a 5′ end of formula (III) as defined herein, wherein the 5′ end is optionally deuterated (D) an RNA molecule comprising at least three nucleotides and comprising a 5′ terminal acyclonucleoside, wherein the acyclonucleoside comprises a linear unbranched or linear single-branched structure of formula (II) instead of a ribose, wherein the 5′ terminal acyclonucleoside is optionally deuterated (E) an in vitro method for synthesizing an RNA molecule, (F) the RNA molecule obtained thereby, (G) compositions comprising the RNA molecule, (H) kits comprising the compound of formula (I) or the cap analog, (J) uses as well as (K) a process for preparing and (L) methods as outlined herein.

### TECHNICAL FIELD

The present invention relates to a compound of formula (I) as defined herein or a salt, stereoisomer, tautomer or deuterated version thereof. The present invention further relates to a cap analog comprising a 5′ terminal acyclonucleoside, wherein the acyclonucleoside comprises a linear unbranched or linear single-branched structure of formula (II) as defined herein instead of a ribose, wherein the cap analog is a Cap1 analog or a Cap2 analog and wherein the 5′ terminal acyclonucleoside is optionally deuterated. The present invention further relates to an RNA molecule comprising at least three nucleotides and comprising a 5′ end of formula (III) as defined herein, wherein the 5′ end is optionally deuterated, and an RNA molecule comprising at least three nucleotides and comprising a 5′ terminal acyclonucleoside, wherein the acyclonucleoside comprises a linear unbranched or linear single-branched structure of formula (II) as defined herein instead of a ribose, and wherein the 5′ terminal acyclonucleoside is optionally deuterated. Further, the present invention relates to an in vitro method for synthesizing an RNA molecule as well as the RNA molecule obtained thereby. Compositions comprising the RNA molecule, kits comprising the compound of formula (I) or the cap analog, uses as well as a process for preparing and methods as outlined in the following are also part of the present invention.

### BACKGROUND OF THE INVENTION

Eukaryotic mRNA has a cap structure at its 5-terminus, wherein this cap structure consists of 7-methyl guanosine (m⁷G) and a triphosphate bridge (ppp) linking the 5′OH of the m⁷G to the 5′OH of the 5′-terminal nucleotide (N). This structure can be referred to as m⁷G(5′)pppN. The cap structure of an mRNA is inter alia implicated in eukaryotic cells in the assembly of the translation initiation complex by binding to the eukaryotic translation initiation factor 4E (eIF-4E). It is therefore essential to maintain a cap structure in mRNAs that are produced in vitro and that are intended to be used in pharmaceutical products. In such products, the mRNAs are translated in and by the cells of the subject to be treated into the encoded peptides or proteins. Typically, such mRNAs are produced in in vitro transcription reactions using a DNA template and a DNA-dependent RNA polymerase, such as in particular T7 or SP6 DNA-dependent RNA polymerase. The capping can either be carried out co-transcriptionally or after the transcription reaction.

For co-transcriptional capping reactions, m⁷G(5′)ppp was found to be used by T7 or SP6 DNA-dependent RNA polymerase in vitro to initiate the transcription reaction. However, m⁷G(5′)ppp has the disadvantage of having to compete with the guanine nucleotide (G) as the initiating nucleophile for transcription elongation such that less than half of the in vitro produced mRNAs have a cap structure at their 5-termini if m⁷G(5′)ppp is used.

Dinucleotide-cap analogs have also been developed and described (E. Darzynkiewicz and A. J. Shatkin, Biochemistry 1985, 24, 7, 1701-1707), in particular the cap analog m⁷G(5′)ppp(5′)G. m⁷G(5′)ppp(5′)G has been successfully used in in vitro transcription reactions as initiator of transcription to produce cap structures co-transcriptionally. However, m⁷G(5′)ppp(5′)G has the disadvantage that the 3′-OH group of either the m⁷G or the G moiety can serve as the initiating nucleophile for transcriptional elongation. Accordingly, two different RNAs are produced, namely m⁷G(5′)pppG(pN)n (with the correct orientation of the cap) and G(5′)pppm⁷G(pN)n (with the reverse orientation of the cap), with one third to half of the cap structures oriented in the reverse direction. In order to render the reverse orientation impossible during the in vitro reaction, so-called anti-reverse cap analogs (ARCAs) have been developed, where the 3′-OH group of the m⁷G moiety is replaced with hydrogen or OCH₃ (J Stepinski and R E Rhoads; RNA. 2001 October; 7(10): 1486-1495. PMID: 116808539).

Further cap analogs that aim at increasing the binding efficiency towards eIF-4E and the expression level have been developed. Modification sites were the N7-Position of the cap (WO 2016/098028), the ribose of the m⁷G (WO 2017/066797; U.S. Pat. No. 7,074,596), the triphosphate bridge (WO 2009/149253; WO 2017/066781; WO 2017/066791; A. M Rydzik, J. Jemielity, Bioorg Med Chem. 2012; 20(5):1699-710 PMID: 22316555; J. Kowalska and J. Jemielity Nucleic Acids Res. 2014; 42(16):10245-64 PMID: 25150148; B. A Wojtczak, J. Jemielity, J Am Chem Soc. 2018 May 9,140(18):5987-5999 PMID: 29676910) and the first translated nucleotide (S. Akichika and T. Suzuki Science. 2019 Jan. 11; 363(6423):eaav0080 PMID: 30467178; M. Kopcial and J. Jemielity, Molecules. 2019 May 17; 24(10):1899 PMID: 31108861).

A general disadvantage of the afore-mentioned analogs is the recognition of these structures by IFIT1 and IFIT3 proteins, resulting in immunostimulation (B. Johnson and G. K. Amarasinghe, 2018 Mar. 20:48(3):487-499 PMID: 29525521). In order to reduce the immunostimulation, trinucleotide analogs have been developed, which are also suitable for co-transcriptional capping. An example of such analogs is m⁷GpppNmpN, where the 2′-OH group of the first translated nucleotide is methylated (Nm). Such cap analogs show a high capping efficiency and lead to a high expression of the resulting mRNA (WO 2017/053297; P. J Sikorski and J. Jemielity Nucleic Acids Res. 2020 Feb. 28; 48(4):1607-1626 PMID: 31984425).

There remains a need to provide cap analogs that have inter alia a high efficiency as regards the co-transcriptional capping in in vitro transcription reactions and that result in in high expression levels of capped RNAs produced by in vitro reactions using such cap analogs.

### SUMMARY OF THE INVENTION

The inventors solved the above need in that they surprisingly found new cap analogs as described herein.

In the first aspect, the present invention relates to a compound of formula (I):

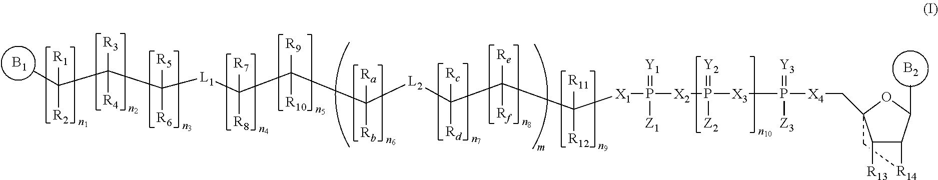

or a salt, stereoisomer, tautomer, or deuterated version thereof, wherein
R₁₃ is

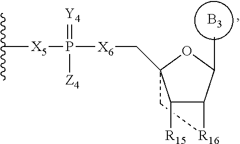

wherein
R₁₅ is OH or

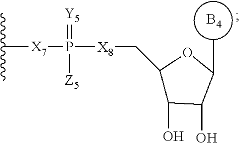

ring B₁ is guanine, a modified guanine or a guanine analog;
each of ring B₂ through ring B₄ is independently a nucleobase, a modified nucleobase or a nucleobase analog;
n₁ and n₉ are independently selected from an integer ranging from 1 to 10;
each of R₁ and R₂ is independently H, OH, SH, NH₂ or halogen, wherein, if n₁ is greater than 1, R₁ may be same or different in each repeating unit, and R₂ may be same or different in each repeating unit;
each of R₁₁ and R₁₂ is independently H, OH, SH, NH₂ or halogen, wherein, if n₉ is greater than 1, R₁₁ may be same or different in each repeating unit, and R₁₂ may be same or different in each repeating unit;
each of n₃, n₄, n₆, and n₇ is independently selected from an integer ranging from 0 to 5; wherein, if m is greater than 1, n₆ may be same or different in each repeating unit, and n₇ may be same or different in each repeating unit;
each of R₅ and R₆, if present, is independently H, OH, SH, NH₂ or halogen, wherein, if n₃ is greater than 1, R₅ may be same or different in each repeating unit, and R₆ may be same or different in each repeating unit;
each of R₇ and R₈, if present, is independently H, OH, SH, NH₂ or halogen, wherein, if n₄ is greater than 1, R₇ may be same or different in each repeating unit, and R₈ may be same or different in each repeating unit;
each of Rₐ and R_{b}, if present, is independently H, OH, SH, NH₂ or halogen, wherein, if n₆ is greater than 1, Rₐ may be same or different in each repeating unit, and R_{b} may be same or different in each repeating unit;
each of R_{c} and R_{d}, if present, is independently H, OH, SH, NH₂ or halogen, wherein, if n₇ is greater than 1, R_{c} may be same or different in each repeating unit, and R_{d} may be same or different in each repeating unit;
n₂, n₅ and n₈ are independently selected from 0 or 1; wherein, if m is greater than 1, n₈ may be same or different in each repeating unit;
one of R₃ and R₄, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl- R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group, and the other one of R₃ and R₄, if present, is selected from the group consisting of H, OH, SH, NH₂ and halogen; one of R₉ and R₁₀, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl- R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group; and the other one of R₉ and R₁₀, if present, is selected from the group consisting of H, OH, SH, NH₂ and halogen; one of Rₑ and R_{f}, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R³, C₁-C₃-alkyl-S—S—R^{S}, C₁-C₃-alkyl-NR^{N1}R^{N2}, C₁-C₃-alkyl- R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group; and the other one of Rₑ and R_{f}, if present, is selected from the group consisting of H, OH, SH, NH₂ and halogen;
m is selected from an integer ranging from 0 to 10, with the proviso that (i) if m is 0, one of n₂ and n₅ is 1 and the other one of n₂ and n₅ is 0; (ii) if m is 1, (a) n₂, n₅ and n₈ are 0 or (b) one of n₂, n₅ and n₈ is 1 and the other two of n₂, n₅ and n₈ are 0; and (iii) if m is an integer ranging from 2 to 10, (a) n₂, n₅ and each n₈ are 0, (b) one of n₂ and n₅ is 1 and the other one of n₂ and n₅ as well as each n₈ are 0, or (c) one n₅ is 1 and each other n₈ as well as n₂ and n₅ are 0;
n₁₀ is selected from 0, 1 or 2;
L₁ is selected from the group consisting of CH₂, O, S, SO, SO₂, NR^{L}, CH(OH), CH(SH) and CH(halogen), with the proviso that, if m is 1 and n₂, n₅ and n₈ are 0, L₁ is selected from the group consisting of O, S, SO, SO₂ and NR^{L};
L₂ is selected from the group consisting of O, S, SO, SO₂ and NH; wherein, if m is greater than 1, L₂ may be same or different in each repeating unit;
each of X₁ through X₈ is independently O, S, NH or CH₂, wherein, if n₁₀ is 2, X₃ may be same or different in each repeating unit;
each of Y₁ through Y₅ is independently O, S or Se, wherein, if n₁₀ is 2, Y₂ may be same or different in each repeating unit;
each of Z₁ through Z₅ is independently OH, SH, SeH or BH₃, wherein, if n₁₀ is 2, Z₂ may be same or different in each repeating unit;
R₁₄ is (i) selected from the group consisting of H, OH, OC₁-C₃-alkyl, and Opropargyl, wherein the dashed methylene bridge between R₁₄ and the 4′ C is absent, or (ii) O, wherein the dashed methylene bridge between R₁₄ being O and the 4′ C is present;
R₁₆ is (i) selected from the group consisting of H, OH, OC₁-C₃-alkyl, and Opropargyl, wherein the dashed methylene bridge between R₁₆ and the 4′ C is absent, or (ii) O, wherein the dashed methylene bridge between R₁₆ being O and the 4′ C is present;
R^{L} is (i) H, if one of n₂, n₅ and n₈ is 1, or (ii) selected from the group consisting of H, C₁-C₃-alkyl, C₁-C₃-alkyl-O—R^{C}, C₁-C₃-alkyl-S—R^{S}, C₁-C₃-alkyl-NR^{N1}R^{N2}, C₁-C₃-alkyl-R^{P} and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized, and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, if n₂, n₅ and n₈ are 0;
R^{O}, R^{S}, R^{N1} and R^{N2} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl- O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁- C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl- NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{D} and R^{E} are each independently selected from the group consisting of C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl- O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁- C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{N3} and R^{N4} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl- NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, with the proviso that R^{N3} and R^{N4} are not both H; and
R^{P} is

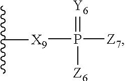

wherein
X₉ is O, S, NH or CH₂;
Y₆ is O, S or Se; and
each of Z₆ and Z₇ is independently OH, SH, SeH or BH₃.

In one embodiment of the first aspect, the compound of the present invention comprises a linear single-branched structure, i.e., a single carbon-containing substituent or a phosphate substituent is present on the linear structural element between B₁ and X₁ of formula (I). Thus, in one embodiment, m is an integer ranging from 0 to 10, one of n₂ and n₅ is 1 and the other one of n₂ and n₅ as well as each n₈, if present, are 0, or one n₈, if present, is 1 and each other n₈, if present, as well as n₂ and n₅ are 0. In another embodiment, m is 0, one of n₂ and n₅ is 1 and the other one of n₂ and n₅ is 0. And in another embodiment, m is 1, one of n₂, n₅ and n₈ is 1, and the other two of n₂, n₅ and n₈ are 0. In other words, one of CR₃R₄, CR₉R₁₀, and CRₑR_{f} is present and the other two of CR₃R₄, CR₉R₁₀, and CRₑR_{f} are absent.

In another embodiment of the first aspect, the compound of the present invention comprises a linear unbranched structure, i.e., no carbon-containing substituent or phosphate substituent is present on the linear structural element between B₁ and X₁ of formula (I). Thus, in one embodiment, m is 1 and n₂, n₅ and n₈ are 0. As indicated above, in this embodiment, L₁ is selected from the group consisting of O, S, SO, SO₂ and NR^{L} so that the linear structural element between B₁ and X₁ of formula (I) comprises two heteroatoms. In another embodiment, m is selected from an integer ranging from 2 to 10 and n₂, n₅ and n₈ are 0 so that the linear structural element between B₁ and X₁ of formula (I) comprises at least two heteroatoms because L₂ is present at least two times.

In the following, preferred embodiments of the substituents and number of repeating units (n₁ to n₁₀) in the above formula (I) are described in further detail. It is to be understood that each preferred embodiment is relevant on its own as well as in combination with other preferred embodiments. Further, it is to be understood that the preferences in each case also apply to the salts, stereoisomers, tautomers, or deuterated versions of the compounds of the invention. Furthermore, it is to be understood that each of the definitions for the substituent or the number of a repeating unit applies only if the substituent or repeating unit is present. For example, the substituent meaning for R_{c} only applies for compounds of formula (I), wherein m and n₇ are not 0. Similarly, the meaning of, e.g., n₆ and n₇ only applies for compounds of formula (I), wherein m is not 0. If embodiments include a list of different substituent(s) or repeating unit(s), which comprises present and non-present substituent(s) or repeating unit(s), the definition applies to all substituent(s) or repeating unit(s), which are present in the respective compound of formula (I).

As indicated above, in the compound of formula (I)

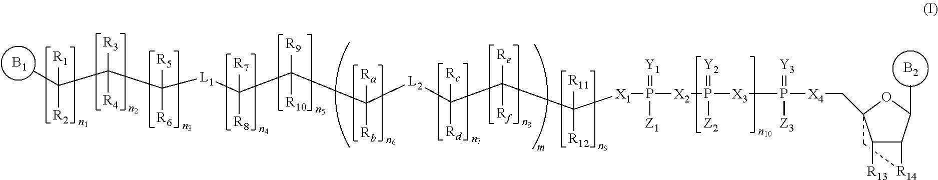

m can be an integer ranging from 0 to 10.

Preferably, m is an integer ranging from 0 to 9, more preferably 0 to 8, even more preferably 0 to 7, even more preferably 0 to 6, even more preferably 0 to 5, even more preferably 0 to 4, even more preferably 0 to 3, even more preferably 0 to 2. In a particularly preferred embodiment, m is 0 or 1.

Thus, in a preferred embodiment, the compound of formula (I) is a compound of formula (Ia), where m is 0, or (Ib), where m is 1, as shown below.

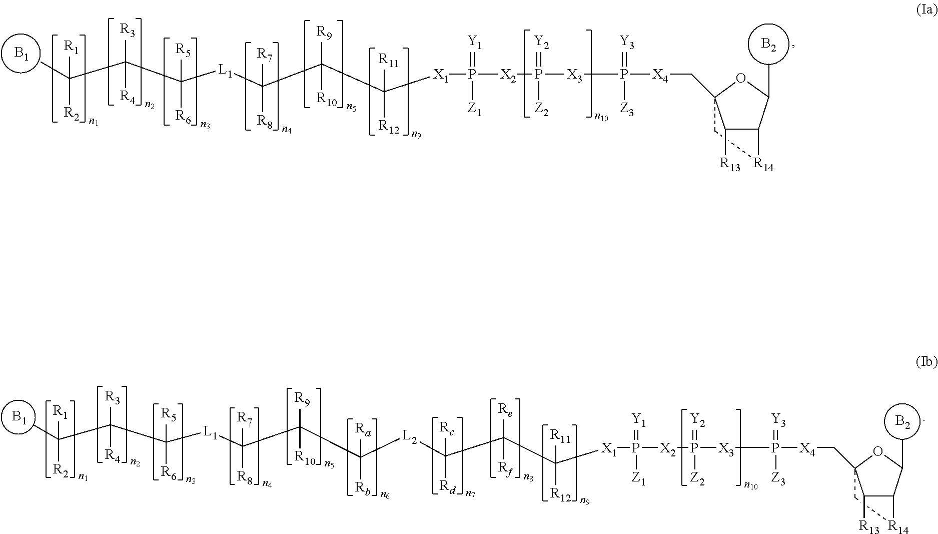

In a preferred embodiment, the compound of formula (I) is a compound of formula (Ia).

Further, as indicated above, the compound of formula (I) may comprise a linear unbranched structure or a linear single-branched structure.

In particular, if m is 0, i.e., if the compound of formula (I) is a compound of formula (Ia), the compound of formula (I) comprises a linear single-branched structure. Thus, in one embodiment, m is 0, one of n₂ and n₅ is 1 and the other one of n₂ and n₅ is 0.

If m is an integer ranging from 1 to 10, the compound of formula (I) comprises a linear single-branched structure or a linear unbranched structure, wherein the linear unbranched structure comprises at least 2 heteroatoms in the linear structural element. Thus, in one embodiment, m is an integer ranging from 1 to 10, one of n₂, n₅ and n₈ is 1, and the other two of n₂, n₅ and n₈ are 0. In another embodiment, m is an integer ranging from 1 to 10 and n₂, n₅ and each n₈ are 0, wherein, if m is 1, L₁ is selected from the group consisting of O, S, SO, SO₂ and NR^{L}.

If m is 1, i.e., if the compound of formula (I) is a compound of formula (Ib), the compound of formula (I) comprises a linear single-branched structure or a linear unbranched structure, wherein the linear unbranched structure comprises 2 heteroatoms in the linear structural element. Thus, in one embodiment, m is 1, one of n₂, n₅ and n₈ is 1, and the other two of n₂, n₅ and n₈ are 0. In another embodiment, m is 1, n₂, n₅ and n₈ are 0, and L₁ is selected from the group consisting of O, S, SO, SO₂ and NR^{L}.

If m is an integer ranging from 2 to 10, the compound of formula (I) comprises a linear single-branched structure or a linear unbranched structure, wherein the linear unbranched structure comprises at least 2 heteroatoms in the linear structural element. Thus, in one embodiment of the linear single-branched structure, m is an integer ranging from 2 to 10, one of n₂ and n₅ is 1 and the other one of n₂ and n₅ as well as each n₈ are 0. In another embodiment of the single-branched structure, m is an integer ranging from 2 to 10, one n₄ is 1 and each other n₈ as well as n₂ and n₅ are 0. In an embodiment of the linear unbranched structure, m is an integer ranging from 2 to 10 and n₂, n₅ and each n₈ are 0.

In connection with the compounds of formula (I), as well as in connection with the compounds of formula (Ia) and (Ib), the following embodiments are relevant.

As indicated above, the compound of the present invention may comprise a linear single-branched structure. Thus, in one embodiment, n₂ is 1. In another embodiment, n₅ is 1. In yet another embodiment, m is an integer ranging from 1 to 10 and one n₈ is 1 and each other n₈ is 0, wherein preferably m is 1 and n₄ is 1. In a preferred embodiment, n₅ is 1. In an even more preferred embodiment, m is 0 and n₅ is 1.

As indicated above, the compound of the present invention may comprise a linear unbranched structure. Thus, in one embodiment, m is an integer ranging from 1 to 10, preferably 1 to 5, more preferably 1 to 3, even more preferably 1 or 2, and n₂, n₅ and each n₈ are 0. In a particularly preferred embodiment, m is 1 and n₂, n₅ and n₈ are 0.

As indicated above, n₁ and n₉ are independently selected from an integer ranging from 1 to 10, and each of R₁ and R₂ is independently H, OH, SH, NH₂ or halogen, wherein, if n₁ is greater than 1, R₁ may be same or different in each repeating unit, and R₂ may be same or different in each repeating unit; and each of R₁₁ and R₁₂ is independently H, OH, SH, NH₂ or halogen, wherein, if n₉ is greater than 1, R₁₁ may be same or different in each repeating unit, and R₁₂ may be same or different in each repeating unit.

In a preferred embodiment, each of R₁ and R₂ is the same in each repeating unit if n₁ is greater than 1, and each of R₁₁ and R₁₂ is the same in each repeating unit if n₉ is greater than 1.

In another preferred embodiment, one of R₁ and R₂ is H and one of R₁, and R₁₂ is H. In a more preferred embodiment, each of R₁, R₂, R₁₁, and R₁₂ is H.

In another preferred embodiment, each of n₁ and no is independently selected from 1, 2 or 3, preferably 1 or 2, more preferably n₁ and n₉ are 1.

In a particularly preferred embodiment, n₁ and n₉ are 1 and each of R₁, R₂, R₁₁, and R₁₂ is H.

Thus, the compound of formula (I) is preferably a compound of formula (I*) containing (I**) as the linear structural element between B₁ and X₁, wherein the left wavy line marks the connection to B₁ of the remainder of the molecule and the right wavy line marks the connection to X₁ of the remainder of the molecule:

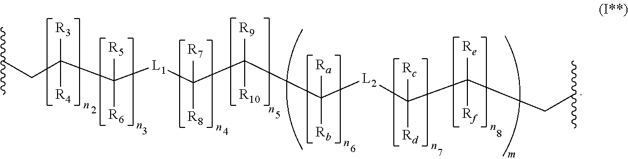

In preferred embodiments, the compound of formula (I) is a compound of formula (Ia-1), (Ia-2), (Ib-1), (Ib-2), (Ib-3), or (Ib-4) containing (Ia-1′), (Ia-2′), (Ib-1′), (Ib-2′), (Ib-3′), or (Ib-4′), respectively, as the linear structural element between B₁ and X₁, wherein the left wavy line in each case marks the connection to B₁ of the remainder of the molecule and the right wavy line in each case marks the connection to X₁ of the remainder of the molecule:

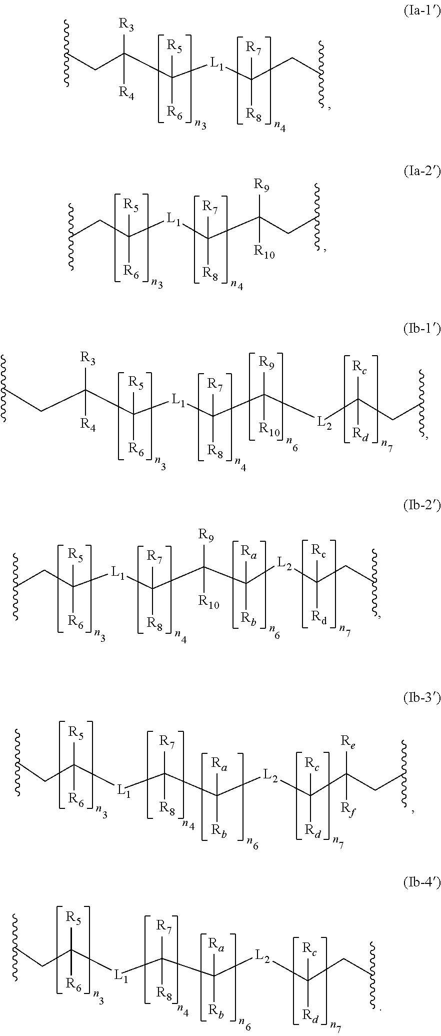

Preferably, the compound of formula (I) is a compound of formula (Ia-2) or (Ib-4). In one particularly preferred embodiment, the compound of formula (I) is a compound of formula (Ia-2). In another particularly preferred embodiment, the compound of formula (I) is a compound of formula (Ib-4).

As indicated above, each of n₃, n₄, n₆, and n₇ is independently selected from an integer ranging from 0 to 5, wherein, if m is greater than 1, n₆ may be same or different in each repeating unit and n₇ may be same or different in each repeating unit; and
each of R₅ and R₆ is independently H, OH, SH, NH₂ or halogen, wherein, if n₃ is greater than 1, R₅ may be same or different in each repeating unit, and R₆ may be same or different in each repeating unit;
each of R₇ and R₈ is independently H, OH, SH, NH₂ or halogen, wherein, if n₄ is greater than 1, R₇ may be same or different in each repeating unit, and R₈ may be same or different in each repeating unit;
each of Rₐ and R_{b} is independently H, OH, SH, NH₂ or halogen, wherein, if n₆ is greater than 1, Rₐ may be same or different in each repeating unit, and R_{b} may be same or different in each repeating unit;
each of R_{c} and R_{d} is independently H, OH, SH, NH₂ or halogen, wherein, if n₇ is greater than 1, R_{c} may be same or different in each repeating unit, and R_{d} may be same or different in each repeating unit;

In a preferred embodiment, each of R₅ and R₆ is the same in each repeating unit if n₃ is greater than 1, each of R₇ and R₃ is the same in each repeating unit if n₄ is greater than 1, each of Rₐ and R_{b} is the same in each repeating unit if n₆ is greater than 1, and each of R_{c} and R_{d} is the same in each repeating unit if n₇ is greater than 1.

In another preferred embodiment, one of R₅ and R₆ is H, one of R₇ and Rₐ is H, one of Rₐ and R_{b} is H and one of R_{c} and R_{d} is H. In a more preferred embodiment, each of R₅, R₆, R₇, R₃, Rₐ, R_{b}, R_{c}, and R_{d} is H.

Thus, in one preferred embodiment, each of R₁, R₂, R₅, R₆, R₇, R₈, Rₐ, R_{b}, Rₑ, R_{d}, R₁₁, and R₁₂ is the same in each repeating unit.

In another preferred embodiment, one of R₁ and R₂, one of R₅ and R₆, one of R₇ and R₈, one of Rₐ and R_{b}, one of R_{c} and R_{d}, and one of R₁₁ and R₁₂ is H. In a particularly preferred embodiment, each of R₁, R₂, R₅, R₆, R₇, R₃, Rₐ, R_{b}, R_{c}, R_{d}, R₁₁, and R₁₂ is H.

In another preferred embodiment, each of n₃, n₄, n₆ and n₇ is independently selected from 0, 1, or 2, preferably from 0 or 1; wherein, if m is greater than 1, n₆ may be same or different in each repeating unit, and n₇ may be same or different in each repeating unit.

In a more preferred embodiment, each of R₁, R₂, R₅, R₆, R₇, R₈, Rₐ, R_{b}, R_{c}, R_{d}, R₁₁, and R₁₂ is H, and each of n₃, n₄, ne and n₇ is independently selected from 0 or 1; wherein, if m is greater than 1, n₆ may be same or different in each repeating unit, and n₇ may be same or different in each repeating unit.

In a particularly preferred embodiment, n₃, n₄, each n₆, and each n₇ are 0. In another particularly preferred embodiment, n₃, n₄, each n₆, and each n₇ are 1 In particular when the compound of the present invention comprises a linear unbranched structure, i.e., n₂, n₅ and each n₈ are 0, then n₃, n₄, each n₆, and each n₇ are preferably 1.

In particularly preferred embodiments, the compound of formula (I) is thus a compound of formula (Ia-1-a), (Ia-2-a), (Ib-1-a), (Ib-2-a), (Ib-3-a), (Ib-2-b) or (Ib-4-a) containing (Ia-1-a′), (Ia-2-a′), (Ib-1-a′), (Ib-2-a′), (Ib-3-a′), (Ib-2-b′) or (Ib-4-a′), respectively, as the linear structural element between B₁ and X₁, wherein the left wavy line in each case marks the connection to B₁ of the remainder of the molecule and the right wavy line in each case marks the connection to X₁ of the remainder of the molecule:

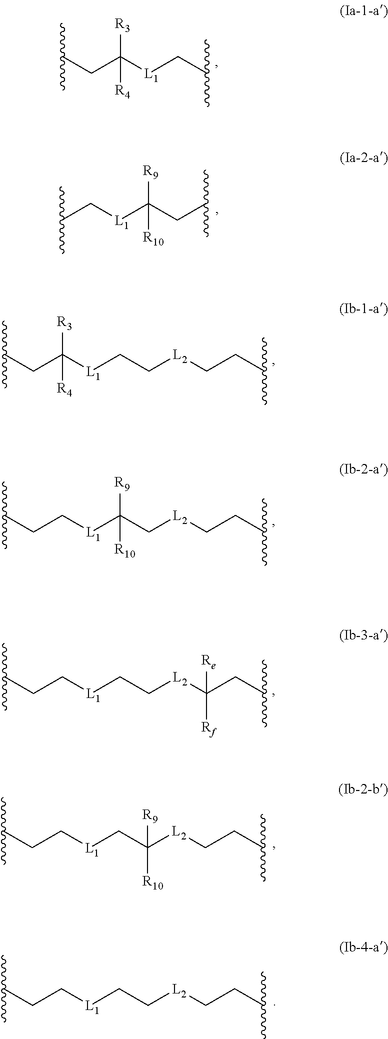

Preferably, the compound of formula (I) is a compound of formula (Ia-2-a) or (Ib-4-a). In a particularly preferred embodiment, the compound of formula (I) is a compound of formula (Ia-2-a). In another particularly preferred embodiment, the compound of formula (I) is a compound of formula (Ib-4-a).

In connection with the compounds of formula (I), as well as in connection with the compounds of formula (I*), (Ia), (Ib), (Ia-1), (Ia-2), (Ib-1), (Ib-2), (Ib-3), (Ib-4), (Ia-1-a), (Ia-2-a), (Ib-1-a), (Ib-2-a), (Ib-3-a), (Ib-2-b), and (Ib-4-a), and the corresponding structures (1**), (Ia-1′), (Ia-2′), (Ib-1′), (Ib-2′), (Ib-3′), (Ib-4′), (Ia-1-a′), (Ia-2-a′), (Ib-1-a′), (Ib-2-a′), (Ib-3-a′), (Ib-2-b′), and (Ib-4-a′), the following embodiments regarding L₁ and L₂ are relevant.

As indicated above, L₁ is selected from the group consisting of CH₂, O, S, SO, SO₂, NR^{L}, CH(OH), CH(SH) and CH(halogen), with the proviso that, if m is 1 and n₂, n₅ and n₈ are 0, L₁ is selected from the group consisting of O, S, SO, SO₂ and NR^{L}, and
R^{L} is (i) H, if one of n₂, n₅ and n₈ is 1, or (ii) selected from the group consisting of H, C₁-C₃-alkyl, C₁-C₃-alkyl-O—R^{O}, C₁-C₃-alkyl-S—R^{S}, C₁-C₃-alkyl-NR^{N1}R^{N2}, C₁-C₃-alkyl-R^{P} and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized, and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, if n₂, n₅ and n₈ are 0.

In a preferred embodiment, R^{L} is H.

Thus, in a preferred embodiment, L₁ is selected from the group consisting of CH₂, O, S, SO, SO₂, NH, CH(OH), CH(SH) and CH(halogen), with the proviso that, if m is 1 and n₂, n₅ and n₈ are 0, L₁ is selected from the group consisting of O, S, SO, SO₂ and NH.

In a more preferred embodiment, L₁ is selected from the group consisting of CH₂, O, CH(OH), CH(SH) and CH(halogen), with the proviso that, if m is 1 and n₂, n₅ and n₈ are 0, L₁ is O or S, preferably O.

In an even more preferred embodiment, L₁ is CH₂ or O, with the proviso that, if m is 1 and n₂, n₅ and n₈ are 0, L₁ is 0.

Thus, if m is 1 and n₂, n₅ and n₈ are 0, L₁ is preferably selected from the group consisting of O, S, SO, SO₂ and NH. More preferably, if m is 1 and n₂, n₅ and n₈ are 0, L₁ is O or S. Most preferably, if m is 1 and n₂, n₅ and n₈ are 0, L₁ is O.

As indicated above, L₂ is selected from the group consisting of O, S, SO, SO₂ and NH, wherein, if m is greater than 1, L₂ may be same or different in each repeating unit. In a preferred embodiment, L₂ is selected from the group consisting of O, S, SO, SO₂ and NH; wherein, if m is greater than 1, L₂ is the same in each repeating unit.

In a more preferred embodiment, each L₂ is O. In an even more preferred embodiment, L₁ is O and each L₂ is O.

As indicated above, the compounds of formula (I) may comprise a linear single-branched structure, wherein one of n₂ and n₅ is 1 and the other two of n₂ and n₅ as well as each n₈, if present, are 0, or one n₄ is 1 and each other n₈, if present, as well as n₂ and n₅ are 0. In particular for the compound of formula (I) comprising a linear single-branched structure, if m is 1, one of n₂, n₅ and n₈ may be 1, and the other two of n₂, n₅ and n₈ are 0. Or, if m is 0, one of n₂ and n₅ may be 1, and the other one of n₂ and n₅ is 0.

In this context,
one of R₃ and R₄ is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group; and the other one of R₃ and R₄ is selected from the group consisting of H, OH, SH, NH₂ and halogen; one of R₉ and R₁₀ is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group; and the other one of R₉ and R₁₀ is selected from the group consisting of H, OH, SH, NH₂ and halogen; one of Rₑ and R_{f} is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group; and the other one of Rₑ and R_{f} is selected from the group consisting of H, OH, SH, NH₂ and halogen.

In a preferred embodiment, if n₂ is 1, one of R₃ and R₄ is H, if n₅ is 1, one of R₉ and R₁₀ is H, and if n₄ is 1, one of Rₑ and R_{f} is H.

In a more preferred embodiment,
if n₂ is 1, one of R₃ and R₄ is selected from the group consisting of C₁-alkyl-O—R^{O}, C₁-alkyl-S—R^{S}, C₁-alkyl-S—S—R^{S}, C₁-alkyl-R^{P}, O—R^{D}, R^{P}, and 5- or 6-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl; and the other one of R₃ and R₄ is H;
if n₅ is 1, one of R₉ and R₁₀ is selected from the group consisting of C₁-alkyl-O—R^{O}, C₁-alkyl-S—R^{S}, C₁-alkyl-S—S—R^{S}, C₁-alkyl-R^{P}, O—R^{D}, R^{P}, and 5- or 6-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl; and the other one of R₉ and R₁₀ is H; and if n₄ is 1, one of Rₑ and R_{f} is selected from the group consisting of C₁-alkyl-O—R^{O}, C₁-alkyl-S—R^{S}, C₁-alkyl-S—S—R^{S}, C₁-alkyl-R^{P}, O—R^{D}, R^{P}, and 5- or 6-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl; and the other one of R₃ and R₄ is H.

In an even more preferred embodiment,
if n₂ is 1, one of R₃ and R₄ is selected from the group consisting of C₁-alkyl-O—R^{O}, C₁-alkyl-S—R^{S}, C₁-alkyl-S—S—R^{S}, C₁-alkyl-R^{P}, O—R^{D}, R^{P}; and the other one of R₃ and R₄ is H;
if n₅ is 1, one of R₉ and R₁₀ is selected from the group consisting of C₁-alkyl-O—R^{O}, C₁-alkyl-S—R^{S}, C₁-alkyl-S—S—R^{S}, C₁-alkyl-R^{P}, O—R^{D}, R^{P}; and the other one of R₉ and R₁₀ is H; and
if n₈ is 1, one of Rₑ and R_{f} is selected from the group consisting of C₁-alkyl-O—R^{O}, C₁-alkyl-S—R^{S}, C₁-alkyl-S—S—R^{S}, C₁-alkyl-R^{P}, O—R^{D}, R^{P}; and the other one of R₃ and R₄ is H.

In one particularly preferred embodiment, if n₂ is 1, one of R₃ and R₄ is C₁-alkyl-O—R^{O} and the other one of R₃ and R₄ is H; if n₅ is 1, one of R₉ and R₁₀ is C₁-alkyl-O—R^{O} and the other one of R₉ and R₁₀ is H; and if n₈ is 1, one of Rₑ and R_{f} is C₁-alkyl-O—R^{O} and the other one of Rₑ and R_{f} is H.

In another particularly preferred embodiment, if n₂ is 1, one of R₃ and R₄ is C₁-alkyl-S—R^{S} or C₁-alkyl-S—S—R^{S}, preferably C₁-alkyl-S—R^{S}, and the other one of R₃ and R₄ is H; if n₅ is 1, one of R₉ and R₁₀ is C₁-alkyl-S—R^{S} or C₁-alkyl-S—S—R^{S}, preferably C₁-alkyl-S—R^{S}, and the other one of R₉ and R₁₀ is H; and if n₄ is 1, one of Rₑ and R_{f} is C₁-alkyl-S—R^{S} or C₁-alkyl-S—S—R^{S}, preferably C₁-alkyl-S—R^{S}, and the other one of Rₑ and R_{f} is H.

In another particularly preferred embodiment, if n₂ is 1, one of R₃ and R₄ is C₁-alkyl-R^{P} or R^{P} and the other one of R₃ and R₄ is H; if n₅ is 1, one of R₉ and R₁₀ is C₁-alkyl-R^{P} or R^{P} and the other one of R₉ and R₁₀ is H; and if n₈ is 1, one of Rₑ and R_{f} is C₁-alkyl-R^{P} or R^{P} and the other one of Rₑ and R_{f} is H.

As indicated above, R^{O}, R^{S}, R^{N1} and R^{N2} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group.

In a preferred embodiment, R^{O}, R^{S}, R^{N1} and R^{N2} are each independently selected from the group consisting of H, C₁-C₄-alkyl, and 5- or 6-membered saturated, partially or fully unsaturated, or aromatic carbocyclyl, carbocyclyl-C₁-C₂-alkyl, heterocyclyl, or heterocyclyl-C₁-C₂-alkyl, wherein the aforementioned heterocyclic rings comprise one or two, same or different heteroatoms selected from O, N, or S, wherein said N- and/or S-atoms are independently oxidized or non-oxidized; and wherein each substitutable carbon or heteroatom in the aforementioned groups is independently unsubstituted or substituted with one substituent R^{X}, wherein R^{X} is halogen, CN, OH, SH, NH₂, or C₁-C₂-alkyl.

In a more preferred embodiment, R^{O}, R^{S}, R^{N1} and R^{N2} are each independently selected from the group consisting of H, C₁-C₃-alkyl, C₁-C₂-cyanoalkyl, and 6-membered saturated, partially or fully unsaturated, or aromatic carbocyclyl, carbocyclyl-C₁-alkyl, heterocyclyl, or heterocyclyl-C₁-alkyl, wherein the aforementioned heterocyclic rings comprise one or two heteroatoms selected from O, N, or S, wherein said N- and/or S-atoms are independently oxidized or non-oxidized; and wherein each substitutable carbon or heteroatom in the aforementioned groups is independently unsubstituted or substituted with one substituent R^{X}, wherein R^{X} is halogen.

As indicated above, R^{D} and R^{E} are each independently selected from the group consisting of C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl- NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized; and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group.

In a preferred embodiment, R^{D} and R^{E} are each independently selected from the group consisting of C₁-C₄-alkyl, and 5- or 6-membered saturated, partially or fully unsaturated, or aromatic carbocyclyl, carbocyclyl-C₁-C₂-alkyl, heterocyclyl, or heterocyclyl-C₁-C₂-alkyl, wherein the aforementioned heterocyclic rings comprise one or two, same or different heteroatoms selected from O, N, or S, wherein said N- and/or S-atoms are independently oxidized or non-oxidized; and wherein each substitutable carbon or heteroatom in the aforementioned groups is independently unsubstituted or substituted with one substituent R^{X}, wherein R^{X} is halogen, CN, OH, SH, NH₂, or C₁-C₂-alkyl.

In a more preferred embodiment, R^{D} and R^{E} are each independently selected from the group consisting of C₁-C₄-alkyl, C₁-C₂-cyanoalkyl, and 6-membered saturated, partially or fully unsaturated, or aromatic carbocyclyl, carbocyclyl-C₁-alkyl, heterocyclyl, or heterocyclyl-C₁-alkyl, wherein the aforementioned heterocyclic rings comprise one or two heteroatoms selected from O, N, or S, wherein said N- and/or S-atoms are independently oxidized or non-oxidized; and wherein each substitutable carbon or heteroatom in the aforementioned groups is independently unsubstituted or substituted with one substituent R^{X}, wherein R^{X} is halogen.

In a particularly preferred embodiment, R^{D} and R^{E} are each independently C₁-C₂-alkyl, preferably CH₃.

As indicated above, R^{N3} and R^{N4} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, with the proviso that R^{N3} and R^{N4} are not both H.

In a preferred embodiment, one of R^{N3} and R^{N4} is H and the other one of R^{N3} and R^{N4} is selected from the group consisting of C₁-C₄-alkyl, and 5- or 6-membered saturated, partially or fully unsaturated, or aromatic carbocyclyl, carbocyclyl-C₁-C₂-alkyl, heterocyclyl, or heterocyclyl-C₁-C₂-alkyl, wherein the aforementioned heterocyclic rings comprise one or two, same or different heteroatoms selected from O, N, or S, wherein said N- and/or S-atoms are independently oxidized or non-oxidized; and wherein each substitutable carbon or heteroatom in the aforementioned groups is independently unsubstituted or substituted with one substituent R^{X}, wherein R^{X} is halogen, CN, OH, SH, NH₂, or C₁-C₂-alkyl.

In a more preferred embodiment, one of R^{N3} and R^{N4} is H and the other one of R^{N3} and R^{N4} is selected from the group consisting of C₁-C₄-alkyl, C₁-C₂-cyanoalkyl, and 6-membered saturated, partially or fully unsaturated, or aromatic carbocyclyl, carbocyclyl-C₁-alkyl, heterocyclyl, or heterocyclyl-C₁-alkyl, wherein the aforementioned heterocyclic rings comprise one or two heteroatoms selected from O, N, or S, wherein said N- and/or S-atoms are independently oxidized or non-oxidized; and wherein each substitutable carbon or heteroatom in the aforementioned groups is independently unsubstituted or substituted with one substituent R^{X}, wherein R^{X} is halogen.

As indicated above, R^{P} is

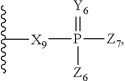

wherein
X₉ is O, S, NH or CH₂; Y₆ is O, S or Se; and each of Z₆ and Z₇ is independently OH, SH, SeH or BH₃. In a preferred embodiment, X₉ is 0 or CH₂; Y₆ is 0 or S; and each of Z₆ and Z₇ is OH.

In another preferred embodiment, X₉ is O; Y₆ is O or S; and each of Z₆ and Z₇ is OH.

In another preferred embodiment, X₉ is CH₂; Y₆ is 0 or S; and each of Z₆ and Z₇ is OH.

In one particularly preferred embodiment, m is 0, n₁ and n₉ are 1; each of n₂, n₃ and n₄ is 0; L₁ is O or CH₂; each of R₁, R₂, R₁₁, and R₁₂ is H; n₅ is 1; and one of R₉ and R₁₀ is selected from the group consisting of C₁-alkyl-O—R^{O}, C₁-alkyl-S—R^{S}, C₁-alkyl-S—S—CH₃, C₁-alkyl-R^{P}; and the other one of R₉ and R₁₀ is H;
wherein
R^{O} and R^{S} are each independently selected from the group consisting of H, C₁-C₃-alkyl, C₁-C₂-cyanoalkyl, phenyl, pyridyl, cyclohexyl-C₁-alkyl, wherein each substitutable carbon in the aforementioned rings is independently unsubstituted or 1 substitutable carbon in the aforementioned rings is substituted with one substituent R^{X}, wherein
R^{X} is halogen;
R^{P} is

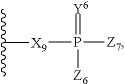

wherein
X₉ is O; Y₆ is O or S; and each of Z₆ and Z₇ is OH.

In another particularly preferred embodiment, m is 0, n₁ and n₉ are 1; L₁ is O, each of R₁, R₂, R₁₁, and R₁₂ is H; and either
(i) n₂ is 1; n₃ is 0; n₃ is 0, n₄ is 1 and R₇ and Rₐ are H; and one of R₃ and R₄ is selected from the group consisting of C₁-alkyl-O—R^{O}, C₁-alkyl-R^{P}, O—R^{O}, and R^{P}; and the other one of R₃ and R₄ is H;
or
(ii) n₅ is 1; n₂ is 0; n₄ is 0, n₃ is 1 and R₅ and R₆ are H; and one of R₉ and R₁₀ is selected from the group consisting of C₁-alkyl-O—R^{O}, C₁-alkyl-R^{P}, O—R^{D}, and R^{P}; and the other one of R₉ and R₁₀ is H;
wherein
R^{O} is selected from the group consisting of H and C₁-C₂-alkyl, preferably H and CH₃;
R^{D} is C₁-C₂-alkyl, preferably CH₃; and
R^{P} is

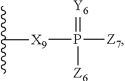

wherein
X₉ is O; Y₆ is O; and each of Z₆ and Z₇ is OH.

Thus, in one particularly preferred embodiment, m is 0, n₁ and n₉ are 1; each of n₂, n₃ and n₄ is 0; L₁ is O or CH₂; each of R₁, R₂, R₁₁, and R₁₂ is H; n₅ is 1; and one of R₉ and R₁₀ is C₁-alkyl-O—R^{O}; and the other one of R₉ and R₁₀ is H; wherein
R^{O} is selected from the group consisting of H, C₁-C₃-alkyl, C₁-C₂-cyanoalkyl, phenyl, pyridyl, cyclohexyl-C₁-alkyl, wherein each substitutable carbon in the aforementioned rings is independently unsubstituted or 1 substitutable carbon in the aforementioned rings is substituted with one substituent R^{X}, wherein R^{X} is halogen.

In another particularly preferred embodiment, m is 0, n₁ and n₉ are 1; each of n₂, n₃ and n₄ is 0; L₁ is O or CH₂; each of R₁, R₂, R₁₁, and R₁₂ is H; n₅ is 1; and one of R₉ and R₁₀ is C₁-alkyl-S—R^{S}; and the other one of R₉ and R₁₀ is H; wherein
R^{S} is selected from the group consisting of H, CH₃, C₁-C₂-cyanoalkyl, phenyl, pyridyl, cyclohexyl-C₁-alkyl, wherein each substitutable carbon in the aforementioned rings is independently unsubstituted or 1 substitutable carbon in the aforementioned rings is substituted with one substituent R^{X}, wherein R^{X} is halogen.

Thus, in one particularly preferred embodiment, m is 0, n₁ and n₉ are 1; each of n₂, n₃ and n₄ is 0; L₁ is O; each of R₁, R₂, R₁₁, and R₁₂ is H; n₅ is 1; and one of R₉ and R₁₀ is C₁-alkyl-O—R^{O}; and the other one of R₉ and R₁₀ is H; wherein R^{O} is H.

In another particularly preferred embodiment, m is 0, n₁ and n₉ are 1; each of n₂, n₃ and n₄ is 0; L₁ is CH₂; each of R₁, R₂, R₁₁, and R₁₂ is H; n₅ is 1; and one of R₉ and R₁₀ is C₁-alkyl-O—R^{O}; and the other one of R₉ and R₁₀ is H; wherein R^{O} is H.

In another particularly preferred embodiment, m is 0, n₁ and n₉ are 1; each of n₂, n₃ and n₄ is 0; L₁ is O; each of R₁, R₂, R₁₁, and R₁₂ is H; n₅ is 1; and one of R₉ and R₁₀ is C₁-alkyl-O—R^{O}; and the other one of R₉ and R₁₀ is H; wherein R^{O} is cyanoethyl.

In another particularly preferred embodiment, m is 0, n₁ and n₉ are 1; each of n₂, n₃ and n₄ is 0; L₁ is O; each of R₁, R₂, R₁₁, and R₁₂ is H; n₅ is 1; and one of R₉ and R₁₀ is C₁-alkyl-O—R^{O}; and the other one of R₉ and R₁₀ is H; wherein R^{O} is C₃-alkyl, preferably iso-propyl.

In another particularly preferred embodiment, m is 0, n₁ and n₉ are 1; each of n₂, n₃ and n₄ is 0; L₁ is O; each of R₁, R₂, R₁₁, and R₁₂ is H; n₅ is 1; and one of R₉ and R₁₀ is C₁-alkyl-O—R^{O}; and the other one of R₉ and R₁₀ is H; wherein R^{O} is phenyl.

In another particularly preferred embodiment, m is 0, n₁ and n₉ are 1; each of n₂, n₃ and n₄ is 0; L₁ is O; each of R₁, R₂, R₁₁, and R₁₂ is H; n₅ is 1; and one of R₉ and R₁₀ is C₁-alkyl-O—R^{O}; and the other one of R₉ and R₁₀ is H; wherein R^{O} is pyridyl substituted with bromine, preferably 5-bromo-2-pyridyl.

In another particularly preferred embodiment, m is 0, n₁ and n₉ are 1; each of n₂, n₃ and n₄ is 0; L₁ is O; each of R₁, R₂, R₁₁, and R₁₂ is H; n₅ is 1; and one of R₉ and R₁₀ is C₁-alkyl-O—R^{O}; and the other one of R₉ and R₁₀ is H; wherein R^{O} is CH₃.

In another particularly preferred embodiment, m is 0, n₁ and n₉ are 1; each of n₂, n₃ and n₄ is 0; L₁ is O; each of R₁, R₂, R₁₁, and R₁₂ is H; n₅ is 1; and one of R₉ and R₁₀ is C₁-alkyl-O—R^{O}; and the other one of R₉ and R₁₀ is H; wherein R^{O} is cyclohexylmethyl.

In another particularly preferred embodiment, m is 0, n₁ and n₉ are 1; each of n₂, n₃ and n₄ is 0; L₁ is O; each of R₁, R₂, R₁₁, and R₁₂ is H; n₅ is 1; and one of R₉ and R₁₀ is C₁-alkyl-S—R^{S}; and the other one of R₉ and R₁₀ is H; wherein R^{S} is cyanoethyl.

In another particularly preferred embodiment, m is 0, n₁ and n₉ are 1; each of n₂, n₃ and n₄ is 0; L₁ is O; each of R₁, R₂, R₁₁, and R₁₂ is H; n₅ is 1; and one of R₉ and R₁₀ is C₁-alkyl-S—R^{S}; and the other one of R₉ and R₁₀ is H; wherein R^{S} is CH₃.

In another particularly preferred embodiment, m is 0, n₁ and n₉ are 1; each of n₂, n₃ and n₄ is 0; L₁ is O; each of R₁, R₂, R₁₁, and R₁₂ is H; n₅ is 1; and one of R₉ and R₁₀ is C₁-alkyl-S—R^{S}; and the other one of R₉ and R₁₀ is H; wherein R^{S} is phenyl.

In another particularly preferred embodiment, m is 0, n₁ and n₉ are 1; each of n₂, n₃ and n₄ is 0; L₁ is O; each of R₁, R₂, R₁₁, and R₁₂ is H; n₅ is 1; and one of R₉ and R₁₀ is C₁-alkyl-S—R^{S}; and the other one of R₉ and R₁₀ is H; wherein R^{S} is pyridyl substituted with bromine, preferably 5-bromo-2-pyridyl.

In another particularly preferred embodiment, m is 0, n₁ and n₉ are 1; each of n₂, n₃ and n₄ is 0; L₁ is O; each of R₁, R₂, R₁₁, and R₁₂ is H; n₅ is 1; and one of R₉ and R₁₀ is C₁-alkyl-S—S—CH₃; and the other one of R₉ and R₁₀ is H.

In another particularly preferred embodiment, m is 0, n₁ and n₉ are 1; each of n₂, n₃ and n₄ is 0; L₁ is O; each of R₁, R₂, R₁₁, and R₁₂ is H; n₅ is 1; and one of R₉ and R₁₀ is C₁-alkyl-S—R^{S}; and the other one of R₉ and R₁₀ is H; wherein R³ is cyclohexylmethyl.

In another particularly preferred embodiment, m is 0, n₁ and n₉ are 1; each of n₂, n₃ and n₄ is 0; L₁ is O; each of R₁, R₂, R₁₁, and R₁₂ is H; n₅ is 1; and one of R₉ and R₁₀ is C₁-alkyl-R^{P}; and the other one of R₉ and R₁₀ is H; wherein
R^{P} is

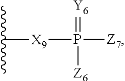

wherein X₉ is O; Y₆ is O; and each of Z₆ and Z₇ is OH.

In another particularly preferred embodiment, m is 0, n₁ and n₉ are 1; each of n₂, n₃ and n₄ is 0; L₁ is CH₂; each of R₁, R₂, R₁₁, and R₁₂ is H; n₅ is 1; and one of R₉ and R₁₀ is C₁-alkyl-R^{P}; and the other one of R₉ and R₁₀ is H; wherein
R^{P} is

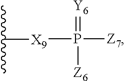

wherein X₉ is O; Y₆ is O; and each of Z₆ and Z₇ is OH.

In another particularly preferred embodiment, m is 0, n₁ and n₉ are 1; each of n₂, n₃ and n₄ is 0; L₁ is O; each of R₁, R₂, R₁₁, and R₁₂ is H; n₅ is 1; and one of R₉ and R₁₀ is C₁-alkyl-R^{P}; and the other one of R₉ and R₁₀ is H; wherein
R^{P} is

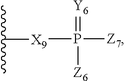

wherein X₉ is O; Y₆ is S; and each of Z₆ and Z₇ is OH.

In another particularly preferred embodiment, m is 0, n₁ and n₉ are 1; L₁ is O; each of R₁, R₂, R₁₁, and R₁₂ is H; n₂ is 1; n₅ is 0; n₃ is 0, n₄ is 1 and R₇ and R₈ are H; and one of R₃ and R₄ is C₁-alkyl-O—R^{O}; and the other one of R₃ and R₄ is H, wherein R^{O} is H.

In another particularly preferred embodiment, m is 0, n₁ and n₉ are 1; L₁ is O; each of R₁, R₂, R₁₁, and R₁₂ is H; n₂ is 1; n₅ is 0; n₃ is 0, n₄ is 1 and R₇ and R₈ are H; and one of R₃ and R₄ is C₁-alkyl-O—R^{O}; and the other one of R₃ and R₄ is H, wherein R^{O} is CH₃.

In another particularly preferred embodiment, m is 0, n₁ and n₉ are 1; L₁ is O; each of R₁, R₂, R₁₁, and R₁₂ is H; and n₅ is 1; n₂ is 0; n₄ is 0, n₃ is 1 and R₅ and R₆ are H; and one of R₉ and R₁₀ is C₁-alkyl-O—R^{O}; and the other one of R₉ and R₁₀ is H, wherein R^{O} is H.

In another particularly preferred embodiment, m is 0, n₁ and n₉ are 1; L₁ is O; each of R₁, R₂, R₁₁, and R₁₂ is H; and n₅ is 1; n₂ is 0; n₄ is 0, n₃ is 1 and R₅ and R₆ are H; and one of R₉ and R₁₀ is C₁-alkyl-O—R^{O}; and the other one of R₉ and R₁₀ is H; wherein R^{O} is CH₃.

In another particularly preferred embodiment, m is 0, n₁ and n₉ are 1; L₁ is O; each of R₁, R₂, R₁₁, and R₁₂ is H; n₂ is 1; n₅ is 0; n₃ is 0, n₄ is 1 and R₇ and R₈ are H; and one of R₃ and R₄ is C₁-alkyl-R^{P}; and the other one of R₃ and R₄ is H; wherein
R^{P} is

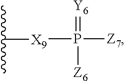

wherein X₉ is O; Y₆ is O; and each of Z₆ and Z₇ is OH.

In another particularly preferred embodiment, m is 0, n₁ and n₉ are 1; L₁ is O; each of R₁, R₂, R₁₁, and R₁₂ is H; and n₅ is 1; n₂ is 0, n₄ is 0, n₃ is 1 and R₅ and R₆ are H, and one of R₉ and R₁₀ is C₁-alkyl-R^{P}, and the other one of R₉ and R₁₀ is H; wherein
R^{P} is

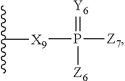

wherein X₉ is O; Y₆ is O; and each of Z₆ and Z₇ is OH.

In another particularly preferred embodiment, m is 0, n₁ and n₉ are 1; L₁ is O; each of R₁, R₂, R₁₁, and R₁₂ is H; n₂ is 1; n₅ is 0; n₃ is 0, n₄ is 1 and R₇ and R₈ are H; and one of R₃ and R₄ is O—R^{D}; and the other one of R₃ and R₄ is H; wherein R^{D} is C₁-C₂-alkyl, preferably CH₃.

In another particularly preferred embodiment, m is 0, n₁ and n₉ are 1; L₁ is O; each of R₁, R₂, R₁₁, and R₁₂ is H; and n₅ is 1; n₂ is 0; n₄ is 0, n₃ is 1 and R₅ and R₆ are H; and one of R₉ and R₁₀ is O—R^{D}; and the other one of R₉ and R₁₀ is H; wherein R^{D} is C₁-C₂-alkyl, preferably CH₃.

In another particularly preferred embodiment, m is 0, n₁ and n₉ are 1; L₁ is O; each of R₁, R₂, R₁₁, and R₁₂ is H; n₂ is 1; n₅ is 0; n₃ is 0, n₄ is 1 and R₇ and R₈ are H; and one of R₃ and R₄ is O—R^{P}; and the other one of R₃ and R₄ is H; wherein
R^{P} is

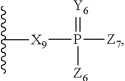

wherein X₉ is O; Y₆ is O; and each of Z₆ and Z₇ is OH.

In another particularly preferred embodiment, m is 0, n₁ and n₉ are 1; L₁ is O; each of R₁, R₂, R₁₁, and R₁₂ is H; and n₅ is 1; n₂ is 0; n₄ is 0, n₃ is 1 and R₅ and R₆ are H; and one of R₉ and R₁₀ is O—R^{P}; and the other one of R₉ and R₁₀ is H; wherein
R^{P} is

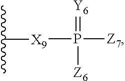

wherein X₉ is O; Y₆ is O; and each of Z₆ and Z₇ is OH.

In another preferred embodiment of the compounds of the present invention, the compound of formula (I) comprises a linear unbranched structure, i.e., m is an integer ranging from 1 to 10 and n₂, no and each n₅ are 0.

In this context, the compound of formula (I) is a compound of formula (Ic),

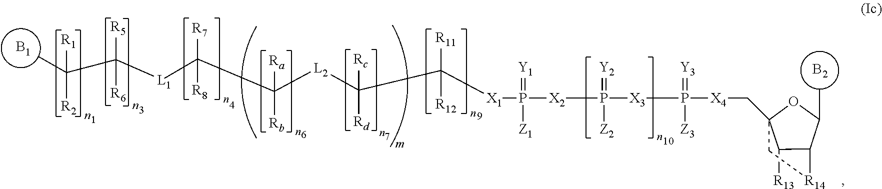

wherein n₁, n₅, n₇, n₉, R₁, R₂, R₅, R₆, R₇, R₈, R₁₁, R₁₂, Rₐ, R_{b}, R_{c}, R_{d}, L₁, and L₂ are as defined in formula (I), preferably as defined in the preferred embodiments above.

Accordingly, the compound of formula (Ic) is preferably a compound of formula (Ic*), more preferably a compound of formula (Ic**), even more preferably a compound of formula (Ic***):

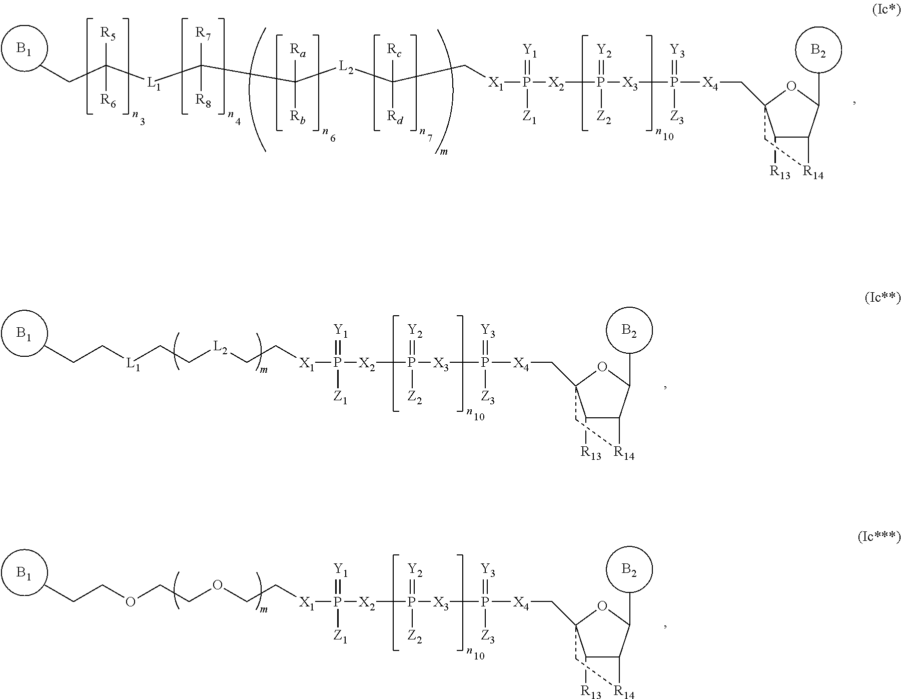

wherein m is an integer ranging from 1 to 10, preferably 1 to 9, more preferably 1 to 8, even more preferably 1 to 7, even more preferably 1 to 6, even more preferably 1 to 5, even more preferably 1 to 4, even more preferably 1 to 3, even more preferably 1 or 2. In a particularly preferred embodiment m is 1.

In a particularly preferred embodiment, m is 1, n₂, n₅ and n₈ are 0, n₁, n₃, n₄, n₆, n₇, and n₉ are 1; L₁ is O; L₂ is O; and each of R₁, R₂, R₅, R₆, R₇, R₃, R₁₁, R₁₂, Rₐ, R_{b}, R_{c}, and R_{d}, is H.

In connection with the compounds of formula (I), as well as in connection with the compounds of formula (Ia), (Ib), (I*), (Ic), (Ic*), (Ic**), (Ic***), (Ia-1), (Ia-2), (Ib-1), (Ib-2), (Ib-3), (Ib-4), (Ia-1-a), (Ia-2-a), (Ib-1-a), (Ib-2-a), (Ib-3-a), (Ib-2-b), and (Ib-4-a), and the corresponding structures (I**), (Ia-1′), (Ia-2′), (Ib-1′), (Ib-2′), (Ib-3′), (Ib-4′), (Ia-1-a′), (Ia-2-a′), (Ib-1-a′), (Ib-2-a′), (Ib-3-a′), (Ib-2-b′) and (Ib-4-a′), in particular in connection with the embodiments as defined above, the following embodiments regarding the remaining substituents R₁₃ through R₁₆, X₁ through X₃, Y₁ through Y₈, X₁ through X₈, ring B₁ through ring B₄, and n₁₀ are relevant.

As indicated above,
R₁₃ is

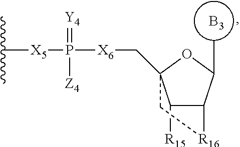

wherein
R₁₅ is OH or

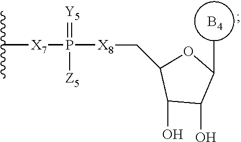

and
R₁₄ is (i) selected from the group consisting of H, OH, OC₁-C₃-alkyl, and Opropargyl, wherein the dashed methylene bridge between R₁₄ and the 4′ C is absent, or (ii) O, wherein the dashed methylene bridge between R₁₄ being O and the 4′ C is present; and
R₁₆ is (i) selected from the group consisting of H, OH, OC₁-C₃-alkyl, and Opropargyl, wherein the dashed methylene bridge between R₁₆ and the 4′ C is absent, or (ii) O, wherein the dashed methylene bridge between R₁₆ being O and the 4′ C is present.

In a preferred embodiment, R₁₄ is selected from the group consisting of H, OH, OC₁-C₃-alkyl, and Opropargyl, wherein the dashed methylene bridge between R₁₄ and the 4′ C is absent.

In another preferred embodiment, R₁₆ is selected from the group consisting of H, OH, OC₁-C₃-alkyl, and Opropargyl, wherein the dashed methylene bridge between R₁₀ and the 4′ C is absent.

In yet another preferred embodiment, R₁₄ is selected from the group consisting of H, OH, OC₁-C₃-alkyl, and Opropargyl, wherein the dashed methylene bridge between R₁₄ and the 4′ C is absent; and R₁₄ is selected from the group consisting of H, OH, OC₁-C₃-alkyl, and Opropargyl, wherein the dashed methylene bridge between R₁₆ and the 4′ C is absent.

In another embodiment, R₁₃ is

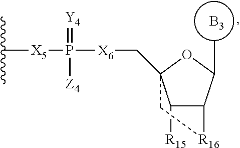

wherein R₁₅ and R₁₆ are each OH. In this embodiment, the compound of the present invention is a trinucleotide-like compound (interalia since it comprises three nucleobases, namely rings B₁ to B₃). It can be preferred in this embodiment that R₁₄ is H or OC₁-C₃-alkyl, wherein it can be especially preferred that R₁₄ is OCH₃, which may also be referred to as having a Cap1 structure/being a Cap1 analog. It can also be preferred in this embodiment that R₁₄ is 0, wherein the dashed methylene bridge between R₁₄ being O and the 4′ C is present.

When it comes to a Cap1 structure, a particular embodiment relates to a compound of formula (I) as defined herein, wherein n₁ is 1; n₂ is 0; n₃ is 0; n₄ is 0; n₅ is 1; n₉ is 1; m is 0; L₁ is O; each of R₁, R₂, R₉, R₁₁, and R₁₂ is H; R₁₀ is C₁-C₄-alkyl, wherein the C₁-C₄-alkyl is substituted with OH; R₁₃ is

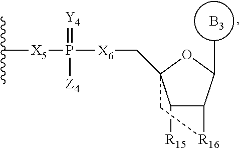

wherein R₁₅ and R₁₆ are each OH; R₁₄ is OCH₃; each of X₁ through X₆ is O, or X₁ is CH₂ and each of X₂ through X₆ is O; each of Z₁ through Z₄ is OH; and each of Y₁ through Y₄ is O. It can further be preferred in this embodiment that B₁ is a modified guanine, in particular N⁷-methylguanine; B₂ is adenine; and B₃ is guanine. An exemplary compound in this respect can in particular be the compound of formula (IV) as shown in the following (in a specific salt form, any other forms and salts are understood to be encompassed as well):

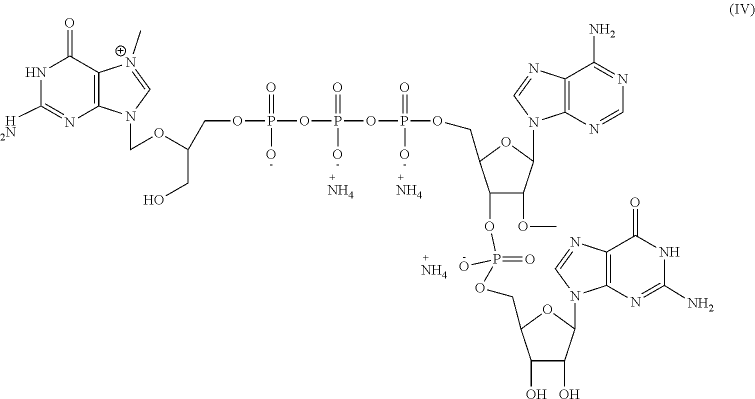 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC(CO)OCn2c[n+](C)c3c(=O)[nH]c(N)nc32)OC1n1cnc2c(N)ncnc21 ; [NH4+] ; [NH4+] ; [NH4+]]

When it comes to a Cap1 structure, another particular embodiment relates to a compound of formula (I) as defined herein, wherein n₁ is 1; n₂ is 0; n₃ is 0; n₄ is 0; n₅ is 1; n₅ is 1; m is; L₁ is 0; each of R₁, R₂, R₉, R₁₁, and R₁₂ is H; R₁₀ is C₁-C₄-alkyl, wherein the C₁-C₄-alkyl is substituted with OH; R₁₃ is

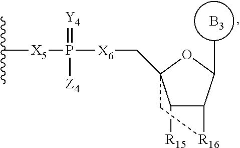

wherein R₁₅ and R₁₆ are each OH; R₁₄ is O, wherein the dashed methylene bridge between R₁₄ being O and the 4′ C is present; each of X₁ through X₆ is O, or X₁ is CH₂ and each of X₂ through X₆ is O; each of Z₁ through Z₄ is OH; and each of Y₁ through Y₄ is O. It can further be preferred in this embodiment that B₁ is a modified guanine, in particular N⁷-methylguanine; B₂ is adenine; and B₃ is guanine. An exemplary compound in this respect can in particular be the compound of formula (V) as shown in the following (in a specific salt form, any other forms and salts are understood to be encompassed as well):

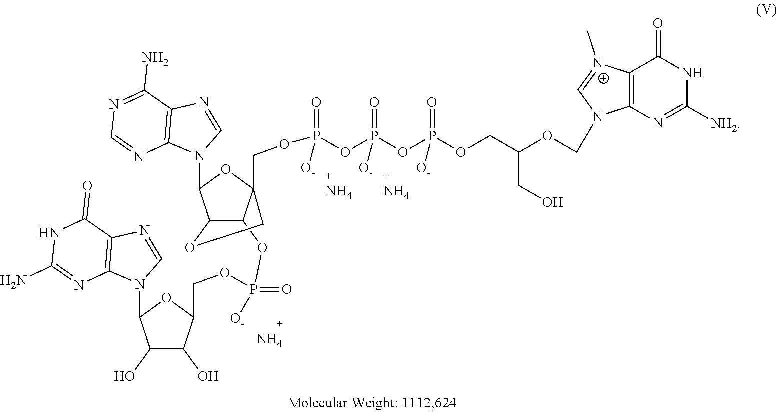

In yet another embodiment, R₁₃ is

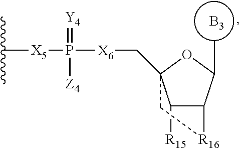

wherein R₁₅ is

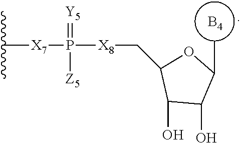

In this embodiment, the compound of the present invention is a tetranucleotide-like compound (inter alia since it comprises four nucleobases, namely rings B₁ to B₄).

It can be preferred in this embodiment that R₁₄ is H or OC₁-C₃-alkyl, wherein the dashed methylene bridge between R₁₄ and the 4′ C is absent, preferably wherein R₁₄ is OCH₃ (wherein also in this preferred embodiment the dashed methylene bridge between R₁₄ and the 4′ C is absent); and R₁₄ is H or OC₁-C₃-alkyl, wherein the dashed methylene bridge between R₁₄ and the 4′ C is absent, preferably wherein Rie is OCH₃ (wherein also in this preferred embodiment the dashed methylene bridge between R₁₆ and the 4′ C is absent), which may also be referred to as having a Cap2 structure/being a Cap2 analog.

Alternatively, it can be preferred in this embodiment that R₁₄ is OH, wherein the dashed methylene bridge between R₁₄ and the 4′ C is absent; and Rie is OC₁-C₃-alkyl, wherein the dashed methylene bridge between R₁₆ and the 4′ C is absent, which may also be referred to as having a Cap2-1 structure/being a Cap2-1 analog. Cap2-1 structures and effects associated therewith are inter alia disclosed in Drazkowska et al., Nucleic Acids Research, 2022, Vol. 50, No. 16 9051-9071.

Yet alternatively, it can be preferred in this embodiment that R₁₄ is O, wherein the dashed methylene bridge between R₁₄ being O and the 4′ C is present, and/or that R₁₆ is O, wherein the dashed methylene bridge between R₁₆ being O and the 4′ C is present.

As indicated above, n₁₀ is selected from 0, 1 or 2, and each of X₁ through X₃ is independently O, S, NH or CH₂, wherein, if n₁₀ is 2, X₃ may be same or different in each repeating unit;
each of Y₁ through Y₅ is independently O, S or Se, wherein, if n₁₀ is 2, Y₂ may be same or different in each repeating unit;
each of Z₁ through Z₅ is independently OH, SH, SeH or BH₃, wherein, if n₁₀ is 2, Z₂ may be same or different in each repeating unit.

In a preferred embodiment, n₁₀ is 1.

In another preferred embodiment, X₁ is CH₂ or O. Thus, in one preferred embodiment, X₁ is O. In a more preferred embodiment, X₁ is CH₂. In yet another preferred embodiment, each of X₂ through X₃ is O. Thus, in one preferred embodiment, X₁ is CH₂ or O and each of X₂ through X₃ is O. In another preferred embodiment, X₁ is O and each of X₂ through X₃ is O. In a more preferred embodiment, X₁ is CH₂ and each of X₂ through X₃ is O.

In another preferred embodiment, each of Y₁ through Y₅ is O. In another preferred embodiment, one of Y₁ through Y₅, preferably Y₁, is S and each of the remaining ones of Y₁ through Y₅ is O.

In another preferred embodiment, Z₁ through Z₅ is OH.

As indicated above, ring B₁ is guanine, a modified guanine or a guanine analog; and
each of ring B₂ through ring B₄ is independently a nucleobase, a modified nucleobase or a nucleobase analog.

In a preferred embodiment, B₂ is selected from the group consisting of guanine, a modified guanine, a guanine analog, adenine, a modified adenine, and an adenine analog.

In another preferred embodiment, B₃ is guanine, a modified guanine or a guanine analog.

In one particularly preferred embodiment, B₂ is selected from the group consisting of guanine, a modified guanine, a guanine analog, adenine, a modified adenine, and an adenine analog; and B₃ is guanine, a modified guanine or a guanine analog.

In another preferred embodiment, ring B₁ is a modified guanine, preferably selected from the group consisting of N⁷-methylguanine, N⁷-4-chloro-benzyl-guanine, N⁷-biphenyl-methylene-guanine, N⁷-naphtyl-methylene-guanine and N⁷-3,5-dimethyl-benzyl-guanine. It can be especially preferred that ring B₁ is N⁷-methylguanine.

It can be particularly preferred in the first aspect that the compound of formula (I) is selected from the group consisting of

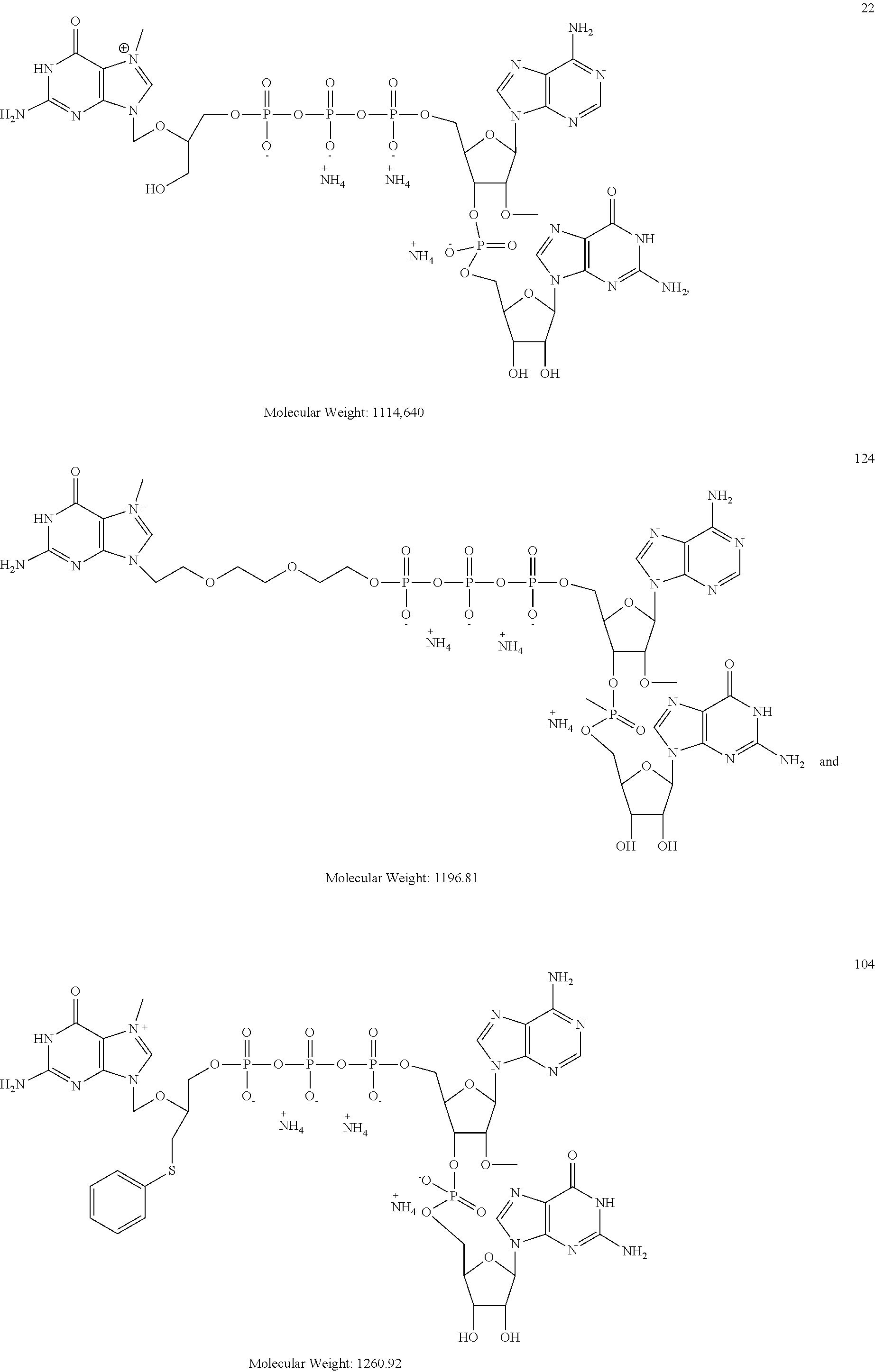

wherein not only the specific NH₄⁺ salt of any of the compounds as indicated above is encompassed but any other salt as well.

In Aspect A of the first aspect, the compound of formula (I) is a compound of formula (Ic***)

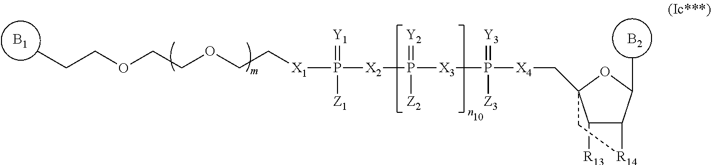

or a salt, stereoisomer, tautomer, or deuterated version thereof, wherein
R₁₃ is

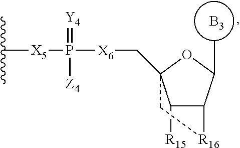

wherein
R₁₅ is OH or

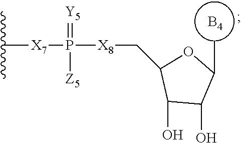

ring B₁ is guanine, a modified guanine or a guanine analog, preferably N⁷-methylguanine;
each of ring B₂ through ring B₄ is independently a nucleobase, a modified nucleobase or a nucleobase analog, preferably a nucleobase;
m is selected from an integer ranging from 1 to 10, preferably ranging from 1 to 5, more preferably ranging from 1 to 3, and most preferably is 1;
n₁₀ is selected from 0, 1 or 2, preferably 1;
each of X₁ through X₈ is independently O, S, NH or CH₂, wherein, if n₁₀ is 2, X₃ may be same or different in each repeating unit, preferably each of X₁ through X₈ is O;
each of Y₁ through Y₅ is independently O, S or Se, wherein, if n₁₀ is 2, Y₂ may be same or different in each repeating unit, preferably each of Y₁ through Y₅ is O;
each of Z₁ through Z₅ is independently OH, SH, SeH or BH₃, wherein, if n₁₀ is 2, Z₂ may be same or different in each repeating unit, preferably each of Z₁ through Z₅ is OH;
R₁₄ is (i) selected from the group consisting of H, OH, OC₁-C₃-alkyl, and Opropargyl, wherein the dashed methylene bridge between R₁₄ and the 4′ C is absent, preferably OCH₃, or (ii) O, wherein the dashed methylene bridge between R₁₄ being O and the 4′ C is present; and
R₁₆ is (i) selected from the group consisting of H, OH, OC₁-C₃-alkyl, and Opropargyl, wherein the dashed methylene bridge between R₁₆ and the 4′ C is absent, preferably OCH₃, or (ii) O, wherein the dashed methylene bridge between R₁₆ being O and the 4′ C is present.

In an aspect related to the first aspect, the present invention relates to a compound of formula (I) as defined in the first aspect with the only difference that R₁₃ is defined as OH and not as the structure of the first aspect, or a salt, stereoisomer, tautomer, or deuterated version thereof, wherein the following two compounds are excluded

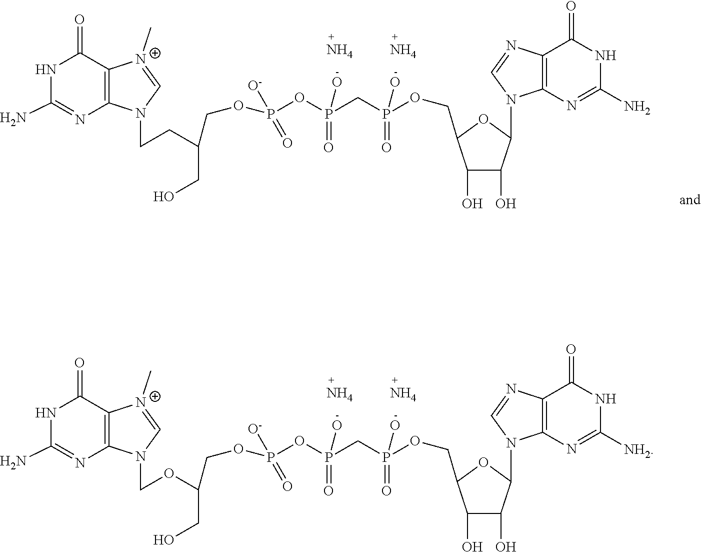 [SMILES: C[n+]1cn(CCC(CO)COP(=O)([O-])OP(=O)([O-])CP(=O)([O-])OCC2OC(n3cnc4c(=O)[nH]c(N)nc43)C(O)C2O)c2nc(N)[nH]c(=O)c21 ; [NH4+] ; [NH4+] ; C[n+]1cn(COC(CO)COP(=O)([O-])OP(=O)([O-])CP(=O)([O-])OCC2OC(n3cnc4c(=O)[nH]c(N)nc43)C(O)C2O)c2nc(N)[nH]c(=O)c21 ; [NH4+] ; [NH4+]]

This results in a dinucleotide-like compound (inter alia since it comprises two nucleobases, namely rings B₁ and B₂). In an embodiment thereof, R₁₃ is OH and R₁₄ is OH, which may also be referred to as having a cap0 structure/being a cap0 analog. Except for embodiments of the first aspect relating to R₁₃, all embodiments of the first aspect equally apply for the present aspect related to the first aspect.

In the second aspect, the present invention relates to a cap analog comprising a 5′ terminal acyclonucleoside, wherein the acyclonucleoside comprises a linear unbranched or linear single-branched structure instead of a ribose, wherein the cap analog is preferably a Cap1 analog or a Cap2 analog or a Cap2-1 analog, wherein the 5′ terminal acyclonucleoside is optionally deuterated, and wherein the linear unbranched or linear single-branched structure has the structure of formula (II):

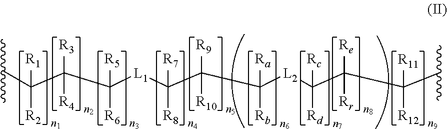

wherein
n₁ and n₉ are independently selected from an integer ranging from 1 to 10;
each of R₁ and R₂ is independently H, OH, SH, NH₂ or halogen, wherein, if n₁ is greater than 1, R₁ may be same or different in each repeating unit, and R₂ may be same or different in each repeating unit;
each of R₁₁ and R₁₂ is independently H, OH, SH, NH₂ or halogen, wherein, if n₉ is greater than 1, R₁₁ may be same or different in each repeating unit, and R₁₂ may be same or different in each repeating unit;
each of n₃, n₄, n₆, and n₇ is independently selected from an integer ranging from 0 to 5; wherein, if m is greater than 1, n₆ may be same or different in each repeating unit, and n₇ may be same or different in each repeating unit;
each of R₅ and R₆, if present, is independently H, OH, SH, NH₂ or halogen, wherein, if n₃ is greater than 1, R₆ may be same or different in each repeating unit, and R₆ may be same or different in each repeating unit;
each of R₇ and R₈, if present, is independently H, OH, SH, NH₂ or halogen, wherein, if n₄ is greater than 1, R₇ may be same or different in each repeating unit, and R₈ may be same or different in each repeating unit;
each of Rₐ and R_{b}, if present, is independently H, OH, SH, NH₂ or halogen, wherein, if n₆ is greater than 1, Rₐ may be same or different in each repeating unit, and R_{b} may be same or different in each repeating unit;
each of R_{c} and R_{d}, if present, is independently H, OH, SH, NH₂ or halogen, wherein, if n₇ is greater than 1, R_{c} may be same or different in each repeating unit, and R_{d} may be same or different in each repeating unit;
n₂, n₅ and n₆ are independently selected from 0 or 1; wherein, if m is greater than 1, ne may be same or different in each repeating unit;
one of R₃ and R₄, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N}, C₁-C₃-alkyl- R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group; and the other one of R₃ and R₄, if present, is selected from the group consisting of H, OH, SH, NH₂ and halogen; one of R₉ and R₁₀, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl- R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of R₉ and R₁₀, if present, is selected from the group consisting of H, OH, SH, NH₂ and halogen; one of Rₑ and R_{f}, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl- R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group; and the other one of Rₑ and R_{f}, if present, is selected from the group consisting of H, OH, SH, NH₂ and halogen;
m is selected from an integer ranging from 0 to 10, with the proviso that (i) if m is 0, one of n₂ and n₅ is 1 and the other one of n₂ and n₅ is 0, (ii) if m is 1, (a) n₂, n₅ and n₈ are 0 or (b) one of n₂, n₅ and n₈ is 1 and the other two of n₂, n₅ and n₈ are 0, and (iii) if m is an integer ranging from 2 to 10, (a) n₂, n₅ and each n₈ are 0, (b) one of n₂ and n₅ is 1 and the other one of n₂ and n₅ as well as each n₈ are 0, or (c) one n₃ is 1 and each other n₈ as well as n₂ and n₅ are 0;
L₁ is selected from the group consisting of CH₂, O, S, SO, SO₂, NR^{L}, CH(OH), CH(SH) and CH(halogen), with the proviso that, if m is 1 and n₂, n₅ and n₈ are 0, L₁ is selected from the group consisting of O, S, SO, SO₂ and NR^{L}; L₂ is selected from the group consisting of O, S, SO, SO₂ and NH; wherein, if m is greater than 1, L₂ may be same or different in each repeating unit;
R^{L} is (i) H, if one of n₂, n₅ and n₈ is 1, or (ii) selected from the group consisting of H, C₁-C₃-alkyl, C₁-C₃-alkyl-O—R^{O} C₁-C₃-alkyl-S—R^{S}, C₁-C₃-alkyl-NR^{N1}R^{N2}, C₁-C₃-alkyl-R^{P} and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized, and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, if n₂, n₅ and n₈ are 0;
R^{O}, R^{S}, R^{N1} and R^{N2} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl- O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁- C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl- NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{D} and R^{E} are each independently selected from the group consisting of C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl- O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁- C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{N3} and R^{N4} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl- NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, with the proviso that R^{N3} and R^{N4} are not both H;
R^{P} is

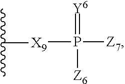

wherein
X₉ is O, S, NH or CH₂,
Y₆ is O, S or Se; and
each of Z₆ and Z₇ is independently OH, SH, SeH or BH₃;
and wherein the linear unbranched or linear single-branched structure is optionally deuterated.

In one embodiment of the second aspect, m is selected from 0 or 1.

In one embodiment of the second aspect, the acyclonucleoside comprises a linear unbranched structure of formula (II), wherein m is selected from an integer ranging from 1 to 10, preferably 1, n₂, n₅ and each n₈ are 0, and L₁ is selected from the group consisting of O, S, SO, SO and NR^{L}, preferably L₁ is O.

In another embodiment of the second aspect, the acyclonucleoside comprises a linear single-branched structure of formula (II), wherein
(i) m is 0, one of n₂ and n₅ is 1, and the other one of n₂ and n₅ is 0; (ii) m is 1, one of n₂, n₅ and n₈ is 1, and the other two of n₂, n₅ and n₈ are 0; (iii) m is an integer ranging from 2 to 10, one of n₂ and n₅ is 1 and the other one of n₂ and n₅ as well as each n₈ are 0, or (iv) m is an integer ranging from 2 to 10, one n₈ is 1 and each other n₈ as well as n₂ and n₅ are 0; and
L₁ is selected from the group consisting of CH₂, O, S, SO, SO₂, NR^{L}, CH(OH), CH(SH) and CH(halogen), preferably L₁ is CH₂ or O.

In a preferred embodiment thereof, the acyclonucleoside comprises a linear single-branched structure of formula (II), wherein m is 0, one of n₂ and n₅ is 1, and the other one of n₂ and n₅ is 0.

In one preferred embodiment, each of n₁ and no is independently selected from 1 or 2, preferably n₁ is 1 and n₉ is 1; and each of n₃, n₄, and, if present, each of n₆ and n₇, is independently selected from 0 or 1.

In another preferred embodiment, one of R₃ and R₄ if present, one of R₉ and R₁₀ if present, or one of Rₑ and R_{f} if present, is selected from the group consisting of C₁-alkyl-O—R^{O}, C₁-alkyl-S—R^{S}, C₁-alkyl-S—S—R^{S}, C₁-alkyl-R^{P}; wherein
R^{O} and R^{S} are each independently selected from the group consisting of H, C₁-C₄-alkyl, C₁-C₂-cyanoalkyl, and 5- or 6-membered saturated, partially or fully unsaturated, or aromatic carbocyclyl, carbocyclyl-C₁-alkyl, heterocyclyl, or heterocyclyl-C₁-alkyl, wherein the aforementioned heterocyclic rings comprise one or two, same or different heteroatoms selected from O, N, or S, wherein said N- and/or S-atoms are independently oxidized or non-oxidized; and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₂-alkyl, preferably halogen;
R^{P} is

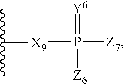

wherein
X₉ is O; Y₆ is O or S; and each of Z₅ and Z₇ is OH; and
the other one of R₃ and R₄ if present, R₉ and R₁₀ if present, Rₑ and R_{f} if present, is H, and wherein preferably each of R₁, R₂, R₅, R₆, R₇, R₈, Rₐ, R_{b}, R_{c}, R_{d}, R₁₁, and R₁₂, if present, is H.

In yet another preferred embodiment, the acyclonucleoside comprises as the nucleobase guanine, a modified guanine or a guanine analog.

Of course, all embodiments disclosed in connection with formula (I) of the first aspect, in particular regarding each of R, through R₁₂, each of Rₐ through R_{f}, R^{D}, R^{S}, R^{P}, R^{N1}, R^{N2}, R^{N3}, R^{N4}, R^{L}, R^{D}, R^{E}, each of n₁ through n₉, L₁, L₂, and m, also apply to formula (II) of the second aspect.

In preferred embodiments relating to the first aspect, the second aspect and the aspect related to the first aspect as outlined above, the compound and cap analog, respectively, has at least one of the following characteristics as compared to, e.g., (i) mCap, (ii) CleanCap, or (iii) enzymatic capping, in particular post-transcriptional enzymatic capping:
(a) increased capping efficiency during RNA in vitro transcription;
(b) increased incorporation efficiency of the compound and cap analog, respectively, during RNA in vitro transcription (i.e., a lower amount of the compound and cap analog, respectively, is required);
(c) reduced generation of undesired side-products (in particular a reverse cap product) during RNA in vitro transcription; and
(d) reduced generation of dsRNA species during RNA in vitro transcription; In the third aspect, the present invention relates to an RNA molecule comprising at least three nucleotides and comprising a 5′ end of formula (III):

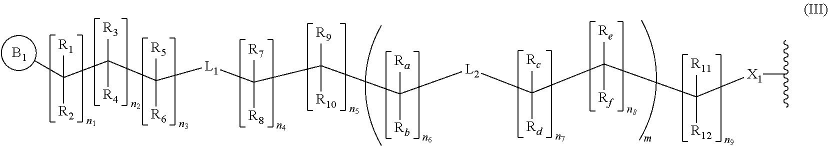

wherein
ring B₁ is guanine, a modified guanine or a guanine analog;
n₁ and n₉ are independently selected from an integer ranging from 1 to 10;
each of R₁ and R₂ is independently H, OH, SH, NH₂ or halogen, wherein, if n₁ is greater than 1, R₁ may be same or different in each repeating unit, and R₂ may be same or different in each repeating unit;
each of R₁₁ and R₁₂ is independently H, OH, SH, NH₂ or halogen, wherein, if n₉ is greater than 1, R₁₁ may be same or different in each repeating unit, and R₁₂ may be same or different in each repeating unit;
each of n₃, n₄, n₆, and n₇ is independently selected from an integer ranging from 0 to 5; wherein, if m is greater than 1, ne may be same or different in each repeating unit, and n₇ may be same or different in each repeating unit;
each of R₅ and R₈, if present, is independently H, OH, SH, NH₂ or halogen, wherein, if n₄ is greater than 1, R₅ may be same or different in each repeating unit, and R₆ may be same or different in each repeating unit;
each of R₇ and R₈, if present, is independently H, OH, SH, NH₂ or halogen, wherein, if n₄ is greater than 1, R₇ may be same or different in each repeating unit, and R₈ may be same or different in each repeating unit;
each of Rₐ and R_{b}, if present, is independently H, OH, SH, NH₂ or halogen, wherein, if n₆ is greater than 1, Rₐ may be same or different in each repeating unit, and R_{b} may be same or different in each repeating unit;
each of R_{c} and R_{d}, if present, is independently H, OH, SH, NH₂ or halogen, wherein, if n₇ is greater than 1, R_{c} may be same or different in each repeating unit, and R_{d} may be same or different in each repeating unit;
n₂, n₅ and n₈ are independently selected from 0 or 1, wherein, if m is greater than 1, n₈ may be same or different in each repeating unit;
one of R₃ and R₄, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl- R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group; and the other one of R₃ and R₄, if present, is selected from the group consisting of H, OH, SH, NH₂ and halogen; one of R₉ and R₁₀, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl- R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of R₉ and R₁₀, if present, is selected from the group consisting of H, OH, SH, NH₂ and halogen; one of Rₑ and R_{f}, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl- R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group; and the other one of Rₑ and R_{f}, if present, is selected from the group consisting of H, OH, SH, NH₂ and halogen;
m is selected from an integer ranging from 0 to 10, with the proviso that (i) if m is 0, one of n₂ and n₅ is 1 and the other one of n₂ and n₅ is 0, (ii) if m is 1, (a) n₂, n₅ and n₈ are 0 or (b) one of n₂, n₅ and n₈ is 1 and the other two of n₂, n₅ and n₈ are 0; and (iii) if m is an integer ranging from 2 to 10, (a) n₂, n₅ and each n₈ are 0, (b) one of n₂ and n₅ is 1 and the other one of n₂ and n₅ as well as each n₈ are 0, or (c) one n₈ is 1 and each other n₈ as well as n₂ and n₄ are 0;
L₁ is selected from the group consisting of CH₂, O, S, SO, SO₂, NR^{L}, CH(OH), CH(SH) and CH(halogen), with the proviso that, if m is 1 and n₂, n₅ and n₈ are 0, L₁ is selected from the group consisting of O, S, SO, SO₂ and NR^{L};
L₂ is selected from the group consisting of O, S, SO, SO₂ and NH; wherein, if m is greater than 1, L₂ may be same or different in each repeating unit;
X₁ is O, S, NH or CH₂,
R^{L} is (i) H, if one of n₂, n₅ and n₈ is 1, or (ii) selected from the group consisting of H, C₁-C₃-alkyl, C₁-C₃-alkyl-O—R^{C}, C₁-C₃-alkyl-S—R^{S}, C₁-C₃-alkyl-NR^{N1}R^{N2}, C₁-C₃-alkyl-R^{P} and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized, and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, if n₂, n₅ and n₈ are 0,
R^{O}, R^{S}, R^{N1} and R^{N2} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl- O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁- C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl- NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{D} and R^{E} are each independently selected from the group consisting of C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl- O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁- C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{N3} and R^{N4} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl- NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, with the proviso that R^{N3} and R^{N4} are not both H,
R^{P} is

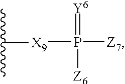

wherein
X₉ is O, S, NH or CH₂;
Y₆ is O, S or Se; and
each of Z₆ and Z₇ is independently OH, SH, SeH or BH₃;
and wherein the 5′ end is optionally deuterated.

In the third aspect, formula (III) corresponds to the cap nucleoside at the 5′ end. This cap nucleoside is typically linked to the remainder of the RNA molecule via a triphosphate bridge, wherein the triphosphate bridge connects X₁ of formula (III) [as indicated in formula (III)] and the 5′ position of the ribose of the first regular nucleotide of the RNA, i.e. the remainder of the RNA molecule.

In an embodiment of the third aspect, m is selected from 0 or 1.

In an embodiment of the third aspect, m is is selected from an integer ranging from 1 to 10, preferably 1, n₂, n₅ and each n₈ are 0, and L₁ is selected from the group consisting of O, S, SO, SO₂ and NR^{L}, preferably L₁ is O. In this embodiment, formula (III) corresponds to a cap nucleoside at the 5′ end, which comprises a linear unbranched structure.

In another embodiment of the third aspect, (i) m is 0, one of n₂ and n₅ is 1, and the other one of n₂ and n₅ is 0; (ii) m is 1, one of n₂, n₅ and n₈ is 1, and the other two of n₂, n₅ and n₈ are 0, (iii) m is an integer ranging from 2 to 10, one of n₂ and n₅ is 1 and the other one of n₂ and n₅ as well as each n₈ are 0, or (iv) m is an integer ranging from 2 to 10, one n₄ is 1 and each other ne as well as n₂ and n₅ are 0; and L₁ is selected from the group consisting of CH₂, O, S, SO, SO₂, NR^{L}, CH(OH), CH(SH) and CH(halogen), preferably L₁ is CH₂ or O. In this embodiment, formula (III) corresponds to a cap nucleoside at the 5′ end, which comprises a linear single-branched structure.

In a preferred embodiment thereof, m is 0, one of n₂ and n₅ is 1, and the other one of n₂ and n₅ is 0.

In one preferred embodiment, each of n₁ and no is independently selected from 1 or 2, preferably n₁ is 1 and n₉ is 1; and each of n₃, n₄, and, if present, each of ne and nm, is independently selected from 0 or 1.

In another preferred embodiment, one of R₃ and R₄ if present, one of R₉ and R₁₀ if present, or one of Rₑ and R_{f} if present, is selected from the group consisting of C₁-alkyl-O—R^{O}, C₁-alkyl-S—R^{S}, C₁-alkyl-S—S—R^{S}, C₁-alkyl-R^{P}; wherein
R^{O} and R^{S} are each independently selected from the group consisting of H, C₁-C₄-alkyl, C₁-C₂-cyanoalkyl, and 5- or 6-membered saturated, partially or fully unsaturated, or aromatic carbocyclyl, carbocyclyl-C₁-alkyl, heterocyclyl, or heterocyclyl-C₁-alkyl, wherein the aforementioned heterocyclic rings comprise one or two, same or different heteroatoms selected from O, N, or S, wherein said N- and/or S-atoms are independently oxidized or non-oxidized; and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₂-alkyl, preferably halogen;
R^{P} is

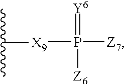

wherein
X₉ is O; Y₆ is O or S; and each of Z₅ and Z₇ is OH; and
the other one of R₃ and R₄ if present, R₉ and R₁₀ if present, Rₑ and R_{f} if present, is H; and wherein preferably each of R₁, R₂, R₅, R₆, R₇, R₈, Rₐ, R_{b}, R_{c}, R_{d}, R₁₁, and R₁₂, if present, is H.

In an especially preferred embodiment of the third aspect, ring B₁ is a modified guanine, preferably N⁷-methylguanine.

Of course, all embodiments disclosed in connection with formula (I) of the first aspect, in particular regarding each of R₁ through R₁₂, each of Rₐ through R_{f}, R^{O}, R^{S}, R^{P}, R^{N1}, R^{N2}, R^{N3}, R^{N4}, R^{L}, R^{D}, R^{E}, B¹, and X¹, each of n₁ through ng, L₁, L₂, and m, also apply to formula (III) of the third aspect.

In another embodiment of the third aspect, the RNA molecule comprises a 5′ end of formula (I):

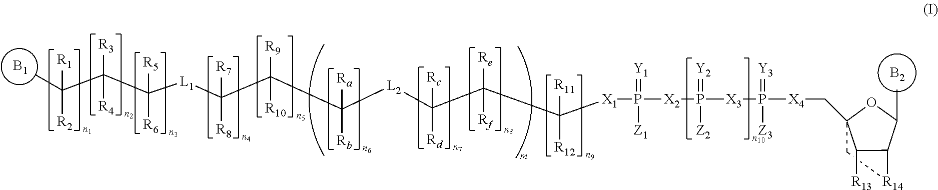

wherein
R₁₃ is

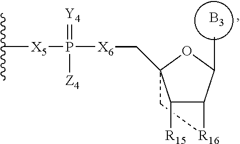

wherein
R₁₅ is

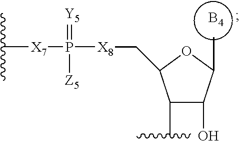

and wherein
n₁₀ is selected from 0, 1 or 2;
each of X₂ through Xe is independently O, S, NH or CH₂, wherein, if n₁ is 2, X₃ may be same or different in each repeating unit;
each of Y₁ through Y₅ is independently O, S or Se, wherein, if n₁₀ is 2, Y₂ may be same or different in each repeating unit;
each of Z₁ through Ze is independently OH, SH, SeH or BH₃, wherein, if n₁₀ is 2, Z₂ may be same or different in each repeating unit;
R₁₄ is (i) selected from the group consisting of H, OH, OC₁-C₃-alkyl, and Opropargyl, wherein the dashed methylene bridge between R₁₄ and the 4′ C is absent, or (ii) O, wherein the dashed methylene bridge between R₁₄ being O and the 4′ C is present;
R₁₄ is (i) selected from the group consisting of H, OH, OC₁-C₃-alkyl, and Opropargyl, wherein the dashed methylene bridge between Rie and the 4′ C is absent, or (ii) O, wherein the dashed methylene bridge between R₁₆ being O and the 4′ C is present;
each of ring B₂ through ring B₄ is independently a nucleobase, a modified nucleobase, or a nucleobase analog.

In an embodiment thereof, R₆ is OC₁-C₃-alkyl, preferably wherein R₆ is OCH₃, wherein the dashed methylene bridge between R₆ and the 4′ C is absent.

In another embodiment thereof, R₈ is OC₁-C₃-alkyl, preferably wherein R₈ is OCH₃, wherein the dashed methylene bridge between R₈ and the 4′ C is absent.

In yet another embodiment thereof, R₆ is OC₁-C₃-alkyl, preferably wherein R₆ is OCH₃, wherein the dashed methylene bridge between R₆ and the 4′ C is absent; and R₈ is OC₁-C₃-alkyl, preferably wherein R₈ is OCH₃, wherein the dashed methylene bridge between R₈ and the 4′ C is absent.

In still another embodiment thereof, (i) n₁₀ is 1; (ii) each of X₂ through X₈ is O; (iii) each of Y₁ through Y₅ is O; or one of Y₁ through Y₅, preferably Y₁, is S and each of the remaining ones of Y₁ through Y₅ is O; (iv) each of Z₁ through Z₅ is OH; and (v) each of ring B₂ through ring B₄ is a nucleobase.

Of course, all embodiments disclosed in connection with formula (I) of the first aspect also apply to formula (I) of the third aspect.

In the fourth aspect, the present invention relates to an RNA molecule comprising at least three nucleotides and comprising a 5′ terminal acyclonucleoside, wherein the acyclonucleoside comprises a linear unbranched or linear single-branched structure instead of a ribose, and wherein the 5′ terminal acyclonucleoside is optionally deuterated, wherein the linear unbranched or linear single-branched structure has the structure of formula (II):

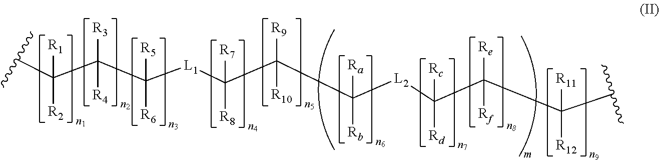

wherein
n₁ and n₉ are independently selected from an integer ranging from 1 to 10;
each of R₁ and R₂ is independently H, OH, SH, NH₂ or halogen, wherein, if n₁ is greater than 1, R₁ may be same or different in each repeating unit, and R₂ may be same or different in each repeating unit;
each of R₁₁ and R₁₂ is independently H, OH, SH, NH₂ or halogen, wherein, if n₉ is greater than 1, R₁₁ may be same or different in each repeating unit, and R₁₂ may be same or different in each repeating unit;
each of n₃, n₄, n₆, and n₇ is independently selected from an integer ranging from 0 to 5; wherein, if m is greater than 1, no may be same or different in each repeating unit, and n₇ may be same or different in each repeating unit;
each of R₅ and R₆, if present, is independently H, OH, SH, NH₂ or halogen, wherein, if n₃ is greater than 1, R₅ may be same or different in each repeating unit, and R₆ may be same or different in each repeating unit;
each of R₇ and R₈, if present, is independently H, OH, SH, NH₂ or halogen, wherein, if n₄ is greater than 1, R₇ may be same or different in each repeating unit, and R₈ may be same or different in each repeating unit;
each of Rₐ and R_{b}, if present, is independently H, OH, SH, NH₂ or halogen, wherein, if n₆ is greater than 1, Rₐ may be same or different in each repeating unit, and R_{b} may be same or different in each repeating unit;
each of R_{c} and R_{d}, if present, is independently H, OH, SH, NH₂ or halogen, wherein, if n₇ is greater than 1, R_{c} may be same or different in each repeating unit, and R_{d} may be same or different in each repeating unit;
n₂, n₅ and n₈ are independently selected from 0 or 1; wherein, if m is greater than 1, ne may be same or different in each repeating unit;
one of R₃ and R₄, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N}, C₁-C₃-alkyl- R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group; and the other one of R₃ and R₄, if present, is selected from the group consisting of H, OH, SH, NH₂ and halogen; one of R₉ and R₁₀, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl- R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group; and the other one of R₉ and R₁₀, if present, is selected from the group consisting of H, OH, SH, NH₂ and halogen; one of Rₑ and R_{f}, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl- R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group; and the other one of Rₑ and R_{f}, if present, is selected from the group consisting of H, OH, SH, NH₂ and halogen;
m is selected from an integer ranging from 0 to 10, with the proviso that (i) if m is 0, one of n₂ and n₅ is 1 and the other one of n₂ and n₅ is 0, (ii) if m is 1, (a) n₂, n₅ and n₈ are 0 or (b) one of n₂, n₅ and n₈ is 1 and the other two of n₂, n₅ and n₈ are 0; and (iii) if m is an integer ranging from 2 to 10, (a) n₂, n₅ and each n₈ are 0, (b) one of n₂ and n₅ is 1 and the other one of n₂ and n₅ as well as each n₈ are 0, or (c) one n₃ is 1 and each other n₈ as well as n₂ and n₅ are 0;
L₁ is selected from the group consisting of CH₂, O, S, SO, SO₂, NR^{L}, CH(OH), CH(SH) and CH(halogen), with the proviso that, if m is 1 and n₂, n₅ and n₈ are 0, L₁ is selected from the group consisting of O, S, SO, SO₂ and NR^{L};
L₂ is selected from the group consisting of O, S, SO, SO₂ and NH; wherein, if m is greater than 1, L₂ may be same or different in each repeating unit;
R^{L} is (i) H, if one of n₂, n₅ and n₈ is 1, or (ii) selected from the group consisting of H, C₁-C₃-alkyl, C₁-C₃-alkyl-O—R^{C}, C₁-C₃-alkyl-S—R^{S}, C₁-C₃-alkyl-NR^{N1}R^{N2}, C₁-C₃-alkyl-R^{P} and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized, and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, if n₂, n₅ and n₈ are 0;
R^{O}, R^{S}, R^{N1} and R^{N2} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl- O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁- C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl- NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{D} and R^{E} are each independently selected from the group consisting of C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl- O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁- C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{N3} and R^{N4} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl- NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, with the proviso that R^{N3} and R^{N4} are not both H;
R^{P} is

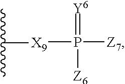

wherein
X₉ is O, S, NH or CH₂,
Y₆ is O, S or Se; and
each of Z₆ and Z₇ is independently OH, SH, SeH or BH₃;
and wherein the linear unbranched or linear single-branched structure is optionally deuterated.

In an embodiment of the fourth aspect, m is selected from 0 or 1.

In one embodiment of the fourth aspect, the acyclonucleoside comprises a linear unbranched structure of formula (II), wherein m is selected from an integer ranging from 1 to 10, preferably 1, n₂, n₅ and each n₈ are 0, and L₁ is selected from the group consisting of O, S, SO, SO and NR^{L}, preferably L₁ is O.

In another embodiment of the fourth aspect, the acyclonucleoside comprises a linear single-branched structure of formula (II), wherein
(i) m is 0, one of n₂ and n₅ is 1, and the other one of n₂ and n₅ is 0; (ii) m is 1, one of n₂, n₅ and n₈ is 1, and the other two of n₂, n₅ and n₈ are 0, (iii) m is an integer ranging from 2 to 10, one of n₂ and n₅ is 1 and the other one of n₂ and n₅ as well as each ne are 0, or (iv) m is an integer ranging from 2 to 10, one n₆ is 1 and each other ne as well as n₂ and n₅ are 0; and
L₁ is selected from the group consisting of CH₂, O, S, SO, SO₂, NR^{L}, CH(OH), CH(SH) and CH(halogen), preferably L₁ is CH₂ or O.

In a preferred embodiment thereof, the acyclonucleoside comprises a linear single-branched structure of formula (II), wherein m is 0, one of n₂ and n₅ is 1, and the other one of n₂ and n₅ is 0.

In one preferred embodiment, each of n₁ and no is independently selected from 1 or 2, preferably n₁ is 1 and n₉ is 1; and each of n₃, n₄, and, if present, each of n₆ and n₇, is independently selected from 0 or 1.

In another preferred embodiment, one of R₃ and R₄ if present, one of R₉ and R₁₀ if present, or one of Rₑ and R_{f} if present, is selected from the group consisting of C₁-alkyl-O—R^{O}, C₁-alkyl-S—R^{S}, C₁-alkyl-S—S—R^{S}, C₁-alkyl-R^{P}; wherein
R^{O} and R^{S} are each independently selected from the group consisting of H, C₁-C₄-alkyl, C₁-C₂-cyanoalkyl, and 5- or 6-membered saturated, partially or fully unsaturated, or aromatic carbocyclyl, carbocyclyl-C₁-alkyl, heterocyclyl, or heterocyclyl-C₁-alkyl, wherein the aforementioned heterocyclic rings comprise one or two, same or different heteroatoms selected from O, N, or S, wherein said N- and/or S-atoms are independently oxidized or non-oxidized; and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₂-alkyl, preferably halogen;
R^{P} is

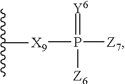

wherein
X₉ is O; Y₆ is O or S; and each of Z₅ and Z₇ is OH; and
the other one of R₃ and R₄ if present, R₉ and R₁₀ if present, Rₑ and R_{f} if present, is H, and
wherein preferably each of R₁, R₂, R₅, R₆, R₇, R₈, Rₐ, R_{b}, R_{c}, R_{d}, R₁₁, and R₁₂, if present, is H.

In a preferred embodiment of the fourth aspect, the acyclonucleoside comprises as the nucleobase guanine, a modified guanine or a guanine analog. It can be especially preferred that the acyclonucleoside comprises as the nucleobase a modified guanine, most preferably N⁷-methylguanine.

Of course, all embodiments disclosed in connection with formula (I) of the first aspect, in particular regarding each of R₁ through R₁₂, each of Rₐ through R_{f}, R^{O}, R^{S}, R^{P}, R^{N1}, R^{N2}, R^{N5}, R^{N4}, R^{L}, R^{D}, R^{E} each of n₁ through n₉, L₁, L₂, and m, also apply to formula (II) of the fourth aspect.

In the fifth aspect, the present invention relates to an RNA molecule whose 5′ end comprises a compound according to the first aspect.

It is noted with respect to the fifth aspect that the compound according to the first aspect mandatorily has an OH-group at the 3-position of the ribose of the 3′ nucleoside as shown in the following, namely due to (i) the definition of R₁₅ being OH or (ii) the alternative definition of R₁₅:

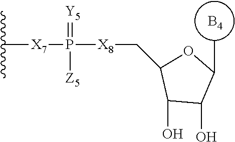

It is at this OH-group at the 3-position of the ribose where the further nucleotides in 3′ direction of the RNA molecule form a covalent bond, as shown here:

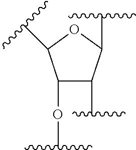 [SMILES: *OC1C(*)OC(*)C1*]

Thus, the RNA molecule of the fifth aspect comprises the compound according to the first aspect at its 5′ end such that this compound is covalently bound to the remainder of the RNA molecule, wherein the compound according to the first aspect is comprised in the cap structure of the RNA molecule.

Of course, all embodiments of the first aspect as outlined above also apply for the compounds that are comprised in the RNA molecule of the fifth aspect.

In the sixth aspect, the present invention is concerned with an in vitro method for synthesizing an RNA molecule, the method comprising reacting nucleotides, (i) the compound according to the first aspect or (ii) the cap analog according to the second aspect, and a DNA template in the presence of a DNA-dependent RNA polymerase under conditions suitable for the transcription of the DNA template into an RNA molecule by the DNA-dependent RNA polymerase.

The sixth aspect may alternatively be formulated as an in vitro method for synthesizing a capped RNA molecule, the method comprising reacting nucleotides, (i) a compound according to the first aspect or (ii) a cap analog according to the second aspect, and a DNA template in the presence of a DNA-dependent RNA polymerase under conditions suitable for the transcription of the DNA template into an RNA molecule by the DNA-dependent RNA polymerase.

In another preferred embodiment of the sixth aspect, the nucleotides are ATP, CTP, GTP and UTP or modified UTP, preferably N1-methylpseudouridine. If the RNA is artificial RNA, modified nucleotides as set out below in the detailed description of the present invention may alternatively or additionally be used. Such nucleotides comprise at least one chemical modification that will also be present in the resulting RNA such that the resulting RNA is an artificial RNA according to the below definition.

The ratio of the compound according to the first aspect to the nucleotide GTP used in the method according to the sixth aspect may vary from 10:1 to 1:1 in order to balance the percentage of capped RNA products with the efficiency of the transcription reaction. Preferably, a ratio of the compound according to the first aspect to GTP of 4:1-6:1 is used.

In some embodiments of the sixth aspect, the method comprises at least one step of purifying the obtained capped RNA molecule. Suitable methods for purification may comprise RP-HPLC, Oligo-dT purification, anion exchange chromatography, cellulose-purification (such as e.g. the purification method using a cellulose material as disclosed in WO 2017/182525) and/or TFF, or a combination of at least two purification methods.

In a preferred embodiment of the sixth aspect, the DNA-dependent RNA polymerase is the T7, modified T7, T3 or SP6 polymerase.

In yet another preferred embodiment of the sixth aspect, the DNA template is a linearized DNA template with a promoter sequence that has a high binding affinity for its respective RNA polymerase.

In another preferred embodiment of the sixth aspect, the conditions suitable for the transcription of the DNA template into an RNA molecule comprise a suitable buffer, where the suitable buffer is preferably capable of maintaining a suitable pH value and may contain antioxidants (e.g. DTT), and/or polyamines such as spermidine at optimal concentrations. It can further be preferred that the buffer contains divalent cations, most preferably MgCl₂.

In yet another preferred embodiment of the sixth aspect, the method may further comprise adding a ribonuclease inhibitor. In another embodiment of the sixth aspect, the method may further comprises adding a pyrophosphatase.

Of course, all embodiments of the first aspect as outlined above also apply for the compounds of the first aspect that are used in the method of the sixth aspect. The same applies with respect to the embodiments of the second aspect as outlined above that apply for the cap analog of the second aspect that is used in the method of the sixth aspect.

In the seventh aspect, the present invention is concerned with an RNA molecule obtained by the method according to the sixth aspect, including all embodiments thereof.

In an embodiment of the seventh aspect, about 60%, about 65%, about 70%, about 75%, about 80%, about 85%, about 90%, or about 95% or more of the RNA molecules obtained by the method according to the sixth aspect comprises a cap structure derived from the compound according to the first aspect as determined using a capping assay. In preferred embodiments, less than about 30%, about 25%, about 20%, about 15%, about 10%, about 5%, about 4%, about 3%, about 2%, or about 1% of the RNA molecules obtained by the method according to the sixth aspect does not comprises a cap structure, determined using a capping assay. The capping assay may be carried out along the lines as shown herein in Example 3.

In another embodiment of the seventh aspect, the RNA molecule is characterized by an absence of reverse cap structures as compared to, e.g., RNA that has been generated using mCap. The structure of mCap (which may also be referred to as “mCap analog”) is shown in example 2 herein. The capping assay may essentially be carried out as shown herein in Example 3.

In another embodiment of the seventh aspect, the RNA molecule is characterized by an absence of reverse cap structures as compared to, e.g., RNA that has been generated using Cap1. The structure of Cap1 (which may also be referred to as “CleanCap analog”) is shown in example 2 herein. The capping assay may essentially be carried out as shown herein in Example 3.

In another embodiment of the seventh aspect, the RNA molecule has a reduced dsRNA content as compared to, e.g., RNA that has been generated using mCap, CleanCap or RNA that has been generated by a post-transcriptional enzymatic capping reaction. The dsRNA content may be determined along the lines as shown herein in Example 4.

In embodiments relating to the third, fourth, fifth and seventh aspect, the RNA molecule comprises at least one chemical modification. The chemical modification may in particular be selected from the group consisting of a base modification, a sugar modification and a backbone modification. Such modifications are set out in detail in the detailed description of the present invention below. At least one chemical modification may in particular be a base modification, wherein the base modification is preferably selected from the group consisting of pseudouracil (psi or y), N1-methylpseudouracil (N1Mpsi or N1Mψ), 1-ethylpseudouracil, 2-thiouracil (s2U), 4-thiouracil, 5-methylcytosine, 5-methyluracil, 5-methoxyuracil, and any combination thereof. It can also be preferred that the base modification is selected from the group consisting of pseudouracil (ψ), N1-methylpseudouracil (m1ψ), 5-methylcytosine and 5-methoxyuracil. Most preferably, the base modification is N1-methylpseudouracil (m1ψ).

In other embodiments relating to the third, fourth, fifth and seventh aspect, the RNA molecule does not comprise at least one chemical modification (i.e. no additional modification to the cap structure).

In particularly preferred embodiments relating to the third, fourth, fifth and seventh aspect, the RNA molecule is a coding RNA comprising at least one coding sequence. Most preferably, the coding RNA is an mRNA.

In preferred embodiments relating to the third, fourth, fifth and seventh aspect, the RNA molecule comprises at least one poly(A) sequence, and/or at least one poly(C) sequence, and/or at least one histone stem-loop and/or at least one 5′-UTR and/or at least one 3′-UTR. Most preferably the RNA molecule comprises at least one poly(A) tail (A100), optionally one histone stem-loop and at least one 5′UTR (HSD17B4) and one 3′UTR (PSMB3).

In preferred embodiments relating to the third, fourth, fifth and seventh aspect, the RNA molecule is a therapeutic mRNA. As used herein, the term “therapeutic mRNA” refers to an RNA that encodes a therapeutic protein. Therapeutic proteins mediate a variety of effects in a host cell or a subject in order to treat a disease or ameliorate the signs and symptoms of a disease.

In preferred embodiments relating to the third, fourth, fifth and seventh aspect, the RNA molecule has an increased translation efficiency as compared to, e.g., natural RNA or RNA that has been generated using mCap.

In other preferred embodiments relating to the third, fourth, fifth and seventh aspect, the RNA molecule has an increased half-life as compared to, e.g., natural RNA or RNA that has been generated using mCap.

In still other preferred embodiments relating to the third, fourth, fifth and seventh aspect, the RNA molecule has an increased resistance to degradation as compared to, e.g., natural RNA or RNA that has been generated using mCap.

In still other preferred embodiments relating to the third, fourth, fifth and seventh aspect, the RNA molecule has an increased stability as compared to, e.g., RNA that has been generated using mCap or RNA that has been generated by a post-transcriptional enzymatic capping reaction.

In still other preferred embodiments relating to the third, fourth, fifth and seventh aspect, the RNA molecule exhibits reduced immunostimulation as compared to, e.g., RNA that has been generated using mCap or RNA that has been generated by a post-transcriptional enzymatic capping reaction.

In still other preferred embodiments relating to the third, fourth, fifth and seventh aspect, the RNA molecule has at least one of the following characteristics as compared to, e.g., (i) RNA with the same sequence that has been generated using mCap, (ii) RNA with the same sequence that has been generated using CleanCap, or (iii) RNA with the same sequence that has been capped using enzymatic capping, in particular a post-transcriptional enzymatic capping reaction:
(a) increased translation efficiency when administered to a cell or a subject;
(b) prolonged translation of the encoded protein when administered to a cell or a subject;
(c) reduced de-capping in cells;
(d) increased half-life when administered to a cell or a subject;
(e) increased resistance to degradation when administered to a cell or a subject;
(f) increased stability when administered to a cell or a subject;
(g) reduced immunostimulation when administered to a cell or a subject;
(h) increased translation efficiency when administered intravenously to a cell or a subject;
(i) prolonged translation of the encoded protein when administered intravenously to a cell or a subject;
(j) increased half-life when administered intravenously to a cell or a subject;
(k) increased resistance to degradation when administered intravenously to a cell or a subject;
(l) increased stability when administered intravenously to a cell or a subject;
(m) reduced immunostimulation when administered intravenously to a cell or a subject;
(n) increased translation efficiency when administered intramuscularly to a cell or a subject;
(o) prolonged translation of the encoded protein when administered intramuscularly to a cell or a subject;
(p) increased half-life when administered intramuscularly to a cell or a subject;
(q) increased resistance to degradation when administered intramuscularly to a cell or a subject;
(r) increased stability when administered intramuscularly to a cell or a subject; and
(s) reduced immunostimulation when administered intramuscularly to a cell or a subject

The characteristics mentioned above can be determined by standard assays known to the skilled person, in particular corresponding assays in cell culture or model species such as mice.

In the eighth aspect, the present invention relates to a composition comprising the RNA molecule according to any of the third, fourth or fifth aspect, including all embodiments thereof as outlined above. The composition may also comprise a plurality of RNA molecules according to any of the third, fourth or fifth aspect, including all embodiments thereof as outlined above.

In an embodiment of the eight aspect, the RNA comprised in the composition is formulated in at least one cationic or polycationic compound, e.g. cationic or polycationic peptides, cationic or polycationic proteins, cationic or polycationic lipids, cationic or polycationic polysaccharides and/or cationic or polycationic polymers. In a preferred embodiment thereof, the RNA is formulated in lipid-based carriers, preferably wherein the lipid-based carriers encapsulate the RNA. In most preferred embodiments thereof, the lipid-based carriers are liposomes, lipid nanoparticles, lipoplexes, lipid complexes and/or nanoliposomes. In preferred embodiments, the lipid-based carriers of the pharmaceutical composition are lipid nanoparticles (LNPs), in particular LNPs as described and disclosed in WO2022207862, page 41 paragraph 14 ff. Lipid-based carriers can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter. Suitably, the lipid-based carriers of the composition comprise at least one aggregation-reducing lipid (e.g. a PEG-lipid), at least one cationic lipid, at least one neutral lipid, and/or at least one steroid or steroid analog.

In preferred embodiments, the lipid-based carrier is selected from a lipid nanoparticle (LNP). In further preferred embodiments, the lipid-based carrier, preferably the LNP, comprising the RNA molecule according to any of the third, fourth or fifth aspect, preferably the mRNA according to any of the third, fourth or fifth aspect, comprises
(i) at least one cationic lipid;
(ii) at least one or two (e.g. two different) neutral lipids;
(iii) at least one steroid or steroid analog; and
(iv) at least one aggregation reducing lipid, preferably a polymer conjugated lipid.

Suitable cationic lipids, neutral lipids, steroid or steroid analog, and aggregation reducing lipids are defined in particular in WO2022207862, page 41 paragraph 31 ff.

In preferred embodiments of the eight aspect, the composition is a pharmaceutical composition, in particular in the embodiments of the third, fourth or fifth aspect, where the RNA is therapeutic mRNA. The pharmaceutical composition comprises at least one pharmaceutically acceptable carrier.

In another preferred embodiment of the eight aspect, the composition is a pharmaceutical composition and comprises the RNA molecule according to any of the third, fourth or fifth aspect, wherein the RNA is therapeutic mRNA, wherein the RNA is formulated in a lipid-based carrier, preferably an LNP.

In the ninth aspect, the present invention relates to a kit comprising (i) the compound according to the first aspect or (ii) the cap analog of the second aspect, and a DNA-dependent RNA polymerase, wherein it can be preferred that the DNA-dependent RNA polymerase is the T7, modified T7, T3 or SP6 polymerase. This kit is suitable for producing a capped RNA. Of course, all embodiments of the first aspect as outlined above also apply for the compounds comprised in the kit of the ninth aspect, and all embodiments of the second aspect as outlined above also apply for the cap analog comprised in the kit of the ninth aspect.

In an embodiment of the ninth aspect, the kit further comprises nucleotides, preferably ATP, CTP, GTP and UTP or modified UTP. If the RNA is artificial RNA, modified nucleotides as set out below in the detailed description of the present invention may alternatively or additionally be comprised in the kit. Such nucleotides comprise at least one chemical modification that will also be present in the resulting RNA such that the resulting RNA is an artificial RNA according to the below definition.

In yet another embodiment of the ninth aspect, the kit further comprises a ribonuclease inhibitor. In another embodiment of the ninth aspect, the kit further comprises a pyrophosphatase.

In still another embodiment of the ninth aspect, the kit further comprises a buffer. Preferably, this buffer is capable of maintaining a suitable pH value and may contain antioxidants (e.g. DTT), and/or polyamines such as spermidine at optimal concentrations. It can be preferred that the buffer contains divalent cations, most preferably MgCl₂.

In the tenth aspect, the present invention relates to the use of (i) the compound according to the first aspect or (ii) the cap analog of the second aspect in an in vitro transcription reaction for producing a capped RNA molecule.

The tenth aspect may alternatively be formulated as the use of (i) the compound according to the first aspect or (ii) the cap analog of the second aspect in an in vitro transcription reaction for co-transcriptionally producing capped RNA.

Of course, all embodiments of the first aspect as outlined above also apply for the compounds that are used according to the tenth aspect. Accordingly, all embodiments of the second aspect as outlined above also apply for the cap analogs that are used according to the tenth aspect.

In the eleventh aspect, the present invention relates to a method of synthesizing the compound according to the first aspect. Preferred methods of synthesizing the compound according to the first aspect can be found in example 1 herein below.

In the twelfth aspect, the present invention relates a method of increasing the translation of an in vitro transcribed RNA in a cell or subject, the method comprising at least the steps of (i) synthesizing an RNA molecule according to the method of the sixth aspect and (ii) applying the obtained RNA molecule to a cell or subject. Typically, a step of formulating the RNA molecule, preferably in at least one cationic or polycationic compound, more preferably in lipid-based carries such as LNPs, is comprised in the method as well, wherein the step of formulating the RNA molecule is between step (i) and (ii). Further, after the step of formulating the RNA molecule, there may be a step (preferably an automated step) of fill and finish, taking place prior to step (ii). Insofar step (ii) is concerned with the application to a subject, step (ii) is preferably carried out by administering the (optionally filled and finished formulated) RNA molecule intravenously or intramuscularly to the subject. The obtained RNA molecule may also be referred to as capped RNA.

In the thirteenth aspect, the present invention relates a method of increasing the half-life of an in vitro transcribed RNA in a cell or subject, the method comprising at least the steps of (i) synthesizing an RNA molecule according to the method of the sixth aspect and (ii) applying the obtained RNA molecule to a cell or subject. Typically, a step of formulating the RNA molecule, preferably in at least one cationic or polycationic compound, more preferably in lipid-based carries such as LNPs, is comprised in the method as well, wherein the step of formulating the RNA molecule is between step (i) and (ii). Further, after the step of formulating the RNA molecule, there may be a step (preferably an automated step) of fill and finish, taking place prior to step (ii). Insofar step (ii) is concerned with the application to a subject, step (ii) is preferably carried out by administering the (optionally filled and finished formulated) RNA molecule intravenously or intramuscularly to the subject. The obtained RNA molecule may also be referred to as capped RNA.

In the fourteenth aspect, the present invention relates a method of increasing resistance to degradation of an in vitro transcribed RNA in a cell or subject, the method comprising at least the steps of (i) synthesizing an RNA molecule according to the method of the sixth aspect and (ii) applying the obtained RNA molecule to a cell or subject. Typically, a step of formulating the RNA molecule, preferably in at least one cationic or polycationic compound, more preferably in lipid-based carries such as LNPs, is comprised in the method as well, wherein the step of formulating the RNA molecule is between step (i) and (ii). Further, after the step of formulating the RNA molecule, there may be a step (preferably an automated step) of fill and finish, taking place prior to step (ii). Insofar step (ii) is concerned with the application to a subject, step (ii) is preferably carried out by administering the (optionally filled and finished formulated) RNA molecule intravenously or intramuscularly to the subject. The obtained RNA molecule may also be referred to as capped RNA.

In the fifteenth aspect, the present invention relates a method of increasing stability of an in vitro transcribed RNA in a cell or subject, the method comprising at least the steps of (i) synthesizing an RNA molecule according to the method of the sixth aspect and (ii) applying the obtained RNA molecule to a cell or subject. Typically, a step of formulating the RNA molecule, preferably in at least one cationic or polycationic compound, more preferably in lipid-based carries such as LNPs, is comprised in the method as well, wherein the step of formulating the RNA molecule is between step (i) and (ii). Further, after the step of formulating the RNA molecule, there may be a step (preferably an automated step) of fill and finish, taking place prior to step (ii). Insofar step (ii) is concerned with the application to a subject, step (ii) is preferably carried out by administering the (optionally filled and finished formulated) RNA molecule intravenously or intramuscularly to the subject. The obtained RNA molecule may also be referred to as capped RNA.

In the sixteenth aspect, the present invention relates a method of reducing immunostimulation of an in vitro transcribed RNA in a cell or subject, the method comprising at least the steps of (i) synthesizing an RNA molecule according to the method of the sixth aspect and (ii) applying the obtained RNA molecule to a cell or subject. Typically, a step of formulating the RNA molecule, preferably in at least one cationic or polycationic compound, more preferably in lipid-based carries such as LNPs, is comprised in the method as well, wherein the step of formulating the RNA molecule is between step (i) and (ii). Further, after the step of formulating the RNA molecule, there may be a step (preferably an automated step) of fill and finish, taking place prior to step (ii). Insofar step (ii) is concerned with the application to a subject, step (ii) is preferably carried out by administering the (optionally filled and finished formulated) RNA molecule intravenously or intramuscularly to the subject. The obtained RNA molecule may also be referred to as capped RNA.

All embodiments of the sixth aspect of course also apply for the methods of the twelfth to the sixteenth aspect.

In the seventeenth aspect, the present invention relates to a transcription initiation complex comprising (i) the compound according to the first aspect or (ii) the cap analog according to the second aspect, and a DNA template. It can be preferred that the DNA template is a linearized DNA template.

Of course, all embodiments of the first aspect as outlined above also apply for the compounds that are comprised in the complex according to the seventeenth aspect. Accordingly, all embodiments of the second aspect as outlined above also apply for the cap analogs that are comprised in the complex according to the seventeenth aspect.

In the eighteenth aspect, the present invention is concerned with an in vitro method for synthesizing an RNA molecule, the method comprising
(A) reacting (i) nucleotides, (ii) a compound of formula (I)

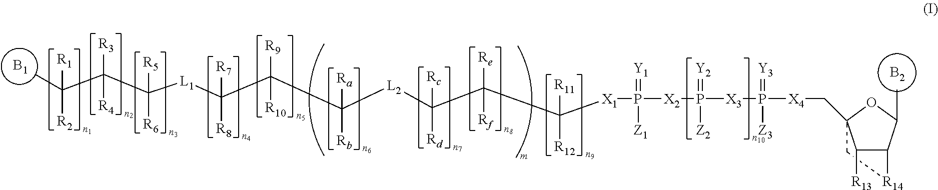

or a salt, stereoisomer, tautomer or deuterated version thereof, wherein
   R₁₃ is OH;
   R₁₄ is OH, wherein the dashed methylene bridge between R₁₄ and the 4′ C is absent;
   n₁ and n₉ are independently selected from an integer ranging from 1 to 10;
   each of R₁ and R₂ is independently H, OH, SH, NH₂ or halogen, wherein, if n₁ is greater than 1, R₁ may be same or different in each repeating unit, and R₂ may be same or different in each repeating unit;
   each of R₁₁ and R₁₂ is independently H, OH, SH, NH₂ or halogen, wherein, if n₉ is greater than 1, R₁₁ may be same or different in each repeating unit, and R₁₂ may be same or different in each repeating unit;
   each of n₃, n₄, n₆, and n₇ is independently selected from an integer ranging from 0 to 5; wherein, if m is greater than 1, ne may be same or different in each repeating unit, and n₇ may be same or different in each repeating unit;
   each of R₅ and R₆, if present, is independently H, OH, SH, NH₂ or halogen, wherein, if n₃ is greater than 1, R₅ may be same or different in each repeating unit, and R₆ may be same or different in each repeating unit;
   each of R₇ and R₈, if present, is independently H, OH, SH, NH₂ or halogen, wherein, if n₄ is greater than 1, R₇ may be same or different in each repeating unit, and R₈ may be same or different in each repeating unit;
   each of Rₐ and R_{b}, if present, is independently H, OH, SH, NH₂ or halogen, wherein, if n₅ is greater than 1, Rₐ may be same or different in each repeating unit, and R_{b} may be same or different in each repeating unit;
   each of R_{c} and R_{d}, if present, is independently H, OH, SH, NH₂ or halogen, wherein, if n₇ is greater than 1, R_{d} may be same or different in each repeating unit, and R_{d} may be same or different in each repeating unit;
   n₂, n₅ and n₈ are independently selected from 0 or 1; wherein, if m is greater than 1, n₆ may be same or different in each repeating unit;
   one of R₃ and R₄, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{O}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
   and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
   and the other one of R₃ and R₄, if present, is selected from the group consisting of H, OH, SH, NH₂ and halogen;
   one of R₉ and R₁₀, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
   and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
   and the other one of R₉ and R₁₀, if present, is selected from the group consisting of H, OH, SH, NH₂ and halogen;
   one of Rₑ and R_{f}, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
   and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
   and the other one of Rₑ and R_{f}, if present, is selected from the group consisting of H, OH, SH, NH₂ and halogen;
   m is selected from an integer ranging from 0 to 10, with the proviso that (i) if m is 0, one of n₂ and n₅ is 1 and the other one of n₂ and n₅ is 0, (ii) if m is 1, (a) n₂, n₅ and n₈ are 0 or (b) one of n₂, n₅ and n₃ is 1 and the other two of n₂, n₅ and n₈ are 0; and (iii) if m is an integer ranging from 2 to 10, (a) n₂, n₅ and each n₈ are 0, (b) one of n₂ and n₅ is 1 and the other one of n₂ and n₅ as well as each n₈ are 0, or (c) one n₅ is 1 and each other n₈ as well as n₂ and n₅ are 0;
   n₁₀ is selected from 0, 1 or 2,
   L₁ is selected from the group consisting of CH₂, O, S, SO, SO₂, NR^{L}, CH(OH), CH(SH) and CH(halogen), with the proviso that, if m is 1 and n₂, n₅ and n₈ are 0, L₁ is selected from the group consisting of O, S, SO, SO₂ and NR^{L};
   L₂ is selected from the group consisting of O, S, SO, SO₂ and NH; wherein, if m is greater than 1, L₂ may be same or different in each repeating unit;
   each of X₁ through X₄ is independently O, S, NH or CH₂, wherein, if n₁₀ is 2, X₃ may be same or different in each repeating unit;
   each of Y₁ through Y₃ is independently O, S or Se, wherein, if n₁₀ is 2, Y₂ may be same or different in each repeating unit;
   each of Z₁ through Z₃ is independently OH, SH, SeH or BH₃, wherein, if n₁₀ is 2, Z₂ may be same or different in each repeating unit;
   R^{L} is (i) H, if one of n₂, n₅ and n₈ is 1, or (ii) selected from the group consisting of H, C₁-C₃-alkyl, C₁-C₃-alkyl-O—R^{O}, C₁-C₃-alkyl-S—R^{S}, C₁-C₃-alkyl-NR^{N1}R^{N2}, C₁-C₃-alkyl-R^{P} and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized, and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, if n₂, n₅ and n₈ are 0;
   R^{O}, R^{S}, R^{N1} and R^{N2} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
   and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
   R^{D} and R^{E} are each independently selected from the group consisting of C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, CO—C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
   and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
   R^{N3} and R^{N4} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl- O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
   and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, with the proviso that R^{N3} and R^{N4} are not both H; and
   R^{P} is

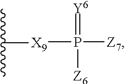

wherein
X₉ is O, S, NH or CH₂;
Y₆ is O, S or Se; and
each of Z₅ and Z₇ is independently OH, SH, SeH or BH₃; and
ring B₂ is a nucleobase, a modified nucleobase or a nucleobase analog;
and (iii) a DNA template in the presence of a DNA-dependent RNA polymerase under conditions suitable for the transcription of the DNA template into an RNA molecule by the DNA-dependent RNA polymerase;
(B) reacting the resulting RNA molecule in the presence of an RNA-methyltransferase that catalyzes the methylation of the OH-group at R₁₄ to arrive at OCH₃ under conditions suitable for this methylation reaction; and
(C) thereby arriving at an RNA molecule.

The eighteenth aspect may alternatively be formulated as an in vitro method for synthesizing a capped RNA molecule with a Cap1 structure, the method comprising
(A) reacting (i) nucleotides, (ii) a compound of formula (I)

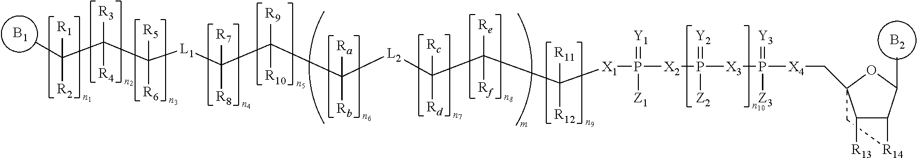

or a salt, stereoisomer, tautomer or deuterated version thereof, wherein
R₁₃ is OH;
R₁₄ is OH, wherein the dashed methylene bridge between R₁₄ and the 4′ C is absent;
n₁ and n₉ are independently selected from an integer ranging from 1 to 10;
each of R₁ and R₂ is independently H, OH, SH, NH₂ or halogen, wherein, if n₁ is greater than 1, R₁ may be same or different in each repeating unit, and R₂ may be same or different in each repeating unit;
each of R₁₁ and R₁₂ is independently H, OH, SH, NH₂ or halogen, wherein, if n₉ is greater than 1, R₁₁ may be same or different in each repeating unit, and R₁₂ may be same or different in each repeating unit;
each of n₃, n₄, n₆, and n₇ is independently selected from an integer ranging from 0 to 5; wherein, if m is greater than 1, n₆ may be same or different in each repeating unit, and n₇ may be same or different in each repeating unit;
each of R₅ and R₆, if present, is independently H, OH, SH, NH₂ or halogen, wherein, if n₃ is greater than 1, R₅ may be same or different in each repeating unit, and R₆ may be same or different in each repeating unit;
each of R₇ and R₈, if present, is independently H, OH, SH, NH₂ or halogen, wherein, if n₄ is greater than 1, R₇ may be same or different in each repeating unit, and R₈ may be same or different in each repeating unit;
each of Rₐ and R_{b}, if present, is independently H, OH, SH, NH₂ or halogen, wherein, if n₆ is greater than 1, Rₐ may be same or different in each repeating unit, and R_{b} may be same or different in each repeating unit;
each of R_{c} and R_{d}, if present, is independently H, OH, SH, NH₂ or halogen, wherein, if n₇ is greater than 1, R_{d} may be same or different in each repeating unit, and R_{d} may be same or different in each repeating unit;
n₂, n₅ and n₈ are independently selected from 0 or 1; wherein, if m is greater than 1, n₈ may be same or different in each repeating unit;
one of R₃ and R₄, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{O}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of R₃ and R₄, if present, is selected from the group consisting of H, OH, SH, NH₂ and halogen;
one of R₉ and R₁₀, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of R₉ and R₁₀, if present, is selected from the group consisting of H, OH, SH, NH₂ and halogen;
one of Rₑ and R_{f}, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of Rₑ and R_{f}, if present, is selected from the group consisting of H, OH, SH, NH₂ and halogen;
m is selected from an integer ranging from 0 to 10, with the proviso that (i) if m is 0, one of n₂ and n₅ is 1 and the other one of n₂ and n₅ is 0, (ii) if m is 1, (a) n₂, n₅ and n₈ are 0 or (b) one of n₂, n₅ and n₈ is 1 and the other two of n₂, n₅ and n₈ are 0; and (iii) if m is an integer ranging from 2 to 10, (a) n₂, n₅ and each n₇ are 0, (b) one of n₂ and n₅ is 1 and the other one of n₂ and n₅ as well as each n₈ are 0, or (c) one n₅ is 1 and each other n₈ as well as n₂ and n₅ are 0;
n₁₀ is selected from 0, 1 or 2;
L₁ is selected from the group consisting of CH₂, O, S, SO, SO₂, NR^{L}, CH(OH), CH(SH) and CH(halogen), with the proviso that, if m is 1 and n₂, n₅ and n₈ are 0, L₁ is selected from the group consisting of O, S, SO, SO₂ and NR^{L};
L₂ is selected from the group consisting of O, S, SO, SO₂ and NH; wherein, if m is greater than 1, L₂ may be same or different in each repeating unit;
each of X₁ through X₄ is independently O, S, NH or CH₂, wherein, if n₁₀ is 2, X₃ may be same or different in each repeating unit;
each of Y₁ through Y₃ is independently O, S or Se, wherein, if n₁₀ is 2, Y₂ may be same or different in each repeating unit;
each of Z₁ through Z₃ is independently OH, SH, SeH or BH₃, wherein, if n₁₀ is 2, Z₂ may be same or different in each repeating unit;
R^{L} is (i) H, if one of n₂, n₅ and n₈ is 1, or (ii) selected from the group consisting of H, C₁-C₃-alkyl, C₁-C₃-alkyl-O—R^{O}, C₁-C₃-alkyl-S—R^{S}, C₁-C₃-alkyl-NR^{N1}R^{N2}, C₁-C₃-alkyl-R^{P} and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized, and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, if n₂, n₅ and n₈ are 0;
R^{O}, R^{S}, R^{N1} and R^{N2} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{D} and R^{E} are each independently selected from the group consisting of C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{N3} and R^{N4} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl- O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, with the proviso that R^{N3} and R^{N4} are not both H; and
R^{P} is

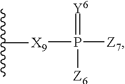

wherein
X₉ is O, S, NH or CH₂;
Y₆ is O, S or Se; and
each of Z₆ and Z₇ is independently OH, SH, SeH or BH₃; and
ring B₂ is a nucleobase, a modified nucleobase or a nucleobase analog;
and (iii) a DNA template in the presence of a DNA-dependent RNA polymerase under conditions suitable for the transcription of the DNA template into an RNA molecule by the DNA-dependent RNA polymerase;
(B) reacting the resulting RNA molecule in the presence of an RNA-methyltransferase that catalyzes the methylation of the OH-group at R₁₄ to arrive at OCH₃ under conditions suitable for this methylation reaction; and
(C) thereby arriving at a capped RNA molecule with a Cap1 structure.

In a preferred embodiment of the eighteenth aspect (relating to both above formulations thereof), m is selected from 0 or 1.

In another preferred embodiment of the eighteenth aspect, the nucleotides are ATP, CTP, GTP and UTP. If the RNA is artificial RNA, modified nucleotides as set out below in the detailed description of the present invention may alternatively or additionally be used. Such nucleotides comprise at least one chemical modification that will also be present in the resulting RNA such that the resulting RNA is an artificial RNA according to the below definition.

The ratio of the compound according to formula (I) to the nucleotide GTP used in the method according to the eighteenth aspect may vary from 10:1 to 1:1 in order to balance the percentage of capped RNA products with the efficiency of the transcription reaction. Preferably, a ratio of the compound according to the first aspect to GTP of 4:1-6:1 is used.

In some embodiments of the eighteenth aspect, the method comprises at least one step of purifying the obtained capped RNA molecule, optionally purifying the capped RNA molecule obtained after step (A) or purifying the capped RNA molecule with a Cap1 structure obtained after step (B). Suitable methods for purification may comprise RP-HPLC, Oligo-dT purification, anion exchange chromatography, cellulose-purification (such as e.g. the purification method using a cellulose material as disclosed in WO 2017/182525) and/or TFF, or a combination of at least two purification methods.

In a preferred embodiment of the eighteenth aspect, the DNA-dependent RNA polymerase is the T7, modified T7, T3 or SP6 polymerase.

In yet another preferred embodiment of the eighteenth aspect, the DNA template is a linearized DNA template with a promoter sequence that has a high binding affinity for its respective RNA polymerase.

In another preferred embodiment of the eighteenth aspect, the conditions suitable for the transcription of the DNA template into an RNA molecule comprise a suitable buffer, where the suitable buffer is preferably capable of maintaining a suitable pH value and may contain antioxidants (e.g. DTT), and/or polyamines such as spermidine at optimal concentrations. It can further be preferred that the buffer contains divalent cations, most preferably MgCl₂.

In yet another preferred embodiment of the eighteenth aspect, the method may further comprise adding a ribonuclease inhibitor. In another embodiment of the sixth aspect, the method may further comprises adding a pyrophosphatase.

In a preferred embodiment of the eighteenth aspect, the RNA-methyltransferase that catalyzes the methylation of the OH-group at R₁₄ to arrive at OCH₃ is a 2′-O-Methyltransferase, for example a 2′-O-Methyltransferase derived from Vaccinia virus (e.g. ScriptCap from Cellscript).

In another preferred embodiment of the eighteenth aspect, the conditions suitable for the methylation of the OH-group at R₁₄ to arrive at OCH₃ comprise a suitable buffer, where the suitable buffer is preferably a 1× ScriptCap capping buffer from Cellscript with an optional addition of RNase inhibitor and 20 mM S-Adenosyl methionine.

Of course, all embodiments disclosed in connection with formula (I) of the first aspect, if present, also apply to formula (I) of the eighteenth aspect.

In the nineteenth aspect, the present invention is concerned with a process for preparing a compound of formula (I):

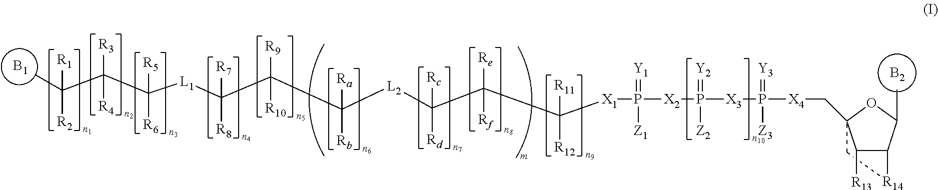

or a salt, stereoisomer, tautomer, or deuterated version thereof, wherein
R₁₃ is

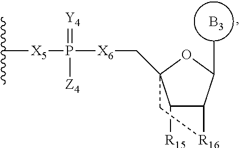

wherein
R₁₅ is OH or

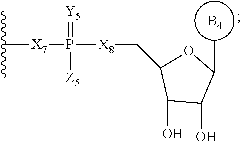

ring B₁ is guanine, a modified guanine or a guanine analog;
n₁ and n₉ are independently selected from an integer ranging from 1 to 10;
each of R₁ and R₂ is independently H, OH, SH, NH₂ or halogen, wherein, if n₁ is greater than 1, R₁ may be same or different in each repeating unit, and R₂ may be same or different in each repeating unit;
each of R₁₁ and R₁₂ is independently H, OH, SH, NH₂ or halogen, wherein, if n₉ is greater than 1, R₁₁ may be same or different in each repeating unit, and R₁₂ may be same or different in each repeating unit;
each of n₃, n₄, n₆, and n₇ is independently selected from an integer ranging from 0 to 5; wherein, if m is greater than 1, n₆ may be same or different in each repeating unit, and n₇ may be same or different in each repeating unit;
each of R₅ and R₆, if present, is independently H, OH, SH, NH₂ or halogen, wherein, if n₃ is greater than 1, R₅ may be same or different in each repeating unit, and R₆ may be same or different in each repeating unit;
each of R₇ and R₈, if present, is independently H, OH, SH, NH₂ or halogen, wherein, if n₄ is greater than 1, R₇ may be same or different in each repeating unit, and R₈ may be same or different in each repeating unit;
each of Rₐ and R_{b}, if present, is independently H, OH, SH, NH₂ or halogen, wherein, if n₆ is greater than 1, Rₐ may be same or different in each repeating unit, and R_{b} may be same or different in each repeating unit;
each of R_{c} and R_{d}, if present, is independently H, OH, SH, NH₂ or halogen, wherein, if n₇ is greater than 1, R_{c} may be same or different in each repeating unit, and R_{d} may be same or different in each repeating unit;
n₂, n₅ and n₈ are independently selected from 0 or 1; wherein, if m is greater than 1, n₈ may be same or different in each repeating unit;
one of R₃ and R₄, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl- R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group; and the other one of R₃ and R₄, if present, is selected from the group consisting of H, OH, SH, NH₂ and halogen; one of R₉ and R₁₀, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl- R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group, and the other one of R₉ and R₁₀, if present, is selected from the group consisting of H, OH, SH, NH₂ and halogen; one of Rₑ and R_{f}, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl- R^{P}, O—R^{O}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group; and the other one of Rₑ and R_{f}, if present, is selected from the group consisting of H, OH, SH, NH₂ and halogen;
m is selected from an integer ranging from 0 to 10, with the proviso that (i) if m is 0, one of n₂ and n₅ is 1 and the other one of n₂ and n₅ is 0, (ii) if m is 1, (a) n₂, n₅ and n₈ are 0 or (b) one of n₂, n₅ and n₈ is 1 and the other two of n₂, n₅ and n₅ are 0, and (iii) if m is an integer ranging from 2 to 10, (a) n₂, n₅ and each n₈ are 0, (b) one of n₂ and n₅ is 1 and the other one of n₂ and n₅ as well as each n₈ are 0, or (c) one n₅ is 1 and each other n₈ as well as n₂ and n₈ are 0;
n₁₀ is selected from 0, 1 or 2;
L₁ is selected from the group consisting of CH₂, O, S, SO, SO₂, NR^{L}, CH(OH), CH(SH) and CH(halogen), with the proviso that, if m is 1 and n₂, n₅ and n₈ are 0, L₁ is selected from the group consisting of O, S, SO, SO₂ and NR^{L};
L₂ is selected from the group consisting of O, S, SO, SO₂ and NH; wherein, if m is greater than 1, L₂ may be same or different in each repeating unit;
each of X₁ through X₈ is independently O, S, NH or CH₂, wherein, if n₁₀ is 2, X₃ may be same or different in each repeating unit;
each of Y₁ through Y₅ is independently 0, S or Se, wherein, if n₁₀ is 2, Y₂ may be same or different in each repeating unit;
each of Z₁ through Z₅ is independently OH, SH, SeH or BH₃, wherein, if n₁₀ is 2, Z₂ may be same or different in each repeating unit;
R₁₄ is (i) selected from the group consisting of H, OH, OC₁-C₃-alkyl, and Opropargyl, wherein the dashed methylene bridge between R₁₄ and the 4′ C is absent, or (ii) O, wherein the dashed methylene bridge between R₁₄ being O and the 4′ C is present;
R₁₆ is (i) selected from the group consisting of H, OH, OC₁-C₃-alkyl, and Opropargyl, wherein the dashed methylene bridge between R₁₆ and the 4′ C is absent, or (ii) O, wherein the dashed methylene bridge between R₁₆ being O and the 4′ C is present;
R^{L} is (i) H, if one of n₂, n₅ and n₈ is 1, or (ii) selected from the group consisting of H, C₁-C₃-alkyl, C₁-C₃-alkyl-O—R^{O}, C₁-C₃-alkyl-S—R^{S}, C₁-C₃-alkyl-NR^{N1}R^{N2}, C₁-C₃-alkyl-R^{P} and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized, and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, if n₂, n₅ and n₈ are 0;
R^{O}, R^{S}, R^{N1} and R^{N2} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl- O—C₁-C₄alkyl-O—C₁-C₄alkyl, C₁-C₄alkyl-S—C₁-C₄alkyl, C₁-C₄alkyl-S—C₁-C₄alkyl-S—C₇-C₄alkyl, C₁-C₄alkyl-S—C₁- C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl- NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{D} and R^{E} are each independently selected from the group consisting of C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl- OC₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁- C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{N3} and R^{N4} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl- NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, with the proviso that R^{N3} and R^{N4} are not both H;
R^{P} is

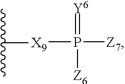

wherein
X₉ is O, S, NH or CH₂;
Y₆ is O, S or Se; and
each of Z₆ and Z₇ is independently OH, SH, SeH or BH₃; and
each of ring B₂ through ring B₄ is independently a nucleobase, a modified nucleobase or a nucleobase analog;
wherein the process comprises reacting
a compound of formula (IV)

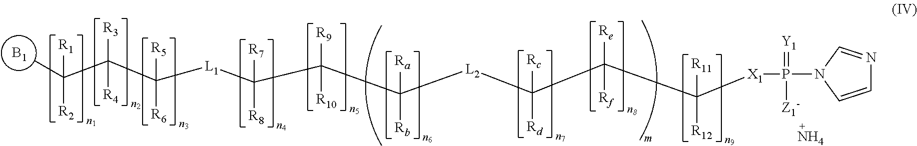

wherein B₁, R₁, R₂, R₃, R₄, R₅, R₆, R₇, R₈, R₉, R₁₀, R₁₁, R₁₂, Rₐ, R_{b}, R_{c}, R_{d}, Rₑ, R_{f}, n₁, n₂, n₃, n₄, n₅, n₆, n₇, n₈, n₉, m, L₁, L₂, X₁, Y₁, Z₁ are as defined above for formula (I);
with a compound of formula (V)

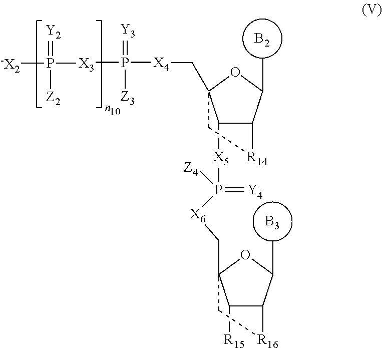

wherein X₂, Y₂, Z₂, X₃, Y₃, Z₃, X₄, Y₄, Z₄, X₅, X₆, R₁₄, R₁₅, R₁₆, B₂, and B₃ are as defined above for formula (I).

It has surprisingly been found that the activation of the dinucleotide or trinucleotide of formula (V) to form an activated dinucleotide or trinucleotide in order to react with an inactivated B₁-linker moiety of formula (VI) as shown below led to undesired intramolecular side reactions of the activated dinucleotide or activated trinucleotide.

However, it has been found that the compound of formula (I) can be prepared by activating the B₁-linker moiety (formula VI) with imidazole and reacting the activated B₁-linker moiety of formula (IV) with an inactivated dinucleotide or trinucleotide. This allows for easy condensation of the inactivated dinucleotide or trinucleotide with various different B₁-linker moieties.

In a preferred embodiment of the nineteenth aspect, the reaction is performed in the presence of a metal chloride, preferably zinc chloride, manganese chloride or magnesium chloride, more preferably magnesium chloride. Preferably, the metal chloride is used in excess compared to the compound of formula (IV), wherein an excess refers to at least 5 equivalents, preferably at least 10 equivalents compared to the compound of formula (IV). Performing the reaction in the presence of magnesium chloride increases the yield of the product.

In another preferred embodiment of the nineteenth aspect, the reaction is performed in an aqueous solution and/or an organic solvent, preferably in a mixture of water and acetonitrile or in a mixture of water and N-methylmorpholine.

In another preferred embodiment of the nineteenth aspect, the compound of formula (IV) is reacted with the compound of formula (V) in equimolar amounts.

In another preferred embodiment of the nineteenth aspect, the product is desalted and purified by reverse-phase HPLC.

In another preferred embodiment of the nineteenth aspect, the process further comprises preparing the compound of formula (IV) comprising reacting a compound of formula (VI)

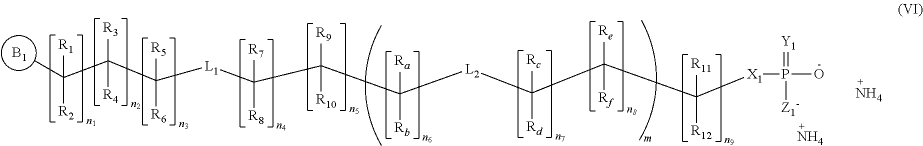

wherein B₁, R₁, R₂, R₃, R₄, R₅, R₆, R₇, R₈, R₉, R₁₀, R₁₁, R₁₂, Rₐ, R_{b}, R_{c}, R_{d}, Rₑ, R_{f}, n₁, n₂, n₃, n₄, n₅, n₆, n₇, n₈, n₉, m, L₁, L₂, X₁, Y₁, Z₁ are as defined above for formula (IV); with carbonyldiimidazole.

It has surprisingly been found that using imidazole together with PPh3 and dipyridyldisulfide was not suitable for imidazole activation of the compound of formula (VI). However, using carbonyldiimidazole allows for imidazole activation of the compound of formula (VI) forming the compound of formula (IV). In particular, performing the reaction in DMSO provides high yields within 24-72 h, while performing the reaction in DMF is more slowly.

Thus, in another preferred embodiment of the nineteenth aspect, the reaction of a compound of formula (VI) with carbonyldiimidazole is performed in DMSO.

In another preferred embodiment of the nineteenth aspect, the compound of formula (VI) is reacted with an excess of carbonyldiimidazole. Using an excess of carbonyldiimidazole increases the yield of the compound of formula (IV). Preferably, the compound of formula (VI) is reacted with an excess of carbonyldiimidazole, wherein the excess refers to 2 to 40 equivalents, more preferably 10 to 25 equivalents, even more preferably 20 to 30 equivalents of carbonyldiimidazole relative to the compound of formula (VI). The excess of carbonyldiimidazole is preferably quenched after the reaction is finished.

In another preferred embodiment of the nineteenth aspect, excess carbonyldiimidazole is quenched with water. Surprisingly it has been found that quenching excess carbonyldiimidazole with water provides the desired product. In contrast, quenching excess carbonyldiimidazole with methanol does not lead to an observable product formation.

In one embodiment of the nineteenth aspect, the compound of formula (I), formula (IV), and formula (VI) comprises a linear unbranched structure, wherein m is selected from an integer ranging from 1 to 10, preferably 1, n₂, n₅ and each n₈ are 0, and L₁ is selected from the group consisting of O, S, SO, SO₂ and NR^{L}, preferably L₁ is O.

In another embodiment of the nineteenth aspect, the compound of formula (I), formula (IV), and formula (VI) comprises a linear single-branched structure, wherein
(i) m is 0, one of n₂ and n₅ is 1, and the other one of n₂ and n₅ is 0; (ii) m is 1, one of n₂, n₅ and n₈ is 1, and the other two of n₂, n₅ and n₈ are 0, (iii) m is an integer ranging from 2 to 10, one of n₂ and n₅ is 1 and the other one of n₂ and n₅ as well as each n₈ are 0, or (iv) m is an integer ranging from 2 to 10, one n₄ is 1 and each other n₈ as well as n₂ and n₅ are 0; and
L₁ is selected from the group consisting of CH₂, O, S, SO, SO₂, NR^{L}, CH(OH), CH(SH) and CH(halogen), preferably L₁ is CH₂ or O.

In a preferred embodiment thereof, the compound of formula (I), formula (IV), and formula (VI) comprises a linear single-branched structure, wherein m is 0, one of n₂ and n₅ is 1, and the other one of n₂ and n₅ is 0.

In one preferred embodiment, n₁₀ is 1;
each of n₁ and n₉ is independently selected from 1 or 2, preferably n₁ is 1 and n₉ is 1;
each of n₃, n₄, and, if present, each of n₆ and n₇, is independently selected from 0 or 1;
R₁₄ is H or OC₁-C₃-alkyl, wherein the dashed methylene bridge between R₆ and the 4′ C is absent, preferably wherein R₁₄ is OCH₃;
R₁₆ is H, OH, or OC₁-C₃-alkyl, wherein the dashed methylene bridge between R₃ and the 4′ C is absent, preferably wherein R₁₆ is OH,
R₁₅ is OH;
X₁ is CH₂ or O, and each of X₂ through X₆ is O;
each of Y₁ through Y₄ is O; and
each of Z₁ through Z₄ is OH.

In another preferred embodiment, one of R₃ and R₄ if present, one of R₉ and R₁₀ if present, or one of Rₑ and R_{f} if present, is selected from the group consisting of C₁-alkyl-O—R^{O}, C₁-alkyl-S—R^{S}, C₁-alkyl-S—S—R^{S}, C₁-alkyl-R^{P}; wherein
R^{O} and R^{S} are each independently selected from the group consisting of H, C₁-C₄-alkyl, C₁-C₂-cyanoalkyl, and 5- or 6-membered saturated, partially or fully unsaturated, or aromatic carbocyclyl, carbocyclyl-C₁-alkyl, heterocyclyl, or heterocyclyl-C₁-alkyl, wherein the aforementioned heterocyclic rings comprise one or two, same or different heteroatoms selected from O, N, or S, wherein said N- and/or S-atoms are independently oxidized or non-oxidized; and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₂-alkyl, preferably halogen;
R^{P} is

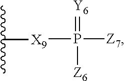

wherein
X₉ is O; Y₆ is O or S; and each of Z₆ and Z₇ is OH; and
the other one of R₃ and R₄ if present, R₉ and R₁₀ if present, Rₑ and R_{f} if present, is H; and
wherein preferably each of R₁, R₂, R₅, R₆, R₇, R₈, Rₐ, R_{b}, R_{c}, R_{d}, R₁₁, and R₁₂, if present, is H.

In yet another preferred embodiment, ring B₁ is a modified guanine or a guanine analog and/or each of ring B₂ through ring B₄ is a nucleobase.

In a preferred embodiment of the nineteenth aspect, the preferred embodiments disclosed in connection with formula (I) of the first aspect also apply to formula (I), formula (IV), formula (V), and formula (VI) of the nineteenth aspect.

It is understood that the process for preparing a compound of formula (I) of the nineteenth aspect is suitable for preparing all embodiments outlined above in the first aspect. Thus, the nineteenth aspect may also be formulated as a process for preparing a compound of formula (I) as defined above in the present aspect and/or all embodiments of formula (I) as described above in the first aspect.

### BRIEF DESCRIPTION OF THE DRAWINGS

FIG. 1 shows the structures of exemplary compounds obtained and obtainable by the synthesis route I described in examples 1.1.

FIG. 2 shows the structures of exemplary compounds obtained and obtainable by the synthesis route II described in example 1.2.

FIG. 3 shows the structures of exemplary compounds obtained and obtainable by the synthesis route Ill described in example 1.3.

FIG. 4 shows the structures of exemplary compounds obtained and obtainable by the synthesis route IV described in example 1.4.

FIG. 5 shows the structures of exemplary compounds obtained and obtainable by the synthesis route VI described in example 1.6.

FIG. 6 shows the structures of exemplary compounds obtained and obtainable by the synthesis route VII described in example 1.7.

FIG. 7 shows PpLuc expression of 50 ng Ganciclovir-linked (compound 19) and Ganciclovir-phosphate-linked (compound 18) capped mRNA and an untransfected negative control in HeLa (unfilled bars) and HDF cells (striped bars) (see Examples 2 for details as regards the preparation of the mRNAs and Example 5 for further details as regards the cellular assay).

FIG. 8 shows the structures of exemplary compounds obtained and obtainable by the synthesis route I″ described in example 1.1.2

FIG. 9 shows the structures of exemplary compounds obtained and obtainable by the synthesis route VIII described in example 1.8.

FIG. 10 shows the structures of exemplary compounds obtained and obtainable by the synthesis route IX and the synthesis route X described in example 1.9 and example 1.10 respectively.

FIG. 11 shows the structures of exemplary compounds obtained and obtainable by the synthesis route XI described in example 1.11.

FIG. 12 shows the structures of exemplary compounds obtained and obtainable by the synthesis route XII and the synthesis route XIII described in example 1.12 and example 1.13 respectively.

FIG. 13 shows the structures of exemplary compounds obtained and obtainable by the synthesis route XIV and the synthesis route XV described in example 1.14 and example 1.15 respectively.

FIG. 14A shows PpLuc expression in HDF cells of 50 ng m7G-TriEG Cap0 (mRNA(xv)) and Cap1 (mRNA(xiv)) as well as 3′-SPhe-m7-Gancyclovir Cap0 (mRNA(vii)) and Cap1 (mRNA(vi)) capped mRNA (see Examples 2 for details as regards the preparation of the mRNAs and Example 6 for further details as regards the cellular assay).

FIG. 14B shows PpLuc expression in HDF cells of 50 ng m7G-TriEG Cap1 (mRNA(xiv)), 3′-SPhe-m7-Gancyclovir Cap1 (mRNA(vi)) and M7 Ganciclovir linked Cap1 (mRNA(viii)) capped mRNA in comparison to CleanCap (mRNA(xvi)) capped mRNA as a control (see Examples 2 for details as regards the preparation of the mRNAs and Example 6 for further details as regards the cellular assay).

FIG. 15A shows PpLuc expression in HDF cells of 50 ng Ethylene glycol Cap0 (mRNA(xix)), Diethylene glycol Cap0 (mRNA(xx)) as well as Cap1 (mRNA(xxi)) and m7G-TriEG Cap1 (mRNA(xiv)) capped mRNA (see Examples 2 for details as regards the preparation of the mRNAs and Example 7 for further details as regards the cellular assay).

FIG. 15B shows PpLuc expression in HDF cells of 50 ng Diethylene glycol Cap1 (mRNA(xxi)) and m7G-TriEG Cap1 (mRNA(xiv)) capped mRNA in comparison to CleanCap (mRNA(xvi)) capped mRNA as control (see Examples 2 for details as regards the preparation of the mRNAs and Example 7 for further details as regards the cellular assay).

FIG. 16 shows PpLuc expression in HDF cells of 50 ng mCap Cap0 (mRNA(i)), UNA Cap0 (mRNA(xvii)), m7G-TriEG Cap1 (mRNA(xiv)) and 3′-SPhe-m7-Ganciclovir Cap1 (mRNA(vi)) capped mRNA in comparison (see Examples 2 for details as regards the preparation of the mRNAs and Example 8 for further details as regards the cellular assay).

### DETAILED DESCRIPTION OF THE INVENTION

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

### Definitions

For the sake of clarity and readability, the following scientific background information and definitions are provided.

Any technical features mentioned herein or disclosed thereby can be part of or may be read on each and every embodiment of the invention. Additional definitions and explanations can be provided in the context of this disclosure.

As used in the specification and the claims, the singular form is of “a” and “an” also include the corresponding plurals unless the context clearly dictates otherwise. The same applies for plural forms used herein, which also include the singular forms unless the context clearly dictates otherwise.

The terms “about” and “approximately” in the context of the present invention denotes an interval of accuracy that a person skilled in the art will understand to still ensure the technical effect of the feature in question. The term typically indicates a deviation from the indicated numerical value of ±10% and preferably ±5%.

It needs to be understood that the term “comprising” is not limiting. For the purposes of the present invention, the term “consisting of” is considered to be a preferred embodiment of the term “comprising of”. If hereinafter a group is defined to comprise at least a certain number of embodiments, this is also meant to encompass a group, which preferably consists of these embodiments only.

The term “compound(s) of the present invention” is to be understood as equivalent to the terms “compound(s) according to the invention” and “compound(s) of the present application, and also covers a salt, stereoisomer, tautomer or N-oxide thereof.

The compounds according to the invention may be amorphous or may exist in one or more different crystalline states (polymorphs), which may have different macroscopic properties such as stability or show different biological properties such as activities. The present invention relates to amorphous and crystalline forms of compounds of formula (I), mixtures of different crystalline states of the compounds of formula (I), as well as amorphous or crystalline salts thereof.

The compounds according to the invention may be present in the form of salts. For example, the groups Z₁ through Z₅ if representing OH may typically be present in deprotonated form, i.e. as “O” carrying a negative charge. At the same time, the nucleobases, modified nucleobases or a nucleobase analogs may, e.g., be present positively charged form. In particular, the group B₁ may, e.g., carry a positive charge, if B₁ represents N⁷-methylguanine. In addition, positively charged counterions may be present, such that pharmaceutically acceptable salts of the compounds according to the invention are formed.

Salts of the compounds according to the invention are preferably pharmaceutically acceptable salts, such as those containing counterions present in drug products listed in the US FDA Orange Book database. They can be formed in a customary manner, e.g., by reacting the compound with an acid of the anion in question, if the compounds according to the invention have a basic functionality, or by reacting acidic compounds according to the invention with a suitable base.

Suitable cationic counterions are in particular the ions of the alkali metals, preferably lithium, sodium and potassium, of the alkaline earth metals, preferably calcium, magnesium and barium, and of the transition metals, preferably manganese, copper, silver, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethyl-ammonium, bis(2-hydroxyethyl)ammonium, benzyltrimethylammonium and benzyltriethylammonium, furthermore the cations of 1,4-piperazine, meglumine, benzathine and lysine.

Suitable anionic counterions are in particular chloride, bromide, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate, furthermore lactate, gluconate, and the anions of poly acids such as succinate, oxalate, maleate, fumarate, malate, tartrate and citrate, furthermore sulfonate anions such as besylate (benzenesulfonate), tosylate (p-toluenesulfonate), napsylate (naphthalene-2-sulfonate), mesylate (methanesulfonate), esylate (ethanesulfonate), and ethanedisulfonate. They can be formed by reacting compounds according to the invention that have a basic functionality with an acid of the corresponding anion.

Suitable counterions may also be introduced by applying ion exchange chromatography and/or using suitable buffers.

If the compounds according to the invention are present in the form of salts, the compounds themselves may contain positive and negative charges, and, in addition, counterions may be present for charge neutrality. For example, the groups Z₁ through Z₅, if representing OH, may typically be present in deprotonated form, i.e. as “O” carrying a negative charge. At the same time, the nucleobases, modified nucleobases or a nucleobase analogs may, e.g., be present positively charged form. In particular, the group B₁ may, e.g., carry a positive charge, if B₁ represents N⁷-methylguanine, due to the attachment to the remainder of the molecule. In addition, positively charged counterions may be present, such that pharmaceutically acceptable salts of the compounds according to the invention are formed.

It is to be understood that also the precursors of the molecules may be present in charged as well as in non-charged form.

Depending on the substitution pattern, the compounds according to the invention may have one or more centers of chirality, including axial chirality. The invention provides both, pure enantiomers or pure diastereomers, of the compounds according to the invention, and their mixtures, including racemic mixtures. Suitable compounds according to the invention also include all possible geometrical stereoisomers (cis/trans isomers or E/Z isomers) and mixtures thereof. E/Z-isomers may be present with respect to, e.g., an alkene, carbon-nitrogen double-bond or amide group.

Tautomers may be formed, if a substituent is present at the compound of formula (I), which allows for the formation of tautomers such as keto-enol tautomers, imine-enamine tautomers, amide-imidic acid tautomers or the like.

The term “is deuterated” as used herein means that at least one of the hydrogen atoms occurring in the respective moiety is replaced by deuterium. Thus, if e.g. a nucleoside is deuterated, at least one of the hydrogen atoms occurring in the sugar and the nucleobase of the nucleoside is replaced by deuterium. The deuteration of a respective moiety may be partial in the sense that one or more but not all hydrogen atoms occurring in the respective moiety is/are replaced by deuterium. The afore-mentioned definition also applies to the term “a deuterated version thereof”, which refers to a compound of a given structure (such as e.g. the structure of formula (I) of the present application), and wherein at least one of the hydrogen atoms occurring in this given structure is replaced by deuterium.

The term “deuterium” as used herein has its common meaning, i.e. it is an isotope of hydrogen. In comparison with hydrogen, deuterium has an additional neutron. It is also referred to as “hydrogen-2” or “heavy hydrogen” and is abbreviated as “D” or “²H”.

A deuteration may have a positive impact, such as e.g. a reduced immunogenicity and/or enhanced expression of an RNA (see WO 2019/158583 for details) or e.g. an increased resistance to thermal and enzymatic hydrolysis (see WO 2022/099411 for details).

The term “substituted”, as used herein, means that a hydrogen atom bonded to a designated atom is replaced with a specified substituent, provided that the substitution results in a stable or chemically feasible compound.

Unless otherwise indicated, a substituted atom may have one or more substituents and each substituent is independently selected.

The term “substitutable”, when used in reference to a designated atom, means that attached to the atom is a hydrogen, which can be replaced with a suitable substituent.

When it is referred to certain atoms being substituted with “one or more” substituents, the term “one or more” is intended to cover at least one substituent, e.g. 1 to 3 substituents, preferably 1 or 2 substituents, more preferably 1 substituent. When neither the term “unsubstituted” nor “substituted” is explicitly mentioned concerning a moiety, said moiety is to be considered as unsubstituted.

The organic moieties mentioned in the above definitions of the variables are—like the term halogen—collective terms for individual listings of the individual group members. The prefix Cn-Cm indicates in each case the possible number of carbon atoms in the group.

The term “halogen” denotes in each case fluorine, bromine, chlorine or iodine, in particular fluorine, chlorine, or bromine.

The term “alkyl” as used herein denotes in each case a straight-chain or branched alkyl group having usually from 1 to 6 carbon atoms, preferably 1 to 5 or 1 to 4 carbon atoms, more preferably 1 to 3 or 1 or 2 carbon atoms.

Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl.

The term “alkenyl” as used herein denotes in each case an unsaturated hydrocarbon group having usually 2 to 4 carbon atoms comprising at least one carbon-carbon double bond in any position, e.g. vinyl (ethenyl), allyl (2-propen-1-yl), 1-propen-1-yl, 2-propen-2-yl, methallyl (2-methylprop-2-en-1-yl), 2-buten-1-yl, 3-buten-1-yl, and the like. If geometric isomers are possible with regard to the double bond, the present invention relates to both, the E- and Z-isomers. The bonding of vinyl is exemplified below:

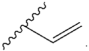 [SMILES: *C=C]

The term “alkinyl” as used herein denotes in each case an unsaturated hydrocarbon group having usually 2 to 4 carbon atoms comprising at least one carbon-carbon triple bond in any position.

The term “haloalkyl” as used herein denotes in each case a straight-chain or branched alkyl group having usually from 1 to 4 carbon atoms, preferably 1 to 3 or 1 or 2 carbon atoms, wherein the hydrogen atoms of this group are partially or totally replaced with halogen atoms. Preferred haloalkyl moieties are selected from C₁-C₄-haloalkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “cyanoalkyl” as used herein denotes in each case a straight-chain or branched alkyl group having usually from 1 to 4 carbon atoms, preferably 1 to 2 carbon atoms, wherein one of the hydrogen atoms of this group is replaced with a cyano group, i.e. a CN group, wherein the CN is attached to the alkyl group via the carbon atom of the CN group. Preferred cyanoalkyl groups are 1-cyanoethyl, 2-cyanoethyl, 1-cyanopropyl, 2-cyanopropyl, 3-cyanopropyl, more preferably 2-cyanoethyl.

The term “carbocyclic”, “carbocyclyl”, or “carbocycle” as used herein includes, unless otherwise indicated, in general a 3- to 10-membered monocyclic or bicyclic ring, preferably a 4- to 8-membered or a 3- to 6-membered or a 5- to 7-membered monocyclic ring, more preferably a 3-, 4-, 5- or 6-membered monocyclic ring, most preferably a 6-membered monocyclic ring, comprising 3 to 10, preferably 4 to 8 or 3 to 6 or 5 to 7, more preferably 3, 4, 5 or 6, most preferably 6 carbon atoms. The carbocycle may be saturated, partially or fully unsaturated, or aromatic, wherein saturated means that only single bonds are present, and partially or fully unsaturated means that one or more double bonds may be present in suitable positions, while the Hückel rule for aromaticity is not fulfilled, whereas aromatic means that the Hückel (4n+2) rule is fulfilled. Also “aryls” are covered by the term “carbocycles”. The term “aryl” or “aromatic carbocycle” refers to aromatic carbocyclic rings based on carbon atoms as ring members, preferably 6-membered aromatic carbocyclic rings based on carbon atoms as ring members. A preferred example is phenyl. Unless otherwise indicated, the term “aryl” further covers “aromatic carbobicycles” as defined herein. The term “carbocyclic” or “carbocyclyl”, unless otherwise indicated, may therefore cover inter alia cycloalkyl, cycloalkenyl, as well as phenyl. Preferably, the term “carbocyclic” or “carbocyclyl” refers to phenyl and cycloalkyl, for example phenyl, cyclopropyl, cyclobutyl, cyclopentyl, and cyclohexyl, more preferably phenyl and cyclohexyl.

The term “carbocyclylalkyl” as used herein, refers to carbocyclyl as defined herein, which is bonded to the remainder of the molecule via an alkyl group having usually from 1 to 3 carbon atoms, preferably 1 or 2 carbon atoms, more preferably 1 carbon atom. For example, carbocyclyl-C₁-alkyl refers to carbocyclyl as defined herein, which is bonded to the remainder of the molecule via a methyl group. Preferably, the term “carbocyclylalkyl” refers to phenylalkyl or cycloalkylalkyl, which refers to phenyl or cycloalkyl groups being bonded to the remainder of the molecule via an alkyl group. Examples of carbocyclylalkyl include benzyl (i.e. phenylmethyl), phenylethyl, cyclopropylmethyl, cyclopropylethyl, cyclobutylmethyl, cyclobutylethyl, cyclopentylmethyl, cyclopentylethyl, cyclohexylmethyl, cyclohexylethyl. Preferred examples are benzyl (i.e. phenylmethyl) and cyclohexylmethyl.

The term “heterocyclic” or “heterocyclyl” as used herein includes, unless otherwise indicated, in general a 3- to 10-membered, preferably a 4- to 8-membered or 5- to 7-membered, more preferably 5- or 6-membered, in particular 6-membered monocyclic ring. The heterocycle may be saturated, partially or fully unsaturated, or aromatic, wherein saturated means that only single bonds are present, and partially or fully unsaturated means that one or more double bonds may be present in suitable positions, while the Hückel rule for aromaticity is not fulfilled, whereas aromatic means that the Hückel (4n+2) rule is fulfilled. The heterocycle typically comprises one or more, e.g. 1, 2, 3, or 4, preferably 1, 2, or 3, more preferably 1 or 2, most preferably 1 heteroatoms selected from N, O and S as ring members, where S-atoms as ring members may be present as S, SO or SO₂. The remaining ring members are carbon atoms. The skilled person is aware that S, SO or SO₂ is to be understood as follows:

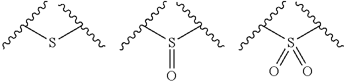

Also “heteroaryls” are covered by the term “heterocycle”. The term “hetaryl” or “heteroaryl” or “aromatic heterocycle” or “aromatic heterocyclic ring” includes monocyclic 5- or 6-membered, preferably 6-membered, aromatic heterocycles comprising as ring members 1, 2, 3 or 4, preferably 1 or 2, more preferably 1, heteroatoms selected from N, O and S, where S-atoms as ring members may be present as S, SO or SO₂. The term “heterocyclic” or “heterocyclyl”, unless otherwise indicated, may therefore cover inter alia saturated heterocycles such as pyrrolidine, tetrahydrothiophene, tetrahydrofuran, piperidine, tetrahydropyran, dioxane, morpholine or piperazine and aromatic heterocycles such as pyridyl (also referred to as pyridinyl), i.e. 2-, 3-, or 4-pyridyl, pyrimidinyl, i.e. 2-, 4- or 5-pyrimidinyl, pyrazinyl, pyridazinyl, i.e. 3- or 4-pyridazinyl, thienyl, i.e. 2- or 3-thienyl, furyl, i.e. 2- or 3-furyl, pyrrolyl, i.e. 2- or 3-pyrrolyl, oxazolyl, i.e. 2-, 3- or 5-oxazolyl, isoxazolyl, i.e. 3-, 4- or 5-isoxazolyl, thiazolyl, i.e. 2-, 3- or 5-thiazolyl, isothiazolyl, i.e. 3-, 4- or 5-isothiazolyl, pyrazolyl, i.e. 1-, 3-, 4- or 5-pyrazolyl, i.e. 1-, 2-, 4- or 5-imidazolyl, oxadiazolyl, e.g. 2- or 5-[1,3,4]oxadiazolyl, 4- or 5-(1,2,3-oxadiazol)yl, 3- or 5-(1,2,4-oxadiazol)yl, 2- or 5-(1,3,4-thiadiazol)yl, thiadiazolyl, e.g. 2- or 5-(1,3,4-thiadiazol)yl, 4- or 5-(1,2,3-thiadiazol)yl, 3- or 5-(1,2,4-thiadiazol)yl, triazolyl, e.g. 1H-, 2H- or 3H-1,2,3-triazol-4-yl, 2H-triazol-3-yl, 1H-, 2H-, or 4H-1,2,4-triazolyl and tetrazolyl, i.e. 1H- or 2H-tetrazolyl, preferably a 5- or 6-membered aromatic heterocycle comprising one or more, e.g. 1, 2, 3, or 4, preferably 1, 2, or 3 heteroatoms selected from N, O and S as ring members, where S-atoms as ring members may be present as S, SO or SO₂. Preferably, the term “heterocyclic” or “heterocyclyl” refers to a 6-membered aromatic heterocycle comprising as ring members 1 or 2, preferably 1, heteroatoms selected from N, O, and S, preferably N, more preferably to pyridinyl. Further, a skilled person is aware that resonance structures of the oxidized forms may be possible.

The term “heterocyclylalkyl” as used herein, refers to heterocyclyl as defined herein, which is bonded to the remainder of the molecule via an alkyl group having usually from 1 to 3 carbon atoms, preferably 1 or 2 carbon atoms, more preferably 1 carbon atom. For example, heterocyclyl-C₁-alkyl refers to heterocyclyl as defined herein, which is bonded to the remainder of the molecule via a methyl group. Examples of heterocyclylalkyl include pyridinylmethyl, pyrimidinylmethyl, pyrazolylmethyl, and piperidinylmethyl.

The term “nucleic acid” means any compound comprising, or preferably consisting of, DNA or RNA. The term may be used for a polynucleotide and/or oligonucleotide.

The term “DNA” is the usual abbreviation for deoxyribonucleic acid. It is a nucleic acid molecule, i.e. a polymer consisting of nucleotide monomers. These nucleotides are usually deoxy-adenosine-monophosphate, deoxy-thymidine-monophosphate, deoxy-guanosine-monophosphate and deoxy-cytidine-monophosphate monomers or analogs thereof which are—by themselves—composed of a sugar moiety (deoxyribose), a base moiety and a phosphate moiety, and polymerize by a characteristic backbone structure. The backbone structure is, typically, formed by phosphodiester bonds between the sugar moiety of the nucleotide, i.e. deoxyribose, of a first and a phosphate moiety of a second, adjacent monomer. The specific order of the monomers, i.e. the order of the bases linked to the sugar/phosphate-backbone, is called the DNA-sequence. DNA may be single stranded or double stranded. In the double stranded form, the nucleotides of the first strand typically hybridize with the nucleotides of the second strand, e.g. by A/T-base-pairing and G/C-base-pairing.

The term “RNA” is the usual abbreviation for ribonucleic acid. It is A nucleic acid molecule, i.e. a polymer consisting of nucleotide monomers. These nucleotides are usually adenosine-monophosphate (AMP), uridine-monophosphate (UMP), guanosine-monophosphate (GMP) and cytidine-monophosphate (CMP) monomers or analogs thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. RNA can be obtained by transcription of a DNA sequence, e.g., inside a cell. In eukaryotic cells, transcription is typically performed inside the nucleus or the mitochondria. In vivo, transcription of DNA usually results in the so-called premature RNA which has to be processed into so-called messenger-RNA, usually abbreviated as mRNA. Processing of the premature RNA, e.g. in eukaryotic organisms, comprises a variety of different posttranscriptional modifications such as splicing, 5-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5′-cap, optionally a 5′UTR, a coding sequence, optionally a 3′UTR and a poly(A) sequence. If RNA molecules are of synthetic origin, as in the present application, the RNA molecules are meant not to be produced in vivo, i.e. inside a cell or purified from a cell, but in an in vitro method. An examples for a suitable in vitro method is in vitro transcription. In addition to messenger RNA, several non-coding types of RNA exist which may be involved in regulation of transcription and/or translation, and immunostimulation and which may also be produced by in vitro transcription. The term “RNA” further encompasses RNA molecules, such as viral RNA, retroviral RNA and replicon RNA, small interfering RNA (siRNA), antisense RNA, saRNA (small activating RNA), CRISPR RNA (small guide RNA, sgRNA), ribozymes, aptamers, riboswitches, immunostimulating RNA, transfer RNA (tRNA), ribosomal RNA (rRNA), transfer-messenger RNA (tmRNA), small nuclear RNA (snRNA), small nucleolar RNA (snoRNA), microRNA (miRNA), and Piwi-interacting RNA (piRNA). A particularly preferred RNA molecule of the present invention is selected from the group consisting of mRNA, snRNA, snoRNA, tRNA, rRNA and tmRNA.

As noted above, in eukaryotic cells, a “cap structure” is typically found at the 5′ end of mRNAs, wherein this cap structure comprises of a “cap” nucleoside being 7-methylguanosine and a triphosphate bridge, wherein the triphosphate bridge forms a 5′ to 5′ triphosphate linkage between the 5′ position of the ribose of the 7-methylguanosine and the 5′ position of the ribose of the first regular nucleotide of the mRNA. The cap structure facilitates translation or localization and/or prevents degradation of the mRNA when present at the 5′ end. If the ribose of the first and second nucleotide following the cap structure is not modified, this structure is referred to as “cap0 structure”. If the ribose of the first nucleotide following the cap structure carries an OCH₃ substituent at the 2′ position, the structure is referred to as “Cap1 structure”. Finally, if the riboses of the first and second nucleotide following the cap structure carry an OCH₃ substituent at the 2′ position, the structure is referred to as “Cap2 structure”; and if only the ribose of the second nucleotide following the cap structure carries an OCH₃ substituent at the 2′ position (and the ribose of the first nucleotide following the cap structure carries on OH substituent at the 2′ position), the structure is referred to as “Cap2-1” structure. The cap structure (alternatively referred to as m⁷G5′ppp5′N) can be depicted as follows, wherein this structure may more particularly be referred to as Cap2 structure:

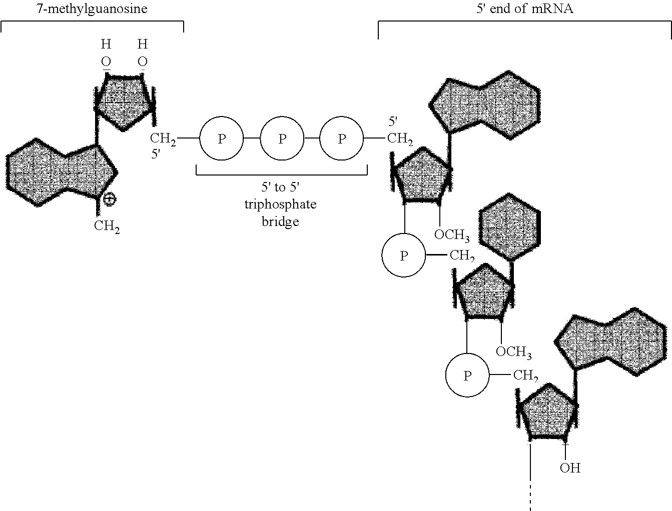

As outlined above, such cap structures can be achieved co-transcriptionally in in vitro transcription assays when using a “cap analog” in the in vitro transcription reaction. Accordingly, a “cap analog” may also be referred to as “cap analog for initiating RNA in vitro transcription”.

A variety of cap analogs has been developed and is commercially available for use in in vitro transcription reactions. Such cap analogs typically have structures corresponding to or mimicking a dinucleotide (also referred to as “cap0 analog”), a trinucleotide, where the ribose of the second nucleotide typically carries an OCH₃ substituent at the 2′ position (also referred to as “Cap1 analog”) or a tetranucleotide, where the riboses of the second and third nucleotide typically carry a OCH₃ substituent at the 2′ position (also referred to as “Cap2 analog”). All cap analogs have in common that they comprise 7-methylguanosine or an analog thereof at the position, where the 7-methylguanosine is found in a natural cap structure (which is at the 5′ terminal position of the cap analog). Accordingly, if a 7-methylguanosine analog is used, this analog is used to mimic the natural 7-methylguanosine, and it is found at the position of the 7-methylguanosine. If an analog of 7-methylguanosine is used in a cap analog, the 7-methylguanosine analog comprises either (i) a ribose or (ii) a cyclic structure different from a ribose or (iii) a linear branched structure (mimicking the ribose) at the position, where a ribose is found in 7-methylguanosine. Examples of such cap analogs are shown in the following, wherein the ribose, cyclic structure or linear branched structure is encircled (the definitions of the specific substituents depicted in the following can be taken from the patent reference as indicated):
(i) The cap analog of WO 2009/149253, in particular the cap analog of claim 1 of WO 2009/149253 with the following structure:

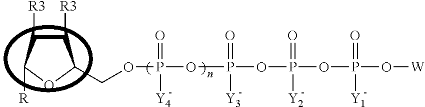

(ii) The cap analog of WO 2017/066781, in particular the cap analog of claim 1 of WO 2017/066781 with the following structure:

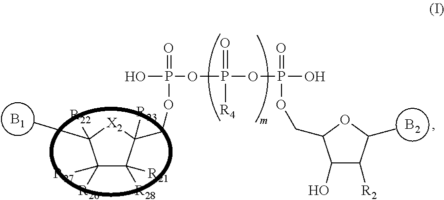

(iii) The cap analog of WO 2017/066782, in particular the cap analog of claim 1 of WO 2017/066782 with the following structure:

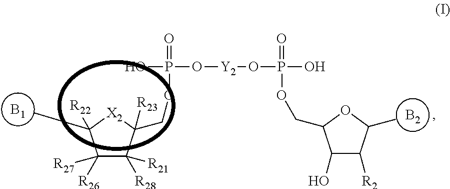

(iv) The cap analog of WO 2017/066789, in particular the cap analog of claim 1 of WO 2017/066789 with the following structure:

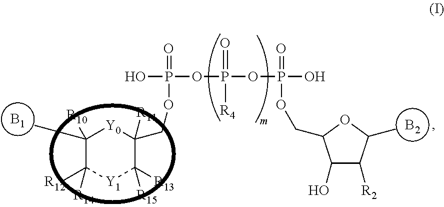

(v) The cap analog of WO 2017/053297, in particular the cap analog of claim 1 of WO 2017/053297 with the following structure:

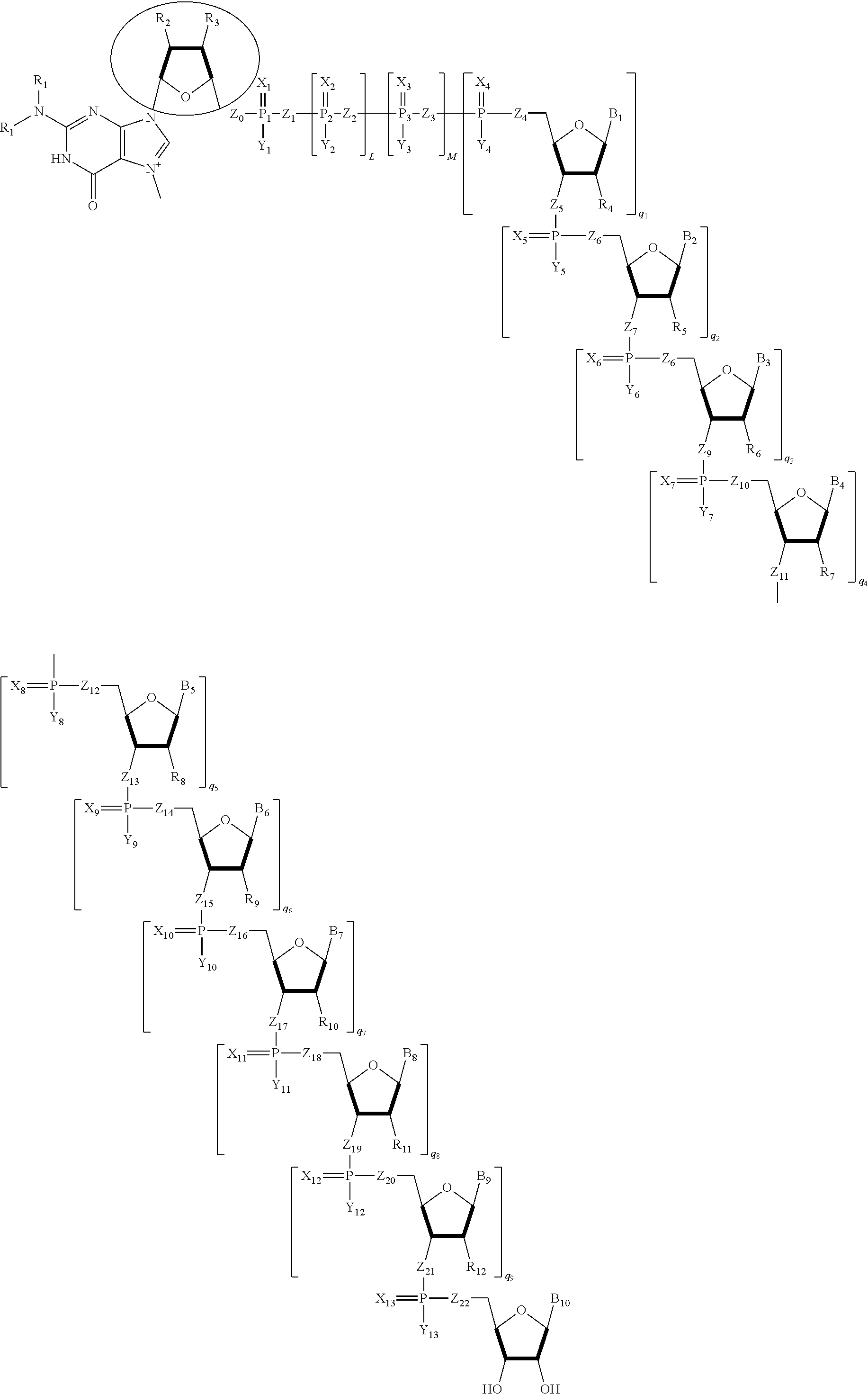

(vi) The cap analog of WO 2018/075827, in particular the cap analog of claim 1 of WO 2018/075827 with the following definition:
   1. A compound of formula m^{T}G(5′)ₚ₃(5′)N₁pN₂,
      wherein m⁷G is a ribonucleotide consisting of N-methylguanine and a ribose;

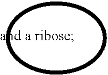

wherein (5)ₚ₃(5′) is a 5′ to 5′ triphosphate linkage, and
wherein N₁ and N₂ are ribonucleotides, wherein one or both of N₁ and N₂ consist of a base selected from N⁶-methyladenine, N¹-methyladenine, pseudouruacil, N¹-methylpseudouracil, 5-iodouracil, 4-thiouracil, 2-thiouracil, 5-methyluracil, pseudoisocytosine, 5-methoxycytosine, 2-thiocytosine, 5-hydroxycytosine, N¹-methylcytosine, 5-hydroxymethylcytosine, hypoxanthine, N¹-methylguanine, O⁶-methylguanine, 1-methyl-guanosine, N²-methyl-guanosine (m2G), N,N-dimethyl-guanosine (m²G), 2-methyl-2′-O-methyl-guanosine (m²Gm), N²,N²-dimethyl-2′-O-methyl-guanosine (m^{2,2}Gm), 1-methyl-2′-O-methyl-guanosine, N²,N⁷-dimethyl-2′-O-methyl-guanosine (m^{2,7}Gm), or isoguanineadenine; and a ribose, a bicyclic (LiNA) ribose, a seco (UNA) ribose, or a modified ribose wherein one or both of the ribose 2′ or 3′ carbons has a fluoro or a C₁-C₆ alkoxy substituent; and
wherein the m⁷G ribonucleotide is linked at its 5-OH to the triphosphate bridge, wherein the triphosphate bridge is linked to a 5-OH of the N₁ ribonucleotide, wherein N₁ nucleotide is linked via its 3-OH to a phosphate, p, wherein the phosphate is linked to a 5′-OH of the N₂ribonucleotide;
or a salt or solvated form thereof.

A “cap analog comprising a 5′ terminal acyclonucleoside” may also be referred to as “cap analog comprising an acyclonucleoside at the position of the 7-methylguanosine” (in other words, at the position, where in a natural cap structure the 7-methylguanosine is found, or in still other words, at the position, where in cap analogs the 7-methylguanosine or an analog thereof is found) or as “cap analog comprising an acyclonucleoside at the position of the 7-methylguanosine and mimicking the 7-methylguanosine”. The cap analog comprising a 5′ terminal acyclonucleoside may be a cap0 analog, a Cap1 analog or a Cap2 analog, wherein a Cap1 analog can be preferred, and the cap analog may be deuterated.

It has been found in the present invention that a ribose or another cyclic structure or a linear multi-branched structure (mimicking the ribose), wherein linear multi-branched structure is to be understood such that the branched structure comprises at least two branches (i.e. carbon-containing substituents or a phosphate substituent, in particular carbon-containing substituents on the linear structural element) and is symmetric (i.e. as in the cap analog of claim 1 of WO 2017/066789, where two symmetric branched carbon units, one carbon unit with substituents R₁₂ and R₁₄ and the other carbon unit with substituents R₁₃ and R₁₅ are present if the dashed bonds and thus Y₁ are absent), at this position is not mandatory in order to provide a functional cap analog. Accordingly, the term “acyclonucleoside” as used in the present application refers to a structure that comprises a nucleobase, which is preferably guanine, a modified guanine or a guanine analog, and a linear unbranched structure or a linear single-branched structure at the position, where otherwise a ribose or another cyclic structure or a linear, (symmetric) multi-branched structure (mimicking the ribose) is found. The term “unbranched” means in this respect that only H, OH, SH, NH₂, or halogen, preferably H, substituents are present on the linear structural element (which is in case of formula (I) as defined herein the section between B₁ and X₁). The “linear unbranched structure” will be selected such that the resulting cap analog can still be used by the polymerase in the in vitro transcription reaction as transcription initiation compound. The term “single-branched” means in this respect that only a single carbon-containing substituent or a single phosphate substituent is present on the linear structural element (which may thus also be referred to as “asymmetric”; contrary to the above symmetric multi-branched structure with at least two carbon-containing substituents or phosphate substituents). The “linear single-branched structure” will be selected such that the resulting cap analog can still be used by the polymerase in the in vitro transcription reaction as transcription initiation compound.

In view of the above, a “cap analog comprising a 5-terminal acyclonucleoside, wherein the acyclonucleoside comprises a linear unbranched structure instead of a ribose” may be any of the above-exemplified cap analogs, wherein the ribose or cyclic structure or linear multi-branched symmetric structure of any of the above-exemplified cap analogs (with the ribose or cyclic structure or linear branched structure being encircled in the above-exemplified cap analogs) is substituted by a linear unbranched structure.

In view of the above, a “cap analog comprising a 5-terminal acyclonucleoside, wherein the acyclonucleoside comprises a linear single-branched structure instead of a ribose” may be any of the above-exemplified cap analogs, wherein the ribose or cyclic structure or linear multi-branched symmetric structure of any of the above-exemplified cap analogs (with the ribose or cyclic structure or linear symmetric branched structure being encircled in the above-exemplified cap analogs) is substituted by a linear single-branched structure.

The term “nucleoside” generally refers to compounds consisting of a sugar, usually ribose or deoxyribose, and a nucleobase, a modified nucleobase or a nucleobase analog as defined below. The nucleobase, modified nucleobase or nucleobase analog is attached to the carbon atom at the 1′ position of the ribose, as in naturally occurring nucleosides and as well-known to the skilled person. The nucleoside may be deuterated.

The term “nucleotide” generally refers to a nucleoside comprising at least one phosphate group, preferably one, two or three phosphate groups, attached to the sugar, in the ribose at the 5′ position.

The term “nucleobase” or simply “base” as used herein refers to the naturally occurring purines and pyrimidines that are present in DNA and RNA, in particular to adenine (A), cytosine (C), guanine (G), thymine (T) and uracil (U). The nucleobases A, G, C and T are found in DNA, whereas A, G, C and U are found in RNA. Accordingly, the nucleobases A, G, C and U are particularly relevant for the present invention. The structures of naturally occurring purines and pyrimidines that are present in DNA and RNA, in particular the structures of A, C, G, T and U, are well known to the skilled person and referred to herein. The nucleobase may be deuterated.

The term “modified nucleobase” as used herein refers to nucleobases as defined above, in particular A, C, G, T and U (with A, G, C and U being preferred for the present invention), which are modified in that the nucleobase carries an additional substituent, such as e.g. an amino group, a thiol group, an alkyl group (in particular a methyl group), or a halo group. Modified nucleobases may or may not be found in nature. For example, the nucleobases can be chemically modified on the major groove face. In some embodiments, the major groove chemical modifications can include an amino group, a thiol group, an alkyl group, or a halo group. Included are e.g. the modified nucleobases N⁶-methyladenine, hypoxanthine, xanthine, 7-methylguanine, 5,6-dihydrouracil, 5-methylcytosine and 5-hydroxymethylcytosine. The modified nucleobase may be deuterated.

The term “modified guanine” is thus a guanine that carried an additional substituent, such as e.g. a methyl-group. The additional substituent may, however, also be an amino group, a thiol group, an alkyl group different from methyl, or a halo group. A particularly preferred modified guanine is 7-methylguanine. The modified guanine may be deuterated. The modified guanine may in particular be selected from the group consisting of N⁷-methylguanine, N⁷-4-chloro-benzyl-guanine, N⁷-biphenyl-methylene-guanine, N⁷-naphtyl-methylene-guanine and N⁷-3,5-dimethyl-benzyl-guanine.

The term “nucleobase analog” as used herein refers to an artificial, i.e. non-natural, nucleobase. A “nucleobase analog” is based on a nucleobase or a modified nucleobase as defined above, wherein not only an additional substituent may (in the case of a modified nucleobase) or may not (in the case of a nucleobase, in particular A, C, G, T or U) be present but at least one substitution can be found in the underlying purine and pyrimidine, respectively, of the nucleobase or modified nucleobase (e.g. a nitrogen in the purine or pyrimidine is substituted by a carbon). A nucleobase analog present in a nucleoside or a nucleotide can nevertheless substitute for a completely natural nucleoside or nucleotide, such as in particular for the nucleotides ATP, UTP, CTP and GTP. The nucleobase analog may be deuterated.

The term “guanine analog” is thus a guanine or a modified guanine, where an atom of the underlying purine structure has been substituted. An example of a guanine analog is 9-deazaguanine, and a particularly preferred guanine analog is 7-methyl-9-deazaguanine. Other examples for guanine analogs are 7-deaza-guanine, 7-cyano-7-deaza-guanine and 7-aminomethyl-7-deaza-guanine. The guanine analog may be deuterated.

In embodiments of the present invention, the modified nucleobase or the nucleobase analog is a nucleobase that is present in a nucleotide selected from the group consisting of 2-amino-6-chloropurineriboside-5′-tri-phosphate, 2-Aminopurine-riboside-5′-triphosphate; 2-aminoadenosine-5′-triphosphate, 2′-Amino-2′-deoxy-cytidine-triphosphate, 2-thiocytidine-5′-triphosphate, 2-thiouridine-5′-triphosphate, 2′-Fluorothymidine-5′-tri-phosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-Iodo-2′-deoxycytidine-5′-triphosphate, 5-iodouridine-5′-triphosphate, 5-Iodo-2′-deoxyuridine-5′-triphosphate, 5-methyl-cytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurine-riboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. As an example, the nucleobase that is present in 5-methyl-cytidine-5′-triphosphate is 5-methylcytosine.

The modified nucleobase or the nucleobase analog is in particular a nucleobase that is present in a nucleotide selected from the group consisting of 5-methylcytidine-5′-triphosphate, 7-deazaguanosine-5′-triphosphate, 5-bromocytidine-5′-triphosphate, and pseudouridine-5′-triphosphate. As an example, the nucleobase that is present in 7-deazaguanosine-5′-triphosphate is 7-deazaguanine.

In some embodiments, the modified nucleobase or the nucleobase analog is a nucleobase that is present in a nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, 1-methoxymethyl-pseudouridine, 1-ethyl-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. As an example, the nucleobase that is present in 5-propynyl-uridine is 5-propynyl-uracil.

The modified nucleobase or the nucleobase analog is in some embodiments a nucleobase that is present in a nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. As an example, the nucleobase that is present in 2-thio-5-methyl-cytidine is 2-thio-5-methyl-cytosine.

In other embodiments, the modified nucleobase or the nucleobase analog is present in a nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. As an example, the nucleobase that is present in N6-glycinylcarbamoyladenosine is N6-glycinylcarbamoyladenine.

In other embodiments, the modified nucleobase or the nucleobase analog is a nucleobase that is present in a nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. As an example, the nucleobase that is present in 6-thio-7-methyl-guanosine is 6-thio-7-methyl-guanine.

In other embodiments of the present invention, the modified nucleobase or the nucleobase analog is a nucleobase that is present in a nucleoside selected from the group consisting of 6-aza-cytidine, 2-thio-cytidine, α-thio-cytidine, Pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, 5-methyl-uridine, Pyrrolo-cytidine, inosine, α-thio-guanosine, 6-methyl-guanosine, 5-methyl-cytidine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, α-thio-adenosine, 8-azido-adenosine, 7-deaza-adenosine. As an example, the nucleobase that is present in 7-deaza-guanosine is 7-deaza-guanine.

In yet other embodiments of the present invention, the modified nucleobase or the nucleobase analog is a nucleobase that is present in a nucleoside selected from the group consisting of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. As an example, the nucleobase that is present in 2-thio-5-aza-uridine is 2-thio-5-aza-uracil.

In a specific embodiment, the modified nucleobase or the nucleobase analog is a nucleobase that is present in a nucleoside or nucleotide selected from the group consisting of pseudouracil (ψ), N1-methylpseudouracil (N1M1ψ), 1-ethylpseudouracil, 2-thiouracil (s2U), 4-thiouracil, 5-methylcytosine, 5-methyluracil, 5-methoxyuracil, and any combination thereof, most preferably the chemical modification is N1-methylpseudouracil (N1M1ψ). As an example, the nucleobase that is present in N1-methylpseudouracil is N1-methyluridine.

The terms “RNA in vitro transcription” or “in vitro transcription” relate to a process wherein RNA is synthesized in a cell-free system (in vitro). DNA, particularly plasmid DNA, is used as template for the generation of RNA transcripts. RNA may be obtained by DNA-dependent in vitro transcription of an appropriate DNA template, which is preferably a linearized plasmid DNA template. The promoter for controlling in vitro transcription can be any promoter for any DNA-dependent RNA polymerase. Particular examples of DNA-dependent RNA polymerases are the T7, T3, and SP6 RNA polymerases. A DNA template for in vitro RNA transcription may be obtained by cloning of a nucleic acid, in particular cDNA corresponding to the respective RNA to be in vitro transcribed, and introducing it into an appropriate vector for in vitro transcription, for example into plasmid DNA. In a preferred embodiment of the present invention the DNA template is linearized with a suitable restriction enzyme, before it is transcribed in vitro. The cDNA may be obtained by reverse transcription of RNA or chemical synthesis. Moreover, the DNA template for in vitro RNA synthesis may also be obtained by gene synthesis.

Methods for in vitro transcription are known in the art (see, e.g., Geall et al. (2013) Semin. Immunol. 25(2): 152-159; Brunelle et al. (2013) Methods Enzymol. 530:101-14). Reagents used in said method typically include:
1) a linearized DNA template with a promoter sequence that has a high binding affinity for its respective RNA polymerase such as bacteriophage-encoded RNA polymerases;
2) ribonucleotides with (optionally modified) triphosphates (NTPs), in particular ATP, CTP, GTP and UTP (or, if modified triphosphates are used, in particular 1-Me-Pseudo-UTP instead of UTP);
3) a DNA-dependent RNA polymerase capable of binding to the promoter sequence within the linearized DNA template (e.g. T7, modified T7, T3 or SP6 RNA polymerase);
4) optionally, a ribonuclease (RNase) inhibitor to inactivate any contaminating RNase;
5) optionally, a pyrophosphatase to degrade pyrophosphate, which may inhibit transcription;
6) MgCl₂, which supplies Mg²⁺ ions as a co-factor for the polymerase;
7) a buffer to maintain a suitable pH value, which can also contain antioxidants (e.g. DTT), and/or polyamines such as spermidine at optimal concentrations; and
8) a cap analog (such as in particular a cap analog of the present invention).

Besides the acyclonucleoside at the position mimicking the 7-methylguanosine, the RNA according to the present application may comprise “artificial RNA”, wherein “artificial RNA” encompasses in particular RNA comprising at least one chemical modification. The chemical modification may be selected from the group consisting of a sugar modification, a backbone modification, and a base modification. A backbone modification in connection with the present invention is a modification in which phosphates of the backbone of the nucleotides contained in an RNA are chemically modified. A sugar modification in connection with the present invention is a chemical modification of the sugar of the nucleotides of the RNA. Furthermore, a base modification in connection with the present invention is a chemical modification of the nucleobase of the nucleotides of the RNA.

The modified nucleotides, which may be incorporated into RNA according to the present application, can be modified in the sugar. Accordingly, at least one sugar of the RNA of the present application may be modified. For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethylene glycols (PEG), —O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., N^{RR}, can be alkylamino, dialkylamino, heterocyclyl, arylamino, diarylamino, heteroarylamino, or diheteroaryl amino, ethylene diamine, polyamino) or aminoalkoxy.

“Deoxy” modifications include hydrogen, amino (e.g. NH₂; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O.

The sugar can also contain one or more carbons that possess the opposite stereochemical configuration than that of the corresponding carbon in ribose. Thus, a modified RNA can include nucleotides containing, for instance, arabinose as the sugar.

The modified nucleotides, which may be incorporated into RNA according to the present application, can be modified in a phosphate group. Accordingly, at least a region of the backbone of the RNA of the present application may be modified. The phosphate groups of the backbone of the RNA according to the present application can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates). The backbone can also be modified in that it comprises or consists of repeating N-(2-aminoethyl)-glycine units linked by peptide bonds (so-called “peptide nucleic acid” or “PNA” backbones), where the nucleobases are linked to the backbone by a methylene bridge and a carbonyl group.

The modified nucleotides, which may be incorporated into RNA according to the present application in the in vitro reaction, can be modified in the nucleobase. Accordingly, at least one nucleobase of the RNA of the present application may be modified. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

In particularly preferred embodiments of the present invention, the modified nucleotides that are used in the in vitro transcription (thus resulting in the corresponding modification in the nucleobase in the resulting RNA) are selected from 2-amino-6-chloropurineriboside-5′-triphosphate, 2-Aminopurine-riboside-5′-triphosphate, 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-Iodo-2′-deoxycytidine-5′-triphosphate, 5-iodouridine-5′-triphosphate, 5-Iodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. Particular preference is given to modified nucleotides selected from the group consisting of 5-methylcytidine-5′-triphosphate, 7-deazaguanosine-5′-triphosphate, 5-bromocytidine-5′-triphosphate, and pseudouridine-5′-triphosphate. In some embodiments, the nucleotide can be modified on the major groove face and can include replacing hydrogen on C-5 of uracil with a methyl group or a halo group.

In some embodiments, the modified nucleotides that are used in the in vitro transcription (thus resulting in the corresponding modification in the nucleobase in the resulting RNA) are nucleotides that comprise modified nucleosides that include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In other embodiments, modified nucleosides include 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In specific embodiments, a modified nucleoside is 5′-O-(1-thiophosphate)-adenosine, 5′-O-(1-thiophosphate)-cytidine, 5′-O-(1-thiophosphate)-guanosine, 5′-O-(1-thiophosphate)-uridine or 5′-O-(1-thiophosphate)-pseudouridine. In further specific embodiments, the modified nucleoside is selected from 6-aza-cytidine, 2-thio-cytidine, α-thio-cytidine, Pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, 5-methyl-uridine, Pyrrolo-cytidine, inosine, α-thio-guanosine, 6-methyl-guanosine, 5-methyl-cytidine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, α-thio-adenosine, 8-azido-adenosine, 7-deaza-adenosine. In further embodiments, the modified nucleoside is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In a specific embodiment, the modified nucleoside is selected from the group consisting of pseudouridine (ψ), N1-methylpseudouridine (N1M1ψ), 1-ethylpseudouridine, 2-thiouridine (s2U), 4-thiouridine, 5-methylcytosine, 5-methyluridine, 5-methoxyuridine, and any combination thereof, most preferably the modified nucleoside is N1-methylpseudouridine (N1M1ψ).

### EXAMPLES

In the following section, particular examples illustrating various embodiments and aspects of the invention are presented. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the claims as disclosed herein.

### Example 1: Synthesis of Compounds of the Present Invention

### Starting Materials, Methods and Analytical Data:

Unless otherwise specified, all starting materials are obtained from commercial suppliers in the highest purity available or are prepared by methods known to the skilled person. If not stated otherwise, all reactions are conducted at room temperature, ambient atmosphere and as indicated in the individual procedures.

The temperatures are expressed as ° C. Solvent removal was carried out using rotary evaporation at 40° C. The conditions for column chromatography and HPLC are specified in each case. MS was conducted using an Exploris 240 (Thermo Fisher) with HESI ion source and orbitrap analyzer, a Waters I-Class UPLC with Acquity ESI QDa mass detector or an Agilent1290/Agilent1260 system equipped with an ESI ion source and a SQ-MSD. ESI-MS using the Exploris system was carried out in positive and negative mode using 5 mM ammonium acetate+90% methanol. The Agilent devices were run with 1% aqueous formic acid+1% formic acid in acetonitrile for the positive ion mode and with 10 mM ammoniumbicarbonate in water+acetonitrile for the negative ion mode. The waters device was run using 10 mM ammoniumbicarbonate in water (pH 9.5)+acetonitrile. NMR spectra were recorded using a Bruker Avance III HDX 400 with a 5 mm BBFO sample head, a Bruker Avance II—Ultrashield 400 MHz, a Bruker Avance Neo—Ascend 400 MHz or an Magritek Ultra 80 MHz spectrometer. Chemical shifts (δ) are reported in ppm relative to the residual solvent signal. ¹H-NMR data are reported as follows: chemical shift (multiplicity, number of hydrogens). Multiplicity is abbreviated as follows: s (singlet), d (doublet), t (triplet), q (quartet), m (multiplet), br (broad signal). For chiral compounds the sum of the integrals for both isomers is reported. Additionally, splitting of peaks for chiral compounds may be observed which is mostly reported as multiplet. All cap analogs were quantified using a Nanodrop 2000c UV/Vis spectrophotometer (Thermo Fischer) in UV/Vis mode with automatic path length option. Cap analogs were dissolved in 1 mL of water. A 1 μL aliquot was diluted 1:10 and 1:100 and measured at 255 nm for Cap1/Cap2 analogs and 254 nm for Cap0 analogs. Concentration and substance amount was calculated according to lambert-beers law with extinction coefficient of 30539 L/mol*cm for Cap1, 46539 L/mol*cm for Cap1 containing a N⁷-biphenyl moiety, 21000 L/mol*cm for Cap0 and 43537 L/mol*cm for Cap2.

Ganciclovir (X1), as used herein, refers to the following structure and is commercially available (Biosynth, UK):

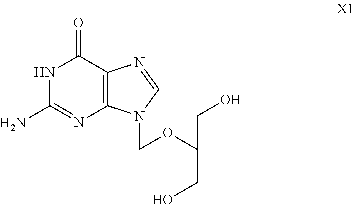 [SMILES: Nc1nc2c(ncn2COC(CO)CO)c(=O)[nH]1]

Penciclovir (X2), as used herein, refers to the following structure and is commercially available (Biosynth, UK):

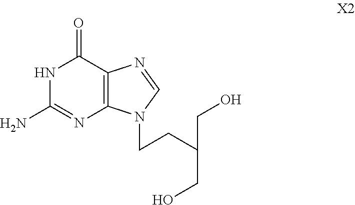 [SMILES: Nc1nc2c(ncn2CCC(CO)CO)c(=O)[nH]1]

5′-O-(4,4-dimethoxytrityl)-N²-isobutyryl-guanosine (X3), as used herein, refers to the following structure and is commercially available (Biosynth UK).

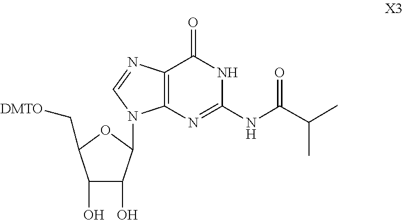

N⁶-benzoyl-5′-O-(4,4-dimethoxytrityl)-2′-OMe-adenosine-3′-(2-cyanoethyl-N,N-diisopropyl)phosphoramidite (X₄), as used herein, refers to the following structure and is commercially available (Biosynth, UK):

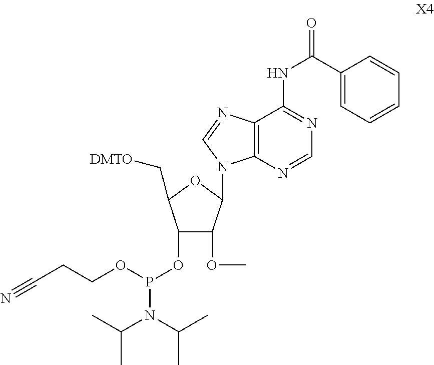

2-Phenyl-1,3-dioxan-5-ol (X5), as used herein, refers to the following structure and is commercially available

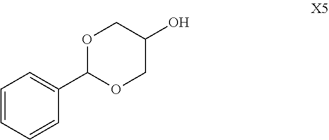 [SMILES: OC1COC(c2ccccc2)OC1]

Acetic acid 2-bromo-ethyl ester (X6), as used herein, refers to the following structure and is commercially available (Sigma Aldrich):

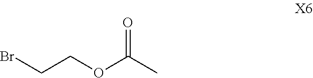

Hydroxyethyl vinyl ether (X7), as used herein, refers to the following structure and is commercially available:

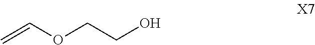

6-Chloro-2-aminopurin (X8) as used herein, refers to the following structure and is commercially available (Biosynth, UK):

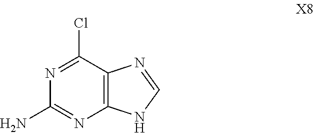 [SMILES: Nc1nc(Cl)c2nc[nH]c2n1]

Triethylene glycol (X9) as used herein, refers to the following structure and is commercially available (Combi-Blocks, Inc.):

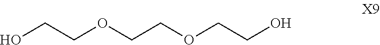

Tetraethylene glycol (X10) as used herein, refers to the following structure and is commercially available (Acros Organics):

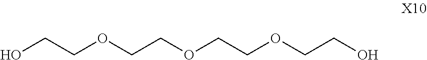 [SMILES: OCCOCCOCCOCCO]

Pentaethylene glycol (X11) as used herein, refers to the following structure and is commercially available (Ambeed):

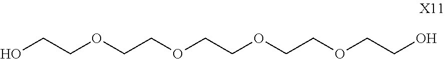 [SMILES: OCCOCCOCCOCCOCCO]

Guanosine monophosphate disodium salt (X12) as used herein, refers to the following structure and is commercially available (Biosynth, UK):

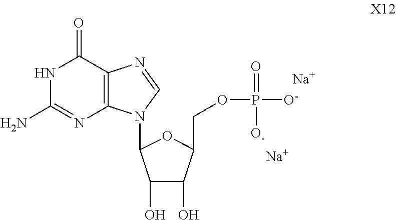

2′,3′-O-diacetyl-N²-isobutyryl guanosine (X13) as used herein, refers to the following structure and is commercially available (WuXi Apptec):

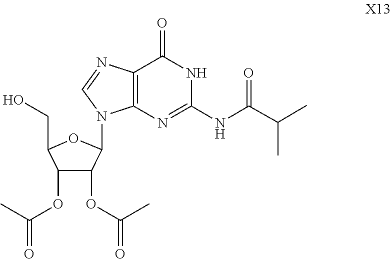 [SMILES: CC(=O)OC1C(CO)OC(n2cnc3c(=O)[nH]c(NC(=O)C(C)C)nc32)C1OC(C)=O]

5′-O-(4,4-di methoxytrityl)-N²-isobutyryl-2-OMe-guanosine-3′-(2-cyanoethyl-N, N-diisopropyl)phosphoramidite (X14) as used herein, refers to the following structure and is commercially available (Biosynth UK):

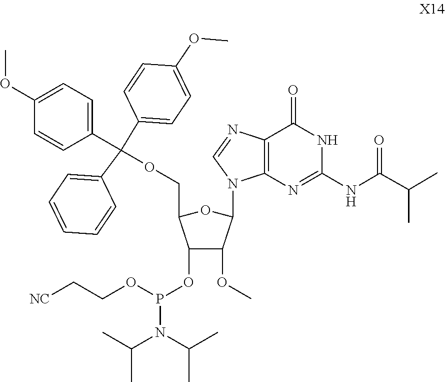 [SMILES: COc1ccc(C(OCC2OC(n3cnc4c(=O)[nH]c(NC(=O)C(C)C)nc43)C(OC)C2OP(OCCC#N)N(C(C)C)C(C)C)(c2ccccc2)c2ccc(OC)cc2)cc1]

N⁶-benzoyl-5′-O-(4,4-dimethoxytrityl)-2′-O-tert-butyldimethylsilyl-adenosine-3′-(2-cyanoethyl-N,N-diisopropyl)phosphoramidite (X15), as used herein, refers to the following structure and is commercially available (BLDpharm):

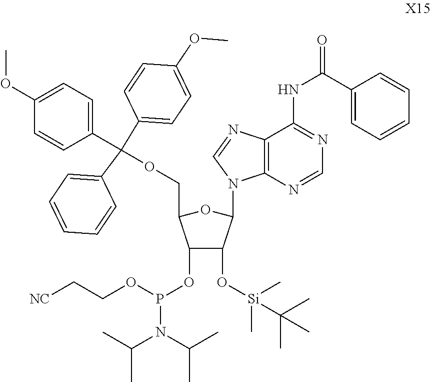 [SMILES: COc1ccc(C(OCC2OC(n3cnc4c(NC(=O)c5ccccc5)ncnc43)C(O[Si](C)(C)C(C)(C)C)C2OP(OCCC#N)N(C(C)C)C(C)C)(c2ccccc2)c2ccc(OC)cc2)cc1]

List of abbreviations

| | |
|---|---|
| DMF | N,N-dimethylformamide |
| TBDPheSiCl | tert-butyl(chloro)diphenylsilan |
| DCM | dichloromethane |
| MeOH | methanol |
| ESI-MS | electrospray ionization mass spectrometry |
| NMR | nuclear magnetic resonance |
| DMAP | 4-(dimethylamino)pyridine |
| Ac₂O | acetic anhydride |
| Na₂SO₄ | sodium sulfate |
| ACN | acetonitrile |
| Et₃N | triethylamine |
| TIPBSCl | 2,4,6-triisopropylbenzenesulfonyl chloride |
| TLC | thin layer chromatography |
| DABCO | 1,4-diazabicyclo[2.2.2]octan |
| DBU | 1,8-diazabicyclo[5.4.0]undec-7-en |
| THF | tetrahydrofuran |
| H₂O | water |
| NaOH | sodium hydroxide |
| NaH | sodium hydride |
| MeI | iodomethane |
| HF | hydrogen fluoride |
| TEAB | triethylammonium bicarbonate |
| TBAF | triethylammonium fluoride |
| tBuOOH | tert-butyl hydroperoxide |
| LC-MS | liquid chromatography mass spectrometry |
| DMSO | dimethyl sulfoxide |
| MgCl₂ | magnesium chloride |
| EDTA | ethylenediaminetetraacetic acid |
| RP-HPLC | reversed-phase high-performance liquid |
| | chromatography |
| NMP | N-methyl-pyrrolidone |
| RP-TLC | reversed-phase thin layer chromatography |
| HCl | hydrochloric acid |
| POCl₃ | phosphorus oxychloride |
| DTT | dithiothreitol |
| DIAD | diisopropyl azodicarboxylate |
| DEAD | diethyl azodicarboxylate |
| TBDMSCl | tert-butyldimethylchlorosilane |
| TFA | trifluoroacetic acid |
| Pd/C | palladium on carbon |
| MnO₂ | manganese(IV) oxide |
| DMTCl | 4,4′-dimethoxytrityl chloride |
| NMM | 1-methylmorpholine |
| Tris | Tris(hydroxymethyl)aminomethan |
| CDI | Carbonyldiimidazole |
| MnCl₂ | manganese chloride |

### General Procedure 1:

The respective compound (1.0 eq.), triphenylphosphine (1-3 eq.) and the nucleophile of choice (1-3 eq.) were suspended in dry dioxane or THE under argon gas atmosphere and stirred at ambient temperature until all compounds were dissolved as good as possible. A suitable azodicarboxylate (e.g. DIAD, DEAD, 1-3 eq.) was added dropwise over the course of 5-30 min and the reaction mixture was left stirring until all starting material was consumed. The solvent was removed under reduced pressure and the crude product was purified by flash chromatography.

### General Procedure 2:

The respective compound (1.0 eq.) was dissolved in dry acetonitrile and acetonitrile containing 0.45 M or 1 M tetrazole under argon gas atmosphere. After 30 min, bis-cyanoethyl-N,N-diisopropyl phosphoramidte (1-4 eq.) was added dropwise and the reaction mixture was allowed to stir at ambient temperature until TLC and/or LC/MS indicates completion of the phosphitylation reaction. Oxidation to the corresponding P(V) species was realized by the addition of 0.1 M iodine in THE/pyridine/water (77:21:2, v/v/v) until a permanent red staining was observed. Then, a 1:1 mixture of aqueous sodium disulfite solution (5 wt. %) and citric acid solution (5 wt. %) was added and the crude product was washed with dichloromethane. All volatiles were evaporated and the residue was dissolved in concentrated aqueous ammonia or in methanol/concentrated ammonia with optional addition of DTT (2.5-5 eq.) and stirred at ambient temperature or 60° C. until TLC and/or LC-MS indicated complete removal of all cyanoethyl moieties and protecting groups. Solvent was evaporated and the crude product was purified by ion exchange chromatography with Macro-Prep-High-Q resin using either a TEAB gradient from 0-1 M or a TEAB gradient from 0-0.5 M. The product fractions can be repurified via C18 RP, using either a Buchi C18, 250×30 mm, 5 μM Column, a Buchi SELECT (30 μm spherical particles)/ECOFLEX (50 μm spherical particles) C18, 120 g or a Flash Pure Buchi (40 um irregular particles) C18, 120 g/40 g. 5 μM column: Buffer A: 5 mM ammonium acetate pH 5.6, Buffer B: 100% MeOH. 30/50/40 μM column: Buffer A: 10 mM ammonium acetate, Buffer B: 100% MeOH. As alternative to the oxidation with 0.1 M iodine in THF/pyridine/water, oxidation was carried out by the dropwise addition of equimolar amounts (relative to the phosphitylation reagent) of 1 M tBuOOH in toluene (prepared form 5.0-6.0 M tBuOOH in decane) at 0° C. The reaction is stirred 5 min at 0° C. and another 5 min at ambient temperature followed by evaporation of the solvent and deprotection and purification of the product as described above. In case of LC-MS reaction monitoring in combination with incomplete or slow oxidation another portion for 1 M tBuOOH in toluene might be added and/or reaction times might be prolonged. All volatiles were removed in vacuo and the remaining residue was deprotected as described above.

### General Procedure 3:

Trimethylphosphate and POCl₃ (2-4 eq.) were precooled to 0° C. and placed in a dry schlenk flask. The respective compound (1.0 eq.) was added at once and the mixture was stirred at 0° C. under protective gas atmosphere. The reaction was quenched after full conversion with TEAB-buffer (0.1 M, pH 8-10), diluted with H₂O and pH was adjusted to 7.0-8.0 using aqueous ammonia solution. The purification was carried out by ion exchange chromatography with Macro-Prep High Q resin using either a TEAB gradient from 0-1 M or a TEAB gradient from 0-0.5 M. Solvent was evaporated from product fractions and the product was obtained as its triethylammonium salt which is directly used in the next step or was repurified by C18 RP, using either a Buchi C18, 250×30 mm, 5 μM Column, a Buchi SELECT (30 μm spherical particles)/ECOFLEX (50 μm spherical particles) C18, 120 g column or a Flash Pure Buchi (40 μm irregular particles) C18, 120 g/40 g. 5 μM column: Buffer A: 5 mM ammonium acetate pH 5.6, Buffer B: 100% MeOH. 30/50/40 μM column: Buffer A: 10 mM ammonium acetate, Buffer B: 100% MeOH.

### General Procedure 4:

MeI (8 eq.) was added to a stirred solution of the respective compound (1.0 eq.) in DMSO (0.1 M). The reaction mixture was diluted with 10 volumes of water as soon as TLC and/or ESI-MS indicates complete consumption of the starting material. Excessive MeI was removed by extensive extraction with diethyl ether and residual MeI in the aqueous layer was inactivated by the addition of a small amount of sodium disulfite. The pH of the colorless aqueous layer was set to 7 using concentrated aqueous ammonia and the crude product was purified by ion exchange chromatography with Macro-Prep High Q resin using either a TEAB gradient from 0-1 M or a TEAB gradient from 0-0.5 M. The solvent was evaporated from product fractions which were repurified by C18 RP-HPLC, using a Phenomexx Gemini C18, 250×21.2 mm, 5 μM column or C18 RP, a Buchi C18, 250×30 mm, 5 μM Column,a Buchi SELECT (30 μm spherical particles)/ECOFLEX (50 μm spherical particles) C18, 120 g or a Flash Pure Buchi (40 μm irregular particles) C18, 120 g/40 g. Buchi 5 μM column. Buffer A: 5 mM ammonium acetate pH 5.6, Buffer B: 100% MeOH. Phenomexx 5 μM column: Buffer A: 5 mM ammonium acetate pH 5.6, Buffer B: 90% MeOH. 30/50/40 μM column: Buffer A: 10 mM ammonium acetate, Buffer B: 100% MeOH.

### General Procedure 5:

Guanosine 5′-imidazolide diphosphate (synthesized according to [A. R. Kore, M. Shanmugasundaram, Current Protocols 2013, 55, 13.13.1-13.13.12]) was added to a vigorously stirred solution of the respective compound (1.0 eq.) in DMSO or DMF or NMM buffer (0.2 M, pH 7.0) or most preferred H₂O/ACN (1/1, v/v). A divalent cation (1-10 eq.) like ZnCl₂, MnCl₂ or most preferred MgCl₂ was introduced to the reaction mixture which was stirred at ambient temperature until LC-MS indicates complete turnover. The reaction was then quenched by the addition of a 250 mM EDTA solution, and the pH of the resulting solution was adjusted to 7 using aqueous ammonia solution.

Purification method A: The purification is carried out by ion exchange chromatography with Macro-Prep High Q resin using a TEAB gradient from 0-1 M and the solvent is evaporated from product fractions. The product fraction is repurified via C18 RP HPLC, using a Phenomexx Gemini C18, 250×21.2 mm, 5 μM Column. Buffer A: 5 mM ammonium acetate pH 5.6, Buffer B: 100% MeOH.

Purification method B: The reaction mixture was desalted using RP-HPLC, using a Phenomexx Gemini C18, 250×21.2 mm, 5 μM Column using a gradient from 0-80% of solvent B in solvent A or the gradient described within the respective experimental description within example 1. Solvent A: 5 mM ammonium acetate in water; solvent B: 90% methanol in water. The desalted reaction mixture was purified by ion exchange chromatography with DNAPac PA200 column (22×250 mm) using a gradient from 0-80% of buffer B in Buffer A or the gradient described within the respective experimental description within example 1. Buffer A: 20 mM Tris, pH 9; Buffer B: 20 mM Tris, 33 mM sodium perchlorate. The product fractions were desalted using RP-HPLC, using a Phenomexx Gemini C18, 250×21.2 mm, 5 μM Column, using a gradient from 0-80% of solvent B or the gradient described within the respective experimental description within example 1. Solvent A: 5 mM ammonium acetate in water; solvent B: 90% methanol in water. The resulting fraction was repurified via C18 RP HPLC, using a Phenomexx Gemini C18, 250×21.2 mm, 5 μM Column, using the gradient described within the respective experimental description within example 1. Buffer A: 5 mM ammonium acetate pH 5.6, Buffer B: 90% MeOH, gradient. The product fractions were lyophilized and dissolved in ultrapure water resulting in a 100 mM solution.

### General Procedure 6:

The respective compound (1.0 eq.) was dissolved in TFA/H₂O (3/1, v/v) and stirred at ambient temperature until consumption of all starting material. All volatiles were removed under reduced pressure and the remining residue was coevaporated trice with ethanol or toluene. The crude product was purified by flash chromatography as indicated in the individual procedures.

Alternatively, the respective compound (1.0 eq.) was dissolved in 1 N aqueous HCl and stirred at 85° C. until the starting material was fully consumed. The pH of the solution was adjusted to 9 using ammonium hydroxide upon which the reaction mixture was cooled for 4-24 h at 0-4° C. whereas the product precipitates as a white powder. The precipitate was collected by filtration and washed with a small amount of water. The liquid phase was once more cooled and filtrated. The combined product was dried in vacuo.

### General Procedure 7:

The cap precursor (1.0 eq.) was dissolved/suspended in dry DMSO (0.5-2.0 mL) and CDI (10.0 eq.) was added. The reaction mixture was stirred at ambient temperature until LC-MS indicated complete formation of the imidazolide intermediate. Optionally, another portion of CDI (10.0 eq.) may be added to achieve complete activation of the starting material. Upon consumption of all starting material, water (10.0-20.0 eq.) was introduced carefully followed by the consecutive addition of 2 M NMM buffer (pH 7, 0.2 M final concentration), ppAmG (compound 28) (1.0 eq.) and MnCl₂ (1.0 eq.). Upon complete product formation, the reaction was quenched by the addition of 250 mM EDTA solution (1.1 eq.). The reaction mixture was then desalted using RP-HPLC (Phenomexx Gemini C18, 250×21.2 mm, 5 μM Column) using individually optimized gradients from 0-95% of solvent B in solvent A. Solvent A: 5 mM ammonium acetate in water; solvent B: 90% methanol in water. The desalted reaction mixture was purified by RP-HPLC (Phenomexx Gemini C18, 250×21.2 mm, 5 μM Column) using 100 mM triethylammonium acetate (pH 7, Buffer A) and 90% MeOH in water (Buffer B) as eluents. The product fractions were desalted using RP-HPLC (Phenomexx Gemini C18, 250×21.2 mm, 5 μM Column) using a gradient from 0-95% of solvent B in solvent A. Solvent A: 5 mM ammonium acetate in water; solvent B: 90% methanol in water or 100% acetonitrile. The product fractions were lyophilized and dissolved in ultrapure water resulting in a 100 mM solution.

### Example 1.1.1: Synthesis Route I′

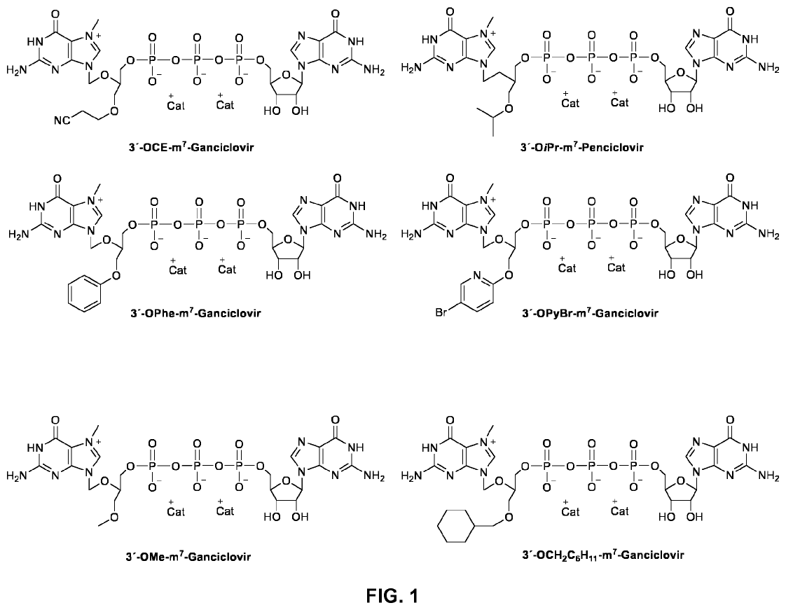

The synthesis of compound 9a (corresponding to 3′-OMe-m⁷-Ganciclovir-ppp-(5′)-Guanosine, referred to in FIG. 1 as “3′-OMe-m⁷-Ganciclovir” compound) when starting from Ganciclovir (X1) is shown in the following. It is noted that the synthesis route for other “3′-O-modified” derivatives also proceeds via the described reaction pathway. Examples of resulting compounds analogous to compound 9a when starting from Ganciclovir (X1) or Penciclovir (X2) are described at the end of the present example and are depicted in FIG. 1.

Synthesis of Compound 1 with the Following Structure

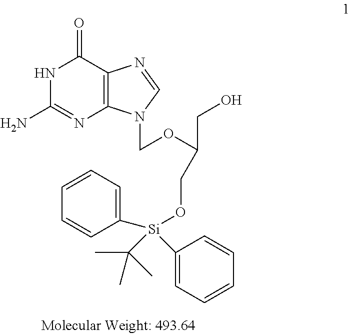 [SMILES: CC(C)(C)[Si](OCC(CO)OCn1cnc2c(=O)[nH]c(N)nc21)(c1ccccc1)c1ccccc1]

Ganciclovir (X1, 1.00 g, 1.0 eq.) was suspended in DMF (40 mL) and was treated with TBDPheSiCl (1.0 eq.) and imidazole (1.0 eq.) under argon atmosphere. The reaction was stirred 12 h at ambient temperature followed by evaporation of the solvent and purification by flash chromatography (silica 40 g, A: DCM, B MeOH, gradient program from Table Ex-1.)

TABLE Ex-1

| Time | | |
|---|---|---|
| total | Flow | % |
| [min] | [mL/min] | B |
| 0 | 45 | 0 |
| 20.3 | 45 | 16 |
| 20.4 | 45 | 16 |
| 21.6 | 45 | 100 |
| 31.6 | 45 | 100 |

Product fractions were analyzed via ESI-MS and NMR. The product (Compound 1) was obtained as a white powder in a yield of 56%.

ESI-MS: [M+H]⁺: 494.222, Calculated 494.222

¹H NMR (80 MHz, d₆-DMSO) δ 10.64 (s, 1H), 7.79 (s, 1H), 7.63-7.36 (m, 10H), 6.49 (s, 2H), 5.45 (s, 2H), 3.86-3.38 (m, 5H), 0.91 (s, 9H).

Synthesis of Compound 2a and 2b with the Following Structures:

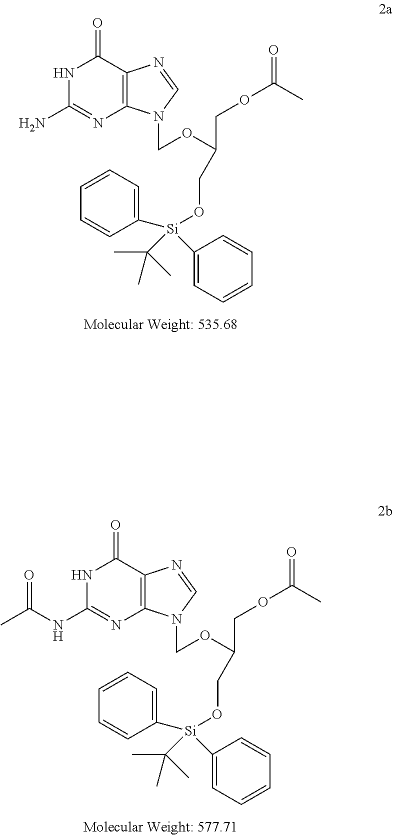

### Method A:

Compound 1 (560 mg, 1.0 eq.) was coevaporated three times with pyridine and was further dried under high vacuum for 30 min. It was then dissolved in pyridine (8 mL) and DMAP (0.1 eq.) was added. Ac₂O (2.5 eq.) was introduced dropwise to the reaction mixture which was stirred 16 h at ambient temperature under argon gas atmosphere. The reaction mixture was diluted with ethyl acetate and was washed three times with saturated sodium bicarbonate solution. The organic layer was dried over Na₂SO₄, was concentrated, and purified by flash chromatography (silica 12 g, A: cyclohexane, B: ethyl acetate, C: methanol, gradient program from Table Ex-2.). Product fractions were analyzed via ESI-MS and NMR.

TABLE Ex-2

| Time | | | |
|---|---|---|---|
| total | Flow | % | % |
| [min] | [mL/min] | B | C |
| 0 | 30 | 0 | 0 |
| 16 | 30 | 100 | 0 |
| 40 | 30 | 100 | 0 |
| 10 | 30 | 90 | 10 |

Compound 2a was obtained as a white powder in a yield of 35%.

ESI-MS: [M+H]⁺: 536.232, Calculated 536.232

¹H NMR (400 MHz, d₆-DMSO) δ 10.62 (s, 1H), 7.79 (s, 1H), 7.56 (m, 4H), 7.43 (m, 6H), 6.45 (s, 2H), 5.41 (m, 2H), 4.17 (m, 1H), 4.02 (m, 2H), 3.59 (m, 2H), 1.87 (s, 3H), 0.93 (s, 9H).

Compound 2b was obtained as a colorless solid in a yield of 63%.

ESI-MS: [M+H]⁺: 578.242, Calculated 578.243

¹H NMR (400 MHz, d₆-DMSO) δ 12.04 (s, 1H), 11.73 (s, 1H), 8.13 (s, 1H), 7.54 (m, 4H), 7.43 (m, 6H), 5.53 (m, 2H), 4.18 (m, 1H), 4.01 (m, 2H), 3.59 (m, 2H), 2.17 (s, 3H), 1.84 (s, 3H), 0.91 (s, 9H).

### Method B:

Compound 1 is suspended in ACN and is treated consecutively with DMAP, Et₃N and Ac₂O under protective gas atmosphere. The reaction mixture is stirred at ambient temperature and is quenched after completion by the addition of MeOH. Solvents are evaporated and the residue is dissolved in ethyl acetate. Extraction with saturated sodium bicarbonate solution is followed by purification via flash chromatography.

It is known by the skilled person that the product distribution can be adjusted by the equivalents of acetic anhydride, the reaction temperature and the presence or absence of the DMAP catalyst for both methods A and B.

Synthesis of Compound 3a and 3b with the Following Structures:

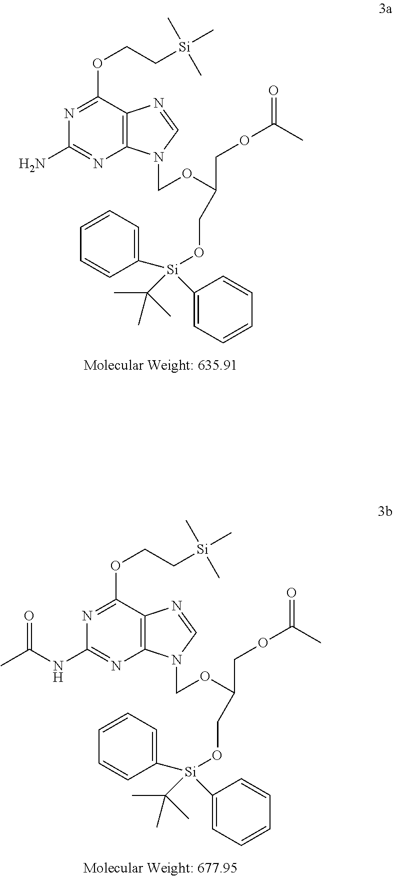

### Method A:

Compound 3a was prepared from compound 2a (325 mg, 1.0 eq.) according to general procedure 1 employing 2.5 eq. of triphenylphosphine, 2-(trimethylsilyl)-ethanol and DIAD in 8 mL of dry dioxane. Flash chromatography: silica 12 g, A: cyclohexane, B: ethyl acetate, gradient program from Table Ex-3. Product fractions were analyzed via ESI-MS and NMR (referenced to cyclohexane impurity since solvent signal was covered by benzene moieties).

TABLE Ex-3

| Time | | |
|---|---|---|
| total | Flow | % |
| [min] | [mL/min] | B |
| 0 | 30 | 0 |
| 16 | 30 | 60 |
| 21 | 30 | 100 |
| 26 | 30 | 100 |

Compound 3a was obtained as a white foam in a yield of 58%.

ESI-MS: [M+H]⁺: 636.302, Calculated 636.303

¹H NMR (80 MHz, CDCl₃) δ 7.73-7.32 (m, 9H), 5.52 (s, 2H), 4.98 (s, 2H), 4.61 (dd, 2H), 4.30-3.67 (m, 5H), 1.88 (s, 3H), 1.25 (m, 2H), 1.06 (s, 9H), 0.10 (s, 9H).

### Method B:

Compound 2b (410 mg, 1.0 eq.) was dried under high vacuum overnight. It was then dissolved in DCM (4.5 mL) under argon gas atmosphere and DMAP (0.2 eq.), Et₃N (4.0 eq.) and TIPBSCl (1.5 eq.) were added consecutively. The reaction mixture was stirred for 1 h at ambient temperature and was then deemed complete by TLC. Dilution with DCM was followed by extraction with 5% sodium bicarbonate solution. The organic layer was dried over Na₂SO₄, the solvent was removed under reduced pressure and activated compound 2b was dried under high vacuum. The latter was dissolved in dioxane (11.8 mL) and was treated with DABCO (2.0 eq.) and activated molecular sieves. After 1 h of stirring, 2-(trimethylsilyl)-ethanol (5.0 eq.) and DBU (2.5 eq.) were added, and the reaction mixture was stirred another 1 h at ambient temperature. Dilution with ethyl acetate was followed by extraction with brine. The organic layer was dried over Na₂SO₄, concentrated, and purified by flash chromatography (silica 12 g, A: cyclohexane, B: ethyl acetate, C: MeOH, gradient program from Table Ex-4.). Product fractions were analyzed via ESI-MS.

TABLE Ex-4

| Time | | | |
|---|---|---|---|
| total | Flow | % | % |
| [min] | [mL/min] | B | C |
| 0 | 30 | 20 | 0 |
| 16 | 30 | 60 | 0 |
| 24 | 30 | 100 | 0 |
| 43 | 30 | 100 | 0 |
| 53 | 30 | 90 | 10 |
| 58 | 30 | 80 | 20 |
| 63 | 30 | 20 | 80 |
| 73 | 30 | 29 | 80 |

ESI-MS: [M+H]⁺: 678.311, Calculated 678.314

Compound 3a and 3b are both used for the following steps which is exemplary shown for compound 3a.

Synthesis of Compound 4 with the Following Structure:

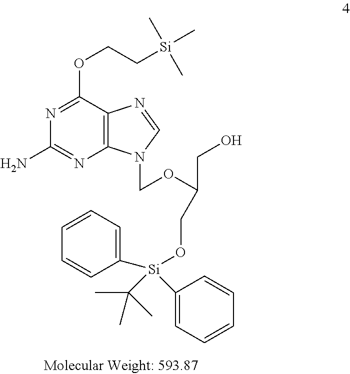 [SMILES: CC(C)(C)[Si](OCC(CO)OCn1cnc2c(OCC[Si](C)(C)C)nc(N)nc21)(c1ccccc1)c1ccccc1]

Compound 3a (223 mg, 1.0 eq.) in THF/MeOH/H₂O (513/1, v/v/v, 12 mL) was treated at 0° C. with a 2 M solution of NaOH in H₂O (0.8 mL). The reaction mixture was neutralized after 5 min via the addition of ammonium DOWEX 50W-X8 (or another suitable Dowex resin) and acetic acid. All solids were removed by filtration, the remnant was dry-loaded on silica and purified by flash chromatography. (silica 12 g, A: ethyl acetate, B: MeOH, gradient program from Table Ex-5.). Product fractions were analyzed via ESI-MS and NMR (referenced to chemical shift of SiCMe₃ of compound 3a since solvent signal was covered by benzene moieties and no referenceable impurities could be identified).

TABLE Ex-5

| Time | | |
|---|---|---|
| total | Flow | % |
| [min] | [mL/min] | B |
| 0 | 30 | 0 |
| 16 | 30 | 15 |
| 17 | 30 | 80 |
| 22 | 30 | 80 |

Compound 4 was obtained as a white powder in a yield of 74%.

ESI-MS: [M+H]⁺: 594.292, Calculated 594.293

¹H NMR (80 MHz, CDCl₃) δ 7.70-7.37 (m, 9H), 5.57 (s, 2H), 5.27 (s, 2H), 4.60 (m, 2H), 4.13 (dd, 1H), 3.94-3.67 (m, 4H), 1.26 (m, 2H), 1.07 (s, 9H), 0.10 (s, 9H).

Synthesis of Compound 5 with the Following Structure:

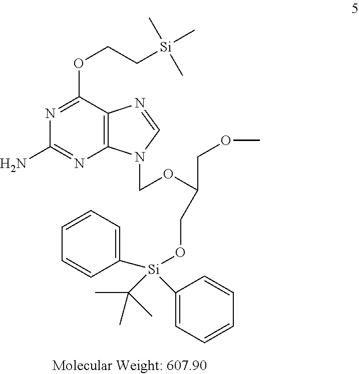 [SMILES: COCC(CO[Si](c1ccccc1)(c1ccccc1)C(C)(C)C)OCn1cnc2c(OCC[Si](C)(C)C)nc(N)nc21]

Compound 4 (153 mg, 1.0 eq.) was dried under high vacuum overnight and was then dissolved in DMF (2 mL) under protective gas atmosphere. Activated molecular sieves were added and the solution was stirred at ambient temperature for 30 min. MeI (3.0 eq.) and 90% NaH (1.5 eq.) were added consecutively. After 60 minutes, additional MeI (3.0 eq) and NaH (1.5 eq.) were added followed by the addition of another portion of NaH (1.5 eq) after 90 minutes. After 2 h total reaction time, the reaction was quenched by the addition of ethanol and diluted with ethyl acetate. Extraction with saturated ammonium chloride solution was followed by drying over Na₂SO₄ and purification by flash chromatography. (silica 12 g, A: cyclohexane, B: ethyl acetate, C: MeCOH, gradient program from Table Ex-6.). Product fractions were analyzed via ESI-MS.

Alternatively, compound 5 is prepared according to the general procedure 1 employing dry methanol as a nucleophile.

TABLE Ex-6

| Time | | | |
|---|---|---|---|
| total | Flow | % | % |
| [min] | [mL/min] | B | C |
| 0 | 30 | 0 | 0 |
| 30 | 30 | 100 | 0 |
| 31 | 30 | 90 | 10 |
| 36 | 30 | 90 | 10 |
| 37 | 30 | 20 | 80 |
| 42 | 30 | 20 | 80 |

ESI-MS: [M+H]⁺: 608.308, Calculated 608.308

Synthesis of Compound 6 with the Following Structure:

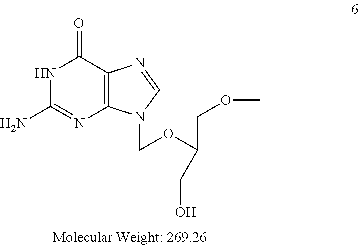 [SMILES: COCC(CO)OCn1cnc2c(=O)[nH]c(N)nc21]

### Method A:

HF-pyridine is diluted with pyridine. The mixture is cooled to 0° C. and added dropwise to an ice-cooled solution of compound 5 in DCM The reaction is deemed complete by TLC and is then diluted with ethyl acetate followed by extraction with 1 M TEAB solution. The product containing layer is evaporated to dryness, the residue is dry-loaded on silica and purified by flash chromatography.

### Method B:

THF containing 1 M TBAF is added to a solution of compound 5 in THF and the resulting solution is stirred at ambient temperature until TLC indicates complete conversion. The reaction is quenched with 1 M TEAB solution and all volatiles are removed under reduced pressure. The residue is dry-loaded on celite and purified by reversed-phase chromatography using C18 column.

Synthesis of Compound 7 with the Following Structure:

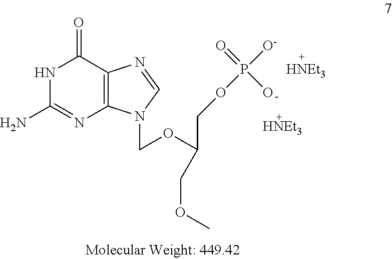 [SMILES: COCC(COP(=O)([O-])O)OCn1cnc2c(=O)[nH]c(N)nc21 ; "HNEt3" ; "HNEt3"]

Compound 7 is prepared from compound 6 according to the general procedure 2 or 3.

Synthesis of Compound 8 with the Following Structure:

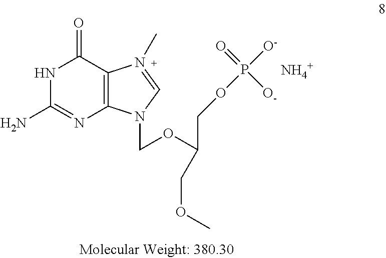

Compound 8 is synthesized from compound 7 according to the general procedure 4.

Synthesis of Compound 9a with the Following Structure:

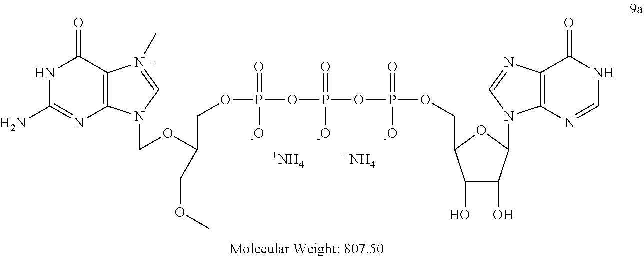

Compound 9a is synthesized from compound 8 according to the general procedure 5. Similar compounds can be synthesized in accordance with the above synthesis route when introducing different 3′-O-modifications and starting from Ganciclovir (X1) or Penciclovir (X2). Exemplary compounds along these lines are shown in FIG. 1, namely i) the “3′-OCE-m⁷-Ganciclovir” compound; ii) the “3′-OiPr-m⁷-Penciclovir” compound; iii) the “3′-OPhe-m⁷-Ganciclovir” compound; iv) the “3′-OPyBr-m⁷-Ganciclovir” compound; and v) the “3′-OCH₂C₅H₁₁-m⁷-Ganciclovir” compound.

The synthesis of the compounds shown above can be optimized/conducted by the following reaction pathway. To demonstrate the scope of these synthesis routes, the synthesis of the corresponding Cap1 (compound 9b) analog is shown in the following.

### Example 1.1.2: Synthesis Route I″

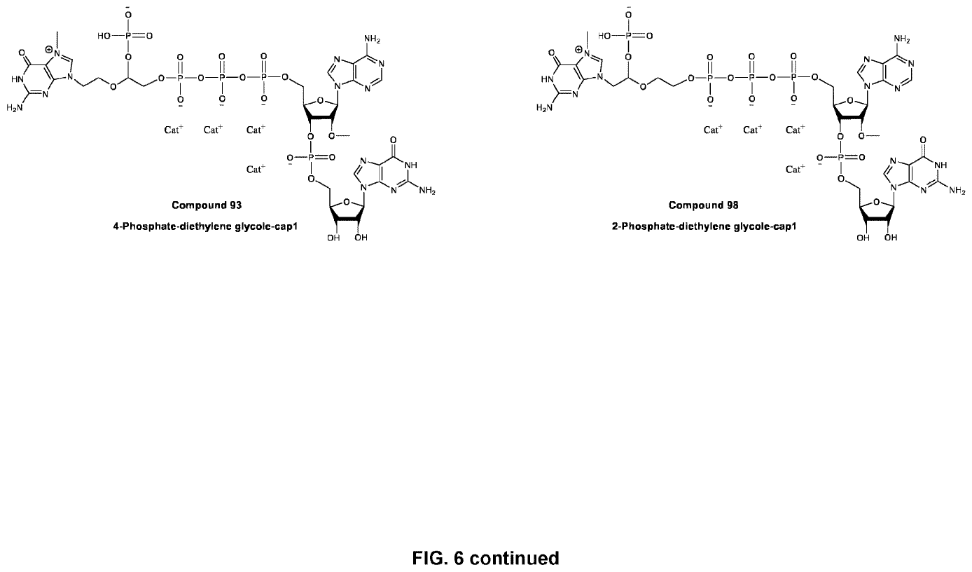

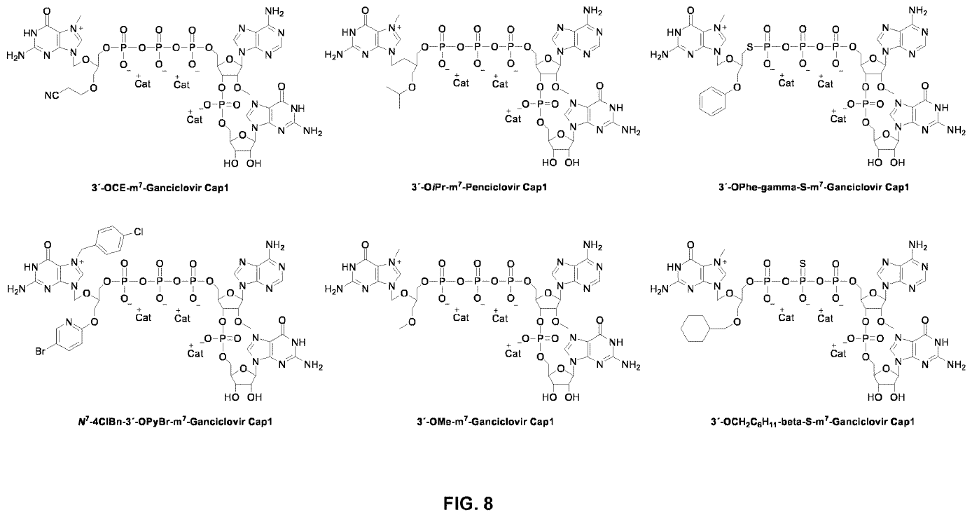

The synthesis of compound 9b (corresponding to 3′-OMe-m⁷-Ganciclovir-ppp-(5′)-AmG, referred to in FIGS. 8 and 10 as “3′-OMe-m⁷-Ganciclovir Cap1” when starting from Ganciclovir (X1) is shown in the following. It is noted that the synthesis route for other “3′-O-modified” derivatives also proceed via the described reaction pathway. Examples of resulting compounds analogous to compound 9b when starting from Ganciclovir (X1), Penciclovir (X2) or other nucleoside-diol compounds are described at the end of the present example and are depicted in FIG. 8.

Synthesis of Compound 1 was performed as described in Example 1.1.1: Synthesis route 1′

Synthesis of Compound 2a with the Following Structure:

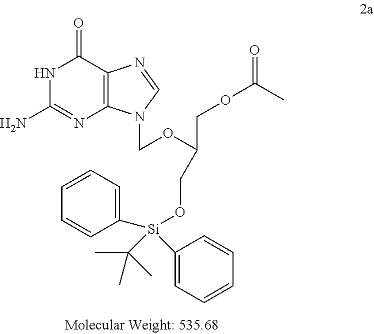 [SMILES: CC(=O)OCC(CO[Si](c1ccccc1)(c1ccccc1)C(C)(C)C)OCn1cnc2c(=O)[nH]c(N)nc21]

Synthesis of Compound 2a was performed as described in Example 1.1.1: Synthesis route 1′. Alternatively, it was prepared as follows:

Compound 1 (3.00 g, 1.0 eq.) was coevaporated three times with pyridine and was further dried under high vacuum for 30 min. It was then dissolved in pyridine (40 mL) and DMAP (0.1 eq.) was added. Ac₂O (1.1 eq.) was introduced dropwise to the reaction mixture which was stirred for 1 h at ambient temperature under argon gas atmosphere. The reaction mixture was then diluted with ethyl acetate and was washed three times with saturated sodium bicarbonate solution. The organic layer was dried over Na₂SO₄, was concentrated and purified by flash chromatography (silica 40 g, A: cyclohexane, B: ethyl acetate, gradient program from Table Ex-7)

TABLE Ex-7

| Time | | |
|---|---|---|
| total | Flow | B |
| [min] | [mL/min] | [%] |
| 0 | 45 | 0 |
| 29 | 45 | 8 |
| 39 | 45 | 80 |
| 44 | 45 | 80 |

Product fractions were analyzed via ESI-MS and NMR (data stated in Example 1.1.1: Synthesis route 1′). The product was obtained as a white powder in a yield of 92%.

Synthesis of Compound 3a was performed as described as described in Example 1.1.1: Synthesis route 1′

Synthesis of Compound 4 with the Following Structure:

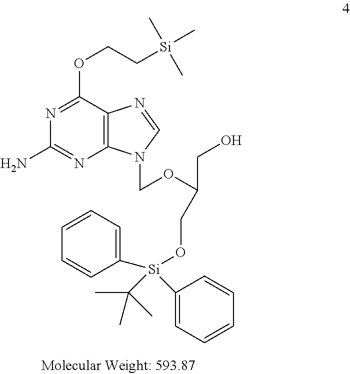 [SMILES: CC(C)(C)[Si](OCC(CO)OCn1cnc2c(OCC[Si](C)(C)C)nc(N)nc21)(c1ccccc1)c1ccccc1]

Synthesis of Compound 4 was performed as described in Example 1.1.1: Synthesis route 1′. Alternatively, it was prepared as follows:

Compound 3a (1.72 g, 1.0 eq) in THF/MeOH/H₂O (5/3/1, v/v/v, 90 mL) was treated at 0° C. with a 2 M solution of NaOH in H₂O (6 mL). The reaction mixture was neutralized after 10 min via the addition of pyridinium DOWEX 50W-X8. All solids were removed by filtration, the remaining liquid was dry-loaded on silica and purified by flash chromatography (silica 40 g, A: cyclohexane, B ethyl acetate, gradient program from Table Ex-8).

TABLE Ex-8

| Time | | |
|---|---|---|
| total | Flow | B |
| [min] | [mL/min] | [%] |
| 0 | 30 | 10 |
| 25 | 30 | 100 |
| 35 | 30 | 100 |

Product fractions were analyzed via ESI-MS and NMR (data stated in Example 1.1.1: Synthesis route 1′). The product was obtained as a white powder in a yield of 74%.

Synthesis of Compound 5 with the Following Structure

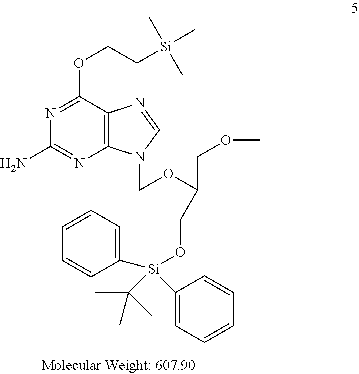 [SMILES: COCC(CO[Si](c1ccccc1)(c1ccccc1)C(C)(C)C)OCn1cnc2c(OCC[Si](C)(C)C)nc(N)nc21]

Synthesis of Compound 5 was performed as described in Example 1.1.1 Synthesis route 1′. Alternatively, it was prepared as follows:

Dry compound 4 (12 g, 1.0 eq.) was dissolved in DMF (120 mL) under protective gas atmosphere. Activated molecular sieves were added and the solution was left stirring for 30 min at ambient temperature and was then cooled to −20° C. using a salt bath. NaH (60%, 2.0 eq.) was added followed by the addition of MeI (1.1 eq.). The reaction was stirred for 10 min after which the salt bath was changed for an ice bath. Stirring was continued for 7 h at −4° C. followed by the addition of another portion of NaH (60%, 0.5 eq.) and MeI (0.7 eq.). After 2 h the reaction mixture was carefully poured into a mixture of water and brine (650 mL each) and extracted trice with ethyl acetate (400 mL each). The combined organic layers were washed with 0.3 M aqueous sodium metabisulfite (300 mL) and 50% brine (2×400 mL) and were then dried over Na₂SO₄. Concentration under reduced pressure yielded a clear oil which was purified twice by flash chromatography using Table Ex-9 and Table Ex-10 (silica 220 g, A: n-heptane, B: ethyl acetate).

TABLE Ex-9

| Time | | |
|---|---|---|
| total | Flow | B |
| [min] | [mL/min] | [%] |
| 0 | 120 | 5 |
| 2 | 120 | 5 |
| 29 | 120 | 50 |
| 32 | 120 | 80 |
| 50 | 120 | 100 |

TABLE Ex-10

| Time | | |
|---|---|---|
| total | Flow | B |
| [min] | [mL/min] | [%] |
| 0 | 120 | 5 |
| 43 | 120 | 45 |
| 53.5 | 120 | 45 |

Product fractions were analyzed via ESI-MS (data stated in Example 1.1.1: Synthesis route 1′) and NMR (referenced to ethyl acetate impurity since solvent signal was covered by benzene moieties). The product was obtained as a beige oil in a yield of 58%.

¹H NMR (400 MHz, CDCl₃) δ 7.68-7.65, 7.43-7.37 (m, 11H), 5.58 (s, 2H), 5.17 (s, 2H), 4.61 (m, 2H), 3.90 (m, 1H), 3.71 (d, 2H), 3.44 (2×dd, 4H), 3.27 (s, 3H), 1.26 (m, 2H), 1.05 (s, 9H), 0.11 (s, 9H).

Synthesis of Compound 6 with the Following Structure:

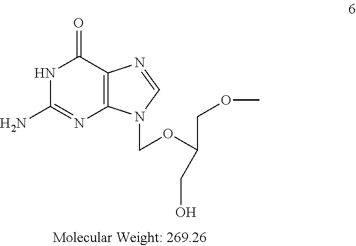 [SMILES: COCC(CO)OCn1cnc2c(=O)[nH]c(N)nc21]

Synthesis of Compound 6 can be performed as described in Example 1.1.1: Synthesis route 1′. Alternatively, it was prepared as follows:

Compound 5 (314 mg, 1.0 eq) was treated with 1 M TBAF in THF (5.0 eq.). The solvent was evaporated after 1.5 h and the oily residue was coevaporated trice with ethanol. The crude product was further dried under high vacuum overnight, was then dissolved in water and was purified via C18 RP, using a Büchi C18, 300×30 mm, 5 μM Column. Buffer A: 5 mM ammonium acetate pH 5.6, Buffer B: 100% MeOH, gradient program from Table Ex-11.

TABLE Ex-11

| Time | | |
|---|---|---|
| total | Flow | B |
| [min] | [mL/min] | [%] |
| 0 | 12 | 0 |
| 10 | 12 | 0 |
| 40 | 12 | 40 |
| 41 | 12 | 80 |
| 51 | 12 | 80 |
| 52 | 12 | 0 |
| 62 | 12 | 0 |

Product fractions were analyzed via ESI-MS and NMR. The product was obtained as a white powder in a yield of 50%.

ESI-MS: [M−H]⁻: 268.105, Calculated 268.105

¹H NMR (400 MHz, d₆-DMSO) δ 7.79 (s, 1H), 6.52 (s br., 2H), 5.42 (s, 2H), 3.70 (m, 1H), 3.39-3.22 (m, 4H), 3.17 (s, 3H).

Synthesis of Compound 7 with the Following Structure:

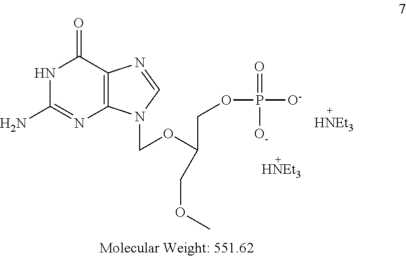

Synthesis of Compound 7 can be performed as described in Example 1.1.1: Synthesis route 1′. Alternatively, it was prepared as follows:

Compound 7 was prepared from compound 6 (3.5 g, 1.0 eq.) according to the general procedure 3 employing 26 mL trimethylphosphate and 2.0 eq. POCl₃. The reaction was quenched after 2 h at 0° C. with TEAB-buffer (130 mL, 0.1 M, pH 8.5). Purification was carried out via IEX column chromatography according to general procedure 3, using the gradient program from table Ex-12 (A: water, B: 0.5 M TEAB).

TABLE Ex-12

| Time | | |
|---|---|---|
| total | Flow | B |
| [min] | [mL/min] | [%] |
| 0 | 25 | 0 |
| 7 | 25 | 0 |
| 47 | 25 | 100 |
| 72 | 25 | 100 |
| 72 | 25 | 0 |
| 97 | 25 | 0 |

Product fractions were lyophilized from water and analyzed via ESI-MS and NMR. The product was obtained as a white powder in a yield of 91% (49% purity, impurities were identified as triethylammonium species).

ESI-MS: [M−H]⁻: 348.071, Calculated 348.072

¹H NMR (400 MHz, D₂O) δ 8.11 (s, 1H), 5.64 (m, 2H), 4.04 (m, 1H), 3.95 (m, 1H), 3.88 (m, 1H), 3.49 (m, 2H), 3.20 (s, 3H).

³¹P-NMR (162 MHz, D₂O) δ 0.24.

Synthesis of Compound 8 with the Following Structure:

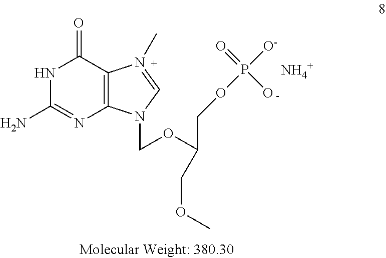

Synthesis of Compound 8 can be performed as described in Example 1.1.1: Synthesis route 1′. Alternatively, it was prepared as follows:

Compound 8 was synthesized from compound 7 (13.3 g, 49% purity, 1.0 eq.) according to the general procedure 4 using 118 mL DMSO and 8 eq. MeI The reaction mixture was stirred for 7 h at ambient temperature. It was then cooled to 0° C. and stirred for 16 h where it was allowed to warm to ambient temperature. Stirring was continued for 8 h at ambient temperature and was then followed by dilution of the reaction mixture with water (1.5 L), extraction with diethyl ether (4×100 mL) and treatment with sodium bisulfite. Remaining diethyl ether in the aqueous layer was removed under reduced pressure. It was then diluted with water to a total volume of 2 L and purification was carried out as described in the general procedure 4, starting with ion exchange chromatography using the gradient program from table Ex-13 (A: water, B: 0.5 M TEAB), followed by repurification by C18 RP using the gradient program from table Ex-14 (Buffer A: 10 mM ammonium acetate, Buffer B: 100% MeOH, Büchi SELECT column) and using the gradient program of table Ex-15 (Buffer A: 10 mM ammonium acetate, Buffer B: 100% MeCOH, Buchi ECOFLEX column).

TABLE Ex-13

| Time | | |
|---|---|---|
| total | Flow | B |
| [min] | [mL/min] | [%] |
| 0 | 25 | 0 |
| 12 | 25 | 0 |
| 82 | 25 | 100 |
| 107 | 25 | 100 |
| 107 | 25 | 0 |
| 132 | 25 | 0 |

TABLE Ex-14

| Colume | Flow | B |
|---|---|---|
| volumes | [mL/min] | [%] |
| 0 | 50 | 1 |
| 1.5 | 50 | 1 |
| 5.5 | 50 | 30 |
| 6.5 | 50 | 100 |
| 9.5 | 50 | 100 |

TABLE Ex-15

| Colume | Flow | % |
|---|---|---|
| volumes | [mL/min] | B |
| 0 | 40 | 0 |
| 2 | 40 | 0 |
| 5.5 | 40 | 30 |
| 6 | 40 | 100 |
| 9 | 40 | 100 |

Product fractions were lyophilized from water and analyzed via ESI-MS and NMR. The product was obtained as a white powder in a yield of 50% (95% purity).

ESI-MS: [M−H]⁻: 364.101, Calculated 364.101

¹H NMR (400 MHz, D₂O) δ 5.78 (m, 2H), 4.12 (s, 3H), 4.10 (m, 1H), 3.92 (m, 1H), 3.83 (m, 1H), 3.58 (dd, 1H), 3.52 (dd, 1H), 3.30 (s, 3H).

³¹P-NMR (162 MHz, D₂O) δ 0.82.

Synthesis of Compound 9b with the Following Structure:

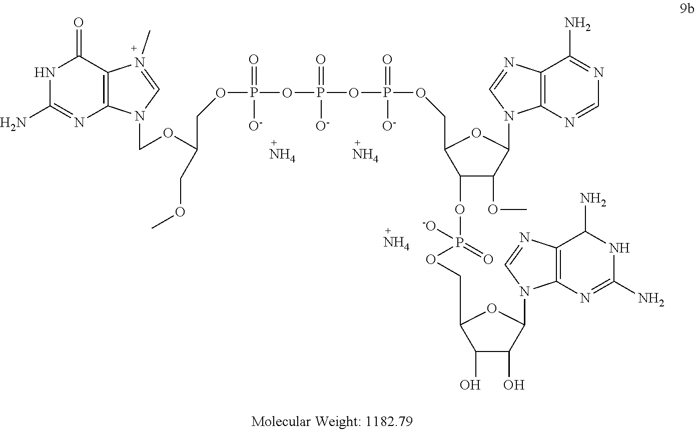

Compound 9b was prepared from compound 8 (100 mg, 1.0 eq.) following the general procedure 7 employing 1 mL DMSO, 20.0 eq CDI (2 portions within 48 h) and 20.0 eq water. Total reaction time: 5 d. Compound 9b was purified according to general procedure 7 using the gradient program of table Ex-16 (A: 5 mM ammonium acetate in water; B: 90% methanol in water), followed by a second purification using the gradient program of table Ex-17 (A: 100 mM triethylammonium acetate (pH 7), B: 90% MeOH in water), followed by a third purification using the gradient program of table Ex-18 (A: 5 mM ammonium acetate in water; B: 90% methanol in water).

TABLE Ex-16

| Time | A | B |
|---|---|---|
| [min] | [%] | [%] |
| 0 | 100 | 0 |
| 10 | 100 | 0 |
| 20 | 80 | 20 |
| 21 | 5 | 95 |
| 29 | 5 | 95 |
| 30 | 100 | 0 |
| 45 | 100 | 0 |

TABLE Ex-17

| Time | A | B |
|---|---|---|
| [min] | [%] | [%] |
| 0 | 100 | 0 |
| 40 | 60 | 40 |
| 41 | 100 | 0 |
| 55 | 100 | 0 |

TABLE Ex-18

| Time | A | B |
|---|---|---|
| [min] | [%] | [%] |
| 0 | 100 | 0 |
| 10 | 100 | 0 |
| 30 | 5 | 95 |
| 35 | 5 | 95 |
| 37 | 100 | 0 |
| 50 | 100 | 0 |

Product fractions were analyzed via ESI-MS and NMR. The product was obtained as a white powder in a yield of 24%.

ESI-MS: [M]⁺: 1118.168, Calculated 1118.164

¹H NMR (400 MHz, D₂O) δ 8.96 (m, 1H), 8.34 (m, 1H), 8.05 (s, 1H), 7.87 (s, 1H), 5.95 (m, 1H), 5.74 (m, 1H), 5.54 (m, 2H), 4.86 (m, 1H), 4.45 (m, 1H), 4.40 (m, 1H), 4.35 (m, 1H), 4.26-4.10 (m, 5H), 3.98 (m, 2H), 3.95 (m, 3H), 3.88 (m, 1H), 3.36 (s, 3H), 3.11 (m, 3H).

³¹P NMR (162 MHz, D₂O) 6-0.89 (s, 1P), −11.42 (m, 2P), −22.87 (m, 1P).

Similar compounds can be synthesized in accordance with the above synthesis route when introducing different 3′-O-modifications and starting from Ganciclovir (X1) or Penciclovir (X2). Exemplary compounds along these lines are shown in FIG. 8, namely i) the “3′-OCE-m⁷-Ganciclovir Cap1” compound; ii) the “3′-OiPr-m⁷-Penciclovir Cap1” compound; iii) the “3′-OPhe-gamma-S-m⁷-Ganciclovir Cap1” compound; iv) the “N⁷-4ClBn-3′-OPyBr-m⁷-Ganciclovir Cap1” compound; and v) the “3-OCH₂C₆H₁₁-beta-S-m⁷-Ganciclovir Cap1” compound.

### Example 1.2: Synthesis Route II

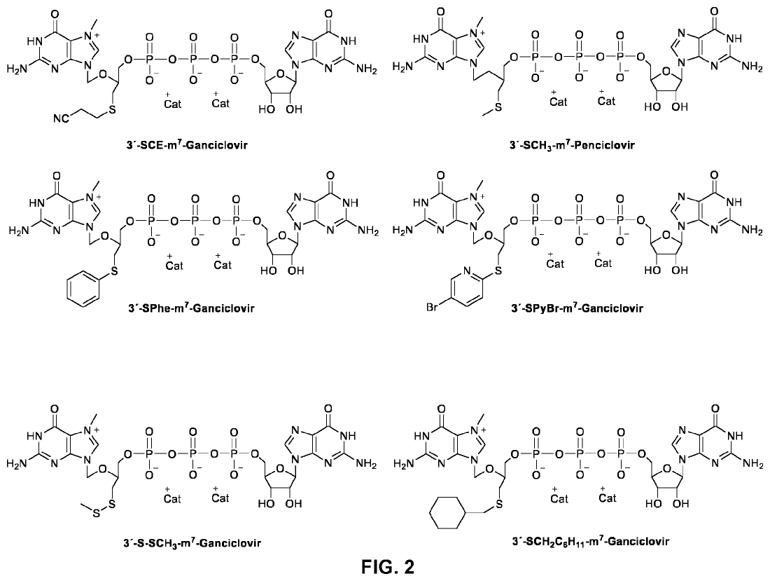

The synthesis of compound 15 (corresponding to 3′-(2-cyanoethylthio)-m⁷-Ganciclovir-ppp-(5′)-Guanosine, referred to the “3′-SCE-m⁷-Ganciclovir” compound in FIG. 2) when starting from compound 1 (example 1.1.1: synthesis route I′) is described in the following. It is noted that other 3′-thio-modified derivatives with the following general structure G1, referred to as “3-SR-m⁷-Ganciclovir”, are synthesized similarly to the described reaction sequence using Ganciclovir (X1) or Penciclovir (X2) as educt.

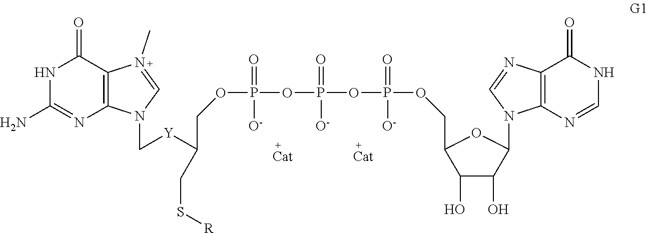

R can be any saturated/unsaturated/semi-saturated/aromatic carbocycle or heterocycle directly attached to the 3′-sulfur or attached to the 3′-sulfur via an alkyl chain, or any linear and/or branched saturated/unsaturated/semi-saturated C1-Cn (n=any number of carbons) alkyl chain, which can include one or more heteroatoms like O, S, N, wherein R can be unsubstituted or substituted with further substituents such as halogen, CN, alkyl, OH, SH, NH₂ or oxo. Cat can be any positively charged counterion like NH₄⁺, Na⁺, Li⁺, K⁺, HEt₃N⁺ or any other positively charged amine species. Y can be O or CH₂. Examples for compounds with the general structure G1 can be synthesized similarly to compound 15 and are depicted in FIG. 2

Synthesis of Compound 10 with the Following Structure:

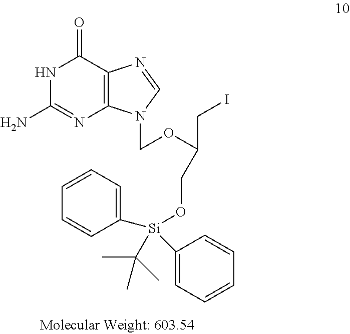 [SMILES: CC(C)(C)[Si](OCC(CI)OCn1cnc2c(=O)[nH]c(N)nc21)(c1ccccc1)c1ccccc1]

Compound 1, triphenylphosphine and imidazole are suspended in NMP and treated with a solution of iodine in NMP over the course of 10 min. Upon completion, the reaction mixture is concentrated, diluted with ethyl acetate, and washed with saturated sodium hydrogencarbonate solution. The organic layer is dried over Na₂SO₄, concentrated, and purified by flash chromatography.

Synthesis of Compound 11 with the Following Structure:

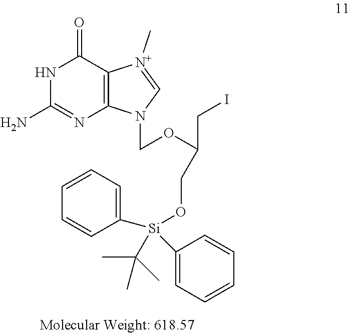 [SMILES: C[n+]1cn(COC(CI)CO[Si](c2ccccc2)(c2ccccc2)C(C)(C)C)c2nc(N)[nH]c(=O)c21]

Compound 11 is prepared from compound 10 according to the general procedure 4 or as described in the following: MeI is added dropwise to a vigorously stirred solution of compound 10 in DMSO. The reaction is deemed complete by TLC and/or ESI-MS and is then concentrated in high vacuum. Compound 11 is precipitated by the addition of diethyl ether and is collected by filtration. The product is washed extensively with diethyl ether, is dried under high vacuum and purified by flash chromatography.

Synthesis of Compound 12 with the Following Structure:

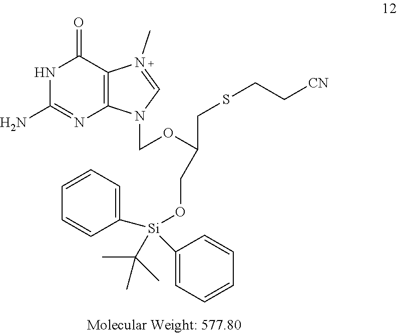

3-Mercaptopropionitrile is freshly prepared by suspending 3,3′-dithiobis(propionitrile) in 2 M HCl. Zinc powder is added carefully and the metallic slurry is stirred 1 h at ambient temperature. The reaction mixture is extracted trice with DCM, the organic layer is dried over Na₂SO₄ and all volatiles are removed under reduced pressure yielding 3-mercaptopropionitrile as a colorless oil. The latter is added dropwise to a solution of compound 11 and N-methyl-pyrrolidine in DMF which is stirred until TLC indicates full consumption of the starting material. The reaction mixture is diluted with water and extracted trice with ethyl acetate. The product containing layer is concentrated and purified by chromatography.

Synthesis of Compound 13 with the Following Structure:

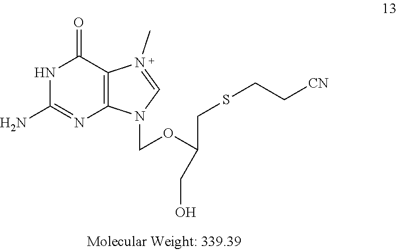

THF containing 1 M TBAF and 0.5 M acetic acid is added to a solution of compound 12 in THF and the resulting mixture is stirred at ambient temperature until TLC and/or (RP-)TLC indicates complete consumption of the starting material. The reaction is quenched with 1 M TEAB solution and all volatiles are removed under reduced pressure. The residue is purified by chromatography.

Synthesis of Compound 14 with the Following Structure:

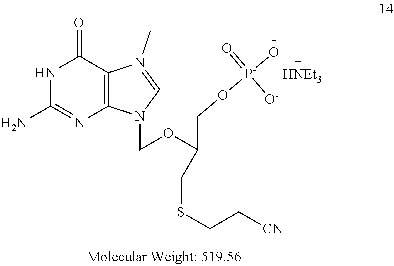

Compound 14 is prepared from compound 13 according to the general procedure 3. Other 3′-thio modified derivatives are synthesized according to the general procedure 2 or 3.

Synthesis of Compound 15 with the Following Structure:

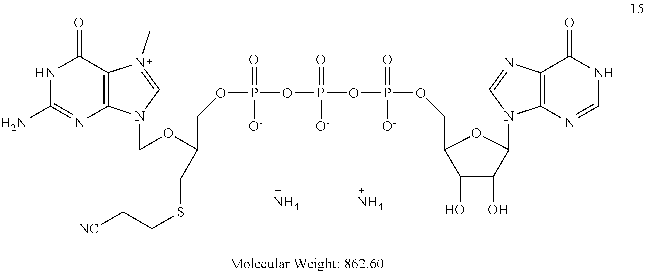

Compound 15 is synthesized from compound 14 according to the general procedure 5.

Similar compounds can be synthesized in accordance with the above synthesis route when introducing different 3′-thio-modifications. Exemplary compounds along these lines are shown in FIG. 2, namely i) the “3′-SCH₃-m⁷-Penciclovir” compound; ii) the “3′-SPhe-m⁷-Ganciclovir” compound; iii) the “3′-SPyBr-m⁷-Ganciclovir” compound; iv) the “3′-S—SCH₃-m⁷-Ganciclovir” compound; and v) the “3′-SCH₂C₆H₁₁-m⁷-Ganciclovir” compound all starting from compound 1.

### Example 1.3: Synthesis Route III

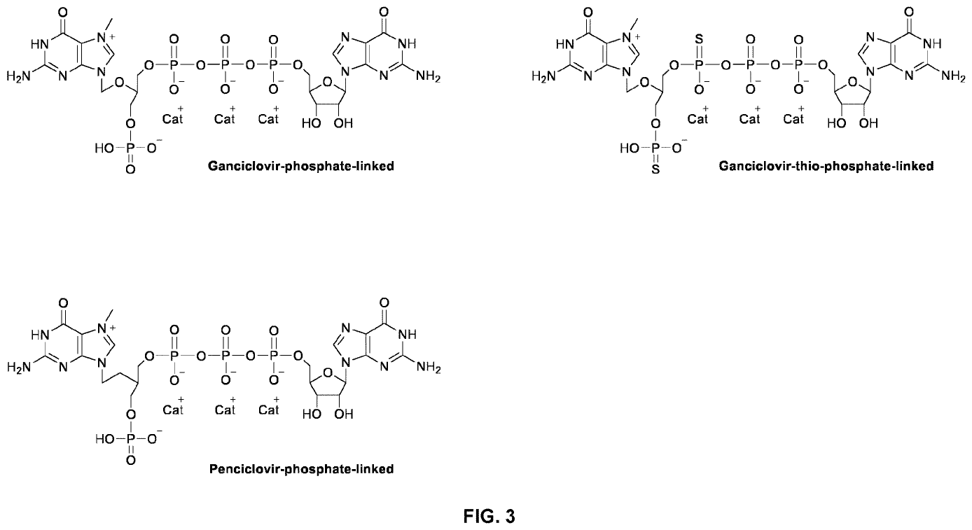

The synthesis of compound 18 (corresponding to [[(m⁷Guanin-9-yl)-methoxy]-propyl-3-phosphate]-(1-ppp-Guanosine)), referred to in FIG. 3 as “Ganciclovir-phosphate-linked” compound) when starting from Ganciclovir (X₁) is shown in the following. It is noted that the synthesis route when starting from other diol nucleotide compounds (such as e.g. Penciclovir (X2)) is identical. Examples of resulting compounds analogous to compound 18 when starting from such other diol compounds are described at the end of the present example and are depicted in FIG. 3.

Synthesis of Compound 16 with the Following Structure:

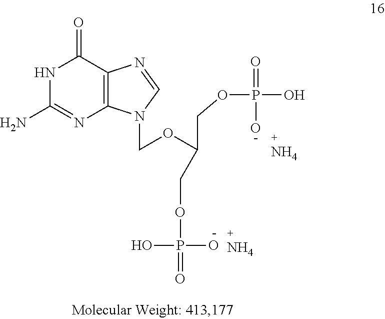

Synthesis of compound 16 was performed according to general procedure 2, starting from Ganciclovir (X1, 1 eq.), bis-cyanoethyl-N,N-diisopropyl-phosphoramidite (2 eq.) and tetrazole (2 eq., 0.45 M in acetonitrile) with oxidation protocol using a 0.1 M iodine solution. Deprotection step with ammonia was performed at 40° C. for 48 h without addition of DTT. Sample was subjected to ion exchange chromatography using a TEAB gradient from 0-1 M within 90 minutes. Final purification step using RP-HPLC with a Büchi C18 250×30 mm, 5 μM column was performed according to gradient program shown in Table Ex-19. Buffer A: 5 mM ammonium acetate pH 5.6, Buffer B: 100% MeOH.

TABLE Ex-19

| Time | | |
|---|---|---|
| total | Flow | % |
| [min] | [mL/min] | B |
| 0 | 12 | 0 |
| 25 | 12 | 12 |
| 26 | 12 | 80 |
| 31 | 12 | 80 |
| 32 | 12 | 0 |
| 42 | 12 | 0 |

Product fractions were analyzed via ESI-MS and NMR. The product was obtained as a white powder in a yield of 59%.

ESI-MS: [M−H]. 414.022, Calculated 414.022

¹H-NMR (80 MHz, D₂O) δ 8.06 (s, 1H), 5.67 (s, 2H), 4.00 (m, 5H).

³¹P-NMR (32.63 MHz, D₂O) δ 0.64 (s).

Synthesis of Compound 17 with the Following Structure:

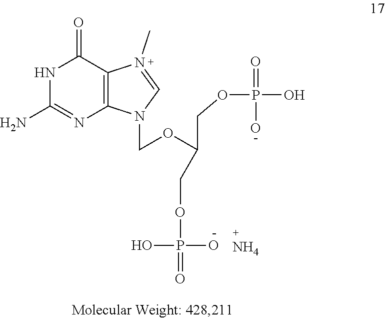

Synthesis of compound 17 was performed according to general procedure 4 with compound 16 as educt. The pH was adjusted to 7.5 after addition of sodium disulfite and subjected to ion exchange chromatography with a linear TEAB gradient from 0-1 M within 90 minutes and a final RP-HPLC purification using a Büchi 250×30 mm, 5 μm column with the gradient according to table Ex-19 above. Buffer A: 5 mM ammonium acetate pH 5.6, Buffer B: 100% MeCOH.

Product fractions were analyzed via ESI-MS and NMR. The product was obtained as a white powder in a yield of 32%.

ESI-MS: [M⁺]: 430.051, Calculated 430.052

¹H-NMR (80 MHz, D₂O) δ 9.11 (s, 1H), 5.81 (s, 2H), 4.13 (s, 3H), 3.90 (m, 5H).

³¹P-NMR (32.63 MHz, D₂O) δ 0.38 (s).

Synthesis of Compound 18 with the Following Structure:

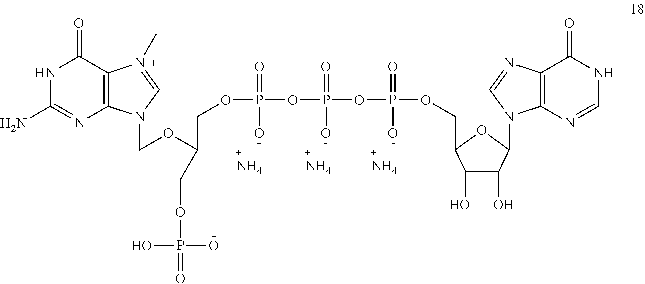

Synthesis of compound 18 was performed according to general procedure 5 starting from compound 17 (1 eq.), MgCl₂ (10 eq.) and H₂O/ACN as solvent. Purification was performed according to method B with the gradients shown in table Ex-20, 21 and 22 as described below.

The reaction mixture was desalted using RP-HPLC using the gradient program in table Ex-20 with solvent A: 5 mM ammonium acetate in water; solvent B: 90% methanol in water.

TABLE Ex-20

| Time | A | B |
|---|---|---|
| [min] | [%] | [%] |
| 0 | 100 | 0 |
| 10 | 100 | 0 |
| 20 | 85 | 15 |
| 21 | 20 | 80 |
| 28 | 20 | 80 |
| 29 | 100 | 0 |
| 36 | 100 | 0 |

The desalted reaction mixture was purified by ion exchange chromatography with DNAPac PA200 column (22×250 mm) using Buffer A: 20 mM Tris, pH9, Buffer B: 20 mM Tris, 330 mM sodium perchlorate. The gradient program is described in table Ex-21.

TABLE Ex-21

| Time | A | B |
|---|---|---|
| [min] | [%] | [%] |
| 0 | 100 | 0 |
| 68 | 83 | 17 |
| 69 | 0 | 100 |
| 74 | 0 | 100 |
| 75 | 100 | 0 |
| 85 | 100 | 0 |

The product fraction was desalted using RP-HPLC using a Phenomexx Gemini C18, 250×21.2 mm, 5 μM Column. Buffer A: 5 mM ammonium acetate pH 5.6, Buffer B: 90% MeOH with the gradient program described in table Ex-22.

TABLE Ex-22

| Time | A | B |
|---|---|---|
| [min] | [%] | [%] |
| 0 | 100 | 0 |
| 10 | 100 | 0 |
| 30 | 70 | 30 |
| 31 | 20 | 80 |
| 40 | 20 | 80 |
| 41 | 100 | 0 |
| 50 | 100 | 0 |

The resulting fraction was repurified via C18 RP HPLC, using a Phenomexx Gemini C18, 250×21.2 mm, 5 μM Column. Buffer A: 5 mM ammonium acetate pH 5.6, Buffer B: 90% MeOH, gradient program in Table Ex-22. The product compound 18 was obtained as white solid in a yield of 8%.

### Analytical Data of Compound 18

¹H NMR (400 MHz, D₂O) δ 8.03 (s, 1H), 5.85 (s, 1H), 5.80 (d, 1H), 5.70 (q, 2H), 4.70 (t, 1H), 4.49 (m, 1H), 4.37 (m, 1H), 4.30 (m, 2H), 4.14 (m, 2H), 4.07 (s, 3H), 4.04 (m, 1H), 3.87 (m, 2H).

³¹P-NMR (162 MHz, D₂O) δ 0.27, −11.22, −23.03.

ESI-MS: [M⁺]: 855.065, Calculated 855.066

The product fractions were lyophilized and dissolved in ultrapure water resulting in a 100 mM solution.

Similar compounds can be synthesized in accordance with the above synthesis route when starting from Ganciclovir (X1), Penciclovir (X2) or similar diol compounds. Exemplary compounds along these lines are shown in FIG. 3, namely i) the “Penciclovir-phosphate-linked” compound, and ii) the “Ganciclovir-thio-phosphate-linked” compound.

### Example 1.4: Synthesis Route IV

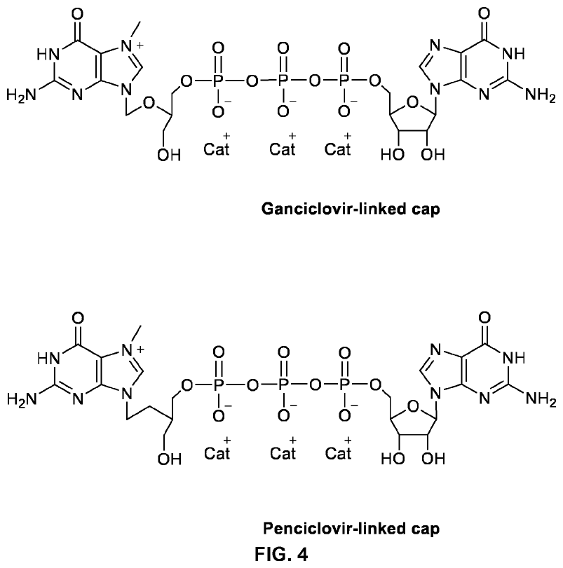

The synthesis of the Ganciclovir-linked cap analog (compound 19, referred to as “Ganciclovir-linked cap” in FIG. 4) is shown in the following. The analogous compound when starting from Penciclovir (X2) is described at the end of the present example and depicted as “Penciclovir-linked cap” in FIG. 4.

Synthesis of Compound 20 with the Following Structure:

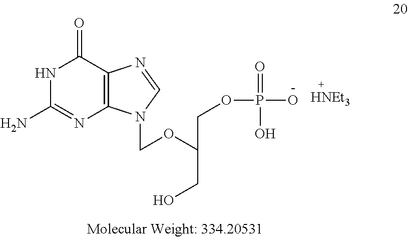 [SMILES: Nc1nc2c(ncn2COC(CO)COP(=O)(O)O)c(=O)[nH]1]

Synthesis of compound 20 was performed according to general procedure 2, starting with compound 1 (1 eq.) bis-cyanoethyl-N,N-diisopropyl-phosphoramidite (2 eq.), tetrazole (2 eq., 0.45M in acetonitrile) and oxidation protocol using 0.1 M iodine solution. Deprotection with ammonia was performed at 60° C. for 48 h. Final purification via IEX-Chromatography with Macro-Prep-High-Q resin (or optional via RP-HPLC) was performed with a TEAB gradient according to table Ex-23 (A: water, B: 1.0 M TEAB) (or optional via a TEAA (0.1 M) to methanol gradient). Compound 20 can also be synthesized as its ammonium salt as described for compound 105 in Example 1.9: Synthesis route IX.

TABLE Ex-23

| Time | Flow | % |
|---|---|---|
| total | [ml/min] | B |
| 0 | 6 | 0 |
| 15 | 60 | 0 |
| 60 | 60 | 100 |
| 70 | 60 | 100 |
| 70.1 | 60 | 0 |
| 77.1 | 60 | 0 |

Product fractions were analyzed via ESI-MS and NMR. The product was obtained as a white powder in a yield of 81%.

ESI-MS: [M−H]: 334.0557, Calculated 334.0558

¹H NMR (80 MHz, D₂O) δ 7.96 (s, 1H), 5.62 (s, 2H), 3.95 (m, 3H), 3.64 (s, 2H).

Synthesis of Compounds 21 and 19 with the Following Structures:

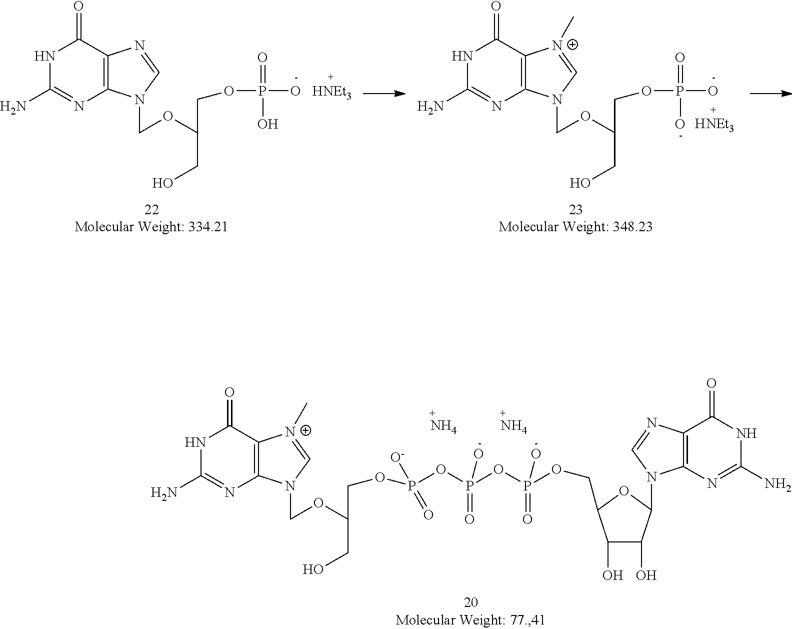

The N⁷-methylation via methyl iodide to compound 21 was performed according to general procedure 4 using compound 20 as starting material with consecutive ion exchange chromatography using the TEAB gradient program described in table Ex-23 and a RP-HPLC purification using a Büchi C18 250×30 mm, 5 μM column with the gradient described in table Ex-19 for final purification step. C18 buffer system: Buffer A: 5 mM ammonium acetate pH 5.6, Buffer B: 100% MeCOH.

Alternatively, Compound 20 can be methylated at N⁷ as described for compound 106 in Example 1.9: Synthesis route IX.

Product fractions were analyzed via ESI-MS and NMR. The product was obtained as a white powder in a yield of 12%.

ESI-MS: [M+H]: 350.0856, Calculated 350.0855

¹H NMR (80 MHz, D₂O) δ 5.81 (s, 2H), 4.14 (s, 3H), 3.95 (m, 3H), 3.71 (m, 2H).

The following magnesium mediated condensation reaction to compound 19 was performed according to general procedure 5 with compound 21 (1 eq.) as educt. Reaction was performed in presence of magnesium chloride (10 eq.) in H₂O/ACN (1/1, v/v) as solvent.

Purification was performed according to method B with the gradients of table Ex-20, 21 and 22 as described above for compound 18.

Product fractions were analyzed via ESI-MS and NMR. The product was obtained as a white powder in a yield of 18%.

ESI-MS: [M−H]. 773.085, Calculated 773.085

¹H NMR (400 MHz, D₂O) δ 8.01 (s, 1H), 5.80 (m, 1H), 5.68 (m, 2H), 4.65 (t, 1H), 4.49 (m, 1H), 4.35 (m, 1H), 4.28 (m, 2H), 4.11 (m, 1H), 4.04 (s, 3H), 4.00 (m, 2H), 3.7-3.5 (m, 2H).

Similar compounds can be synthesized in accordance with the above synthesis route when starting from other starting compounds, e.g Penciclovir (X2), or wherein the substitution pattern (and optionally the chain length of the carbon linker) is different from the pattern (and length) in compound 20. An example of such variants is shown in FIG. 4, namely the “Penciclovir-linked cap” compound.

Examples 1.1.1 to 1.4 show the synthesis of “dinucleotide-like structures”. The following examples 1.5 to 1.7 show the synthesis of “trinucleotide-like structures”. Given this information, the skilled person is readily in a position to e.g. synthesize the trinucleotide-like structures of the dinucleotide-like structures synthesized or shown herein (e.g. as shown in FIGS. 1 to 4). Thus, for compounds as shown in FIGS. 1 to 4, the trinucleotide-like structures are synthesized using the respective m7G-“linker”-monophosphate compound prepared according to the synthesis routes described in Example 1.1.1 to 1.4. The m7G-“linker”-monophosphate compound is activated, e.g., with carbonyldiimidazol, and subsequently condensated with a diphosphorylated AmG-dinucleotide ppAmpG (e.g. compound 28) as described in Example 1.5. It is known to the skilled person that the condensation of the activated m7G-“linker”-monophosphate with pp-trinucleotide compounds like ppAmpGpG or ppAmpGmpG, which are synthesized in a similar manner as the dinucleotide in Example 1.5, will lead to Cap1 or Cap2 tetranucleotide compounds.

### Example 1.5: Synthesis Route V

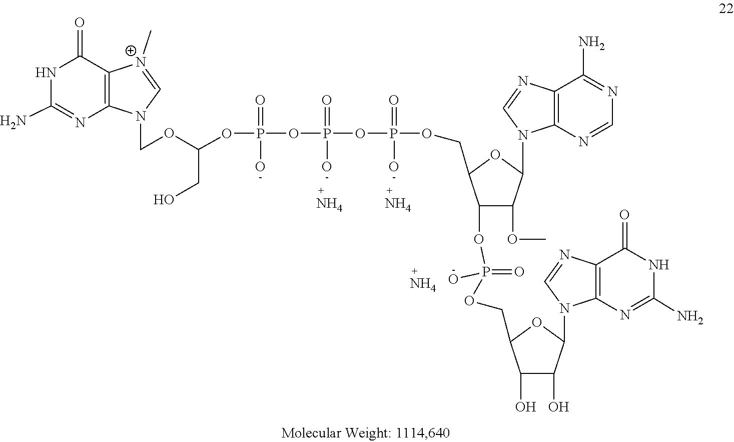 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OC(CO)OCn2c[n+](C)c3c(=O)[nH]c(N)nc32)OC1n1cnc2c(N)ncnc21 ; [NH4+] ; [NH4+] ; [NH4+]]

The synthesis of the Ganciclovir-linked Cap1 trinucleotide (compound 22) is shown in the following, wherein the synthesis inter alia started from commercially available 5′-O-DMT-N2-isobutyrylguanosine, Biosynth, UK with the following structure, referred to herein as X₃, and N⁶-Benzoyl-5′-O-DMT-2′-O-methyladenosine 3′-CE phosphoramidite, Biosynth, UK) with the following structure, referred to herein as X₄:

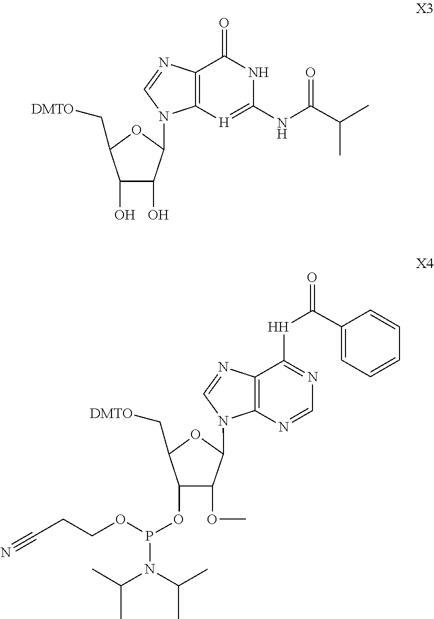

Synthesis of Compound 23 with the Following Structure:

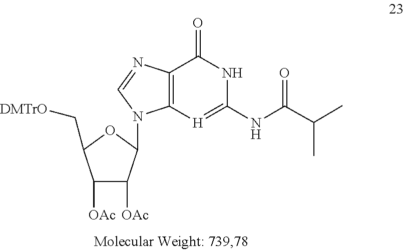

Compound X₃ (1 eq.) was dissolved under argon atmosphere in dry pyridine. 4-(Dimethylamino)-pyridine (0.15 eq.) and acetic anhydride (14 eq.) were added and the reaction mixture was stirred for 6 h. Dichloromethane was added and organic phase was washed three times with aqueous citric acid (5 wt. % in water). Solvent was evaporated and crude product was purified by flash column chromatography (Silica: 330 g, Solvent A: ethyl acetate, Solvent B: MeOH (2nd solvent), linear gradient according to following table Ex-24:)

TABLE Ex-24
Gradient Table

| | Min | Solvents | % 2nd |
|---|---|---|---|
| 1 | 0.0 | AB | 0 |
| 2 | 29.0 | AB | 7 |
| 3 | 1.0 | AB | 80 |
| 4 | 10.0 | AB | 80 |

The product was obtained as pale white foam in a yield of 84%.

¹H NMR (400 MHz, DMSO-d6) δ 12.12 (s, 1H), 11.52 (s, 1H), 8.12 (s, 1H), 7.36 (d, 2H), 7.22 (m, 7H), 6.83 (t, 4H), 6.14 (d, 1H), 5.96 (t, 1H), 5.47 (dd, 1H), 4.27 (m, 1H), 3.72 (s, 6H), 3.53 (dd, 1H), 3.27 (dd, 1H), 2.75 (m, 1H), 2.11 (s, 3H), 2.04 (s, 3H), 1.14 (s, 3H), 1.12 (s, 3H).

Synthesis of Compound 24 with the Following Structure:

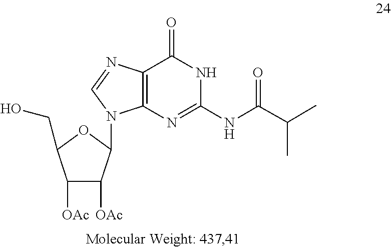 [SMILES: CC(=O)OC1C(CO)OC(n2cnc3c(=O)[nH]c(NC(=O)C(C)C)nc32)C1OC(C)=O]

Compound 23 (1 eq.) was dissolved in aqueous acetic acid (80% acetic acid in water (v/v) and stirred for 30-60 minutes. Solvents were evaporated and residue was coevaporated with methanol. The crude product was purified by flash column chromatography (Silica: 120 g, Solvent A: ethyl acetate, solvent B (2nd solvent): MeOH, linear gradient according to following table Ex-25).

TABLE Ex-25
Gradient Table

| | Min | Solvents | % 2nd |
|---|---|---|---|
| 1 | 0.0 | AB | 0 |
| 2 | 9.5 | AB | 4 |
| 3 | 9.2 | AB | 14 |
| 4 | 1.4 | AB | 81 |

The product was obtained as white foam in a yield of 86%.

¹H NMR (400 MHz, DMSO-d₆) δ 12.11 (s, 1H), 11.68 (s, 1H), 8.31 (s, 1H), 6.06 (d, 1H), 5.74 (dd, 1H), 5.47 (dd, 1H), 5.41 (t, 1H), 4.21 (q, 1H), 3.68 (m, 2H), 2.78 (m, 1H), 2.12 (s, 3H), 1.99 (s, 3H), 1.13 (s, 3H), 1.11 (s, 3H).

Synthesis of Compound 25 with the Following Structure:

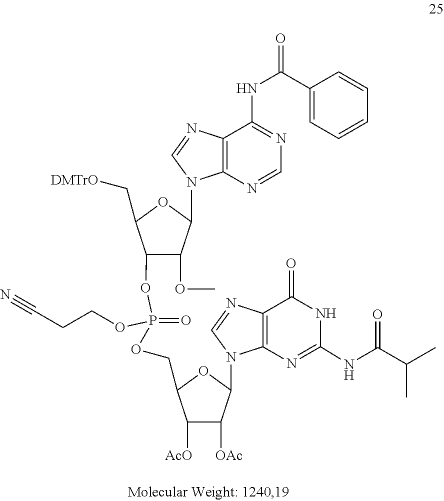

Compound 24 (1 eq.) and N6-Benzoyl-5′-O-DMT-2′-O-methyladenosine 3-CE phosphoramidite (X4, 1.1 eq.) were added to separate dry flasks and dried for 16 h at high vacuum. Flasks were flooded with argon and both educts were dissolved in extra dry acetonitrile for DNA synthesis. Dissolved 24 was added to the flask with dissolved N6-Benzoyl-5′-O-DMT-2′-O-methyladenosine 3-CE. Tetrazole (0.45 M in acetonitrile, 2.5 eq.) was added and the mixture was stirred for 60 minutes. Oxidizer (0.1 M Iodine in THE/Pyridine/water (77:21:2, v/v/v)) was added until the solution was red colored and stayed for 15 minutes without getting yellow again. A 1:1 mixture of aqueous sodium disulfite solution (5 wt. %) and citric acid solution (5 wt. %) was added and crude product was extracted with dichloromethane. Organic layer was washed with brine and dried with sodium sulfate. Solvent was evaporated and crude product was purified by flash column chromatography (Silica: 120 g, solvent A: ethyl acetate, solvent B (2nd solvent): MeOH, linear gradient according to following table Ex-26)

TABLE Ex-26
Gradient Table

| | Min | Solvents | % 2nd |
|---|---|---|---|
| 1 | 0.0 | AB | 0 |
| 2 | 33.5 | AB | 25 |
| 3 | 0.8 | AB | 80 |
| 4 | 3.7 | AB | 80 |

The product was obtained as white foam in a yield of 74%.

¹H-NMR (400 MHz, DMSO-d₆) δ 12.12 (s, 1H), 11.57 (s, 1H), 11.26 (s, 1H), 8.63 (d, 1H), 8.60 (d, 1H), 8.27 (d, 1H), 8.06 (d, 1H), 7.63 (t, 1H), 7.57 (t, 2H), 7.37 (t, 2H), 7.23 (m, 7H), 6.83 (m, 4H), 6.23 (d, 1H), 6.13 (t, 1H), 5.83 (m, 1H), 5.53 (m, 1H), 5.28 (m, 1H), 5.05 (m, 1H), 4.43 (m, 4H), 4.23 (m, 2H), 3.71 (d, 6H), 3.41-3.34 (m, 7H), 2.91 (m, 2H), 2.75 (m, 1H), 2.11 (d, 3H), 2.03 (d, 3H).

³¹P-NMR (162 MHz, DMSO-d6), δ −2.36 (d)

ESI-MS: [M−H]. 334.0557, Calculated 334.0558

Synthesis of Compound 26 with the Following Structure:

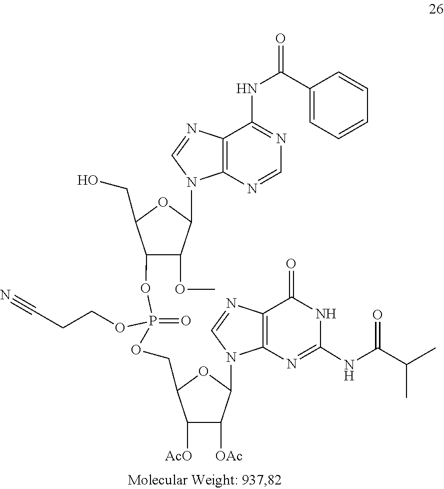 [SMILES: COC1C(OP(=O)(OCCC#N)OCC2OC(n3cnc4c(=O)[nH]c(NC(=O)C(C)C)nc43)C(OC(C)=O)C2OC(C)=O)C(CO)OC1n1cnc2c(NC(=O)c3ccccc3)ncnc21]

Compound 25 (1 eq.) was dissolved in aqueous acetic acid (80% v/v) and reaction mixture was stirred for 1 h. Solvent was evaporated and residual crude product was coevaporated with methanol. Afterwards, the product was purified by flash chromatography (Silica: 120 g, solvent A: ethyl acetate, solvent B (2nd solvent): MeOH, linear gradient according to following table Ex-27

TABLE Ex-27

| | | | % 2^{nd} |
|---|---|---|---|
| | Min | Solvents | solvent |
| 1 | 0.0 | AB | 0 |
| 2 | 5.0 | AB | 15 |
| 3 | 20.0 | AB | 35 |
| 4 | 1.0 | AB | 80 |
| 5 | 3.0 | AB | 80 |

The product was obtained as white foam with a yield of 98%.

¹H NMR (400 MHz, DMSO-d₆) δ 12.13 (s, 1H), 11.59 (d, 1H), 11.26 (d, 1H), 8.77 (m, 2H), 8.29 (m, 1H), 8.05 (d, 2H), 7.65 (t, 1H), 7.55 (t, 1H), 6.20 (m, 1H), 6.14 (m, 1H), 5.85 (m, 1H), 5.55 (m, 1H), 5.40 (m, 1H), 5.21 (m, 1H), 4.83, (q, 1H), 4.48 (m, 3H), 4.33 (m, 1H), 4.28 (m, 2H), 3.64 (m, 2H), 3.42-3.33 (m, 5H), 2.98 (q, 2H), 2.76 (m, 1H), 2.15 (d, 3H), 2.03 (d, 3H), 1.11 (m, 6H).

³¹P-NMR (162 MHz, DMSO-d6), δ −2.38 (d)

Synthesis of Compound 27 with the Following Structure:

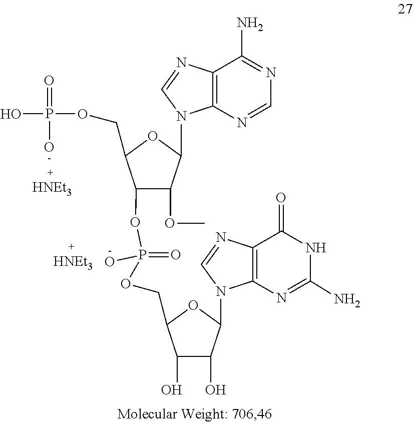 [SMILES: COC1C(OP(=O)(O)OCC2OC(n3cnc4c(=O)[nH]c(N)nc43)C(O)C2O)C(COP(=O)(O)O)OC1n1cnc2c(N)ncnc21]

Compound 26 (1 eq.) was dissolved in dry acetonitrile under protective gas atmosphere. Bis(2-cyanoethyl)-N,N-diisopropylphosphoramidite (2 eq.) and tetrazole (0.45 M in dry acetonitrile, 2 eq.) was added and stirred for 30 minutes. Oxidizer (0.1 M iodine in THE/Pyridine/water (77:21:2, v/v/v)) was added until red color was observed and stayed for 15 minutes. A 1:1 mixture of aqueous sodium disulfite solution (5 wt. %) and citric acid solution (5 wt. %) was added and crude product was extracted with dichloromethane. Solvent was evaporated and the residue was coevaporated with methanol. Protected crude product was dissolved in a 1:1 solution of methanol/concentrated ammonia and stirred for 48 h. Ammonia was evaporated and crude product was dissolved in water. The crude product was purified by ion exchange chromatography with Macro-Prep High-Q resin (BioRad) using solvent A: Water, solvent B (2nd solvent): 1 M TEAB with the following gradient in Table Ex-28:

TABLE Ex-28

| | | | % 2^{nd} |
|---|---|---|---|
| | Min | Solvents | solvent |
| 1 | 0 | AB | 0 |
| 2 | 45 | AB | 100 |
| 3 | 0 | AB | 100 |
| 4 | 10 | AB | 100 |

Product was gained as white powder in a yield of 73%.

¹H NMR (400 MHz, DMSO-d₆) δ 8.48 (s, 1H), 8.15 (s, 1H), 7.96 (s, 1H), 7.32 (s, 2H), 6.69 (s, 2H), 6.02 (d, 1H), 5.70 (d, 1H), 4.88 (m, 1H), 4.58 (t, 1H), 4.54 (t, 1H), 4.32 (m, 1H), 4.22 (m, 1H), 4.02 (m, 1H), 3.96 (m, 2H), 3.90 (s, 2H), 3.35 (s, 3H).

³¹P-NMR (162 MHz, DMSO-d₆), δ −0.84 (s), −1.85 (s)

ESI-MS: [M−H]. 705.1190, Calculated 705.1189

Synthesis of Compound 28 with the Following Structure:

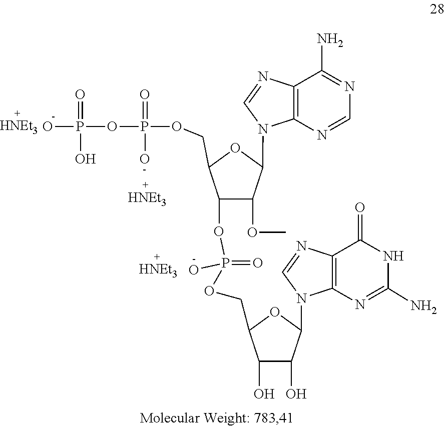 [SMILES: COC1C(OP(=O)(O)OCC2OC(n3cnc4c(=O)[nH]c(N)nc43)C(O)C2O)C(COP(=O)(O)OP(=O)(O)O)OC1n1cnc2c(N)ncnc21]

Compound 27 (1 eq.) was dissolved in dry dimethylsulfoxide. Cyanoethylphosphate-imidazolide (3 eq.) and magnesium chloride (10 eq.) were added and the reaction mixture was stirred for 24 h. DL-Dithiothreitol (3 eq.) and DBU (100 eq.) were added and the reaction was stirred for 5 h. DMSO was removed at reduced pressure. Crude product was dissolved in water and pH was adjusted to 8.0. Purification was carried out by ion exchange chromatography with Macro-Prep High-Q resin using the following gradient in Table Ex-29: Solvent A: Water, Solvent B (2^{nd} Solvent): 1 M TEAB

TABLE Ex-29

| | | | % 2^{nd} |
|---|---|---|---|
| Step | Min | Solvents | Solvent |
| 1 | 0 | AB | 0 |
| 2 | 45 | AB | 100 |
| 3 | 20 | AB | 100 |

The solvent of product fractions was evaporated yielding in 64% as a white powder.

¹H NMR (400 MHz, DMSO-d₆) δ 8.50 (s, 1H), 8.15 (s, 1H), 7.96 (s, 1H), 7.31 (s, 2H), 6.76 (s, 2H), 6.00 (d, 1H), 5.69 (d, 1H), 4.91 (m, 1H), 4.63 (m, 2H), 4.29 (m, 1H), 4.24 (m, 1H), 4.01 (m, 4H), 3.87 (m, 1H), 3.49 (m, 2H), 3.31 (s, 3H).

³¹P-NMR (162 MHz, DMSO-d₆), δ 1.95 (s), −10.70 (d), −11.50 (d)

ESI-MS: [M+H]: 787.098, Calculated 787.099

### Synthesis of Compound 29:

The synthesis of 29 starts from 2-((m⁷G)-methoxy)-3-hydroxypropyl monophosphate (compound 21) that was previously synthesized according to example 1.4.

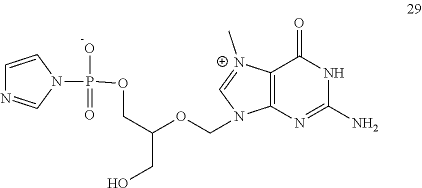 [SMILES: C[n+]1cn(COC(CO)COP(=O)(O)n2ccnc2)c2nc(N)[nH]c(=O)c21]

### Method A:

Compound 21 (1 eq.) is dissolved in DMSO and carbonyl diimidazole (20 eq.) is added in two portions over 24 h. Reaction mixture is stirred for 48-72 h and quenched by addition of water (20 eq.). The crude product is precipitated by pouring on ice cold sodium perchlorate solution in acetone (12 wt. %). The precipitate is sedimented by centrifugation and washed with ice cold acetone.

### Method B:

Compound 21 (1 eq.) is transformed into triethylammonium salt using Dowex-50WX8 (triethylammonium form) and is afterwards dissolved in DMSO. Dipyridyldisulfide (5 eq.), imidazole (10 eq.) and triethylamine (2 eq.) are added. Triphenylphosphine (5 eq.) is added and the reaction is stirred for 24 h. Afterwards another portion of dipyridyldisulfide (5 eq.), imidazole (10 eq.) and triphenylphosphine (5 eq.) is added and the reaction is left stirring for another 24 h. The crude product is precipitated by pouring on ice cold sodium perchlorate solution in acetone (12 wt. %). The precipitate is sedimented by centrifugation and washed with ice cold acetone.

### Synthesis of Compound 22:

Compound 29 (1 eq.) is dissolved in water/acetonitrile (1:1, v/v) or in pure water. The diphosphorylated AmG-dinucleotide 28 (1.0 eq.) is added. After dissolution of the educts, magnesium chloride or zinc chloride (10 eq.) is added and the reaction mixture is stirred for 16-24 h. The reaction is quenched by addition of EDTA (11 eq.) and adjusted to pH 7.0 with aqueous ammonia solution. The reaction mixture is desalted using RP-HPLC using solvent A: 5 mM ammonium acetate, solvent B: 90% Methanol in water with the gradient program in Table Ex-30

TABLE Ex-30

| Time | A | B |
|---|---|---|
| [min] | [%] | [%] |
| 0 | 100 | 0 |
| 10 | 100 | 0 |
| 20 | 85 | 15 |
| 21 | 5 | 95 |
| 31 | 5 | 95 |
| 32 | 100 | 0 |
| 48 | 100 | 0 |

The desalted reaction mixture is purified by RP-HPLC using solvent A: 100 mM triethylammonium acetate (pH7), solvent B: 80% acetonitrile in water. The gradient program is described in table Ex-31.

TABLE Ex-31

| Time | A | B |
|---|---|---|
| [min] | [%] | [%] |
| 0 | 100 | 0 |
| 40 | 60 | 40 |
| 41 | 100 | 0 |
| 51 | 100 | 0 |

The product is dissolved in ultrapure water resulting in a 100 mM solution. Alternatively, compound 22 was synthesized as described in Example 1.9: Synthesis route IX.

Similar compounds can be synthesized in accordance with the above synthesis route when starting from other educt compounds as shown in the following examples.

### Example 1.6: Synthesis Route VI

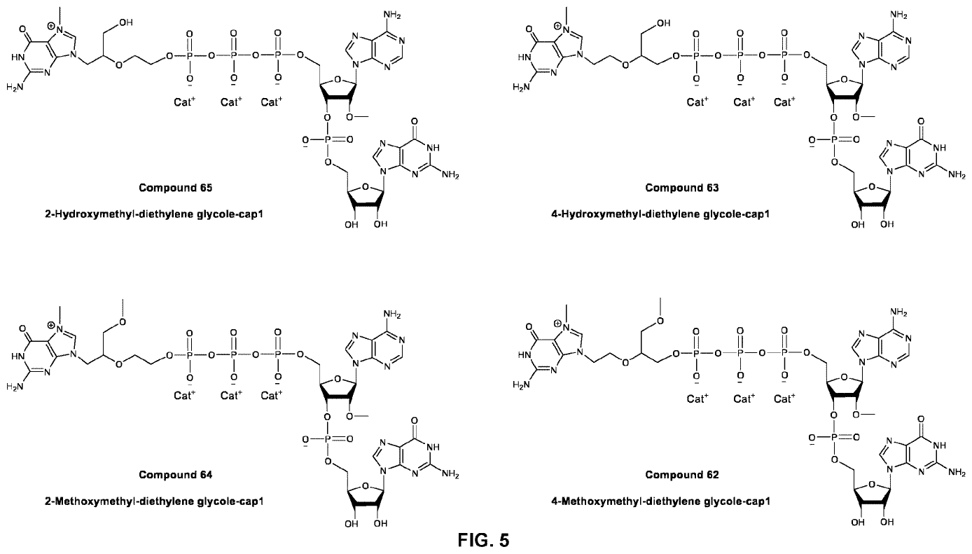

The synthesis of compounds 61-66 (corresponding to “long-branched diethyleneglycol derived Cap1” starting from 2-phenyl-1,3-dioxan-5-ol (X5) and acetic acid 2-bromo-ethyl ester (X6) is shown in the following. It is noted that the synthesis route when starting from other acetal protected triol compounds is mostly identical. Examples of resulting compounds are described at the end of the present example and are depicted in FIG. 5.

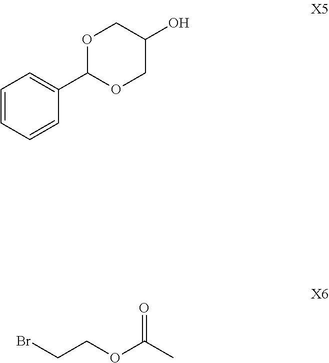

Synthesis of Compound 30 with the Following Structure:

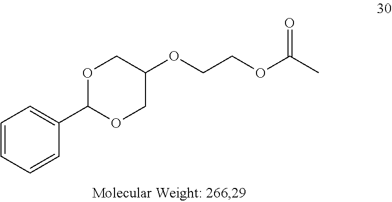 [SMILES: CC(=O)OCCOC1COC(c2ccccc2)OC1]

Dry 2-phenyl-1,3-dioxan-5-ol (X5) is dissolved in anhydrous DMF or THF at 0° C. and under argon gas atmosphere. The solution is treated with NaH in three portions over a period of 30 min. Catalytic amounts of tetrabutylammonium iodide are added followed by the addition of acetic acid 2-bromo-ethyl ester (X6). Upon complete turnover, the reaction mixture is quenched with ice, diluted with DCM and washed with 5% citric acid solution, 5% sodium bicarbonate solution and brine. Drying over Na₂SO₄ is followed by purification via flash chromatography.

Synthesis of Compound 31 with the Following Structure:

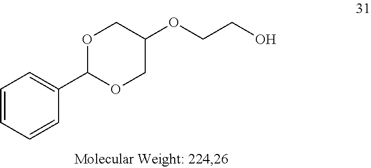 [SMILES: OCCOC1COC(c2ccccc2)OC1]

Synthesis of compound 31 is performed under basic conditions with methylamine in ethanol, methylamine in water, ammonia in methanol, ammonia in water, aqueous NaOH in THF/H₂O/MeOH or a combination thereof. All volatiles are removed under reduced pressure and crude compound 31 is purified by column chromatography.

Synthesis of Compound 32 with the Following Structure:

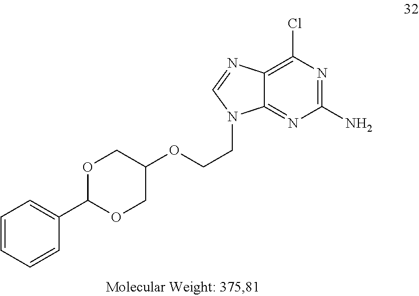 [SMILES: Nc1nc(Cl)c2ncn(CCOC3COC(c4ccccc4)OC3)c2n1]

Compound 32 is prepared from compound 31 according to general procedure 1 employing 2-amino-6-chloropurine.

Synthesis of Compound 33 with the Following Structure:

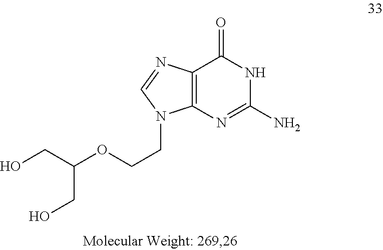 [SMILES: Nc1nc2c(ncn2CCOC(CO)CO)c(=O)[nH]1]

Compound 32 is dissolved in 1 N HCl and heated to 85° C. The reaction progress is monitored by TLC and/or ESI-MS. Upon complete turnover, the reaction mixture is cooled to ambient temperature and pH is adjusted to 9 using aqueous ammonia solution. The reaction flask is stored at 0° C. upon which compound 33 precipitates as a white powder. The precipitate is collected by filtration and is extensively washed with ice-cooled water. Compound 33 can be alternatively prepared by the general procedure 6.

Synthesis of Compound 34 with the Following Structure:

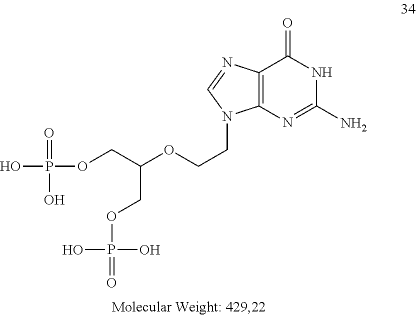 [SMILES: Nc1nc2c(ncn2CCOC(COP(=O)(O)O)COP(=O)(O)O)c(=O)[nH]1]

Compound 34 is synthesized from compound 33 according to the general procedure 2.

Synthesis of Compound 35 with the Following Structure:

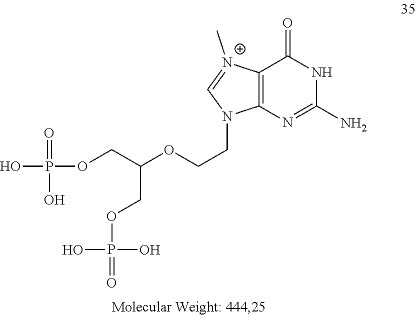 [SMILES: C[n+]1cn(CCOC(COP(=O)(O)O)COP(=O)(O)O)c2nc(N)[nH]c(=O)c21]

Compound 35 is synthesized from compound 34 according to the general procedure 4.

Synthesis of Compound 36 with the Following Structure:

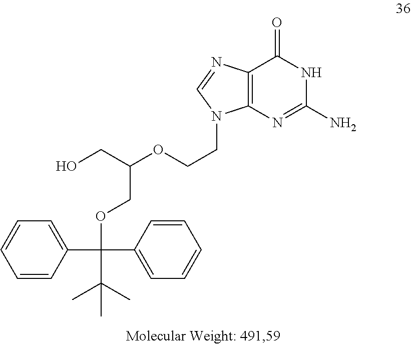 [SMILES: CC(C)(C)C(OCC(CO)OCCn1cnc2c(=O)[nH]c(N)nc21)(c1ccccc1)c1ccccc1]

Compound 36 is synthesized from compound 33 in analogy to the synthesis of compound 1 as described previously.

Synthesis of Compound 37a and 37b with the Following Structures:

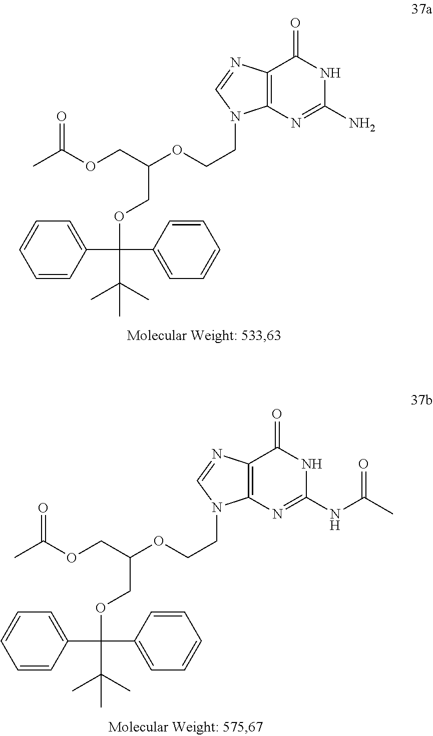 [SMILES: CC(=O)OCC(COC(c1ccccc1)(c1ccccc1)C(C)(C)C)OCCn1cnc2c(=O)[nH]c(N)nc21 ; CC(=O)Nc1nc2c(ncn2CCOC(COC(C)=O)COC(c2ccccc2)(c2ccccc2)C(C)(C)C)c(=O)[nH]1]

Compound 37a and 37b are synthesized from compound 36 in analogy to the synthesis of compound 2a and 2b as described previously. Compound 37a and 37b are both used for the following steps which is exemplary shown for compound 37a.

Synthesis of Compound 38 with the Following Structure:

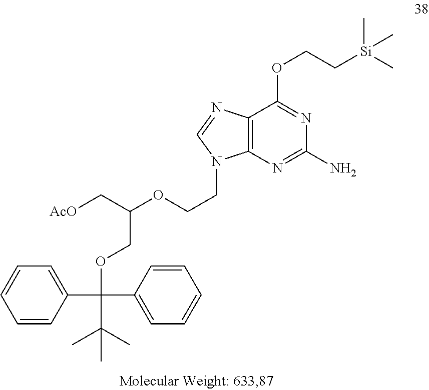 [SMILES: CC(=O)OCC(COC(c1ccccc1)(c1ccccc1)C(C)(C)C)OCCn1cnc2c(OCC[Si](C)(C)C)nc(N)nc21]

Compound 38 is synthesized from compound 37a in analogy to the synthesis of compound 3a and 3b as described previously.

Synthesis of Compound 39 with the Following Structure:

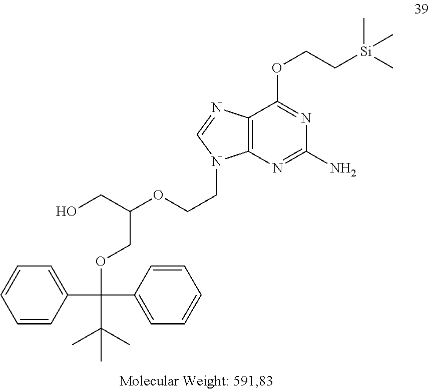 [SMILES: CC(C)(C)C(OCC(CO)OCCn1cnc2c(OCC[Si](C)(C)C)nc(N)nc21)(c1ccccc1)c1ccccc1]

Compound 39 is synthesized from compound 38 in analogy to the synthesis of compound 4 as described previously.

Synthesis of Compound 40 with the Following Structure:

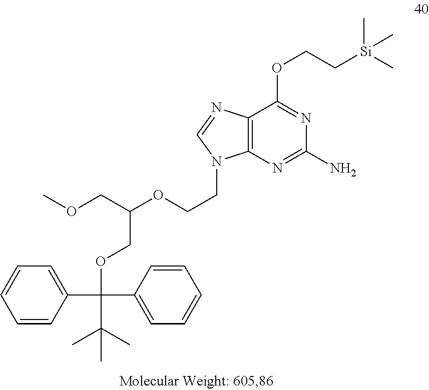 [SMILES: COCC(COC(c1ccccc1)(c1ccccc1)C(C)(C)C)OCCn1cnc2c(OCC[Si](C)(C)C)nc(N)nc21]

Compound 40 is synthesized from compound 39 in analogy to the synthesis of compound 5 as described previously.

Synthesis of Compound 41 with the Following Structure:

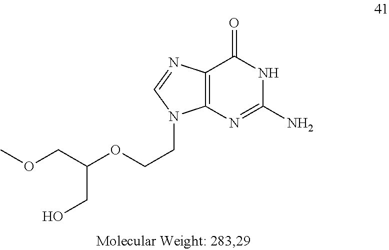 [SMILES: COCC(CO)OCCn1cnc2c(=O)[nH]c(N)nc21]

Compound 41 is synthesized from compound 40 in analogy to the synthesis of compound 6 as described previously.

Synthesis of Compound 42 with the Following Structure:

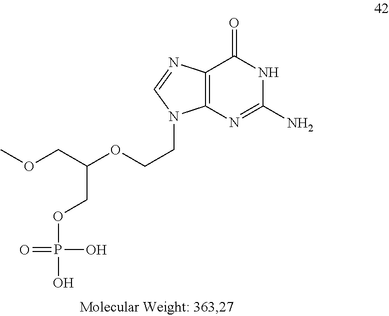 [SMILES: COCC(COP(=O)(O)O)OCCn1cnc2c(=O)[nH]c(N)nc21]

Compound 42 is synthesized from compound 41 according to the general procedure 2 or 3.

Synthesis of Compound 43 with the Following Structure:

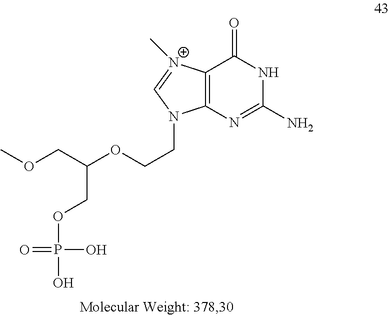 [SMILES: COCC(COP(=O)(O)O)OCCn1c[n+](C)c2c(=O)[nH]c(N)nc21]

Compound 43 is synthesized from compound 42 according to the general procedure 4.

Synthesis of Compound 44 with the Following Structure:

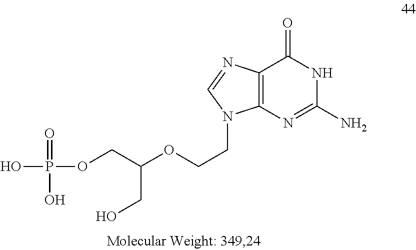 [SMILES: Nc1nc2c(ncn2CCOC(CO)COP(=O)(O)O)c(=O)[nH]1]

Compound 44 is synthesized from compound 36 according to the general procedure 2. Alternatively compound 36 can be phosphorylated according to general procedure 3 followed by one of the desilylation procedures described for compound 6.

Synthesis of Compound 45 with the Following Structure:

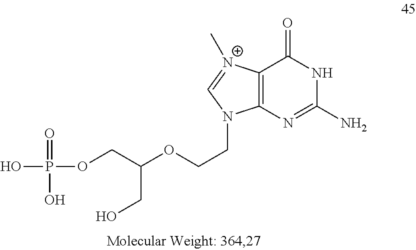 [SMILES: C[n+]1cn(CCOC(CO)COP(=O)(O)O)c2nc(N)[nH]c(=O)c21]

Compound 45 is synthesized from compound 44 according to the general procedure 4.

Synthesis of Compound 46 with the Following Structure:

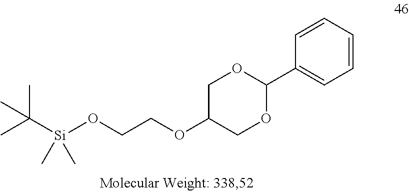 [SMILES: CC(C)(C)[Si](C)(C)OCCOC1COC(c2ccccc2)OC1]

Compound 31 (1.0 eq.) is dissolved in dry DMF and treated consecutively with imidazole and TBDMSCl. The reaction is stirred for 16 h at ambient temperature followed by evaporation of the solvent and purification by flash chromatography.

Synthesis of Compound 47 with the Following Structure:

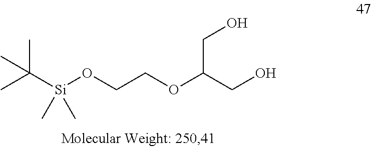 [SMILES: CC(C)(C)[Si](C)(C)OCCOC(CO)CO]

6 N HCl is added dropwise to a solution of compound 46 in THF/H₂O (1/1, v/v). The reaction is deemed complete by TLC and/or ESI-MS and pH is then adjusted to 8 using saturated sodium bicarbonate solution. The reaction mixture is extracted trice with ethyl acetate, the organic layer is dried over Na₂SO₄, concentrated, and purified by flash chromatography.

Synthesis of Compound 48 with the Following Structure:

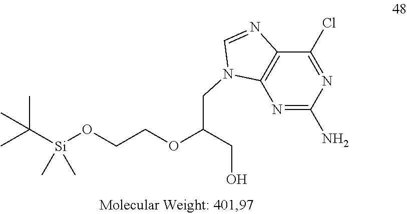 [SMILES: CC(C)(C)[Si](C)(C)OCCOC(CO)Cn1cnc2c(Cl)nc(N)nc21]

Compound 48 is prepared from compound 47 according to general procedure 1 employing 2-amino-6-chloropurine.

Synthesis of Compound 49 with the Following Structure:

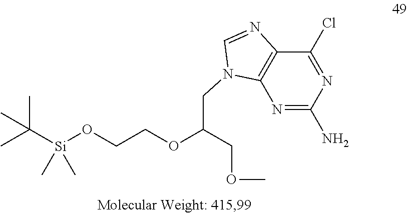 [SMILES: COCC(Cn1cnc2c(Cl)nc(N)nc21)OCCO[Si](C)(C)C(C)(C)C]

Compound 49 is synthesized from compound 48 in analogy to the synthesis of compound 5 as described previously.

Synthesis of Compound 50 with the Following Structure:

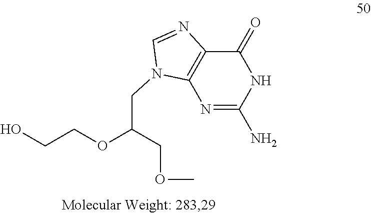 [SMILES: COCC(Cn1cnc2c(=O)[nH]c(N)nc21)OCCO]

Compound 50 is synthesized from compound 49 according to the general procedure 6 or in analogy to the synthesis of compound 33 as described previously.

Synthesis of Compound 51 with the Following Structure:

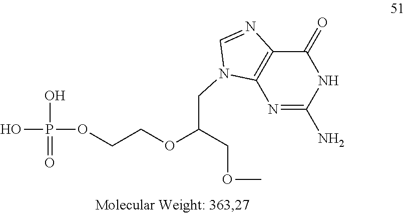 [SMILES: COCC(Cn1cnc2c(=O)[nH]c(N)nc21)OCCOP(=O)(O)O]

Compound 51 is synthesized from compound 50 according to the general procedure 2 or 3.

Synthesis of Compound 52 with the Following Structure:

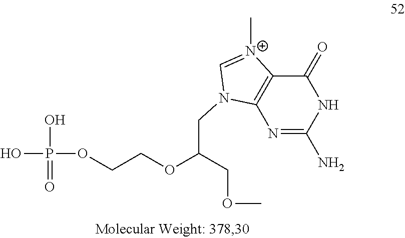 [SMILES: COCC(Cn1c[n+](C)c2c(=O)[nH]c(N)nc21)OCCOP(=O)(O)O]

Compound 52 is synthesized from compound 51 according to the general procedure 4.

Synthesis of Compound 53 with the Following Structure:

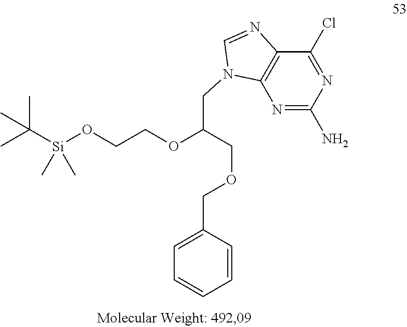 [SMILES: CC(C)(C)[Si](C)(C)OCCOC(COCc1ccccc1)Cn1cnc2c(Cl)nc(N)nc21]

Compound 53 is prepared from compound 48 according to general procedure 1 employing benzyl alcohol. Alternatively Compound 53 can be prepared from compound 48 in analogy to the procedure described for compound 30 with the difference that benzyl bromide is used.

Synthesis of Compound 54 with the Following Structure:

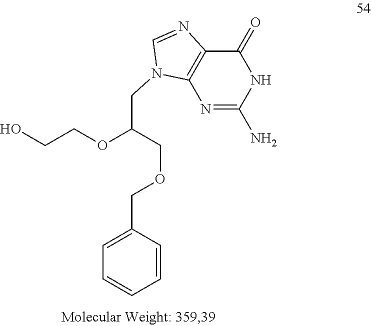 [SMILES: Nc1nc2c(ncn2CC(COCc2ccccc2)OCCO)c(=O)[nH]1]

Compound 54 is synthesized from compound 53 according to the general procedure 6 or in analogy to the synthesis of compound 33 as described previously.

Synthesis of Compound 55 with the Following Structure:

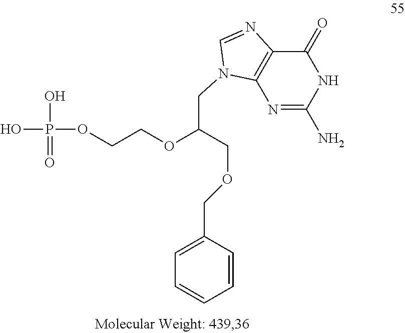 [SMILES: Nc1nc2c(ncn2CC(COCc2ccccc2)OCCOP(=O)(O)O)c(=O)[nH]1]

Compound 55 is synthesized from compound 54 according to the general procedure 2 or 3.

Synthesis of Compound 56 with the Following Structure:

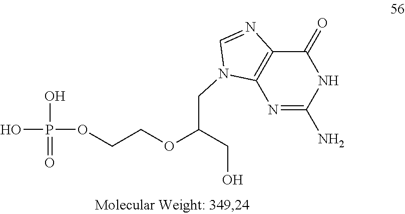 [SMILES: Nc1nc2c(ncn2CC(CO)OCCOP(=O)(O)O)c(=O)[nH]1]

A solution of compound 55 (1.0 eq) and Pd/C in MeOH (or another suitable solvent) is treated with hydrogen (1 atm). Solvent is evaporated after complete deprotection and the residue is resuspended in ACN. The suspension is filtered through a pad of Celite, is then concentrated and used in the next step without further purification. The palladium catalyst can be also removed by reversed phase chromatography.

Synthesis of Compound 57 with the Following Structure:

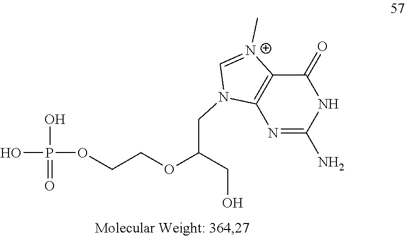 [SMILES: C[n+]1cn(CC(CO)OCCOP(=O)(O)O)c2nc(N)[nH]c(=O)c21]

Compound 57 is synthesized from compound 56 according to the general procedure 4.

Synthesis of Compound 58 with the Following Structure:

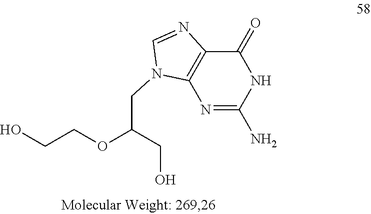 [SMILES: Nc1nc2c(ncn2CC(CO)OCCO)c(=O)[nH]1]

Compound 58 is synthesized from compound 48 according to the general procedure 6 or in analogy to the synthesis of compound 33 as described previously.

Synthesis of Compound 59 with the Following Structure:

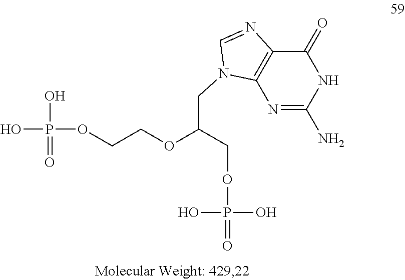 [SMILES: Nc1nc2c(ncn2CC(COP(=O)(O)O)OCCOP(=O)(O)O)c(=O)[nH]1]

Compound 59 is synthesized from compound 58 according to the general procedure 2 or 3. Synthesis of Compound 60 with the Following Structure:

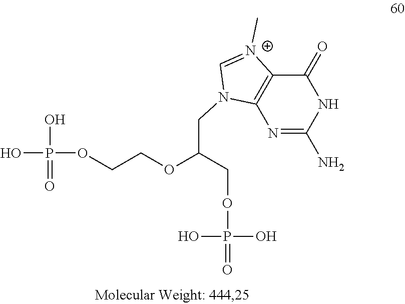 [SMILES: C[n+]1cn(CC(COP(=O)(O)O)OCCOP(=O)(O)O)c2nc(N)[nH]c(=O)c21]

Compound 60 is synthesized from compound 59 according to the general procedure 4.

Synthesis of Compound 61-66 with the Following Structures:

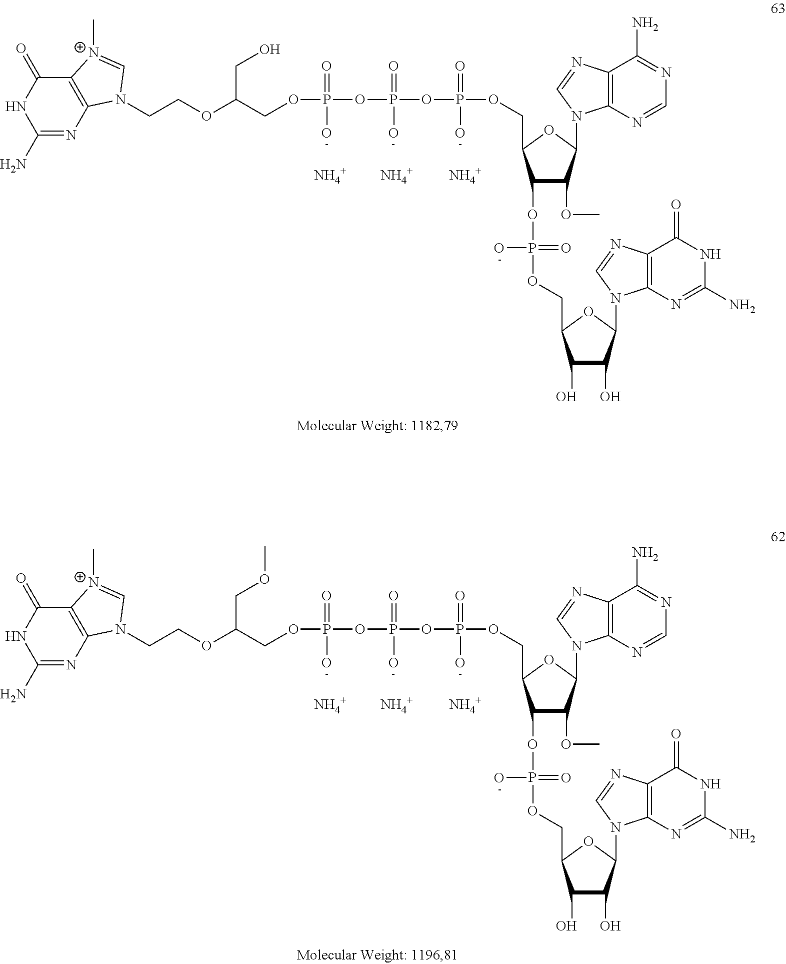

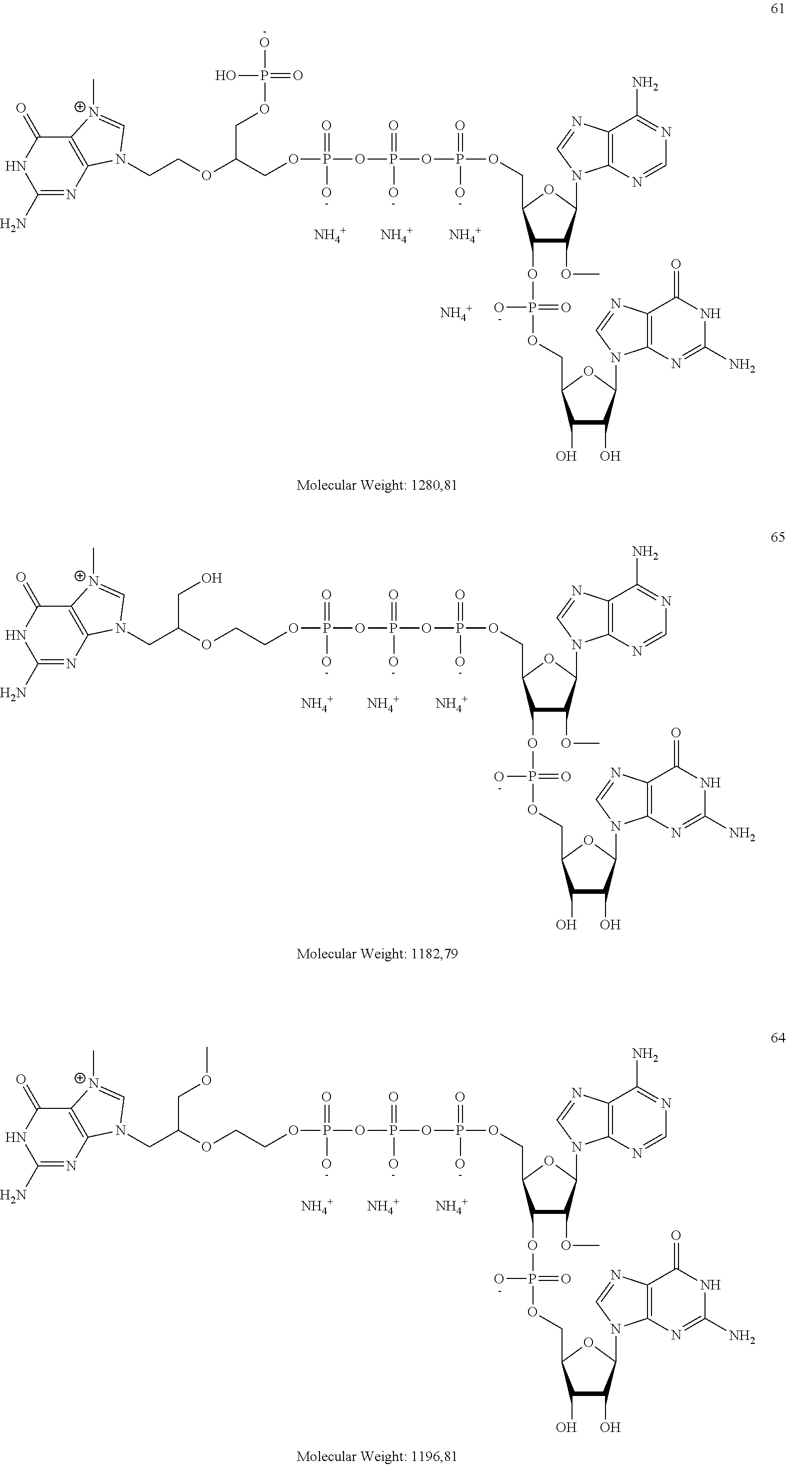

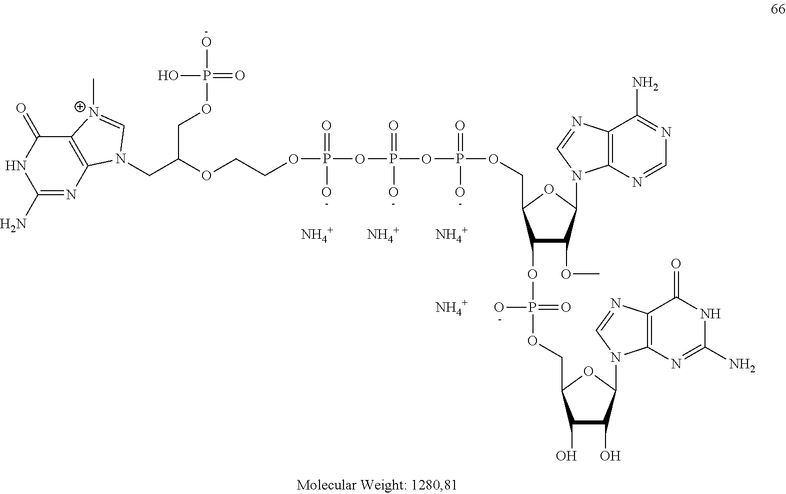

Compounds 61-66 are synthesized from compounds 35, 43, 45, 52, 57 and 60, respectively, according to example 1.5.

Similar compounds can be synthesized in accordance with the above synthesis route when starting from other educt compounds.

### Example 1.7: Synthesis Route VII

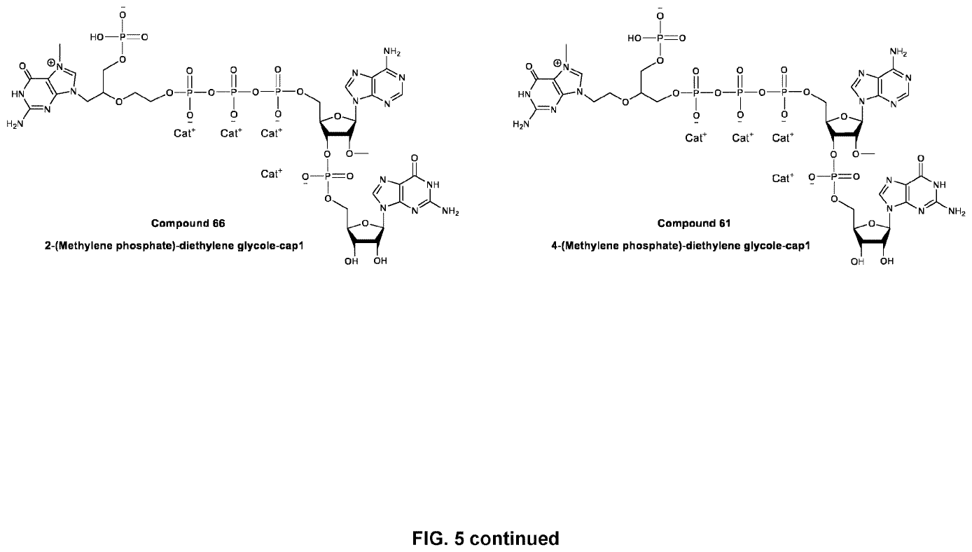

The synthesis of compounds 93-98 (corresponding to “short-branched diethyleneglycol derived Cap1” starting from hydroxyethyl vinyl ether (X7) is shown in the following. It is noted that the synthesis route when starting from other triol compounds with the ability of acetal formation is mostly identical. Examples of resulting compounds are described at the end of the present example and are depicted in FIG. 6.

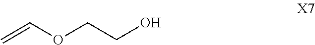

Synthesis of Compound 67 with the Following Structure:

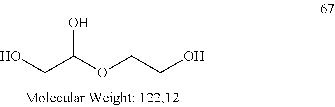

Nafion NR 50 and aqueous hydrogen peroxide are stirred at ambient temperature for 30 min followed by the addition of hydroxyethyl vinyl ether (X7). The reaction flask is equipped with a reflux condenser and the reaction mixture is vigorously stirred at 70° C. until TLC and/or MS indicate complete reaction turnover. The reaction mixture is then cooled to ambient temperature and all solids are removed by filtration. The filtrate is treated with a small amount of MnO₂ in order to remove residual traces of the oxidating agent (starch-iodine paper control). MnO₂ is removed by filtration, the filtrate is concentrated and purified by flash chromatography.

Synthesis of Compound 68 with the Following Structure:

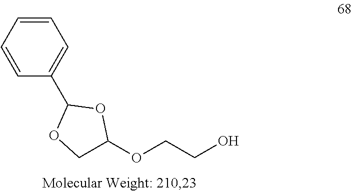 [SMILES: OCCOC1COC(c2ccccc2)O1]

Synthesis of compound 68 is performed by stirring p-toluenesulfonic acid, benzaldehyde and compound 67 under reflux in 1,4-dioxane or toluene using a Dean-Stark apparatus or molecular sieve as water trap. After TLC and/or MS indicate complete reaction turnover the reaction mixture is cooled to ambient temperature and neutralized with sodium hydroxide. The reaction mixture is concentrated and washed trice with water. Purification is performed by flash chromatography.

Synthesis of Compound 69 with the Following Structure:

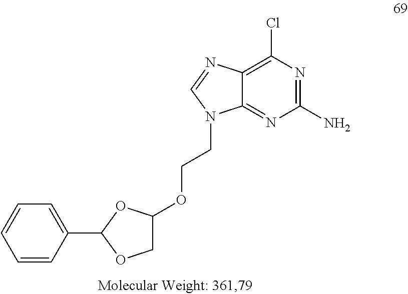 [SMILES: Nc1nc(Cl)c2ncn(CCOC3COC(c4ccccc4)O3)c2n1]

Compound 69 is synthesized from compound 68 according to the general procedure 1. Synthesis of Compound 70 with the Following Structure:

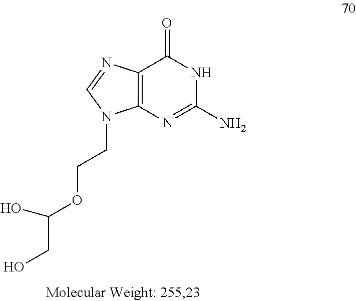 [SMILES: Nc1nc2c(ncn2CCOC(O)CO)c(=O)[nH]1]

Compound 70 is synthesized from compound 69 according to the general procedure 6 or in analogy to the synthesis of compound 33 as described previously.

Synthesis of Compound 71 with the Following Structure:

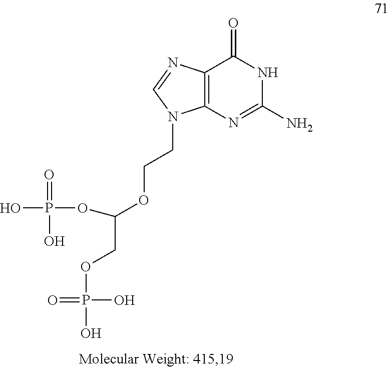 [SMILES: Nc1nc2c(ncn2CCOC(COP(=O)(O)O)OP(=O)(O)O)c(=O)[nH]1]

Compound 71 is synthesized from compound 70 according to the general procedure 2.

Synthesis of Compound 72 with the Following Structure:

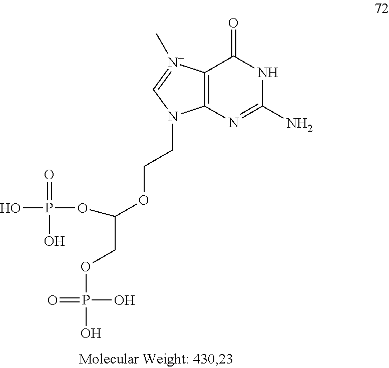 [SMILES: C[n+]1cn(CCOC(COP(=O)(O)O)OP(=O)(O)O)c2nc(N)[nH]c(=O)c21]

Compound 72 is synthesized from compound 71 according to the general procedure 4.

Synthesis of Compound 73 with the Following Structure:

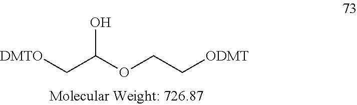

Compound 67 is coevaporated trice with pyridine and is further dried under high vacuum. It is then dissolved in pyridine and treated with a slight excess of DMTCl in three portions over a period of 1 h. The reaction mixture is deemed complete by TLC and quenched by the addition of methanol. The solvent is evaporated and the oily residue is coevaporated trice with toluene. The remnant is taken into ethyl acetate and is washed with saturated sodium bicarbonate solution. The organic layer is dried over Na₂SO₄, is concentrated and purified by flash chromatography.

Synthesis of Compound 74 with the Following Structure:

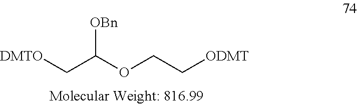

Compound 74 is synthesized from compound 73 according to the general procedure 1 employing benzyl alcohol. Alternatively Compound 74 can be prepared from compound 73 in analogy to the procedure described for compound 30 with the difference that benzyl bromide is used.

Synthesis of Compound 75 with the Following Structure:

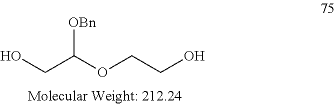 [SMILES: OCCOC(CO)OCc1ccccc1]

Compound 74 is dissolved in an 8/2 mixture of acetic acid/H₂O and stirred at ambient temperature until TLC and/or ESI-MS indicate full consumption of the starting material. Solvent is evaporated, the residue is coevaporated with ethanol and purified by flash chromatography,

Alternatively compound 74 is dissolved 9/1 mixture of DCM/MeOH (v/v) and is cooled to 0° C. A solution of benzenesulfonic acid in the same solvent mixture is added slowly accompanied by the immediate appearance of a bright orange colour. After a few minutes the reaction is deemed complete and quenched by the addition of saturated sodium bicarbonate solution. The reaction mixture is transferred into a separation funnel and the organic layer is washed with additional saturated sodium bicarbonate solution and brine. Drying over Na₂SO₄ is followed by flash chromatography.

Synthesis of Compound 76a and 76b with the Following Structures:

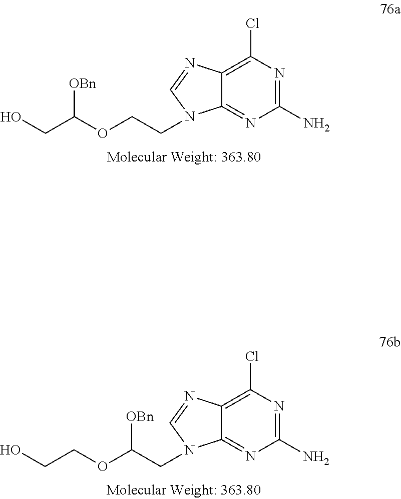 [SMILES: Nc1nc(Cl)c2ncn(CCOC(CO)OCc3ccccc3)c2n1 ; Nc1nc(Cl)c2ncn(CC(OCCO)OCc3ccccc3)c2n1]

Compound 76a and 76b are prepared from compound 75 according to general procedure 1 employing 2-amino-6-chloropurine. The regioisomers are separated by flash chromatography.

Synthesis of Compound 77a and 77b with the Following Structures:

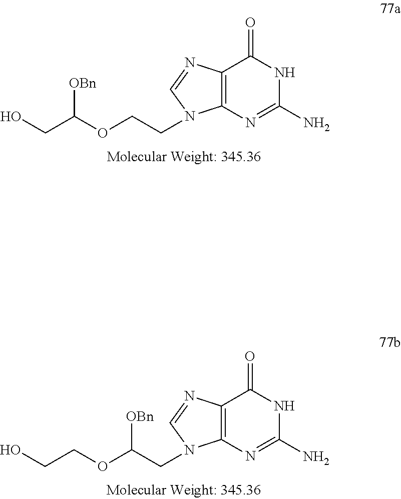 [SMILES: Nc1nc2c(ncn2CCOC(CO)OCc2ccccc2)c(=O)[nH]1 ; Nc1nc2c(ncn2CC(OCCO)OCc2ccccc2)c(=O)[nH]1]

Compound 77a and 77b are prepared from compound 76a and 76b respectively according to the general procedure 6 or in analogy to the synthesis of compound 33 as described previously.

Synthesis of Compound 78a and 78b with the Following Structures:

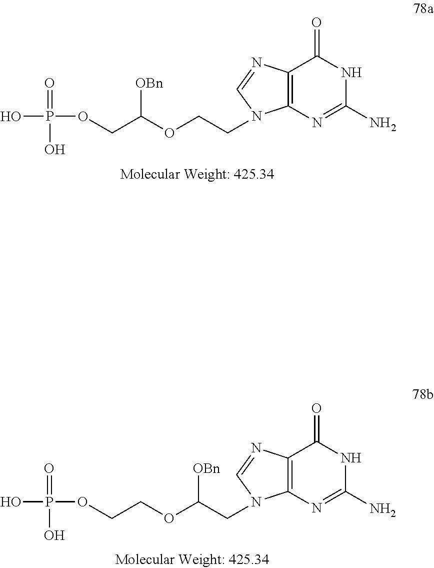 [SMILES: Nc1nc2c(ncn2CCOC(COP(=O)(O)O)OCc2ccccc2)c(=O)[nH]1 ; Nc1nc2c(ncn2CC(OCCOP(=O)(O)O)OCc2ccccc2)c(=O)[nH]1]

Compound 78a and 78b are prepared from compound 77a and 77b respectively according to the general procedure 2 or 3.

Synthesis of Compound 79a and 79b with the Following Structures:

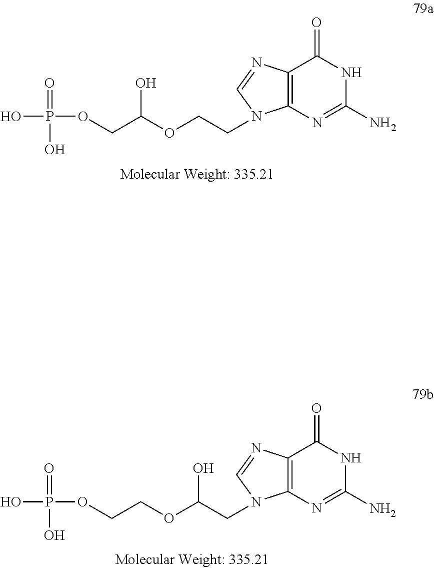 [SMILES: Nc1nc2c(ncn2CCOC(O)COP(=O)(O)O)c(=O)[nH]1 ; Nc1nc2c(ncn2CC(O)OCCOP(=O)(O)O)c(=O)[nH]1]

Compound 79a and 79b are prepared from compound 78a and 78b respectively in analogy to the synthesis of compound 56 as described previously.

Synthesis of Compound 80a and 80b with the Following Structures:

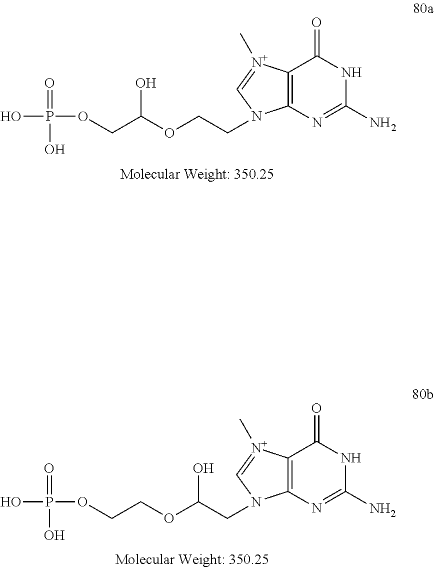 [SMILES: C[n+]1cn(CCOC(O)COP(=O)(O)O)c2nc(N)[nH]c(=O)c21 ; C[n+]1cn(CC(O)OCCOP(=O)(O)O)c2nc(N)[nH]c(=O)c21]

Compound 80a and 80b are prepared from compound 79a and 79b respectively according to the general procedure 4.

Synthesis of Compound 81 with the Following Structure:

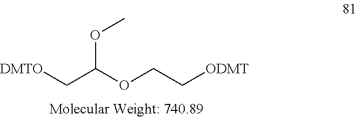

Compound 81 is synthesized from compound 73 in analogy to the synthesis of compound 5 as described previously.

Synthesis of Compound 82 with the Following Structure:

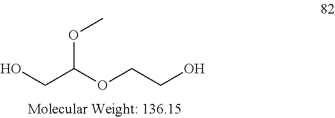

Compound 82 is synthesized from compound 81 in analogy to the synthesis of compound 75 as described previously.

Synthesis of Compound 83a and 83b with the Following Structure:

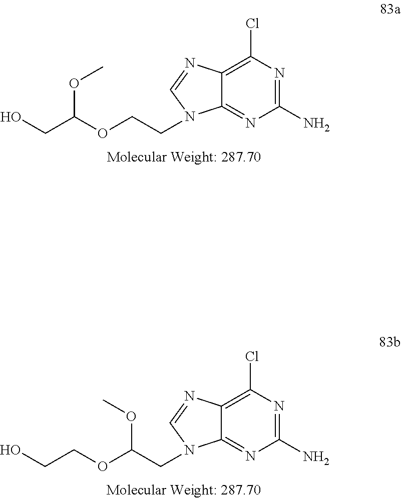 [SMILES: COC(CO)OCCn1cnc2c(Cl)nc(N)nc21 ; COC(Cn1cnc2c(Cl)nc(N)nc21)OCCO]

Compound 83a and 83b are prepared from compound 82 according to general procedure 1 employing 2-amino-6-chloropurine. The regioisomers are separated by flash chromatography.

Synthesis of Compound 84a and 84b with the Following Structures:

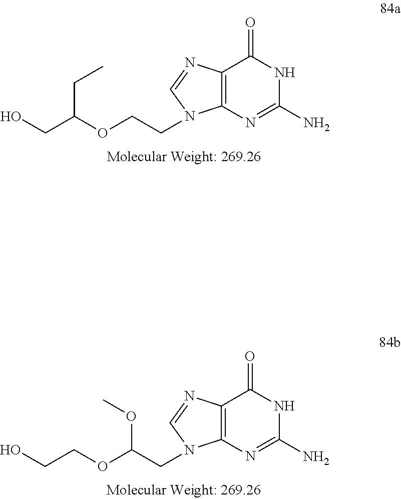 [SMILES: CCC(CO)OCCn1cnc2c(=O)[nH]c(N)nc21 ; COC(Cn1cnc2c(=O)[nH]c(N)nc21)OCCO]

Compound 84a and 84b are prepared from compound 83a and 83b respectively according to the general procedure 6 or in analogy to the synthesis of compound 33 as described previously.

Synthesis of Compound 85a and 85b with the Following Structure:

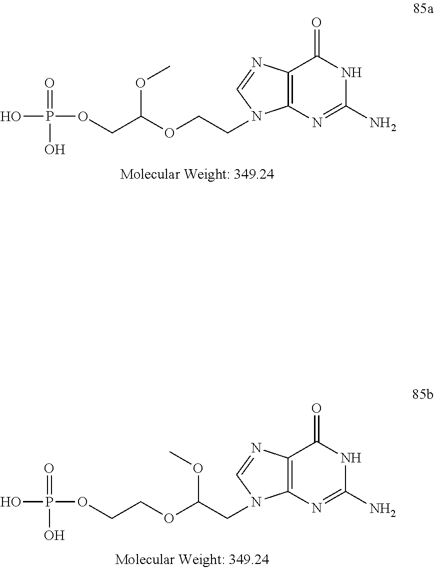 [SMILES: COC(COP(=O)(O)O)OCCn1cnc2c(=O)[nH]c(N)nc21 ; COC(Cn1cnc2c(=O)[nH]c(N)nc21)OCCOP(=O)(O)O]

Compound 85a and 85b are prepared from compound 84a and 84b respectively according to the general procedure 2 or 3.

Synthesis of Compound 86a and 86b with the Following Structures:

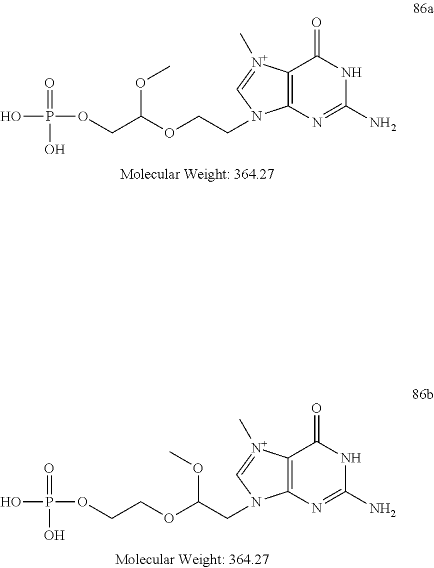 [SMILES: COC(COP(=O)(O)O)OCCn1c[n+](C)c2c(=O)[nH]c(N)nc21 ; COC(Cn1c[n+](C)c2c(=O)[nH]c(N)nc21)OCCOP(=O)(O)O]

Compound 86a and 86b are prepared from compound 85a and 85b respectively according to the general procedure 4

Synthesis of Compound 87 with the Following Structure:

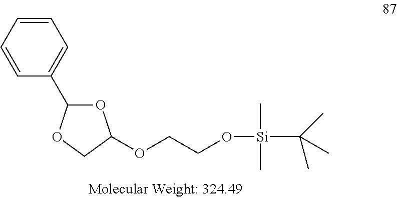 [SMILES: CC(C)(C)[Si](C)(C)OCCOC1COC(c2ccccc2)O1]

Compound 87 is prepared from compound 68 in analogy to the synthesis of compound 46 as described previously.

Synthesis of Compound 88 with the Following Structure:

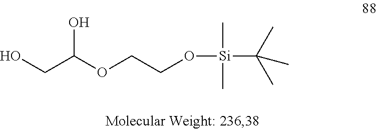

Compound 88 is prepared from compound 87 in analogy to the synthesis of compound 47 as described previously.

Synthesis of Compound 89 with the Following Structure:

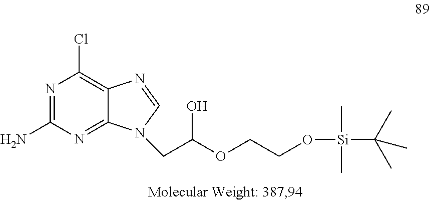 [SMILES: CC(C)(C)[Si](C)(C)OCCOC(O)Cn1cnc2c(Cl)nc(N)nc21]

Compound 89 is prepared from compound 88 according to general procedure 1 employing 2-amino-6-chloropurine.

Synthesis of Compound 90 with the Following Structure:

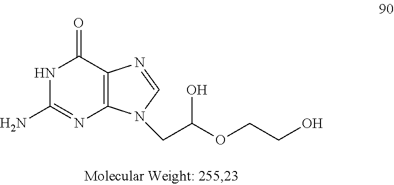 [SMILES: Nc1nc2c(ncn2CC(O)OCCO)c(=O)[nH]1]

Compound 90 is synthesized from compound 89 according to the general procedure 6 or in analogy to the synthesis of compound 33 as described previously.

Synthesis of Compound 91 with the Following Structure:

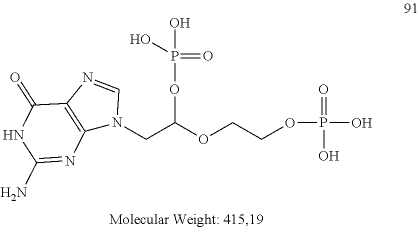 [SMILES: Nc1nc2c(ncn2CC(OCCOP(=O)(O)O)OP(=O)(O)O)c(=O)[nH]1]

Compound 91 is synthesized from compound 90 according to the general procedure 2 or 3.

Synthesis of Compound 92 with the Following Structure:

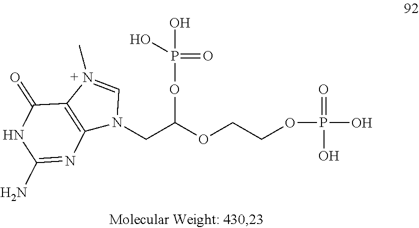

Compound 92 is synthesized from compound 91 according to the general procedure 4.

Synthesis of Compounds 93-98 with the Following Structures:

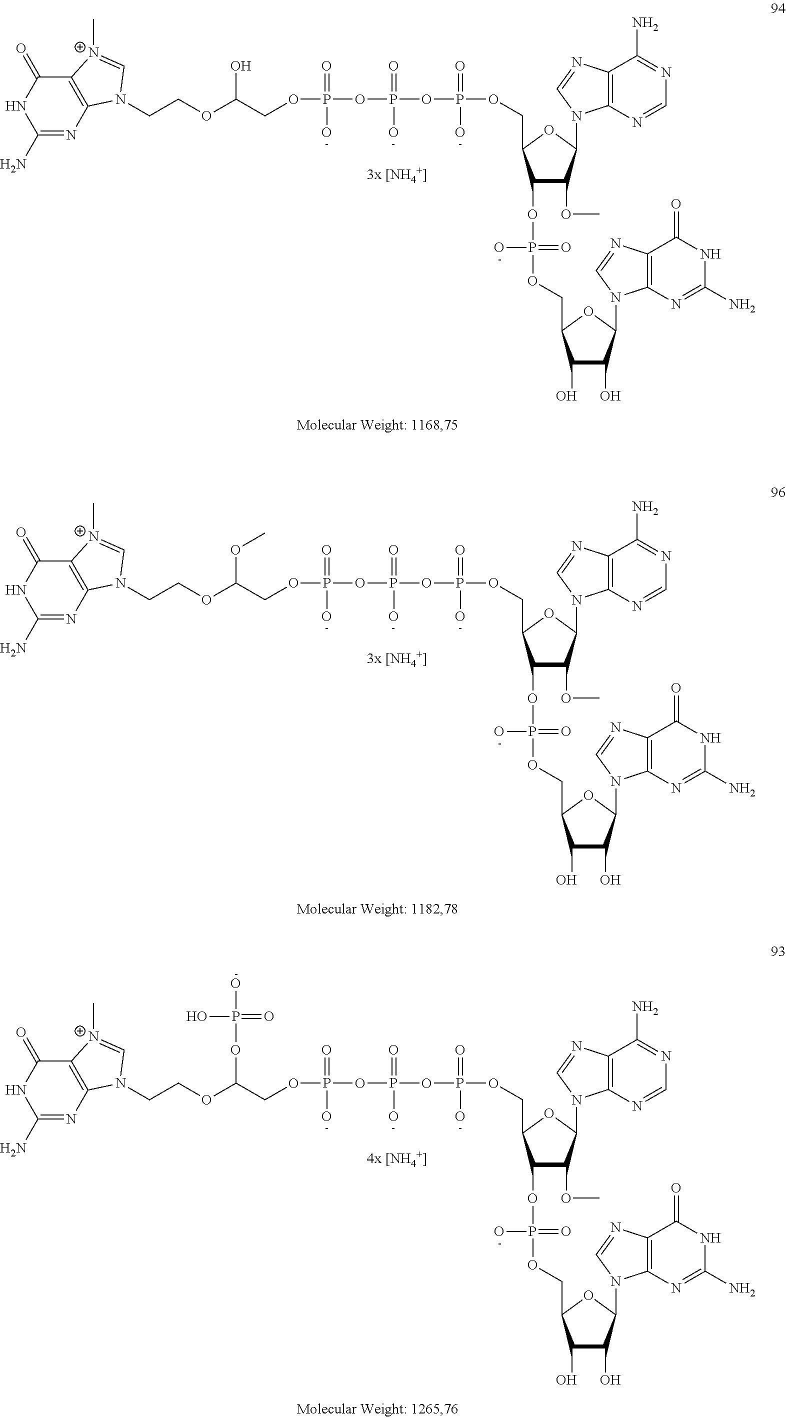

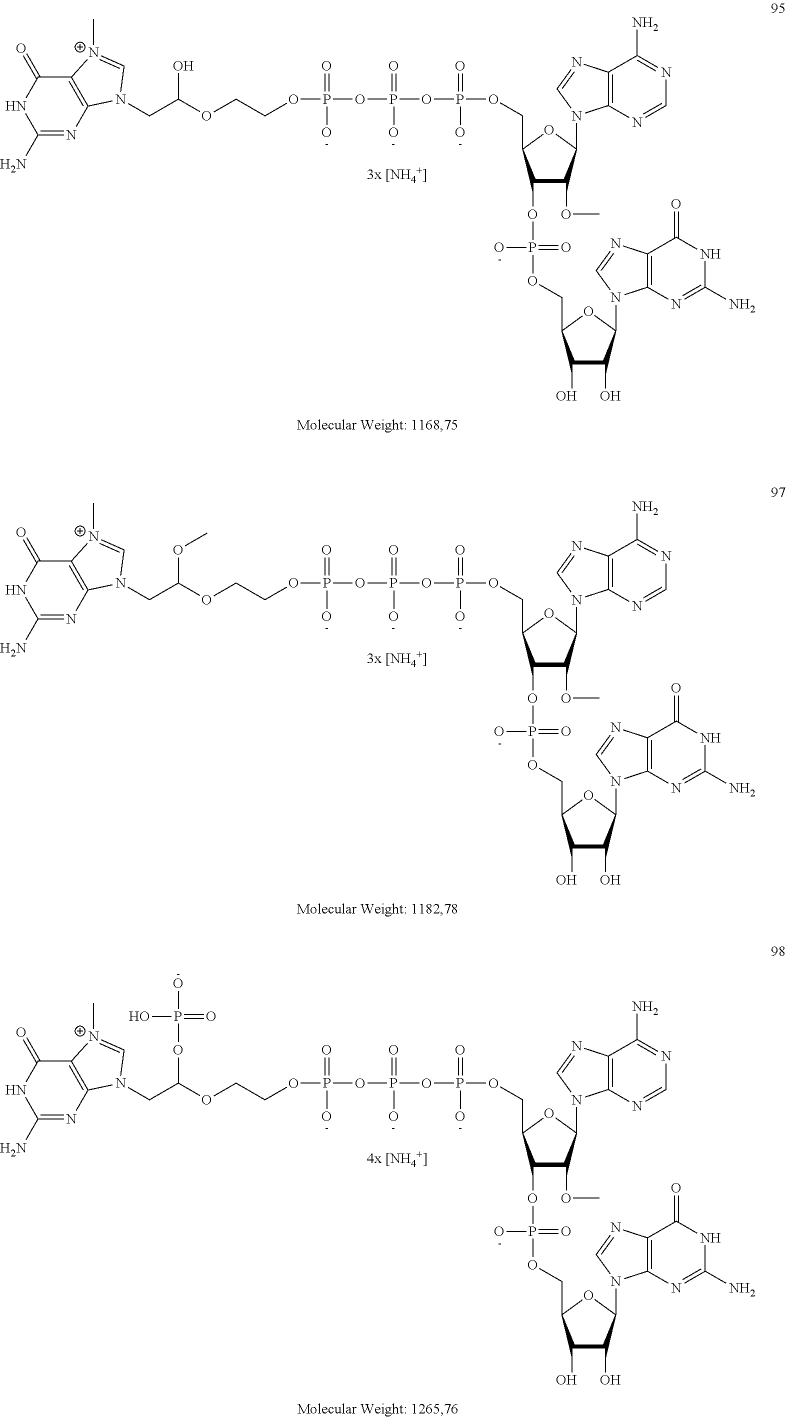

Compounds 93-98 are synthesized from compound 72, 80a, 80b, 86a, 86b and 92, respectively, according to example 1.5.

Similar compounds can be synthesized in accordance with the above synthesis route when starting from other educt compounds.

### Example 1.8: Synthesis Route VIII

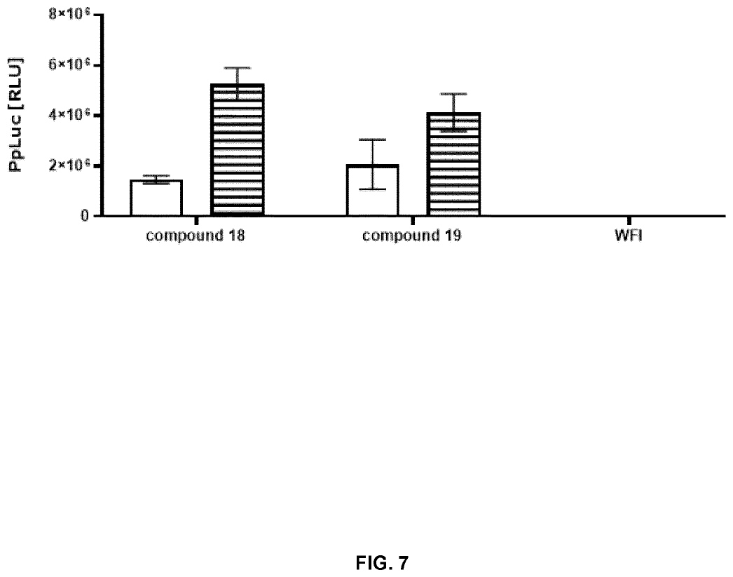

The synthesis of compound 103 (corresponding to 3′-thiophenyl-m⁷-Ganciclovir-ppp-(5′)-Guanosine, referred to the “3′-SPhe-m⁷-Ganciclovir” compound in FIG. 2) and 104 (corresponding to 3′-thiophenyl-m⁷-Ganciclovir-ppp-(5′)-AmG, referred to the “3′-SPhe-m⁷-Ganciclovir Cap1” compound in FIG. 9) when starting from Ganciclovir (X₁) is shown in the following. It is noted that other 3′-thio-modified derivatives with the following general structure G1 (example 1.2: synthesis route II), referred to as “3-SR-m⁷-Ganciclovir”, and G2, “3′-SR-m⁷-Ganciclovir Cap1”, are synthesized similarly to the described reaction sequence using Ganciclovir (X1), Penciclovir (X2) or any other nucleoside diol compound as educt.

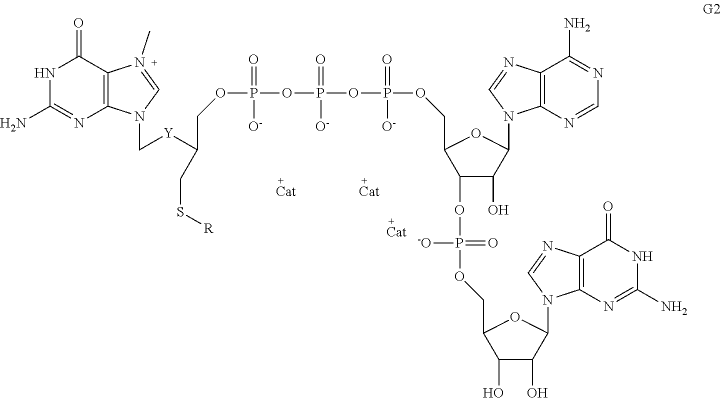

R can be any saturated/unsaturated/semi-saturated/aromatic carbocycle or heterocycle directly attached to the 3′-sulfur or attached to the 3′-sulfur via an alkyl chain, or any linear and/or branched saturated/unsaturated/semi-saturated C1-Cn (n=any number of carbons) alkyl chain, which can include one or more heteroatoms like O, S, N, wherein R can be unsubstituted or substituted with further substituents such as halogen, CN, alkyl, OH, SH, NH₂ or oxo. Cat can be any positively charged counterion like NH₄⁺, Na⁺, Li⁺, K⁺, HEt₃N⁺ or any other positively charged amine species. Y can be O or CH₂. Examples for compounds with the general structure G1 can be synthesized similarly to compound 15 or Compound 103 and are depicted in FIG. 2. Examples for compounds with the general structure G2 can be synthesized similarly to compound 104 and are depicted in FIG. 9.

Synthesis of Compound 99 with the Following Structure:

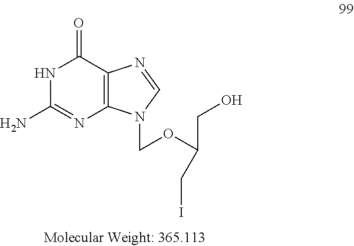 [SMILES: Nc1nc2c(ncn2COC(CO)CI)c(=O)[nH]1]

A suspension of Ganciclovir (X1, 3.00 g, 1.0 eq.) in NMP (45 mL) was treated with triphenylphosphine (1.5 eq.) and imidazole (3.0 eq.). Iodine (1.5 eq.) was added in portions over 10 min followed by stirring at ambient temperature for 3 h. Additional triphenylphosphine (0.5 eq.), imidazole (1.0 eq.) and iodine (0.25 eq) was added and the mixture was stirred for an additional hour. The reaction mixture was then diluted with dichloromethane/water (3/1, v/v, 200 mL) upon which the product precipitates as a white powder. The reaction flask was stored at 4° C. overnight followed by collection of the crude product by filtration and purification via column chromatography (silica 80 g, A: dichloromethane, B: methanol, gradient program from Table Ex-32).

TABLE Ex-32

| Time | | |
|---|---|---|
| total | Flow | B |
| [min] | [mL/min] | [%] |
| 0 | 60 | 0 |
| 38 | 60 | 40 |
| 53 | 60 | 80 |
| 73 | 60 | 80 |

Product fractions were analyzed via ESI-MS and NMR. The product was obtained as a slightly yellow powder in a yield of 62%.

ESI-MS: [M+H]⁺: 366.004, Calculated 336.006

¹H NMR (80 MHz, d₆-DMSO) δ 10.73 (s br., 1H), 7.94 (s, 1H), 6.58 (s br., 2H), 5.43 (s, 2H), 3.36 (m, 5H).

Synthesis of Compound 100 with the Following Structure:

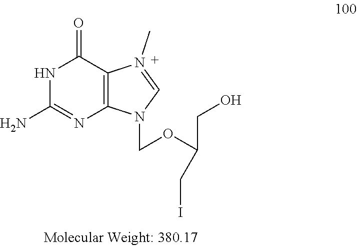

Compound 99 (2.00 g, 1.0 eq.) was suspended in dry DMF (22 mL) and was treated with methyl iodide (8.0 eq.). The reaction mixture was stirred at ambient temperature overnight and was then concentrated. The yellow oil was poured into 200 mL vigorously stirred dichloromethane upon which the product precipitates as a white powder. The reaction flask was stored for 30 min at 0° C. The product was collected by filtration and was washed extensively with cold dichloromethane.

The product was obtained as a slightly yellow powder in a yield of 96%.

ESI-MS: [M+H]⁺: 380.021, Calculated 380.021

¹H NMR (80 MHz, d₆-DMSO) δ 11.68 (s br., 1H), 9.40 (s, 1H), 7.20 (s br., 2H), 5.63 (s, 2H), 4.21 (s br., 1H), 4.03 (s, 3H), 3.63-3.16 (m, 5H).

Synthesis of Compound 101 with the Following Structure:

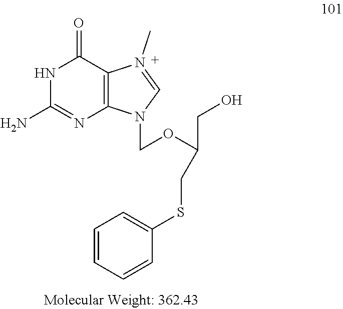

Thiophenol (5.0 eq.) and N,N-diisopropylethylamine (5.0 eq.) were consecutively introduced to a solution of compound 100 (400 mg, 1.0 eq.) in DMF (7.0 mL). The reaction mixture was stirred 1 h at ambient temperature and was then concentrated. The oily residue was coevaporated trice with toluene, was then dry-loaded on silica and purified by flash chromatography (silica 12 g, A: dichloromethane, B: methanol, gradient program from Table Ex-33)

TABLE Ex-33

| Time | | |
|---|---|---|
| total | Flow | B |
| [min] | [mL/min] | [%] |
| 0 | 30 | 0 |
| 16 | 30 | 15 |
| 26 | 30 | 25 |
| 27 | 30 | 80 |
| 32 | 30 | 80 |

Product fractions were analyzed via ESI-MS and NMR. The product was obtained as a slightly yellow powder in a yield of 80%.

ESI-MS: [M]⁺: 362.128, Calculated 362.128

¹H NMR (400 MHz, de-DMSO) b 11.60 (s br., 1H), 9.33 (d, 1H), 7.24 (m, 2H), 7.13 (m, 3H), 5.58 (m, 2H), 4.97 (t, 1H), 3.98 (d, 3H), 3.82 (m, 1H), 3.59 (m, 1H), 3.48 (m, 1H), 3.32 (s br., 2H), 3.21 (dd, 1H), 2.88 (dd, 1H).

Synthesis of Compound 102 with the Following Structure:

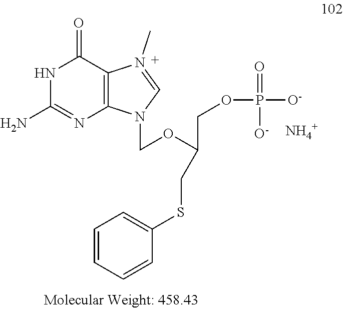

Compound 102 was prepared from compound 101 (300 mg, 1.0 eq.) according to the general procedure 3 employing 2.0 eq. POCl₃, 2.50 mL trimethylphosphate, 2 h reaction time and 5 mL TEAB buffer. Compound 102 was purified by ion exchange chromatography with Macro-Prep High Q resin using a TEAB gradient according to table Ex-34 (A: water, B: 1 M TEAB) followed by repurification by C18 RP, using a Büchi C18, 250×30 mm, 5 μM Column and a gradient program according to table Ex-35 (A: 5 mM ammonium acetate B: 100% MeOH).

TABLE Ex-34

| Time | | |
|---|---|---|
| total | Flow | B |
| [min] | [mL/min] | [%] |
| 0 | 33 | 0 |
| 12 | 33 | 0 |
| 62 | 33 | 100 |
| 72 | 33 | 100 |
| 72 | 33 | 0 |
| 82 | 33 | 0 |

TABLE Ex-35

| Time | | |
|---|---|---|
| total | Flow | B |
| [min] | [mL/min] | [%] |
| 0 | 12 | 0 |
| 10 | 12 | 0 |
| 25 | 12 | 40 |
| 26 | 12 | 80 |
| 33 | 12 | 80 |
| 34 | 12 | 0 |
| 44 | 12 | 0 |

Product fractions were analyzed via ESI-MS and NMR. The product was obtained as a white powder in a yield of 68%.

ESI-MS: [M]⁺: 442.094, Calculated 442.094

¹H NMR (80 MHz, D₂O) δ 7.30 (m, 5H), 4.92 (s, 2H), 3.77 (m, 3H), 3.07 (m, 2H), 2.90 (s, 3H).

³¹P NMR (33 MHz, D₂O) δ 4.53.

Synthesis of Compound 103 with the Following Structure:

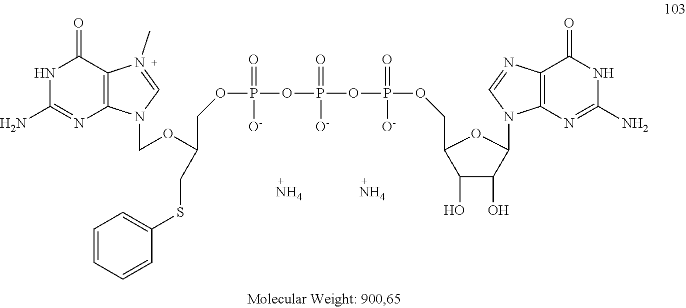

Compound 103 was prepared from compound 102 (370 mg, 1.0 eq.) according to the general procedure 5 using 1.5 eq. guanosine 5′-imidazolide diphosphate, 55 mL H₂O/ACN and 10.0 eq. MgCl₂. After 18 h another 0.2 eq. of guanosine 5′-imidazolide diphosphate were added. The reaction was quenched after 42 h via the addition of 250 mM EDTA-solution (55 mL). The product was purified according to the purification method B using gradient programs from table Ex-36 (Phenomexx Gemini C18, 250×21.2 mm, 5 μM Column. Buffer A: 5 mM ammonium acetate, Buffer B: 90% MeOH), table Ex-37 (DNAPac PA200 column (22×250 mm). Buffer A: 20 mM Tris, pH 9; Buffer B: 20 mM Tris, 33 mM sodium perchlorate), table Ex-38 (Phenomexx Gemini C18, 250×21.2 mm, 5 μM Column. Buffer A: 5 mM ammonium acetate, Buffer B: 90% MeOH), table Ex-39 (Phenomexx Gemini C18, 250×21.2 mm, 5 μM Column. Buffer A: 5 mM ammonium acetate, Buffer B: 90% MeOH) and table Ex-40 (Phenomexx Gemini C18, 250×21.2 mm, 5 μM Column. Buffer A: 5 mM ammonium acetate, Buffer B: 90% MeOH).

TABLE Ex-36

| Time | | |
|---|---|---|
| total | Flow | B |
| [min] | [mL/min] | [%] |
| 0 | 100 | 0 |
| 10 | 100 | 0 |
| 20 | 85 | 15 |
| 21 | 20 | 80 |
| 28 | 20 | 80 |
| 29 | 100 | 0 |
| 43 | 100 | 0 |

TABLE Ex-37

| Time | | |
|---|---|---|
| total | A | B |
| [min] | [%] | [%] |
| 0 | 100 | 0 |
| 30 | 25 | 75 |
| 31 | 0 | 100 |
| 35 | 0 | 100 |
| 36 | 100 | 0 |
| 50 | 100 | 0 |

TABLE Ex-38

| Time | | |
|---|---|---|
| total | A | B |
| [min] | [%] | [%] |
| 0 | 100 | 0 |
| 10 | 100 | 0 |
| 30 | 70 | 30 |
| 31 | 20 | 80 |
| 40 | 20 | 80 |
| 41 | 100 | 0 |
| 55 | 100 | 0 |

TABLE Ex-39

| Time | | |
|---|---|---|
| total | A | B |
| [min] | [%] | [%] |
| 0 | 100 | 0 |
| 10 | 100 | 0 |
| 30 | 70 | 30 |
| 31 | 20 | 80 |
| 40 | 20 | 80 |
| 41 | 100 | 0 |
| 58 | 100 | 0 |

TABLE Ex-40

| Time | | |
|---|---|---|
| total | A | B |
| [min] | [%] | [%] |
| 0 | 100 | 0 |
| 10 | 100 | 0 |
| 30 | 70 | 30 |
| 31 | 20 | 80 |
| 40 | 20 | 80 |
| 41 | 100 | 0 |
| 58 | 100 | 0 |

Product fractions were analyzed via ESI-MS and NMR. The product was obtained as a white powder in a yield of 20%.

ESI-MS: [M]⁺: 867.109, Calculated 867.108

¹H NMR (400 MHz, D₂O) δ 9.01 (m, 1H), 8.02 (m, 1H), 7.14 (m, 2H), 7.03 (m, 1H), 6.83 (m, 1H), 5.80 (dd, 1H), 5.62 (m, 2H), 4.65 (m, 1H), 4.49 (m, 1H), 4.35 (m, 1H), 4.31-4.18 (m, 3H), 4.09 (m, 2H), 3.95 (s, 3H), 3.08 (m, 1H), 2.72 (m, 1H).

³¹P NMR (162 MHz, D₂O) δ −11.38 (m, 2P), −23.07 (m, 1P).

Synthesis of Compound 104 with the Following Structure:

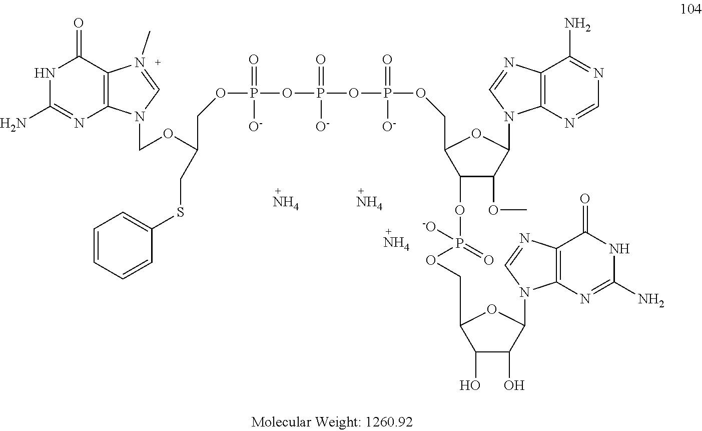

Compound 104 was prepared from compound 102 (122 mg, 1.0 eq.) following the general procedure 7 employing 1.5 mL DMSO, 20.0 eq. CDI (2 portions within 48 h) and 20.0 eq. water. Total reaction time: 6 d. Compound 104 was purified according to according to general procedure 7 using the gradient program of table Ex-41 (A: 5 mM ammonium acetate in water; B: 90% methanol in water), followed by a second purification using the gradient program of table Ex-42 (A: 100 mM triethylammonium acetate (pH 7), B: 90% MeOH in water), followed by a third purification using the gradient program of table Ex-43 (A: 5 mM ammonium acetate in water; B 90% methanol in water).

TABLE Ex-41

| Time | | |
|---|---|---|
| total | A | B |
| [min] | [%] | [%] |
| 0 | 100 | 0 |
| 10 | 100 | 0 |
| 20 | 80 | 20 |
| 21 | 5 | 95 |
| 29 | 5 | 95 |
| 30 | 100 | 0 |
| 45 | 100 | 0 |

TABLE Ex-42

| Time | | |
|---|---|---|
| total | A | B |
| [min] | [%] | [%] |
| 0 | 100 | 0 |
| 40 | 60 | 40 |
| 41 | 20 | 80 |
| 48 | 20 | 80 |
| 49 | 100 | 0 |
| 60 | 100 | 0 |

TABLE Ex-43

| Time | | |
|---|---|---|
| total | A | B |
| [min] | [%] | [%] |
| 0 | 100 | 0 |
| 10 | 100 | 0 |
| 40 | 50 | 50 |
| 41 | 20 | 80 |
| 46 | 20 | 80 |
| 47 | 100 | 0 |
| 55 | 100 | 0 |

Product fractions were analyzed via ESI-MS and NMR. The product was obtained as a white powder in a yield of 48%.

ESI-MS: [M]⁺: 1210.178, Calculated 1210.176

³¹P NMR (162 MHz, D₂O) 6-0.89 (m, 1P), −11.34 (m, 2P), −22.79 (m, 1P).

Similar compounds can be synthesized in accordance with the above synthesis route when introducing different 3′-thio-modifications. Exemplary compounds along these lines are shown in FIG. 2 and FIG. 9, namely i) the “3′-SCE-m⁷-Ganciclovir” compound; ii) the “3′-SCH₃-m⁷-Penciclovir” compound; iii) the “3′-SPyBr-m⁷-Ganciclovir” compound; iv) the “3′-S—SCH₃-m⁷-Ganciclovir” compound; v) the “3′-SCH₂C₆H₁₁-m⁷-Ganciclovir” compound; vi) the “3′-SCE-m⁷-Ganciclovir Cap1” compound; vii) the “3′-SCH₃-m⁷-Penciclovir Cap1” compound; viii) the “3′-SPyBr-m⁷-Ganciclovir Cap1” compound, ix) the “3′-S—SCH₃-m⁷-Ganciclovir Cap1” compound, and x) the “3′-SCH₂C₆H₁₁-m⁷-Ganciclovir Cap1” compound, all starting from Ganciclovir (X1) or Penciclovir (X2).

### Example 1.9: Synthesis Route IX

The synthesis of compound 22 (corresponding to m⁷-Ganciclovir-ppp-AmG), 109 (corresponding to N⁷-(4-chlorobenzyl)-Ganciclovir-ppp-AmG), 110 (corresponding to N⁷-biphenyl methyl-Ganciclovir-ppp-AmG) and 112 (corresponding to m⁷-Penciclovir-ppp-AmG), referred to in FIG. 10 as “m⁷-Ganciclovir Cap1”, “N⁷-4ClBn-Ganciclovir Cap1”, “N⁷-BiPheMe-Ganciclovir Cap1” and “m⁷-Penciclovir Cap1” compound when starting from compound 1 (which is synthesized from Ganciclovir (X1)) or Penciclovir (X2) is shown in the following. It is noted that the synthesis of other N⁷ modified Ganciclovir or Penciclovir derivatives (such as e.g. other substituted phenyl, biphenyl or naphthalene moieties) is identical. Examples of resulting compounds analogous to compound 22, 109, 110 and 112 when starting from compound 1 (which is synthesized from Ganciclovir (X1)) or Penciclovir (X2) are described at the end of the present example and are depicted in FIG. 10.

Synthesis of Compound 105 with the Following Structure:

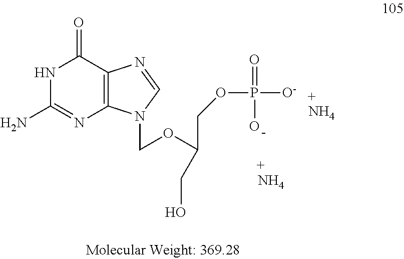

Compound 105 was prepared from compound 1 (3.0 g, 1.0 eq.) according to the general procedure 2 using 30 mL acetonitrile, 27 mL 0.45 M tetrazole solution and 2 eq. of bis-cyanoethyl-N,N-diisopropyl-phosphoramidite. Oxidation was carried out using 2 eq. of a 1 M solution of tBuOOH in dry toluene. Deprotection was conducted using 100 mL of aqueous ammonia in a pressure tube at 60° C. The crude product was purified by ion exchange chromatography with Macro-Prep-High-Q resin using a TEAB gradient according to Table Ex-44 (A: water, B: 1 M TEAB) and was desalted according to Table Ex-45 (Buchi C18, 250×30 mm, 5 μM Column. Buffer A: 5 mM ammonium acetate, Buffer B: 100% MeOH).

TABLE Ex-44

| Time | | |
|---|---|---|
| total | Flow | B |
| [min] | [mL/min] | [%] |
| 0 | 60 | 0 |
| 10 | 60 | 0 |
| 60 | 60 | 100 |
| 80 | 60 | 100 |
| 80 | 60 | 0 |
| 90 | 60 | 0 |

TABLE Ex-45

| Time | | |
|---|---|---|
| total | Flow | B |
| [min] | [mL/min] | [%] |
| 0 | 12 | 0 |
| 10 | 12 | 0 |
| 35 | 12 | 30 |
| 36 | 12 | 80 |
| 41 | 12 | 80 |
| 42 | 12 | 0 |
| 49 | 12 | 0 |

Product fractions were analyzed via ESI-MS and NMR The product was obtained as a white powder in a yield of 99%.

ESI-MS: [M+H]⁺: 336.070, Calculated 336.070

¹H NMR (80 MHz, D₂O) δ 7.93 (s, 1H), 5.59 (s, 2H), 3.89 (m, 3H), 3.62 (s, 2H).

³¹P NMR (33 MHz, D₂O) δ 0.86.

Synthesis of Compound 106 with the Following Structure:

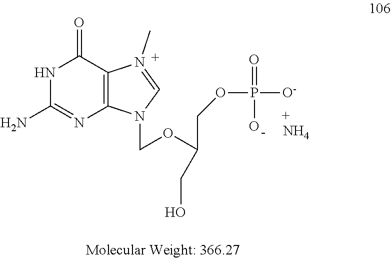

Compound 105 (300 mg. 1.0 eq) was dissolved in water (8.9 mL) and pH was adjusted to 4.0 using glacial acetic acid. Dimethyl sulfate (5.8 eq) was added dropwise over the course of 1 h. Simultaneously the solution was titrated with 1 M NaOH to keep the pH between 3.8 and 4.0. TLC control (iPr/H₂O/NH₃ 7:3:1) after 1.5 h showed complete consumption of the starting material and in the following the reaction mixture was extracted four times with chloroform (40 mL each). Residual chloroform from the aqueous phase was removed under reduced pressure (100 mbar, 20 min). The aqueous layer was diluted with water (1 L) and pH was set to 7 using aqueous ammonium hydroxide. Purification was carried out according to the general procedure 4 using ion exchange chromatography with Macro-Prep High Q resin and the TEAB gradient from table Ex-46 (A: water, B: 1 M TEAB) followed by repurification using a Buchi C18, 250×30 mm, 5 μM Column and the gradient profile from table Ex. 47 (A: 5 mM Ammonium acetate; B: 100% MeCOH).

TABLE Ex-46

| Time | | |
|---|---|---|
| total | Flow | B |
| [min] | [mL/min] | [%] |
| 0 | 28 | 0 |
| 5 | 28 | 0 |
| 30 | 28 | 100 |
| 40 | 28 | 100 |
| 40 | 28 | 0 |
| 45 | 28 | 0 |

TABLE Ex-47

| Time | | |
|---|---|---|
| total | Flow | B |
| [min] | [mL/min] | [%] |
| 0 | 12 | 0 |
| 10 | 12 | 0 |
| 35 | 12 | 30 |
| 36 | 12 | 80 |
| 41 | 12 | 80 |
| 42 | 12 | 0 |
| 49 | 12 | 0 |

Product fractions were analyzed via ESI-MS and NMR The product was obtained as a white powder in a yield of 46%.

ESI-MS: [M+H]⁺: 350.085, Calculated 350.086

¹H NMR (80 MHz, D₂O) δ 9.12 (s br., 1H), 5.81 (s, 2H), 4.14 (s, 3H), 3.96-3.63 (m, 5H).

³¹P NMR (33 MHz, D₂O+3 drops NaOD): δ 4.75.

Synthesis of Compound 22 with the Following Structure:

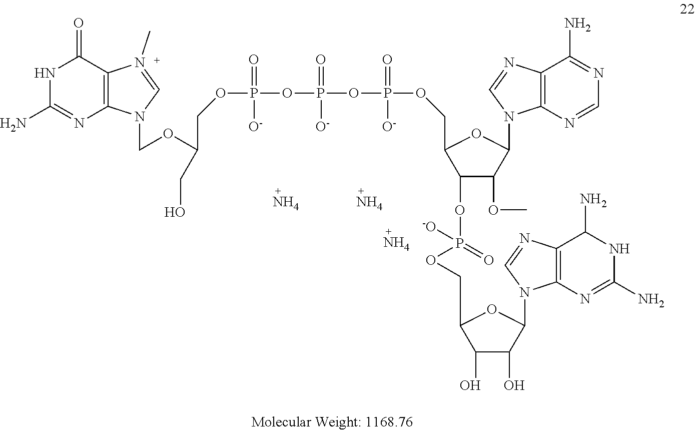

Compound 22 was prepared from compound 106 (100 mg, 1.0 eq.) following the general procedure 7 employing 1.0 mL DMSO, 20.0 eq CDI (2 portions within 48 h) and 20.0 eq water. Total reaction time: 5 d. Compound 22 was purified according to general procedure 7 using the gradient program of table Ex-48 (A: 5 mM ammonium acetate in water; B: 90% methanol in water), followed by a second purification using the gradient program of table Ex-49 (A: 100 mM triethylammonium acetate (pH 7), B: 90% MeOH in water), followed by a third purification using the gradient program of table Ex-50 (A: 5 mM ammonium acetate in water; B: 90% methanol).

TABLE Ex-48

| Time | A | B |
|---|---|---|
| [min] | [%] | [%] |
| 0 | 100 | 0 |
| 10 | 100 | 0 |
| 20 | 80 | 20 |
| 21 | 5 | 95 |
| 29 | 5 | 95 |
| 30 | 100 | 0 |
| 42 | 100 | 0 |

TABLE Ex-49

| Time | A | B |
|---|---|---|
| [min] | [%] | [%] |
| 0 | 100 | 0 |
| 40 | 60 | 40 |
| 41 | 100 | 0 |
| 51 | 100 | 0 |

TABLE Ex-50

| Time | A | B |
|---|---|---|
| [min] | [%] | [%] |
| 0 | 100 | 0 |
| 10 | 100 | 0 |
| 40 | 50 | 50 |
| 41 | 20 | 80 |
| 46 | 20 | 80 |
| 47 | 100 | 0 |
| 55 | 100 | 0 |

Product fractions were analyzed via ESI-MS and NMR. The product was obtained as a white powder in a yield of 15%.

ESI-MS: [M]⁺: 1118.164, Calculated 1118.168

¹H NMR (400 MHz, D₂O) δ 9.04 (m, 1H), 8.40 (m, 1H), 8.10 (s, 1H), 7.95 (s, 1H), 6.03 (dd, 1H), 5.83 (dd, 1H), 5.65 (m, 2H), 4.95 (m, 1H), 4.53 (m, 1H), 4.49 (m, 1H), 4.44 (m, 1H), 4.37-4.18 (m, 5H), 4.09 (m, 1H), 4.02 (s, 3H), 3.98 (m, 2H), 3.64 (m, 1H), 3.54 (m, 1H), 3.45 (m, 3H).

³¹P NMR (162 MHz, D₂O) δ −0.89 (s, 1P), −11.26 (d, 1P), −11.47 (dd, 1P), −22.84 (m, 1P).

Synthesis of Compound 107 with the Following Structure:

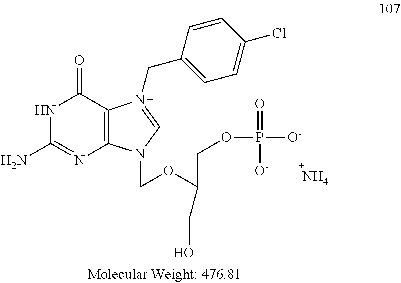

Dry compound 105 (415 mg, 1.0 eq.) was suspended in dry DMSO (12 mL) under argon atmosphere. 4-Chlorobenzyl bromide (8. eq.) was added and the reaction mixture was stirred for 4 h at 45° C. The solution was cooled to ambient temperature, was diluted with water (100 mL) and washed trice with diethyl ether (100 mL). The aqueous phase was isolated and remaining organic solvent was evaporated. The crude product was purified by ion exchange chromatography with Macro-Prep-High-Q resin using a TEAB gradient from 0-1 M (Table Ex-51, A: water, B: TEAB 1.0 M). The product fractions were repurified via C18 RP, using a Buchi C18, 300×30 mm, 5 μM Column. Buffer A: 5 mM ammonium acetate pH 5.6, Buffer B: 100% MeOH (Table Ex-52). The final product was lyophilized from water.

TABLE Ex-51

| Time | | |
|---|---|---|
| total | Flow | B |
| [min] | [mL/min] | [%] |
| 0 | 28 | 0 |
| 5 | 28 | 0 |
| 30 | 28 | 100 |
| 40 | 28 | 100 |
| 40 | 28 | 0 |
| 45 | 28 | 0 |

TABLE Ex-52

| Time total [min] | Flow [mL/min] | B [%] |
|---|---|---|
| 0 | 12 | 0 |
| 10 | 12 | 0 |
| 35 | 12 | 30 |
| 36 | 12 | 80 |
| 41 | 12 | 80 |
| 42 | 12 | 0 |
| 54.5 | 12 | 0 |

Product fractions were analyzed via ESI-MS and NMR The product was obtained as a white powder in a yield of 26%.

ESI-MS: [M]⁺: 460.077, Calculated 460.078

¹H NMR (80 MHz, D₂O) δ 7.40 (s, 4H), 5.80 (s, 2H), 5.63 (s, 2H), 3.96 (m, 3H), 3.67 (s, 2H).

³¹P NMR (33 MHz, D₂O) δ 1.67.

Synthesis of Compound 108 with the Following Structure:

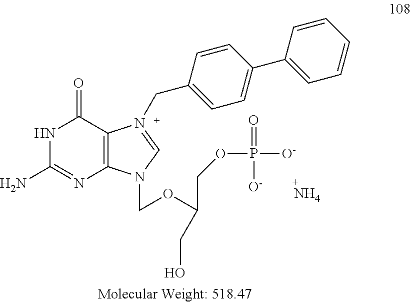

Dry compound 105 (200 mg, 1.0 eq.) was suspended in dry DMSO (5.3 mL) under argon atmosphere. 4-(Bromomethyl)biphenyl (4 eq.) was added and the reaction mixture was stirred for 1 h at 55° C. The solution was cooled to ambient temperature, was diluted with water (3 L), filtered and purified by ion exchange chromatography with Macro-Prep-High-Q resin using a TEAB gradient from 0-1 M (Table Ex-53, A: water, B: TEAB 1.0 M). The product fractions were repurified via C18 RP, using a Buchi C18, 300×30 mm, 5 μM Column. Buffer A: 5 mM ammonium acetate pH 5.6, Buffer B: 100% MeOH (Table Ex-54).

TABLE Ex-53

| Time total [min] | Flow [mL/min] | B [%] |
|---|---|---|
| 0 | 28 | 0 |
| 5 | 28 | 0 |
| 30 | 28 | 100 |
| 40 | 28 | 100 |
| 40 | 28 | 0 |
| 45 | 28 | 0 |

TABLE Ex-54

| Time total [min] | Flow [mL/min] | B [%] |
|---|---|---|
| 0 | 12 | 0 |
| 10 | 12 | 0 |
| 28 | 12 | 30 |
| 29 | 12 | 80 |
| 36 | 12 | 80 |
| 37 | 12 | 0 |
| 49 | 12 | 0 |

Product fractions were analyzed via ESI-MS and NMR The product was obtained as a white powder in a yield of 36%.

ESI-MS: [M]⁺: 502.149, Calculated 502.149

¹H NMR (400 MHz, D₂O+1 drop NaOD): δ 7.63 (m, 2H), 7.56 (m, 2H), 7.46 (m, 2H), 7.36 (m, 3H), 5.07 (m, 0.5H)*, 4.93 (s, 2H), 4.48 (m, 0.5H)*, 4.15 (m, 0.5H)*, 3.75 (m, 3H), 3.57 (s, 2H).

³¹P NMR (161 MHz, D₂O+1 drop NaOD): δ 4.44.

*Chirality leads to pronounced peak splitting

Synthesis of Compound 109 with the Following Structure:

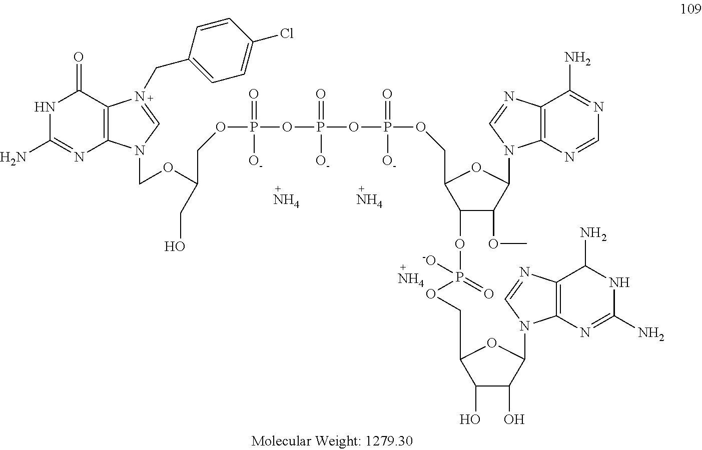

Compound 109 was prepared from compound 107 (30 mg, 1.0 eq.) following the general procedure 7 employing 0.5 mL DMSO, 10.0 eq. CDI and 10.0 eq. water. Total reaction time 3 d. Compound 109 was purified according to general procedure 7 using the gradient program of table Ex-55 (A: 5 mM ammonium acetate in water; B: 90% methanol in water), followed by a second purification using the gradient program of table Ex-56 (A: 100 mM triethylammonium acetate (pH 7), B: 90% MeOH in water), followed by a third purification using the gradient program of table Ex-57 (A: 5 mM ammonium acetate in water; B: 90% methanol). The sample elutes incompletely from the C₁₈ column during the first purification, for which reason washing runs were placed between sample runs (table-Ex-55b, A: 5 mM ammonium acetate in water; B: 90% methanol in water).

TABLE Ex-55a

| Time [min] | A [%] | B [%] |
|---|---|---|
| 0 | 100 | 0 |
| 10 | 100 | 0 |
| 20 | 80 | 20 |
| 21 | 5 | 95 |
| 44 | 5 | 95 |
| 45 | 100 | 0 |
| 60 | 100 | 0 |

TABLE Ex-55b

| Time [min] | A [%] | B [%] |
|---|---|---|
| 0 | 100 | 0 |
| 10 | 100 | 0 |
| 11 | 5 | 95 |
| 44 | 5 | 95 |
| 45 | 100 | 0 |
| 60 | 100 | 0 |

TABLE Ex-56

| Time [min] | A [%] | B [%] |
|---|---|---|
| 0 | 100 | 0 |
| 40 | 5 | 95 |
| 47 | 5 | 95 |
| 48 | 100 | 0 |
| 60 | 100 | 0 |

TABLE Ex-57

| Time [min] | A [%] | B [%] |
|---|---|---|
| 0 | 100 | 0 |
| 10 | 100 | 0 |
| 40 | 5 | 95 |
| 46 | 5 | 95 |
| 47 | 100 | 0 |
| 65 | 100 | 0 |

Product fractions were analyzed via ESI-MS and NMR. The product was obtained as a white powder in a yield of 16%.

ESI-MS: [M]⁺: 1228.160, Calculated 1228.160

¹H NMR (400 MHz, D₂O) δ 8.34 (s, 1H), 8.06 (s, 1H), 7.95 (s, 1H), 7.26 (s, 4H), 5.92 (t, 1H), 5.82 (dd, 1H), 5.63 (m, 2H), 5.49 (m, 2H), 4.95 (m, 1H), 4.51 (m, 2H), 4.42 (m, 1H), 4.34 (m, 2H), 4.24 (m, 3H), 4.10 (m, 1H), 4.00 (m, 2H), 3.61 (m, 1H), 3.52 (m, 1H), 3.43 (m, 3H).

³¹P NMR (162 MHz, D₂O) δ −0.81 (s, 1P), −11.02 (dd, 1P), −11.34 (dd, 1P), −22.49 (m, 1P).

Synthesis of Compound 110 with the Following Structure:

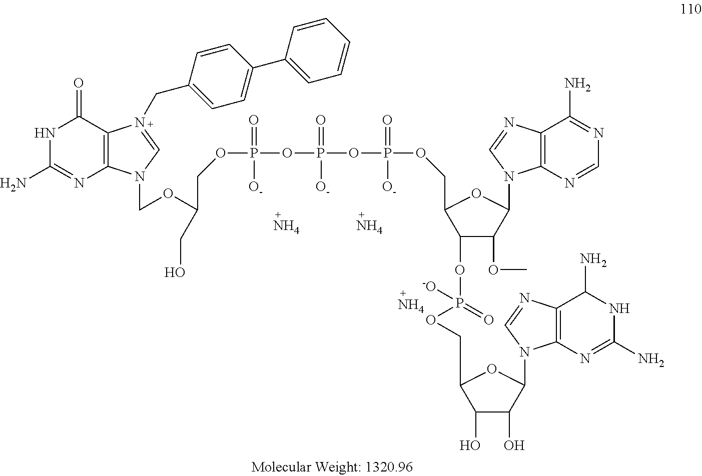

Compound 110 was prepared from compound 108 (100 mg, 1.0 eq.) following the general procedure 7 employing 1.0 mL DMSO, 10.0 eq. CDI and 10.0 eq. water. Total reaction time: 4 d. Compound 110 was purified according to general procedure 7 using the gradient program of table Ex-58 (A: 5 mM ammonium acetate in water; B: 90% methanol in water), followed by a second purification using the gradient program of table Ex-59 (A: 100 mM triethylammonium acetate (pH 7), B: 90% MeOH in water), followed by a third purification using the gradient program of table Ex-60 (A: 5 mM ammonium acetate in water; B: 90% methanol). The sample elutes incompletely from the C₁₈ column during the last purification for which reason washing runs were placed between sample runs (table-Ex-60, A: 5 mM ammonium acetate in water; B: 90% methanol in water).

TABLE Ex-58

| Time [min] | A [%] | B [%] |
|---|---|---|
| 0 | 100 | 0 |
| 10 | 100 | 0 |
| 20 | 80 | 20 |
| 21 | 5 | 95 |
| 29 | 5 | 95 |
| 30 | 100 | 0 |
| 45 | 100 | 0 |

TABLE Ex-59

| Time [min] | A [%] | B [%] |
|---|---|---|
| 0 | 100 | 0 |
| 40 | 5 | 95 |
| 45 | 5 | 95 |
| 46 | 100 | 0 |
| 60 | 100 | 0 |

TABLE Ex-60

| Time [min] | A [%] | B [%] |
|---|---|---|
| 0 | 100 | 0 |
| 10 | 100 | 0 |
| 11 | 5 | 95 |
| 54 | 5 | 95 |
| 55 | 100 | 0 |
| 70 | 100 | 0 |

ESI-MS: [M]⁺: 1270.227, Calculated 1270.231

¹H NMR (400 MHz, D₂O) 59.35 (s br., 1H), 8.31 (s br., 1H), 7.88 (s br., 2H), 7.07 (s br., 9H), 5.82 (s br., 1H), 5.70 (d, 1H), 5.52 (s br., 2H), 5.35 (s br., 2H), 4.92 (s br., 1H), 4.63 (m, 1H), 4.47 (m, 2H), 4.34-4.10 (m, 6H), 4.00 (s br., 2H), 3.66 (m, 1H), 3.56 (m, 1H), 3.33 (s, 3H).

³¹P NMR (162 MHz, D₂O) δ −0.87 (s, 1P), −11.41 (m, 2P), −22.72 (m, 1P).

Synthesis of Compound 111 with the Following Structure:

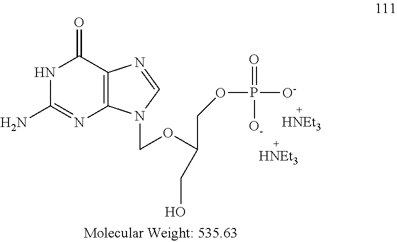

Compound 111 was prepared from Penciclovir (X2, 0.5 g) following the general procedure 2 with the difference that the starting material was dissolved in 20 mL dry DMSO. Other employed reagents: 2.0 eq. acetonitrile containing 0.45 M tetrazole, 2.0 eq. bis-cyanoethyl-N,N-diisopropyl phosphoramidate and 2.0 eq. 1 M tBuOOH in toluene (2 portions within 30 min). Upon complete formation of the oxidation product, DMSO was removed by lyophilization and the residue was transferred into a pressure tube and deprotected using methanol/concentrated ammonia (1:1, 30 mL) at 60° C. The crude product was coevaporated with methanol and was purified by ion exchange chromatography with Macro-Prep-High-Q resin using a TEAB gradient from 0-1 M according to Table ex 61 (A water, B: 1.0 M TEAB).

TABLE Ex-61

| Time total [min] | Flow [mL/min] | B [%] |
|---|---|---|
| 0 | 66 | 0 |
| 10 | 66 | 0 |
| 100 | 66 | 100 |
| 130 | 66 | 100 |
| 130 | 66 | 0 |
| 140 | 66 | 0 |

Product fractions were analyzed via ESI-MS and NMR. The product was obtained as a white powder in a yield of 50%

ESI-MS: [M−H]⁻: 332.076, Calculated 332.077

¹H NMR (80 MHz, D₂O) δ 8.72, (s br., 2H), 7.85 (s, 1H), 4.16 (m, 2H), 3.91 (m, 2H), 3.63 (m, 2H), 1.88 (m, 3H).

³¹P NMR (33 MHz, D₂O) δ 0.90.

Synthesis of Compound 112 with the Following Structure:

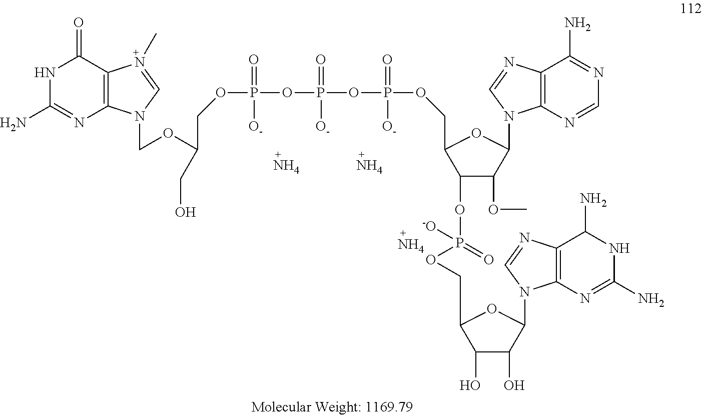

MeI (0.15 mL) was added to a stirred solution of compound 111 (0.14 mmol, 1.0 eq.) in DMSO (3.0 mL). The reaction was deemed complete by LC-MS after 165 min followed by evaporation of the excessive MeI N⁷-methylated compound 111 was then treated with CDI (487 mg) and stirred for 20 h at ambient temperature. Water (110 μl) was introduced to the reaction mixture followed by the consecutive addition of 2 M NMM buffer (350 μl, pH 7), compound 28 (236 mg) and MnCl₂ (38 mg). Upon complete product formation (48 h), the reaction was quenched by the addition of 250 mM EDTA solution (12 mL) and the crude product was purified as described in general procedure 7 using the gradient program of Table Ex 62 (A: 5 mM ammonium acetate in water; B: 90% methanol in water), followed by a second purification using the gradient program of table Ex-63 (A: 100 mM triethylammonium acetate (pH 7), B: 90% MeOH in water), followed by a third purification using the gradient program of table Ex-64 (A: 5 mM ammonium acetate in water; B 90% methanol).

TABLE Ex-62

| Time [min] | A [%] | B [%] |
|---|---|---|
| 0 | 100 | 0 |
| 10 | 100 | 0 |
| 20 | 80 | 20 |
| 21 | 5 | 95 |
| 29 | 5 | 95 |
| 30 | 100 | 0 |
| 45 | 100 | 0 |

TABLE Ex-63

| Time [min] | A [%] | B [%] |
|---|---|---|
| 0 | 100 | 0 |
| 40 | 60 | 40 |
| 42 | 100 | 0 |
| 50 | 100 | 0 |

TABLE Ex-64

| Time [min] | A [%] | B [%] |
|---|---|---|
| 0 | 100 | 0 |
| 10 | 100 | 0 |
| 40 | 50 | 50 |
| 41 | 20 | 80 |
| 48 | 20 | 80 |
| 49 | 100 | 0 |
| 60 | 100 | 0 |

Product fractions were analyzed via ESI-MS and NMR. The product was obtained as a white powder in a yield of 12%.

ESI-MS: [M]⁺: 1116.189, Calculated 1116.189

¹H NMR (400 MHz, D₂O) δ 8.90 (m, 1H), 8.40 (m, 1H), 8.10 (m, 1H), 7.95 (m, 1H), 6.02 (dd, 1H), 5.81 (d, 1H), 4.93 (m, 1H), 4.76 (m, 1H), 4.52 (m, 1H), 4.48 (dd, 1H), 4.42 (q, 1H), 4.36-4.00 (m, 9H), 3.97 (s, 3H), 3.58 (m, 2H), 3.44 (s, 3H), 1.82 (m, 3H).

³¹P NMR (162 MHz, D₂O) δ −0.89 (s, 1P), −10.86 (d, 1P), −11.53 (d, 1P), −22.86 (m, 1P).

Similar compounds can be synthesized in accordance with the above synthesis route when introducing different N⁷-modifications. Exemplary compounds along these lines are shown in FIG. 10, namely i) the “N⁷-Nap-Penciclovir Cap1” compound and ii) the “N⁷-3,5Me₂Bn-Penciclovir Cap1” compound, all starting from compound 1 (which is synthesized from Ganciclovir (X1)) or Penciclovir (X2).

### Example 1.10: Synthesis Route X

The synthesis of compound 116 (corresponding to 3′-OMe-N⁷-(4-chlorobenzyl)-Ganciclovir-ppp-AmG) and 117 (3′-OMe-N⁷-biphenyl methyl-Ganciclovir-ppp-AmG), referred to in FIG. 10 as “3′-OMe-N⁷-4ClBn-Ganciclovir Cap1” and “3′-OMe-N⁷-BiPheMe-Ganciclovir Cap1” compound) when starting from compound 1 ((which is synthesized from Ganciclovir (X1)) or its Penciclovir analog is shown in the following. It is noted that the synthesis of other 3′-OMe-N⁷-modified Ganciclovir or Penciclovir derivatives (such as e.g., other substituted phenyl, biphenyl or naphthalene moieties) is identical. Examples of resulting compounds analogous to compound 116 and 117 when starting from compound 1 ((which is synthesized from Ganciclovir (X1)) or its Penciclovir analog (X2) are described at the end of the present example and are depicted in FIG. 10.

Synthesis of Compound 114 with the Following Structure:

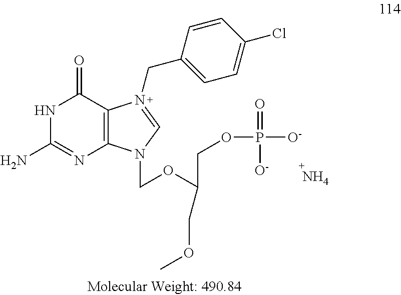

Dry compound 113 (prepared from compound 7 via C18 RP employing 5-10 mM ammonium acetate (Buffer A) and 100% MeOH (Buffer B), 264 mg, 1.0 eq) was suspended in dry DMSO (7.3 mL) under argon atmosphere. 4-Chlorobenzyl bromide (6 eq.) was added, and the reaction mixture was stirred for 1 h at 45° C. The solution was cooled to ambient temperature, was diluted with water and purified by ion exchange chromatography with Macro-Prep-High-Q resin using a TEAB gradient from 0-1 M (Table Ex-65, A: water, B: TEAB 1.0 M). The product fractions were repurified via C18 RP, using a Buchi C18, 300×30 mm, 5 μM Column. Buffer A: 5 mM ammonium acetate pH 5.6, Buffer B: 100% MeOH (Table Ex-66).

TABLE Ex-65

| Time total [min] | Flow [mL/min] | B [%] |
|---|---|---|
| 0 | 28 | 0 |
| 5 | 28 | 0 |
| 30 | 28 | 100 |
| 40 | 28 | 100 |
| 40 | 28 | 0 |
| 45 | 28 | 0 |

TABLE Ex-66

| Time | | |
|---|---|---|
| total | Flow | B |
| [min] | [mL/min] | [%] |
| 0 | 12 | 0 |
| 10 | 12 | 0 |
| 30 | 12 | 30 |
| 31 | 12 | 80 |
| 36 | 12 | 80 |
| 37 | 12 | 0 |
| 49 | 12 | 0 |

Product fractions were analyzed via ESI-MS and NMR The product was obtained as a white powder in a yield of 58%.

ESI-MS: [M]⁺: 474.093, Calculated 474.094

¹H NMR (80 MHz, D₂O) δ 7.32 (s, 4H), 5.76 (s, 2H), 5.58 (s, 2H), 3.93 (m, 3H), 3.50 (m, 2H), 3.13 (s, 3H).

³¹P NMR (33 MHz, D₂O) δ 0.88.

Synthesis of Compound 115 with the Following Structure:

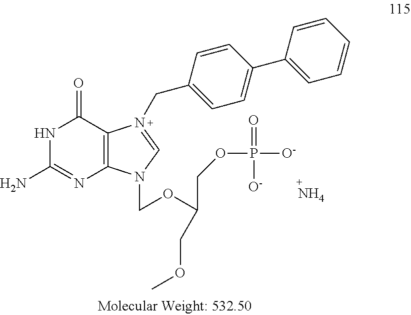

Dry compound 113 (prepared from compound 7 via C18 RP employing 5-10 mM ammonium acetate (Buffer A) and 100% MeOH (Buffer B), 227 mg, 1.0 eq.) was suspended in dry DMSO (6.0 mL) under argon atmosphere. 4-(bromomethyl)biphenyl (3 eq.) was added and the reaction mixture was stirred for 1 h at 55° C. The solution was cooled to ambient temperature, was diluted with water (3 L), filtered and purified by ion exchange chromatography with Macro-Prep-High-Q resin using a TEAB gradient from 0-1 M (Table Ex-67, A: water, B: TEAB 1.0 M). The product fractions were repurified via C18 RP, using a Büchi C18, 300×30 mm, 5 μM Column. Buffer A: 5 mM ammonium acetate pH 5.6, Buffer B: 100% MeOH (Table Ex-68).

TABLE Ex-67

| Time | | |
|---|---|---|
| total | Flow | B |
| [min] | [mL/min] | [%] |
| 0 | 28 | 0 |
| 5 | 28 | 0 |
| 30 | 28 | 100 |
| 40 | 28 | 100 |
| 40 | 28 | 0 |
| 45 | 28 | 0 |

TABLE Ex-68

| Time | | |
|---|---|---|
| total | Flow | B |
| [min] | [mL/min] | [%] |
| 0 | 12 | 0 |
| 10 | 12 | 0 |
| 28 | 12 | 30 |
| 29 | 12 | 80 |
| 36 | 12 | 80 |
| 37 | 12 | 0 |
| 54 | 12 | 0 |

Product fractions were analyzed via ESI-MS and NMR The product was obtained as a white powder in a yield of 43%.

ESI-MS: [M]⁺: 516.164, Calculated 516.164

¹H NMR (400 MHz, D₂O+1 drop NaOD): δ 7.58 (m, 2H), 7.51 (m, 2H), 7.41 (m, 2H), 7.32 (m, 3H), 5.03 (m, 0.5H)*, 4.91 (s, 2H), 4.75 (m, 0.5H)*, 4.43 (m, 0.5H)*, 4.11 (m, 0.5H)*, 3.87 (s, 1H), 3.65 (m, 2H), 3.47 (dd, 1H), 3.39 (dd, 1H), 3.29 (s, 3H).

³¹P NMR (161 MHz, D₂O+1 drop NaOD): δ 4.28.

*Chirality leads to pronounced peak splitting

Synthesis of Compound 116 with the Following Structure:

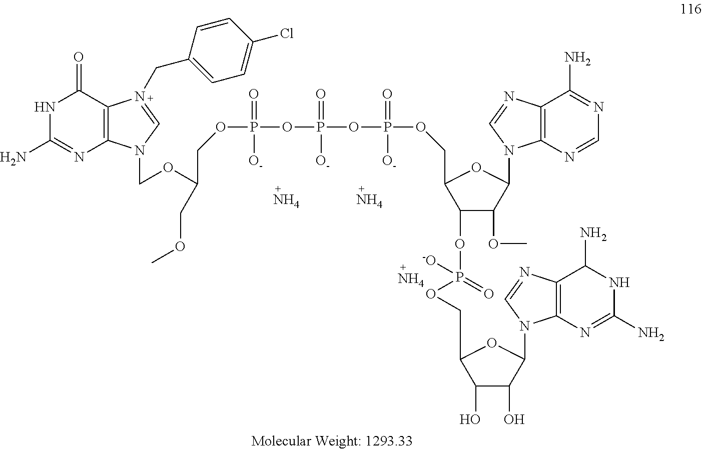

Compound 116 was prepared from compound 114 (100 mg, 1.0 eq.) following the general procedure 7 employing mL DMSO, 10.0 eq. CDI and 10.0 eq. water. Total reaction time 3 d. Compound 116 was purified according to general procedure 7 using the gradient program of table Ex-69 (A: 5 mM ammonium acetate in water; B: 90% methanol in water), followed by a second purification using the gradient program of table Ex-70 (A: 100 mM triethylammonium acetate (pH 7), B: 90% MeOH in water), followed by a third purification using the gradient program of table Ex-71 (A: 5 mM ammonium acetate in water; B: 90% methanol).

TABLE Ex-69

| Time | A | B |
|---|---|---|
| [min] | [%] | [%] |
| 0 | 100 | 0 |
| 10 | 100 | 0 |
| 20 | 80 | 20 |
| 21 | 5 | 95 |
| 29 | 5 | 95 |
| 30 | 100 | 0 |
| 45 | 100 | 0 |

TABLE Ex-70

| Time | A | B |
|---|---|---|
| [min] | [%] | [%] |
| 0 | 100 | 0 |
| 40 | 15 | 85 |
| 45 | 15 | 85 |
| 46 | 100 | 0 |
| 60 | 100 | 0 |

TABLE Ex-71

| Time | A | B |
|---|---|---|
| [min] | [%] | [%] |
| 0 | 100 | 0 |
| 10 | 100 | 0 |
| 25 | 50 | 50 |
| 37 | 5 | 95 |
| 45 | 5 | 95 |
| 46 | 100 | 0 |
| 65 | 100 | 0 |

Product fractions were analyzed via ESI-MS and NMR. The product was obtained as a white powder in a yield of 30%.

ESI-MS: [M]⁺: 1242.173, Calculated 1242.176

¹H NMR (400 MHz, D₂O) δ 8.36 (s br., 1H), 8.05 (m, 1H), 7.94 (s, 1H), 7.21 (m, 2H), 7.16 (m, 2H), 5.95 (d, 1H), 5.80 (dd, 1H), 5.64 (m, 2H), 5.43 (m, 2H), 4.96 (m, 1H), 4.74 (m, 1H), 4.53 (m, 1H), 4.49 (t, 1H), 4.43 (t, 1H), 4.33 (m, 2H), 4.23 (m, 3H), 4.06 (m, 2H), 3.96 (m, 1H), 3.42 (s, 3H), 3.33 (m, 2H), 2.99 (m, 3H).

³¹P NMR (162 MHz, D₂O) 6-0.83 (s, 1P), −11.37 (m, 2P), −22.78 (m, 1P).

Synthesis of Compound 117 with the Following Structure:

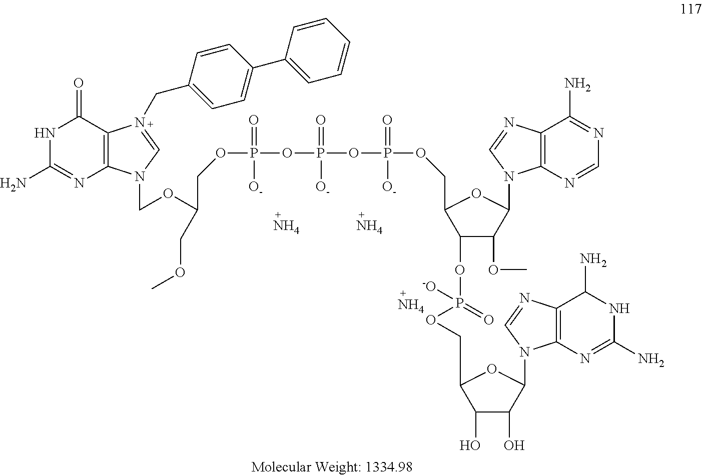

Compound 117 was prepared from compound 115 (100 mg, 1.0 eq.) following the general procedure 7 employing 1.0 mL DMSO, 20.0 eq. CDI (3 portions within 48 h) and 20.0 eq. water. Total reaction time: 5 d. Compound 117 was purified according to general procedure 7 using the gradient program of table Ex-72a (A: 5 mM ammonium acetate in water; B: 90% methanol in water), followed by a second purification using the gradient program of table Ex-73 (A: 100 mM triethylammonium acetate (pH 7), B: 90% MeCOH in water), followed by a third purification using the gradient program of table Ex-74a (A: 5 mM ammonium acetate in water; B: 100% acetonitrile). Sample elutes incompletely from C₁₈ column using ammonium acetate as buffer A for which reason washing runs were placed between sample runs (table Ex-72b, (A: 5 mM ammonium acetate in water; B. 90% methanol in water) and 74b, A: 5 mM ammonium acetate in water, B: 100% acetonitrile).

TABLE Ex-72a

| Time | A | B |
|---|---|---|
| [min] | [%] | [%] |
| 0 | 100 | 0 |
| 10 | 100 | 0 |
| 20 | 80 | 20 |
| 21 | 5 | 95 |
| 35 | 5 | 95 |
| 36 | 100 | 0 |
| 60 | 100 | 0 |

TABLE Ex-72b

| Time | A | B |
|---|---|---|
| [min] | [%] | [%] |
| 0 | 100 | 0 |
| 10 | 100 | 0 |
| 11 | 5 | 95 |
| 65 | 5 | 95 |
| 66 | 100 | 0 |
| 80 | 100 | 0 |

TABLE Ex-73

| Time | A | B |
|---|---|---|
| [min] | [%] | [%] |
| 0 | 100 | 0 |
| 30 | 5 | 95 |
| 50 | 5 | 95 |
| 51 | 100 | 0 |
| 65 | 100 | 0 |

TABLE Ex-74a

| Time | A | B |
|---|---|---|
| [min] | [%] | [%] |
| 0 | 100 | 0 |
| 10 | 100 | 0 |
| 11 | 0 | 100 |
| 56 | 0 | 100 |
| 57 | 100 | 0 |
| 70 | 100 | 0 |

TABLE Ex-74b

| Time | A | B |
|---|---|---|
| [min] | [%] | [%] |
| 0 | 0 | 100 |
| 56 | 0 | 100 |
| 57 | 100 | 0 |
| 70 | 100 | 0 |

Product fractions were analyzed via ESI-MS and NMR. The product was obtained as a white powder in a yield of 29%.

ESI-MS: [M]⁺: 1284.245, Calculated 1284.246

¹H NMR (400 MHz, D₂O) δ 9.37 (s br., 1H), 8.39 (s br., 1H), 7.95 (s br., 1H), 7.90 (s br., 1H), 7.17 (s br., 9H), 5.85 (d br., 1H), 5.73 (d br., 1H), 5.55 (s br., 2H), 5.41 (s br., 2H), 4.94 (s br., 1H), 4.67 (m, 1H), 4.52 (s br., 1H), 4.45 (s br., 1H), 4.37 (s br., 1H), 4.32 (s br., 2H), 4.19 (s br., 3H), 4.06 (s br, 2H), 3.98 (s br., 1H), 3.35 (s, 3H), 3.01 (m, 3H).

³¹P NMR (162 MHz, D₂O) 5-0.87 (s, 1P), −11.42 (m, 2P), −22.70 (m, 1P).

Similar compounds can be synthesized in accordance with the above synthesis route when introducing different N⁷-modifications. Exemplary compounds along these lines are shown in FIG. 10, namely i) the “3′-OMe-m⁷-Penciclovir Cap1” compound; ii) the “3′-OMe-N⁷-Nap-Penciclovir Cap1” compound and iii) the “3′-OMe-N⁷-3,5Me₂Bn-Penciclovir Cap1” compound, all starting from compound 1 (which is synthesized from Ganciclovir (X1)) or Penciclovir (X2).

### Example 1.11: Synthesis Route XI

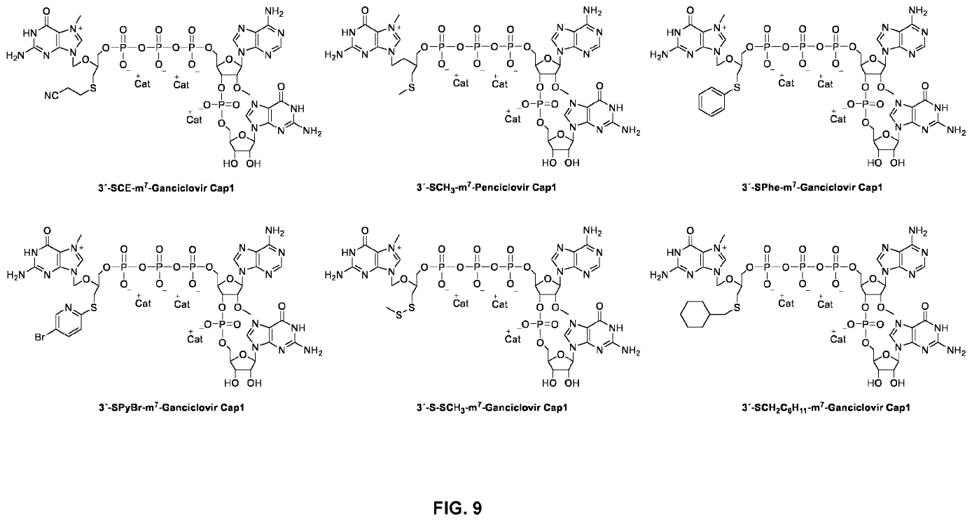

The synthesis of compound 123 (corresponding to m⁷-guanine-triethylene glycol-ppp-(5′)-Guanosine), 124 (corresponding to m⁷-guanine-triethylene glycol-ppp-AmG), 129 (corresponding to m⁷-guanine-tetraethylene glycol-ppp-AmG) and 134 (corresponding to m⁷-guanine-pentaethylene glycol-ppp-AmG) referred to in FIG. 11 as “m⁷G-TriEG Cap0”, “m⁷G-TriEG Cap1”, “m⁷G-TetraEG Cap1” and “m⁷G-PentaEG Cap1” compound when starting from X8 and X9/X10/X11 (or other polyethylene glycol or linear diol containing compounds containing more than one heteroatom within the alkyl chain) is shown in the following. It is noted that the synthesis of other derivatives (such as e.g. the nonaethylene glycol derived compound “m⁷G-NonaEG Cap1” referred to in FIG. 11) is identical. Examples of resulting compounds analogous to compound 123, 124, 129 and 134 when starting from X8 and X9/X10/X11 (or other polyethylene glycol or linear diol containing compounds containing more than one heteroatom) are described at the end of the present example and are depicted in FIG. 11.

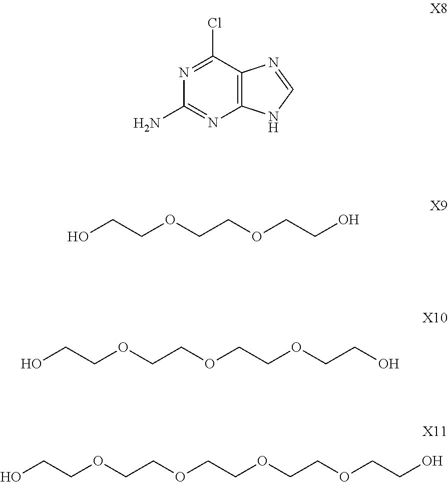 [SMILES: Nc1nc(Cl)c2nc[nH]c2n1 ; OCCOCCOCCO ; OCCOCCOCCOCCO ; OCCOCCOCCOCCOCCO]

Synthesis of Compound 118 with the Following Structure:

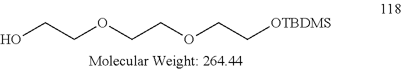 [SMILES: CC(C)(C)[Si](C)(C)OCCOCCOCCO]

Triethylene glycol (X9, 5.0 eq.), Et₃N (1.05 eq.) and DMAP (0.1 eq.) were dissolved in DCM (200 mL) under protective gas atmosphere. The reaction mixture was cooled to 0° C. and a solution of TBDMSCl (20.0 g, 1.0 eq.) in DCM (50 mL) was added dropwise over the course of 10 min. Stirring was continued for 16 h at ambient temperature followed by extraction with saturated ammonium chloride solution (750 mL) and brine (2×750 mL). The aqueous layers were extracted twice with DCM (50 mL each) and all combined organic extracts were dried over Na₂SO₄ and concentrated under reduced pressure.

Product fractions were analyzed via ESI-MS and NMR. The product was obtained as a clear oil in a yield of 66% (contains 28% disilylated triethylene glycol).

ESI-MS: [M+H]⁺: 265.2, Calculated 265.2

¹H NMR (400 MHz, CDCl₃) δ 3.79-3.56 (m, 12H), 2.41 (s br., 1H), 0.89 (s, 9H), 0.07 (s, 6H).

Synthesis of Compound 119 with the Following Structure:

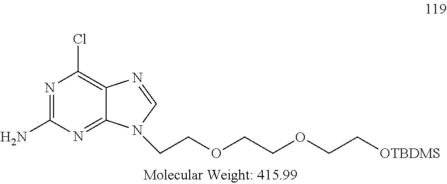 [SMILES: CC(C)(C)[Si](C)(C)OCCOCCOCCn1cnc2c(Cl)nc(N)nc21]

Compound 119 was prepared from compound 118 (32.44 g, 1.0 eq.) following the general procedure 1 employing 1.1 eq. 2-amino-6-chloropurine (X8), 1.2 eq. triphenylphosphine and 294 mL dry dioxane. The reaction mixture was cooled to 5° C. and DIAD (1.1 eq.) was added dropwise over the course of 15 minutes. It was then allowed to warm to ambient temperature and stirred for 72 h. Concentration of the reaction mixture was followed by resuspension in 500 mL ethyl acetate and the addition of MgCl₂ (2.0. eq.). The suspension was stirred for 2.5 h and was then filtered. The filtrate was concentrated, dry loaded on silica and purified by flash chromatography (silica 220 g, A: n-heptane, B: ethyl acetate, gradient program from Table Ex-75).

TABLE Ex-75

| Time | | |
|---|---|---|
| total | Flow | B |
| [min] | [mL/min] | [%] |
| 0 | 120 | 5 |
| 5 | 120 | 5 |
| 6 | 120 | 35 |
| 55 | 120 | 80 |

Product fractions were analyzed via ESI-MS and NMR The product was obtained as a beige powder in a yield of 49% (contains 17% triphenylphosphine oxide).

ESI-MS: [M+H]⁺: 416.2, Calculated 416.2

¹H NMR (400 MHz, d₆-DMSO) δ 8.06 (s, 1H), 6.90 (s br., 2H), 4.19 (t, 2H), 3.75 (t, 2H), 3.62 (t, 2H), 3.51 (m, 2H), 3.48 (m, 2H), 3.37 (t, 2H), 0.82 (s, 9H), −0.01 (s, 6H).

Synthesis of Compound 120 with the Following Structure:

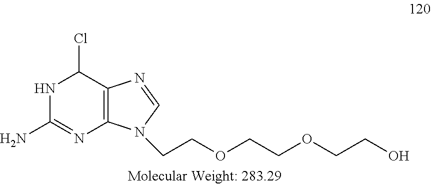 [SMILES: NC1=Nc2c(ncn2CCOCCOCCO)C(Cl)N1]

Compound 120 was prepared from compound 119 (24.4 g, 1.0 eq.) according to the general procedure 6 employing 124 mL TFA/H₂O (3/1) and a reaction time of 2 days. The crude product was purified by flash chromatography (silica 120 g, A: ethyl acetate, B: methanol, gradient program from Table Ex-7)

TABLE Ex-76

| Time | | |
|---|---|---|
| total | Flow | B |
| [min] | [mL/min] | [%] |
| 0 | 85 | 5 |
| 8 | 85 | 5 |
| 38 | 85 | 50 |
| 93 | 85 | 50 |

Product fractions were analyzed via ESI-MS and NMR. The product was obtained as a white solid in a yield of 38%.

ESI-MS: [M+H]⁺: 284.1, Calculated 284.1

¹H NMR (400 MHz, d₆-DMSO) δ 10.53 (s, 1H), 7.66 (s, 1H), 6.43 (s br., 2H), 4.56 (s br., 1H), 4.08 (t, 2H), 3.70 (t, 2H), 3.51 (m, 2H), 3.48 (m, 4H), 3.38 (t, 2H).

Synthesis of Compound 121 with the Following Structure:

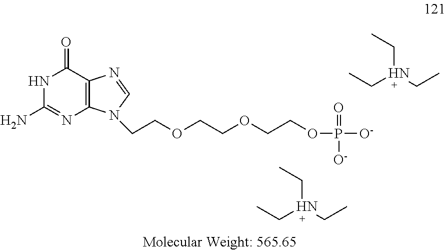

Compound 121 was prepared from compound 120 (4.7 g, 1.0 eq.) according to the general procedure 3 employing 33.5 mL trimethylphosphate, 2.0 eq. POCl₃, 5 h reaction time and 160 mL TEAB buffer (0.1 M, pH 8.5).

The crude product was purified by IEX chromatography with Macro-Prep High Q resin according to Table Ex-77 (A: water, B: 0.5 M TEAB).

TABLE Ex-77

| Time | | |
|---|---|---|
| total | Flow | B |
| [min] | [mL/min] | [%] |
| 0 | 25 | 0 |
| 10 | 25 | 0 |
| 45 | 25 | 100 |
| 70 | 25 | 100 |
| 70 | 25 | 0 |
| 90 | 25 | 0 |

Product fractions were analyzed via ESI-MS and NMR. The product was obtained as a white solid in quantitative yield (53% purity, impurities were identified as triethylamine species).

ESI-MS: [M+H]⁺: 364.0, Calculated 364.1

¹H NMR (400 MHz, D₂O) δ 8.06 (s, 1H), 4.26 (t, 2H), 3.94 (m, 2H), 3.87 (m, 2H), 3.64 (m, 6H).

³¹P NMR (162 MHz, D₂O) δ 0.45.

Synthesis of Compound 122 with the Following Structure:

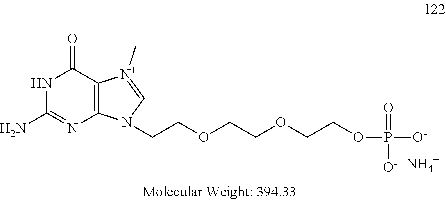 [SMILES: C[n+]1cn(CCOCCOCCOP(=O)([O-])[O-])c2nc(N)[nH]c(=O)c21 ; [NH4+]]

Compound 122 was prepared from compound 121 (10.8 g, 1.0 eq.) according to the general procedure 4 employing 5.0 eq. MeI and 16 h reaction time. The crude product was purified by ion exchange chromatography with Macro-Prep High Q resin according to Table Ex-78 (A: water, B: 0.5 M TEAB) The flow through during sample application was collected separately and applied once more by ion exchange chromatography with Macro-Prep High Q resin according to Table Ex-79 (A: water, B: 0.5 M TEAB). The combined product fractions were repurified via C18 chromatography according to Table Ex-80 (Buchi SELECT column, A: 10 mM ammonium acetate, B: 100% MeOH).

TABLE Ex-78

| Time | | |
|---|---|---|
| total | Flow | B |
| [min] | [mL/min] | [%] |
| 0 | 25 | 0 |
| 15.5 | 25 | 0 |
| 45.5 | 25 | 100 |
| 65.5 | 25 | 100 |
| 65.5 | 25 | 0 |
| 85.5 | 25 | 0 |

TABLE Ex-79

| Time total [min] | Flow [mL/min] | B [%] |
|---|---|---|
| 0 | 25 | 0 |
| 10 | 25 | 0 |
| 55 | 25 | 100 |
| 75 | 25 | 100 |
| 75 | 25 | 0 |
| 95 | 25 | 0 |

TABLE Ex-80

| Colume volumes | Flow [mL/min] | B [%] |
|---|---|---|
| 0 | 40 | 0 |
| 1 | 40 | 0 |
| 4.5 | 40 | 30 |
| 5 | 40 | 100 |
| 6 | 40 | 100 |
| 6 | 40 | 80 |
| 7.5 | 40 | 80 |

Product fractions were analyzed via ESI-MS and NMR. The product was obtained as a white solid in a yield of 33%.

ESI-MS: [M+H]⁺: 378.1, Calculated 378.1

¹H NMR (400 MHz, D₂O) δ 8.95 (s, 1H), 4.43 (dd, 2H), 4.11 (s, 3H), 3.95 (m, 4H), 3.71 (m, 6H).

³¹P NMR (162 MHz, D₂O) 6-1.69.

Synthesis of Compound 123 with the Following Structure:

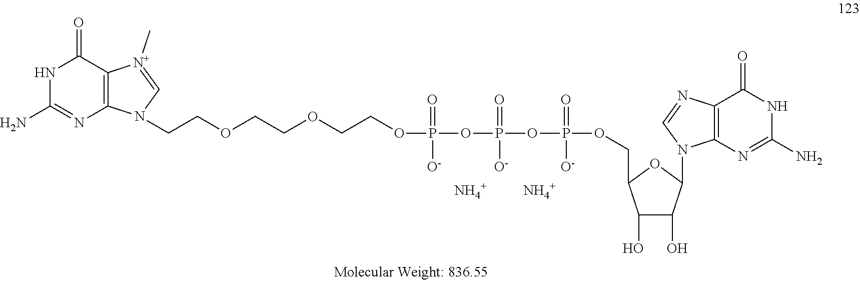 [SMILES: C[n+]1cn(CCOCCOCCOP(=O)([O-])OP(=O)([O-])OP(=O)([O-])OCC2OC(n3cnc4c(=O)[nH]c(N)nc43)C(O)C2O)c2nc(N)[nH]c(=O)c21 ; [NH4+] ; [NH4+]]

Compound 123 was prepared from compound 122 (150 mg, 1.0 eq.) according to the general procedure 5 using 1.5 eq. guanosine 5′-imidazolide diphosphate, 30 mL H₂O/ACN and 10.0 eq. MgCl₂. The reaction was quenched after 24 h via the addition of 250 mM EDTA-solution (30 mL). The product was purified according to the purification method B: The reaction mixture was desalted using RP-HPLC with the gradient profile of table Ex-81 (A: 5 mM ammonium acetate in water; B: 90% methanol in water). The desalted reaction mixture was purified by ion exchange chromatography with DNAPac PA200 column (22×250 mm) using the gradient from table Ex-82, (A: 20 mM Tris, pH 9; B: 20 mM Tris, 33 mM sodium perchlorate). The product fractions were desalted using RP-HPLC using the gradient from table Ex-83 (A: 5 mM ammonium acetate in water; B: 90% methanol in water). The resulting fraction was repurified via C18 RP HPLC, using a Phenomexx Gemini C18, 250×21.2 mm, 5 μM Column and the gradient from table Ex-84 (A: 5 mM ammonium acetate pH 5.6, B: 90% MeOH, gradient).

TABLE Ex-81

| Time total [min] | A [%] | B [%] |
|---|---|---|
| 0 | 100 | 0 |
| 10 | 100 | 0 |
| 20 | 85 | 15 |
| 21 | 5 | 95 |
| 28 | 5 | 95 |
| 29 | 100 | 0 |
| 42 | 100 | 0 |

TABLE Ex-82

| Time total [min] | A [%] | B [%] |
|---|---|---|
| 0 | 100 | 0 |
| 30 | 25 | 75 |
| 31 | 0 | 100 |
| 35 | 0 | 100 |
| 36 | 100 | 0 |
| 40 | 100 | 0 |

TABLE Ex-83

| Time total [min] | A [%] | B [%] |
|---|---|---|
| 0 | 100 | 0 |
| 10 | 100 | 0 |
| 30 | 70 | 30 |
| 31 | 20 | 80 |
| 40 | 20 | 80 |
| 41 | 100 | 0 |
| 55 | 100 | 0 |

TABLE Ex-84

| Time total [min] | A [%] | B [%] |
|---|---|---|
| 0 | 100 | 0 |
| 10 | 100 | 0 |
| 30 | 70 | 30 |
| 31 | 20 | 80 |
| 35 | 20 | 80 |
| 36 | 100 | 0 |
| 45 | 100 | 0 |

Product fractions were analyzed via ESI-MS and NMR. The product was obtained as a white powder in a yield of 19%.

ESI-MS: [M]⁺: 803.130, Calculated 803.131

¹H NMR (400 MHz, D₂O) δ 8.90 (s, 1H), 8.07 (s, 1H), 5.86 (d, 1H), 4.73 (t, 1H), 4.53 (dd, 1H), 4.36 (m, 3H), 4.27 (m, 2H), 4.11 (m, 2H), 4.06 (s, 3H), 3.89 (t, 2H), 3.70 (m, 6H).

³¹P NMR (162 MHz, D₂O) δ −11.18 (d, 1P), −11.46 (d, 1P), −23.12 (t, 1P).

Synthesis of Compound 124 with the Following Structure:

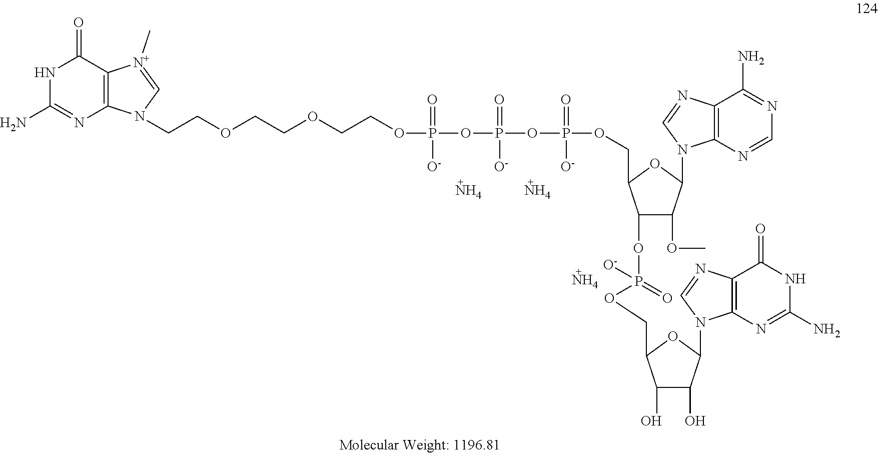

Compound 124 was prepared from compound 122 (50 mg, 1.0 eq.) following the general procedure 7 employing 0.4 mL DMSO and 10.0 eq. CDI. The reaction was quenched after 24 h by the addition of 20.0 eq. water. In the following NMM buffer (63 μl), water (120 μl), compound 28 (1.0 eq.) and MnCl₂ (1.0 eq.) were added consecutively and stirring was continued for another 72 h. The reaction was then quenched by the addition of a 250 mM EDTA solution (1.1 eq.) and the crude product was purified according to general procedure 7 using the gradient program of table Ex-85 (A: 5 mM ammonium acetate in water; B: 90% methanol in water), followed by a second purification using the gradient program of table Ex-86 (A: 100 mM triethylammonium acetate (pH 7), B: 90% MeOH in water), followed by a third purification using the gradient program of table Ex-87 (A: 5 mM ammonium acetate in water; B: 90% methanol in water).

TABLE Ex-85

| Time [min] | A [%] | B [%] |
|---|---|---|
| 0 | 100 | 0 |
| 10 | 100 | 0 |
| 20 | 80 | 20 |
| 21 | 5 | 95 |
| 29 | 5 | 95 |
| 30 | 100 | 0 |
| 45 | 100 | 0 |

TABLE Ex-86

| Time [min] | A [%] | B [%] |
|---|---|---|
| 0 | 100 | 0 |
| 40 | 60 | 40 |
| 41 | 100 | 0 |
| 51 | 100 | 0 |

TABLE Ex-87

| Time [min] | A [%] | B [%] |
|---|---|---|
| 0 | 100 | 0 |
| 10 | 100 | 0 |
| 40 | 50 | 50 |
| 41 | 20 | 80 |
| 48 | 20 | 80 |
| 49 | 100 | 0 |
| 60 | 100 | 0 |

Product fractions were analyzed via ESI-MS and NMR. The product was obtained as a white powder in a yield of 20%.

ESI-MS: [M]⁺: 1146.198, Calculated 1146.199

¹H NMR (400 MHz, D₂O) δ 8.80 (s, 1H), 8.39 (s, 1H), 8.10 (s, 1H), 7.88 (s, 1H), 5.96 (d, 1H), 5.76 (d, 1H), 4.87 (m, 1H), 4.76 (t, 1H), 4.41 (m, 3H), 4.25 (m, 3H), 4.18-4.10 (m, 4H), 3.98 (m, 2H), 3.96 (s, 3H), 3.77 (t, 2H), 3.55 (m, 6H), 3.34 (s, 3H).

³¹P NMR (162 MHz, D₂O) δ −0.91 (s, 1P), −11.10 (d, 1P), −11.61 (d, 1P), −22.86 (m, 1P).

Synthesis of Compound 125 with the Following Structure:

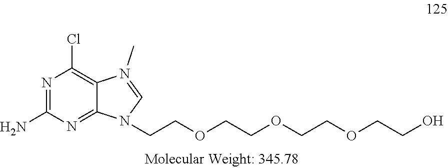

Compound X8 (5.00 q, 1.0 eq.), X10 (2.1 eq.) and triphenylphosphine (1.2 eq.) were suspended in THE (147 mL) and cooled to 0° C. DIAD (1.2 eq.) was added dropwise until the reaction mixture remained permanently yellow. The yellow suspension was allowed to warm to ambient temperature and stirred for 18 h. It was then concentrated and redissolved in water (500 mL) and ethyl acetate (100 mL). The organic layer was isolated and washed with water. The combined aqueous phases were concentrated, dry loaded on a hydromatrix using DCM and purified by flash chromatography within 2 batches (silica 220 g, A: n-heptane, B: ethyl acetate, gradient program from Table Ex-88.)

TABLE Ex-88

| Time total [min] | Flow [mL/min] | B [%] |
|---|---|---|
| 0 | 100 | 5 |
| 4 | 100 | 5 |
| 50 | 100 | 70 |
| 60 | 100 | 70 |

Product fractions were analyzed via ESI-MS and NMR. The product was obtained as an off white solid in a yield of 48%.

ESI-MS: [M+H]⁺: 346.0, Calculated 346.1

¹H NMR (400 MHz, d₆-DMSO) δ 8.09 (s, 1H), 6.91 (s br., 2H), 4.60 (t, 1H), 4.20 (t, 2H), 3.75 (t, 2H), 3.53-3.38 (m, 12H).

Synthesis of Compound 126 with the Following Structure:

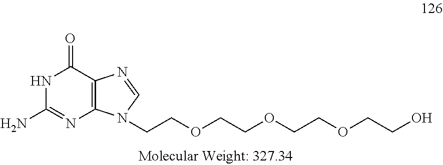 [SMILES: Nc1nc2c(ncn2CCOCCOCCOCCO)c(=O)[nH]1]

Compound 125 (5.0 g, 1.0 eq.) was dissolved in TFA/H₂O (3/1, 68 mL) and stirred at ambient temperature for 2.5 days. The reaction mixture was concentrated under reduced pressure and was then coevaporated trice with water. ACN (75 mL) was added upon which a white precipitate formed. The suspension was stirred for 1.5 h after which the precipitate was collected by filtration and dried at 40° C. overnight.

Product fractions were analyzed via ESI-MS and NMR. The product was obtained as a crystalline solid in a yield of 50%.

ESI-MS: [M−H]⁻: 326.2, Calculated 326.1

¹H NMR (400 MHz, d₆-DMSO) δ 11.00 (s br., 1H), 8.30 (s, 1H), 6.75 (s br., 2H), 4.17 (t, 2H), 3.74 (t, 2H), 3.53 (m, 2H), 3.47 (m, 8H), 3.40 (t, 2H).

Synthesis of Compound 127 with the Following Structure:

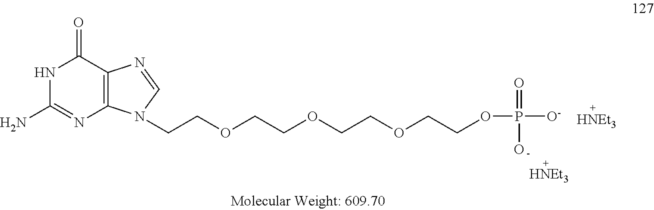 [SMILES: Nc1nc2c(ncn2CCOCCOCCOCCOP(=O)([O-])O)c(=O)[nH]1 ; "HNEt3" ; "HNEt3"]

Compound 126 (3.17 g, 1.0 eq.) was dissolved in water and was then concentrated to dryness.

Trimethylphosphate (14.4 mL) were cooled to −15° C. using an ice bath. POCl₃ (2.0 eq.) and compound 126 were added and the reaction mixture was stirred for 3 h. The reaction was quenched by the addition of 75 mL TEAB (0.1 M, pH 8.5), was warmed to ambient temperature, was then diluted with 100 mL of water and pH was adjusted to >7.5 using aqueous ammonium hydroxide. The solution was filtered, diluted with water (1.25 L) and purified by IEX chromatography with Macro-Prep High Q resin according to Table Ex-89 (A: water, B: 0.5 M TEAB). Product containing fractions were evaporated and repurified via C18 chromatography according to Table Ex-90 (120 g, Flash Pure Buchi, Buffer A: 10 mM ammonium acetate in water, Buffer B: 100% MeOH).

TABLE Ex-89

| Time total [min] | Flow [ml/min] | B [%] |
|---|---|---|
| 0 | 25 | 0 |
| 10 | 25 | 0 |
| 50 | 25 | 100 |
| 80 | 25 | 100 |
| 80 | 25 | 0 |
| 110 | 25 | 0 |

TABLE Ex-90

| Time total [min] | Flow [mL/min] | B [%] |
|---|---|---|
| 0 | 30 | 0 |
| 5.4 | 30 | 0 |
| 11.1 | 85 | 25 |
| 12.1 | 85 | 80 |
| 15.9 | 85 | 80 |

Product fractions were analyzed via ESI-MS and NMR. The product was obtained as a colorless oil in a yield of 69%.

ESI-MS: [M+H]⁺: 408.0, Calculated 408.1

¹H NMR (400 MHz, d₆-DMSO) δ 7.63 (s, 1H), 6.71 (s br., 2H), 4.08 (t, 2H), 3.73 (m, 4H), 3.49 (m, 4H), 3.43 (m, 4H), 3.39 (m, 2H).

Synthesis of Compound 128 with the Following Structure:

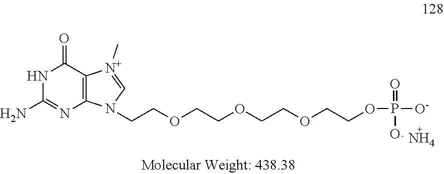

Compound 128 was prepared from compound 127 (6.0 g, 1.0 eq.) according to the general procedure 4. After 4 h, the reaction mixture was diluted with water (1.0 L) and washed with methyl tert-butyl ether (4×100 mL). The slightly yellow phase was decolorized with a few grains of sodium disulfite, diluted with additional 500 mL of water and purified by IEX chromatography with Macro-Prep High Q resin according to Table Ex-91 (A: water, B: 0.5 M TEAB). Product containing fractions were evaporated and repurified via C18 chromatography within 2 batches according to Table Ex-92 (120 g, Flash Pure Buchi, A: 10 mM ammonium acetate, B: 100% MeOH).

TABLE Ex-91

| Time total [min] | Flow [mL/min] | B [%] |
|---|---|---|
| 0 | 25 | 0 |
| 10 | 25 | 0 |
| 50 | 25 | 100 |
| 80 | 25 | 100 |
| 80 | 25 | 0 |
| 110 | 25 | 0 |

TABLE Ex-92

| Time total [min] | Flow [ml/min] | B [%] |
|---|---|---|
| 0 | 30 | 0 |
| 5.4 | 30 | 0 |
| 11.1 | 85 | 25 |
| 12.1 | 85 | 80 |
| 15.9 | 85 | 80 |

Product fractions were analyzed via ESI-MS and NMR. The product was obtained as a white solid in a yield of 61%.

ESI-MS: [M+H]⁺: 422.0, Calculated 422.1

¹H NMR (400 MHz, D₂O) δ 8.93 (s, 1H), 4.42 (t, 2H), 4.09 (s, 3H), 3.98 (m, 2H), 3.92 (t, 2H), 3.72-3.63 (m, 10H).

³¹P NMR (162 MHz, D₂O) δ 2.41.

Synthesis of Compound 129 with the Following Structure:

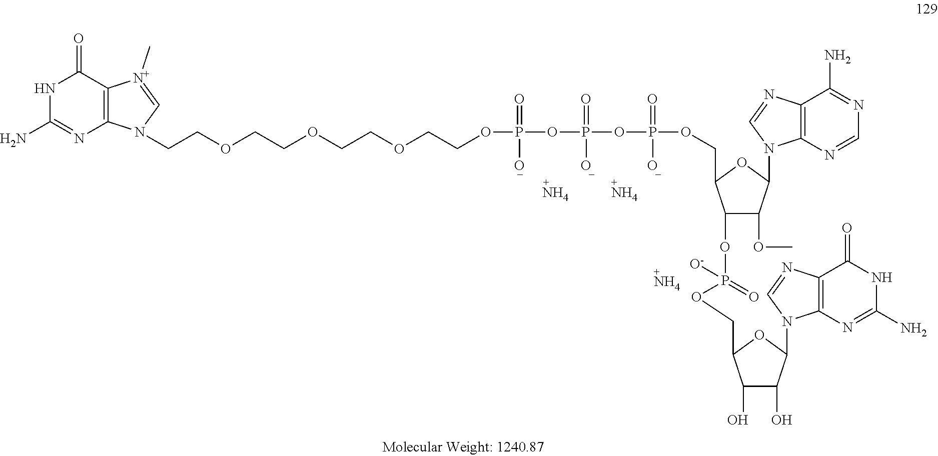

Compound 129 was prepared from compound 128 (100 mg, 1.0 eq.) following the general procedure 7 employing 1.0 mL DMSO, 10.0 eq. CDI and 10.0 eq. water. Total reaction time: 4 d.

ESI-MS: [M+H]²⁺: 595.616, Calculated 595.616

Synthesis of Compound 130 with the Following Structure:

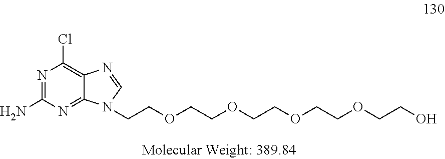 [SMILES: Nc1nc(Cl)c2ncn(CCOCCOCCOCCOCCO)c2n1]

Compound X8 (8.00 q, 1.0 eq.), X11 (2.1 eq.) and triphenylphosphine (1.2 eq.) were suspended in THE (250 mL) and cooled to 0° C. DIAD (1.2 eq.) was added dropwise until the reaction mixture remained permanently yellow. The yellow suspension was allowed to warm to ambient temperature and stirred for 18 h. It was then concentrated and redissolved in water (750 mL) and ethyl acetate (150 mL). The organic layer was isolated and washed with water. The combined aqueous phases were concentrated, dry loaded on a hydromatrix using DCM and purified by flash chromatography within 3 batches (silica 330 g, A: n-heptane, B: ethyl acetate, gradient program from Table Ex-93, 94 and 951.

TABLE Ex-93

| Time total [min] | Flow [mL/min] | B [%] |
|---|---|---|
| 0 | 100 | 5 |
| 4 | 100 | 5 |
| 60 | 100 | 70 |
| 70 | 100 | 70 |
| 90 | 100 | 100 |

TABLE Ex-94

| Time total [min] | Flow [mL/min] | B [%] |
|---|---|---|
| 0 | 100 | 5 |
| 4 | 100 | 5 |
| 60 | 100 | 70 |
| 75 | 100 | 70 |

TABLE Ex-95

| Time total [min] | Flow [mL/min] | B [%] |
|---|---|---|
| 0 | 100 | 5 |
| 4 | 100 | 5 |
| 60 | 100 | 70 |
| 75 | 100 | 70 |

Product fractions were analyzed via ESI-MS and NMR. The product was obtained as a pale green oil in a yield of 47%.

ESI-MS: [M+H]⁺: 390.0, Calculated 390.2

¹H NMR (400 MHz, d₆-DMSO) δ 8.08 (s, 1H), 6.90 (s br., 2H), 4.57 (t, 1H), 4.20 (t, 2H), 3.75 (t, 2H), 3.52-3.40 (m, 16H).

Synthesis of Compound 131 with the Following Structure:

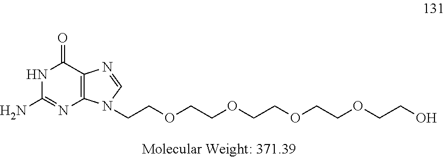 [SMILES: Nc1nc2c(ncn2CCOCCOCCOCCOCCO)c(=O)[nH]1]

Compound 130 (8.6 g, 1.0 eq.) was dissolved in TFA/H₂O (3/1, 100 mL) and stirred at ambient temperature for 40 h and subsequently 3 h at 50° C. The reaction mixture was allowed to cool to ambient temperature, was concentrated under reduced pressure and was coevaporated trice with water.

Product fractions were analyzed via ESI-MS and NMR. The product was obtained as a yellow oil in a yield of 76% (80% purity).

ESI-MS: [M−H]⁻: 372.0, Calculated 372.2

¹H NMR (400 MHz, d₆-DMSO) δ 11.30 (s br., 1H), 8.62 (s, 1H), 6.95 (s br., 2H), 4.21 (t, 2H), 3.77 (t, 2H), 3.53 (m, 2H), 3.48 (m, 12H), 3.41 (m, 2H).

Synthesis of Compound 132 with the Following Structure:

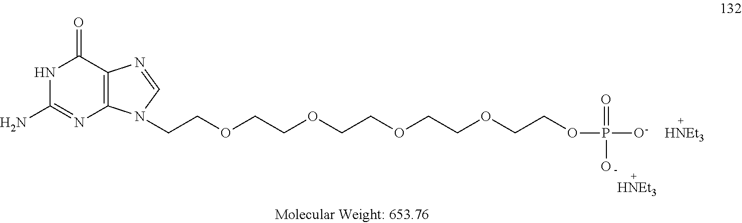 [SMILES: Nc1nc2c(ncn2CCOCCOCCOCCOCCOP(=O)([O-])O)c(=O)[nH]1 ; "HNEt3" ; "HNEt3"]

Compound 131 (1.0 g, 1.0 eq.) was dissolved in water and was then concentrated to dryness. Trimethylphosphate (4.2 mL) were cooled to −15° C. using an ice bath. POCl₃ (2.0 eq.) and compound 131 were added and the reaction mixture was stirred for 4 h. The reaction was quenched by the addition of 10 mL TEAB (0.1 M, pH 8.5), was warmed to ambient temperature, was then diluted with 100 mL of water and pH was adjusted to >7.5 using aqueous ammonium hydroxide. The solution was filtered, diluted with water (125 mL) and purified by IEX chromatography with Macro-Prep High Q resin according to Table Ex-96 (A: water, B: 0.5 M TEAB). Product containing fractions were evaporated and repurified via C18 chromatography according to Table Ex-97 (40 g, Flash Pure Buchi, Buffer A: 10 mM ammonium acetate in water, Buffer B: 100% MeOH).

TABLE Ex-96

| Time total [min] | Flow [mL/min] | B [%] |
|---|---|---|
| 0 | 12 | 0 |
| 4.2 | 12 | 0 |
| 12.6 | 12 | 100 |
| 18.9 | 12 | 100 |
| 18.9 | 12 | 0 |
| 25.2 | 12 | 0 |

TABLE Ex-97

| Time total [min] | Flow [mL/min] | B [%] |
|---|---|---|
| 0 | 15 | 0 |
| 5.4 | 15 | 0 |
| 11.1 | 40 | 25 |
| 12.1 | 40 | 80 |
| 15.9 | 40 | 80 |

Product fractions were analyzed via ESI-MS and NMR. The product was obtained as a colorless oil in a yield of 13%.

ESI-MS: [M+H]⁺: 452.2, Calculated 452.2

¹H NMR (400 MHz, d₆-DMSO) δ 7.62 (s, 1H), 6.80 (s br., 2H), 4.08 (t, 2H), 3.73 (m, 4H), 3.51-3.39 (m, 14H).

³¹P NMR (162 MHz, d₆-DMSO) δ 1.56.

Synthesis of Compound 133 with the Following Structure:

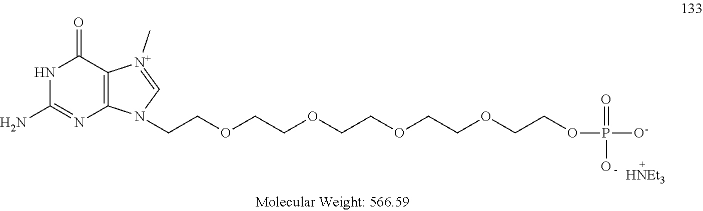 [SMILES: C[n+]1cn(CCOCCOCCOCCOCCOP(=O)([O-])O)c2nc(N)[nH]c(=O)c21 ; "HNEt3"]

Compound 133 was prepared from compound 132 (170 mg, 1.0 eq.) according to the general procedure 4. After 4 h, the reaction mixture was diluted with water (20 mL) and washed with methyl tert-butyl ether (4×50 mL). The slightly yellow phase was decolorized with a few grains of sodium disulfite, diluted with water (60.0 mL total) and purified by IEX chromatography with Macro-Prep High Q resin according to Table Ex-98 (A: water, B: 0.5 M TEAB). Product containing fractions were evaporated and repurified via C18 chromatography to Table Ex-99 (40 g, Flash Pure Buchi, A: 10 mM ammonium acetate, B: 100% MeOH).

TABLE Ex-98

| Time total [min] | Flow [mL/min] | B [%] |
|---|---|---|
| 0 | 12 | 0 |
| 4.2 | 12 | 0 |
| 12.6 | 12 | 100 |
| 18.9 | 12 | 100 |
| 18.9 | 12 | 0 |
| 25.2 | 12 | 0 |

TABLE Ex-99

| Time total [min] | Flow [mL/min] | B [%] |
|---|---|---|
| 0 | 15 | 0 |
| 5.3 | 15 | 0 |
| 10.9 | 40 | 35 |
| 11.9 | 40 | 80 |
| 14.2 | 40 | 80 |
| 14.2 | 40 | 5 |
| 16.5 | 40 | 5 |

Product fractions were analyzed via ESI-MS and NMR The product was obtained as a crystalline solid in a yield of 33% (triethylammonium salt) and 40% (protonated species).

ESI-MS: [M+H]⁺: 466.0, Calculated 466.2

¹H NMR (400 MHz, D₂O) δ 4.43 (t, 2H), 4.11 (s, 3H), 3.95 (m, 4H), 3.73-3.62 (m, 14H).

³¹P NMR (162 MHz, D₂O) δ 2.31.

Synthesis of Compound 134 with the Following Structure:

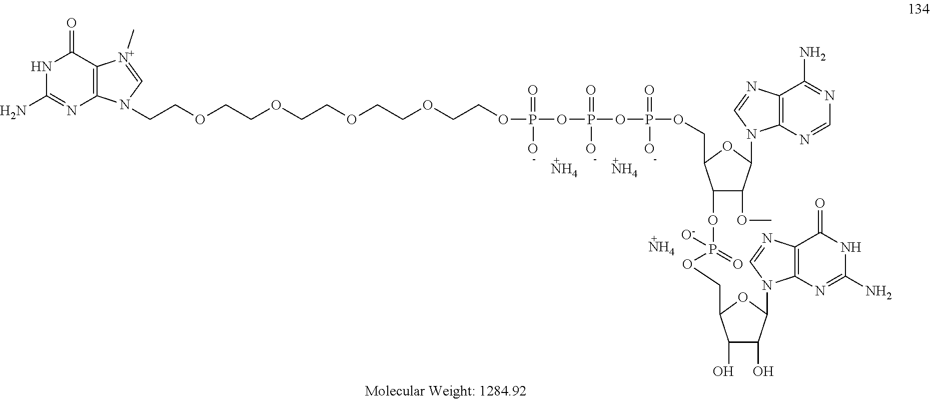

Compound 134 was prepared from compound 133 (54 mg, 1.0 eq.) following the general procedure 7 employing 0.5 mL DMSO, 10.0 eq. CDI and 10.0 eq. water. Total reaction time: 4 d.

ESI-MS: [M+H]²+: 617.628, Calculated 617.629 Similar compounds can be synthesized in accordance with the above synthesis route when starting from other polyethylene glycol or linear diol compounds containing mor than one heteroatom within the alkyl chain. Exemplary compounds along these lines are shown in FIG. 11, namely i) the “m⁷G-NonaEG Cap1” compound.

### Example 1.12: Synthesis Route XII

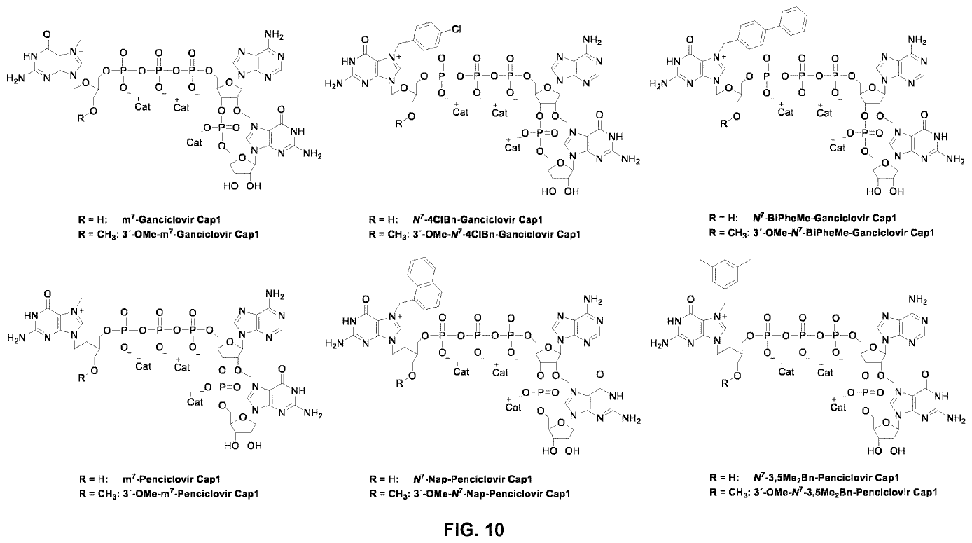

The synthesis of compound 137 (corresponding to UNA-ppp-(5′)-Guanosine, referred to in FIG. 12 as “UNA Cap0” compound) when starting from Guanosine monophosphate disodium salt (X12) is shown in the following.

Synthesis of Compound 135 with the Following Structure:

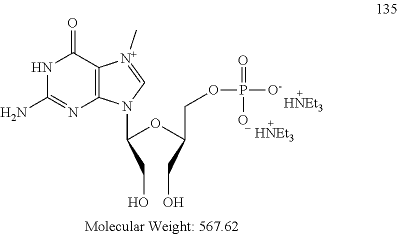 [SMILES: C[n+]1cn([C@H](CO)O[C@H](CO)COP(=O)([O-])O)c2nc(N)[nH]c(=O)c21 ; "HNEt3" ; "HNEt3"]

Guanosine monophosphate disodium salt (X12, 4.00 g, 1.0 eq.) was dissolved in water (45 mL) and sodium periodate (2.0 eq.) was added. The solution was stirred for 2 h at ambient temperature followed by the careful addition of sodium borohydride (3.0 eq.). The reaction mixture was stirred 2 more hours at ambient temperature followed dilution with water and pH adjustment (pH 6.5-7.5) using glacial acetic acid. The crude product was purified by ion exchange chromatography with Macro-Prep High Q resin using a TEAB gradient (table-Ex-100, A: water, B: 1.0 M TEAB). Solvent was evaporated from product fractions and the product was obtained as its triethylammonium salt which is directly used in the next step.

TABLE Ex-100

| Time total [min] | Flow [ml/min]* | B [%] |
|---|---|---|
| 0 | 50-65 | 0 |
| 10 | 50-65 | 0 |
| 85 | 50-65 | 75 |
| 115 | 50-65 | 75 |
| 115 | 50-65 | 100 |
| 135 | 50-65 | 100 |

*varied depending on backpressure

Product fractions were analyzed via ESI-MS and NMR. The product was obtained as a white solid in a yield of 70%.

ESI-MS: [M+H]⁺: 366.080, Calculated 366.081

¹H NMR (80 MHz, D₂O) δ 8.04 (s, 1H), 5.84 (t, 1H), 3.99 (m, 2H), 3.77 (m, 5H).

³¹P NMR (33 MHz, D₂O) δ 2.70.

Synthesis of Compound 136 with the Following Structure:

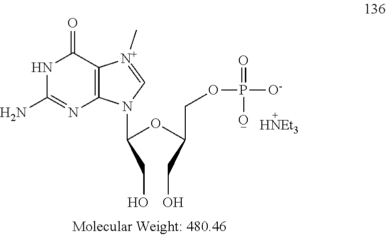 [SMILES: C[n+]1cn([C@H](CO)O[C@H](CO)COP(=O)([O-])O)c2nc(N)[nH]c(=O)c21 ; "HNEt3"]

Compound 136 was prepared from compound 135 (2.0 g, 1.0 eq.) following the general procedure 4. The crude product was purified by ion exchange chromatography with Macro-Prep High Q resin using a TEAB gradient according to table Ex-101 (A: water, B: 1.0 M TEAB).

TABLE Ex-101

| Time total [min] | Flow [mL/min] | B [%] |
|---|---|---|
| 0 | 66 | 0 |
| 15 | 66 | 0 |
| 70 | 66 | 50 |
| 70 | 66 | 100 |
| 85 | 66 | 100 |
| 85 | 66 | 0 |
| 95 | 66 | 0 |

Product fractions were analyzed via ESI-MS and NMR. The product was obtained as a white solid in a yield of 67%.

ESI-MS: [M]⁺: 380.096, Calculated 380.097

¹H NMR (400 MHz, D₂O) δ 8.44 (s, 1H), 6.06 (t, 1H), 4.12 (s, 3H), 4.03 (dd, 1H), 3.94 (m, 2H), 3.84 (m, 3H), 3.74 (d, 1H).

³¹P NMR (162 MHz, D₂O) δ 2.09.

Synthesis of Compound 137 with the Following Structure:

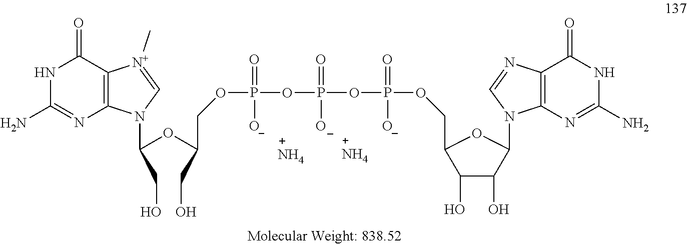 [SMILES: C[n+]1cn([C@H](CO)O[C@H](CO)COP(=O)([O-])OP(=O)([O-])OP(=O)([O-])OCC2OC(n3cnc4c(=O)[nH]c(N)nc43)C(O)C2O)c2nc(N)[nH]c(=O)c21 ; [NH4+] ; [NH4+]]

Compound 137 was prepared from compound 136 (460 mg, 1.0 eq.) according to the general procedure 5 using 1.5 eq. guanosine 5′-imidazolide diphosphate, 90 mL H₂O/ACN and 10.0 eq. MgCl₂. The reaction was quenched after 24 h via the addition of 250 mM EDTA-solution (90 mL) The product was purified according to the purification method B: The reaction mixture was desalted using RP-HPLC using the gradient of table Ex-102 (A: 5 mM ammonium acetate in water; B: 90% methanol in water). The desalted reaction mixture was purified by ion exchange chromatography with DNAPac PA200 column (22×250 mm) using the gradient of table Ex-103 (A: 20 mM Tris, pH 9; B 20 mM Tris, 33 mM sodium perchlorate). The product fractions were desalted using RP-HPLC using the gradient of table Ex-104 (A: 5 mM ammonium acetate in water; B: 90% methanol in water). The resulting fraction was repurified via C18 RP HPLC, using a Phenomexx Gemini C18, 250×21.2 mm, 5 μM Column and the gradient of table Ex-105 (A: 5 mM ammonium acetate pH 5.6, B: 90% MeOH).

TABLE Ex-102

| Time total [min] | A [%] | B [%] |
|---|---|---|
| 0 | 100 | 0 |
| 10 | 100 | 0 |
| 20 | 85 | 15 |
| 21 | 20 | 80 |
| 28 | 20 | 80 |
| 29 | 100 | 0 |
| 36 | 100 | 0 |

TABLE Ex-103

| Time total [min] | A [%] | B [%] |
|---|---|---|
| 0 | 100 | 0 |
| 30 | 25 | 75 |
| 31 | 0 | 100 |
| 35 | 0 | 100 |
| 36 | 100 | 0 |
| 40 | 100 | 0 |

TABLE Ex-104

| Time total [min] | A [%] | B [%] |
|---|---|---|
| 0 | 100 | 0 |
| 10 | 100 | 0 |
| 30 | 70 | 30 |
| 31 | 20 | 80 |
| 40 | 20 | 80 |
| 41 | 100 | 0 |
| 50 | 100 | 0 |

TABLE Ex-105

| Time total [min] | A [%] | B [%] |
|---|---|---|
| 0 | 100 | 0 |
| 10 | 100 | 0 |
| 30 | 70 | 30 |
| 31 | 20 | 80 |
| 40 | 20 | 80 |
| 41 | 100 | 0 |
| 50 | 100 | 0 |

Product fractions were analyzed via ESI-MS and NMR. The product was obtained as a white powder in a yield of 24%.

ESI-MS: [M-2H]⁻: 803.093, Calculated 803.095

¹H NMR (400 MHz, D₂O) δ 9.11 (s, 1H), 8.02 (s, 1H), 6.01 (t, 1H), 5.82 (d, 1H), 4.65 (t, 1H), 4.48 (dd, 1H), 4.31 (m, 1H), 4.29-4.17 (m, 2H), 4.05 (s, 3H), 4.03-3.96 (m, 4H), 3.90 (dd, 1H), 3.84 (m, 1H), 3.72 (m, 1H).

³¹P NMR (162 MHz, D₂O) 6-11.37 (d, 1P), −11.57 (d, 1P), −22.98 (t, 1P).

### Example 1.13: Synthesis Route XIII

The synthesis of compound 138 (corresponding to m⁷G-ethylene glycol-ppp-(5′)-Guanosine, referred to in WO 2023/007019 A1 FIG. 1 as “Ethylene linked” compound and shown as “Ethylene glycol cap0” in FIG. 12) was synthesized when starting from ethylene glycol according to WO2023/007019 A1 Example 1.1.

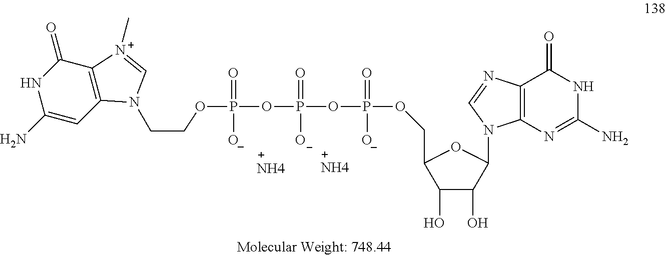 [SMILES: C[n+]1cn(CCOP(=O)([O-])OP(=O)([O-])OP(=O)([O-])OCC2OC(n3cnc4c(=O)[nH]c(N)nc43)C(O)C2O)c2cc(N)[nH]c(=O)c21 ; [NH4+] ; [NH4+]]

¹H NMR (400 MHz, D₂O) δ 8.94 (s, 1H), 7.99 (s, 1H), 5.81 (d, 1H), 4.65 (t, 1H), 4.47 (dd, 1H), 4.37-4.20 (m, 7H), 4.03 (s, 3H).

³¹P NMR (162 MHz, D₂O) 6-11.42 (d, 1P), −11.58 (d, 1P), −23.15 (t, 1P).

The synthesis of compound 139 (corresponding to m⁷G-diethylene glycol-ppp-(5′)-Guanosine, referred to in WO 2023/007019 A1 FIG. 1 as “Diethyleneglycol linked” compound and shown as “Diethylene glycol cap0” in FIG. 12) was synthesized when starting from diethylene glycol according to WO20231007019 A1 Example 1.1.

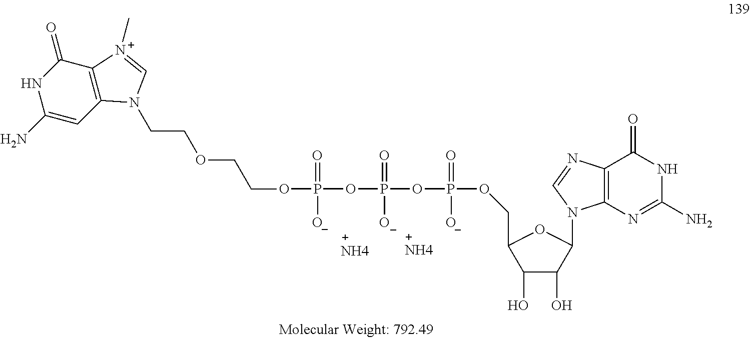 [SMILES: C[n+]1cn(CCOCCOP(=O)([O-])OP(=O)([O-])OP(=O)([O-])OCC2OC(n3cnc4c(=O)[nH]c(N)nc43)C(O)C2O)c2cc(N)[nH]c(=O)c21 ; [NH4+] ; [NH4+]]

¹H NMR (400 MHz, D₂O) δ 8.86 (s, 1H), 8.03 (s, 1H), 5.82 (d, 1H), 4.67 (t, 1H), 4.49 (dd, 1H), 4.37-4.21 (m, 5H), 4.12 (m, 2H), 4.05 (s, 3H), 3.89 (m, 2H), 3.77 (m, 2H).

³¹P NMR (162 MHz, D₂O) 6-11.18 (d, 1P), −11.40 (d, 1P), −23.01 (t, 1P).

The synthesis of compound 140 (corresponding to m⁷G-diethylene glycol-ppp-(5′)-AmG and shown as “Diethylene glycol cap1” in FIG. 12) was synthesized when starting from diethylene glycol according to WO2023/007019 A1 Example 1.6.

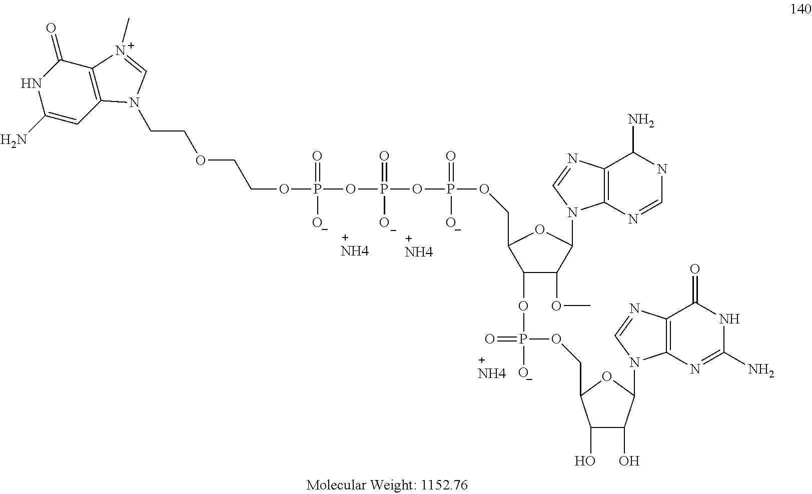 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCCOCCn2c[n+](C)c3c(=O)[nH]c(N)cc32)OC1n1cnc2c1N=CNC2N ; [NH4+] ; [NH4+] ; [NH4+]]

¹H NMR (400 MHz, D₂O) δ 8.82 (s, 1H), 8.37 (s, 1H), 8.08 (s, 1H), 7.93 (s, 1H), 6.01 (d, 1H), 5.78 (d, 1H), 4.92 (m, 1H), 4.72 (t, 1H), 4.49 (m, 2H), 4.41 (t, 1H), 4.33 (m, 1H), 4.30-4.13 (m, 6H), 4.09 (m, 2H), 3.99 (s, 3H), 3.81 (t, 2H), 3.71 (m, 2H), 3.44 (s, 3H),

³¹P NMR (162 MHz, D₂O) δ −0.95 (s, 1P), −11.04 (d, 1P), −11.44 (d, 1P), −22.75 (t, 1P).

### Example 1.14: Synthesis Route XIV

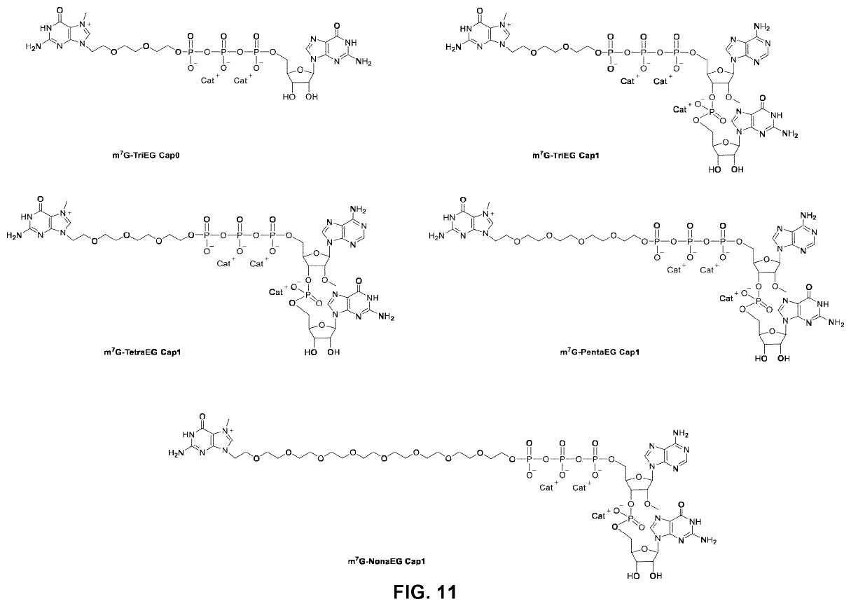

The synthesis of compound 145 (corresponding to m⁷-guanine-triethylene glycol-ppp-(5′)-AmGmG, referred to in FIG. 13 as “m⁷G-TriEG Cap2” compound) when starting from 2′,3′-O-diacetyl-N²-isobutyryl guanosine (X13) and 5′-O-(4,4-dimethoxytrityl)-N2-isobutyryl-2′-OMe-guanosine-3′-(2-cyanoethyl-N,N-diisopropyl)phosphoramidite (X14) is shown in the following. It is noted that the synthesis route for other “Cap2” derivatives also proceeds via the described reaction pathway. Examples of resulting compounds analogous to compound 145 when starting from 2′,3′-O-diacetyl-N²-isobutyryl guanosine (X13) and 5′-O-(4,4-dimethoxytrityl)-N²-isobutyryl-2′-OMe-guanosine-3′-(2-cyanoethyl-N,N-diisopropyl)phosphoramidite (X14) are described at the end of the present example and are depicted in FIG. 13.

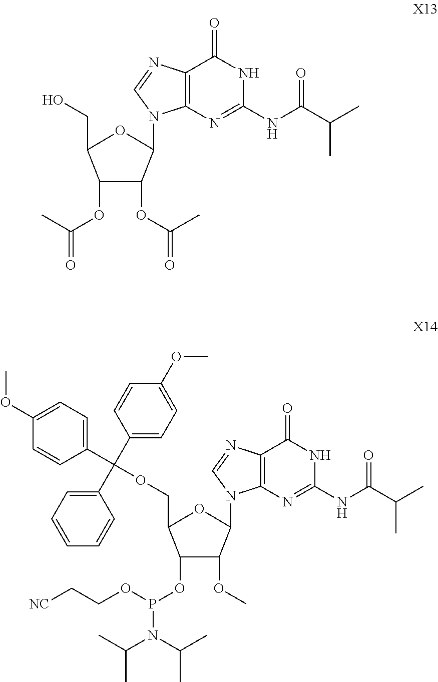 [SMILES: CC(=O)OC1C(CO)OC(n2cnc3c(=O)[nH]c(NC(=O)C(C)C)nc32)C1OC(C)=O ; COc1ccc(C(OCC2OC(n3cnc4c(=O)[nH]c(NC(=O)C(C)C)nc43)C(OC)C2OP(OCCC#N)N(C(C)C)C(C)C)(c2ccccc2)c2ccc(OC)cc2)cc1]

Synthesis of Compound 141 with the Following Structure:

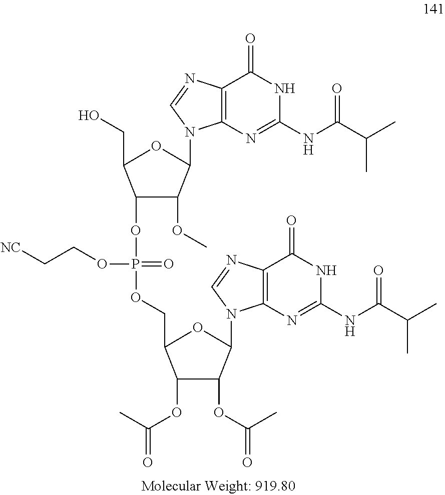 [SMILES: COC1C(OP(=O)(OCCC#N)OCC2OC(n3cnc4c(=O)[nH]c(NC(=O)C(C)C)nc43)C(OC(C)=O)C2OC(C)=O)C(CO)OC1n1cnc2c(=O)[nH]c(NC(=O)C(C)C)nc21]

5′-O-(4,4-dimethoxytrityl)-N²-isobutyryl-2′-OMe-guanosine-3′-(2-cyanoethyl-N,N-diisopropyl)phosphoramidite (X14, 10.0 g, 1.0 eq.) and 2′,3′-O-diacetyl-N²-isobutyryl guanosine (X13, 1.1 eq.) were placed in a round-bottom flask and dried under high vacuum overnight. The starting materials were then dissolved in ACN (60.0 mL) under argon gas atmosphere and acetonitrile containing 0.45 M tetrazole (51.1 mL) was introduced. The reaction mixture was stirred 30 min at ambient temperature and was then cooled to 0° C. A solution of tBuOOH in toluene (1 M, 1.5 eq.) was added dropwise and the reaction mixture was allowed to stir for additional 60 min at 0° C. All volatiles were removed in vacuo and the remaining residue was dried for 5 min under high vacuum. It was then dissolved in 80% aqueous acetic acid (400 mL) and stirred for an additional hour. The solvent was evaporated and the residue was coevaporated trice with methanol. Crude compound 141 was purified by flash chromatography within 2 shots (silica 120 g, A: DCM, B: MeOH, gradient program from Table Ex-106).

TABLE Ex-106

| Time total [min] | Flow [mL/min] | % B |
|---|---|---|
| 0 | 85 | 0 |
| 32 | 85 | 17 |
| 32 | 85 | 80 |
| 37 | 85 | 80 |

Product fractions were analyzed via ESI-MS and NMR. The product was obtained as a white foam in a yield of 92%.

ESI-MS: [M+H]⁺: 920.293, Calculated 920.293

¹H NMR (400 MHz, de-DMSO) δ 12.12 (d br, 1H), 12.09 (s br., 1H), 11.66 (s br., 1H), 11.58 (d br., 1H), 8.31 (m, 1H), 8.27 (s, 1H), 6.13 (dd, 1H), 5.90 (dd, 1H), 5.81 (t, 1H), 5.51 (m, 1H), 5.32 (s br., 1H), 5.10 (m, 1H), 4.60 (m, 1H), 4.44 (m, 3H), 4.24 (m, 3H), 3.58 (dd, 2H), 3.33 (m, 3H), 2.95 (m, 2H), 2.76 (m, 2H), 2.13 (m, 3H), 2.02 (m, 3H), 1.12 (m, 12H).

³¹P NMR (162 MHz, d₆-DMSO) δ −2.23 (d, 1P).

Synthesis of Compound 142 with the Following Structure:

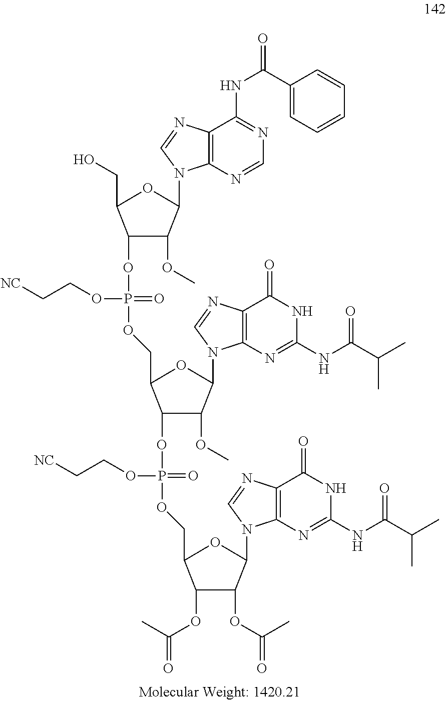 [SMILES: COC1C(OP(=O)(OCCC#N)OCC2OC(n3cnc4c(=O)[nH]c(NC(=O)C(C)C)nc43)C(OC)C2OP(=O)(OCCC#N)OCC2OC(n3cnc4c(=O)[nH]c(NC(=O)C(C)C)nc43)C(OC(C)=O)C2OC(C)=O)C(CO)OC1n1cnc2c(NC(=O)c3ccccc3)ncnc21]

Compound 141 (4.89 g, 1.0 eq.) and N⁶-benzoyl-5′-O-(4,4-dimethoxytrityl)-2′-OMe-adenosine-3′-(2-cyanoethyl-N,N-diisopropyl)phosphoramidite (X4, 1.25 eq.) were placed in a round-bottom flask and dried under high vacuum overnight. The starting materials were then dissolved in ACN (25.0 mL) under argon gas atmosphere and acetonitrile containing 0.45 M tetrazole (29.5 mL, 2.5 eq.) was introduced. The reaction mixture was stirred 45 min at ambient temperature and was then cooled to 0° C. A solution of tBuOOH in toluene (1 M, 3.0 eq.) was added dropwise and the reaction mixture was stirred at 0° C. until full consumption of the starting material (60 min). All volatiles were removed in vacuo and the remaining residue was dried for 5 min under high vacuum. It was then dissolved in 80% aqueous acetic acid (200 mL) and stirred for an additional hour. The solvent was evaporated and the residue was coevaporated trice with methanol. Crude compound 142 was purified by flash chromatography (silica 120 g, A: DCM, B: MeOH, gradient program from Table Ex-107).

TABLE Ex-107

| Time total [min] | Flow [mL/min] | B [%] |
|---|---|---|
| 0 | 85 | 0 |
| 35 | 85 | 30 |
| 35 | 85 | 80 |
| 40 | 85 | 80 |

Product fractions were analyzed via ESI-MS and NMR. The product was obtained as a white foam in a yield of 97%.

ESI-MS: [M+H]⁺: 1420.413, Calculated 1420.414

³¹P NMR (162 MHz, d₆-DMSO) δ −2.20-(−)2-45 (m, 2P).

Synthesis of Compound 143 with the Following Structure:

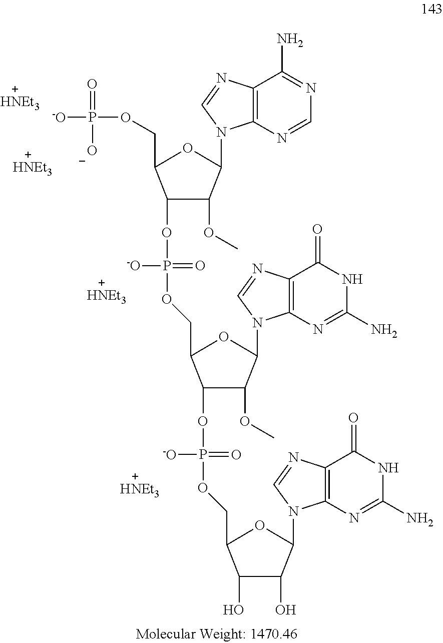

Compound 143 was prepared from compound 142 (10.51 g, 1.0 eq.) according to the general procedure 2 using 29.6 mL ACN, 33.0 mL ACN containing 0.45 M tetrazole, 2.0 eq. bis-cyanoethyl-N,N-diisopropyl phosphoramidite. Oxidation was carried out using 1 M tBuOOH in toluene (4.0 eq.). All volatiles were removed in vacuo and the product was transferred with 20.0 mL methanol into a pressure tube and was deprotected employing 263 mL aqueous ammonia solution at ambient temperature for 48 h. The crude product was then purified by ion exchange chromatography with Macro-Prep-High-Q resin using the gradient program from Table Ex-108 (A: water, B: TEAB 1.0 M).

TABLE Ex-108

| Time total [min] | Flow [mL/min] | % B |
|---|---|---|
| 0 | 66 | 0 |
| 10 | 66 | 0 |
| 130 | 66 | 100 |
| 160 | 66 | 100 |
| 160 | 66 | 0 |
| 170 | 66 | 0 |

Product fractions were analyzed via ESI-MS and NMR The product was obtained as a white powder in a yield of 71%.

ESI-MS: [M−H]⁻: 1064.181, Calculated 1064.182

¹H NMR (400 MHz, d₂O) δ 8.47 (s, 1H), 8.08 (s, 1H), 7.97 (s, 1H), 7.88 (s, 1H), 6.06 (d, 1H), 5.84 (d, 1H), 5.82 (d, 1H), 4.92 (m, 2H), 4.76 (m, 1H), 4.57 (t, 1H), 4.45 (m, 4H), 4.32 (m, 1H), 4.18 (m, 4H), 4.00 (m, 2H), 3.42 (s, 3H), 3.41 (s, 3H).

³¹P NMR (162 MHz, d₂O) δ −0.59 (s, 1P), −3.15 (m, 2P).

Synthesis of Compound 144 with the Following Structure:

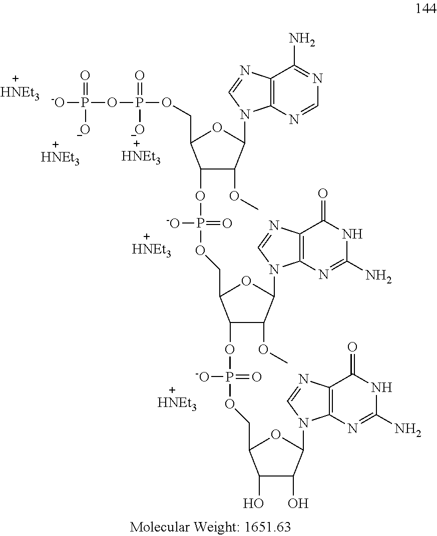

Compound 143 (640 mg, 1.0 eq.) was dissolved in dry dimethylsulfoxide (20 mL). Cyanoethylphosphate-imidazolide lithium salt (3.0 eq.) and magnesium chloride (10.0 eq.) were added and the reaction mixture was stirred for 24 h at ambient temperature. Another portion of cyanoethylphosphate-imidazolide lithium salt (1.0 eq.) was added and stirring was continued for 48 h. Then, DTT (3.0 eq.) and DBU (8 mL) were introduced consecutively, and the reaction was stirred for another 5 h at ambient temperature. DMSO was removed under high vacuum, the remaining residue was dissolved in water and pH was adjusted to 8.0 using concentrated ammonia. Purification was carried out by ion exchange chromatography with Macro-Prep High-Q resin using the following gradient in Table Ex-109: Solvent A: Water, Solvent B: 1 M TEAB.

TABLE Ex-109

| Time total [min] | Flow [mL/min]s | B [%] |
|---|---|---|
| 0 | 66 | 0 |
| 10 | 66 | 0 |
| 60 | 66 | 100 |
| 80 | 66 | 100 |
| 80 | 66 | 0 |
| 90 | 66 | 0 |

Product fractions were analyzed via ESI-MS and NMR. The product was obtained as a white powder in a yield of 47%.

ESI-MS: [M−H]⁻: 1144.148, Calculated 1144.148

¹H NMR (400 MHz, d₂O) δ 8.47 (s, 1H), 8.09 (s, 1H), 7.99 (s, 1H), 7.86 (s, 1H), 6.06 (d, 1H), 5.86 (d, 1H), 5.82 (d, 1H), 4.94 (m, 2H), 4.77 (m, 1H), 4.67 (t, 1H), 4.46 (m, 4H), 4.33 (m, 1H), 4.18 (m, 6H), 3.41 (s, 3H), 3.38 (s, 3H).

³¹P NMR (162 MHz, d₂O) δ −0.83 (s, 1P), −1.00 (s, 1P), −9.23 (d, 1P), −11.31 (d, 1P).

Synthesis of Compound 145 with the Following Structure:

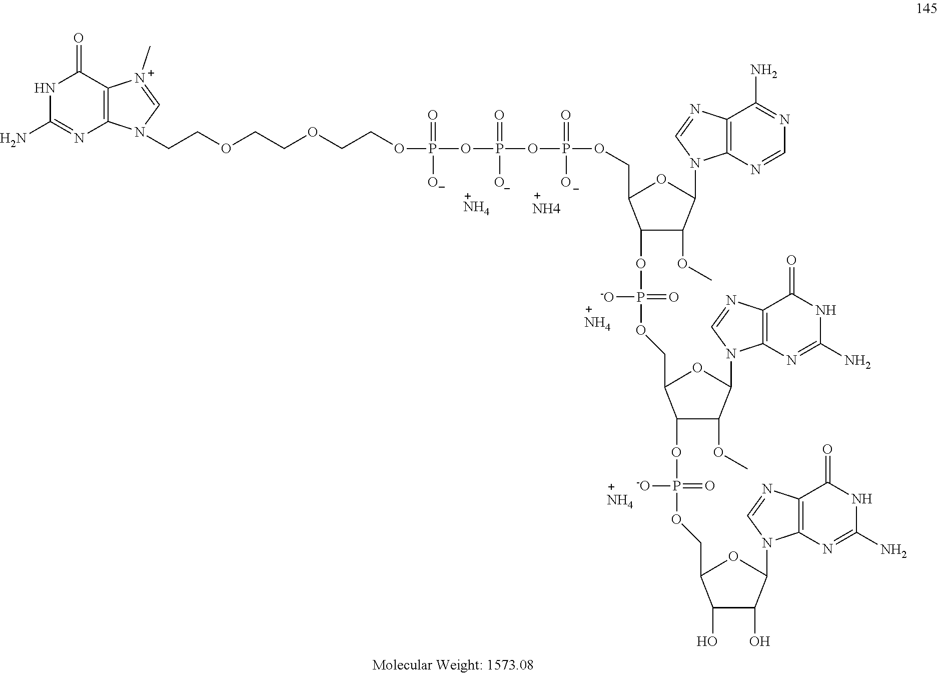

Compound 145 was prepared from compound 122 (108 mg, 1.0 eq.) following the general procedure 7 employing 0.56 mL DMSO and 10.0 eq. CDI. The reaction was quenched after 72 h by the addition of 20.0 eq. water. In the following, NMM buffer (91 μl), water (154 μl), compound 144 (0.67 eq.) and MnCl₂ (0.67 eq.) were added consecutively and stirring was continued. Another 1.0 equivalent of imidazole activated compound 122 was added after 24 h and stirring was continued for another 31 h. The reaction was then quenched by the addition of a 250 mM EDTA solution (1.1 eq.) and the crude product was purified according to general procedure 7 using the gradient program of table Ex-110 (A: 5 mM ammonium acetate in water; B: 90% methanol in water), followed by a second purification using the gradient program of table Ex-111 (A: 100 mM triethylammonium acetate (pH 7), B: 90% MeOH in water), followed by a third purification using the gradient program of table Ex-112 (A: 5 mM ammonium acetate in water; B: 90% methanol in water).

TABLE Ex-110

| Time [min] | A [%] | B [%] |
|---|---|---|
| 0 | 100 | 0 |
| 10 | 100 | 0 |
| 20 | 80 | 20 |
| 21 | 5 | 95 |
| 29 | 5 | 95 |
| 30 | 100 | 0 |
| 50 | 100 | 0 |

TABLE Ex-111

| Time [min] | A [%] | B [%] |
|---|---|---|
| 0 | 100 | 0 |
| 45 | 55 | 45 |
| 46 | 100 | 0 |
| 56 | 100 | 0 |

TABLE Ex-112

| Time [min] | A [%] | B [%] |
|---|---|---|
| 0 | 100 | 0 |
| 10 | 100 | 0 |
| 40 | 50 | 50 |
| 41 | 20 | 80 |
| 46 | 20 | 80 |
| 47 | 100 | 0 |
| 65 | 100 | 0 |

Product fractions were analyzed via ESI-MS and NMR. The product was obtained as a white powder in a yield of 13%.

ESI-MS: [M]⁺: 1505.261, Calculated 1505.262

¹H NMR (400 MHz, D₂O) δ 11.35 (s, 1H), 10.87 (s, 1H), 8.92 (s, 1H), 8.59 (s br., 1H), 8.29 (s, 1H), 8.14 (s, 1H), 6.99 (s, 1H), 6.10 (d, 1H), 5.92 (m, 1H), 5.78 (s, 1H), 5.18 (m, 1H), 4.64 (s, 1H), 4.47-3.00 (m, 30H).

³¹P NMR (162 MHz, D₂O) δ −0.30 (s, 1P), −0.69 (s, 1P), −11.44 (d, 2P), −22.94 (t, 1P).

Similar compounds can be synthesized in accordance with the above synthesis route when using different m⁷G derivatives as described in the previous synthetic pathways. Exemplary compounds along these lines are shown in FIG. 13, namely i) the “3′-OMe-N⁷-BiPheMe-Penciclovir Cap2” compound; ii) the “3′-SPhe-m⁷-Ganciclovir Cap2” compound and iii) the “m⁷-Ganciclovir Cap2” compound, all starting from 2′,3′-O-diacetyl-N²-isobutyryl guanosine (X13) and 5′-O-(4,4-dimethoxytrityl)-N²-isobutyryl-2′-OMe-guanosine-3′-(2-cyanoethyl-N,N-diisopropyl)phosphoramidite (X14).

### Example 1.15: Synthesis Route XV

The synthesis of compound 150 (corresponding to 3′-(2-cyanoethylthio)-m⁷-Ganciclovir-ppp-(5′)-AGmG, referred to in FIG. 13 as “3′-SPhe-m⁷-Ganciclovir Cap2-1” compound) when starting from compound 141 is shown in the following. It is noted that the synthesis route for other “Cap2-1” derivatives also proceeds via the described reaction pathway. Examples of resulting compounds analogous to compound 150 when starting from compound 141 are described at the end of the present example and are depicted in FIG. 13.

Synthesis of Compound 146 with the Following Structure:

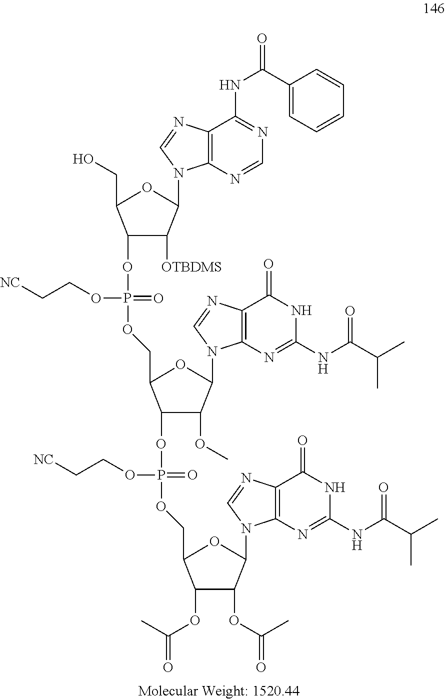 [SMILES: COC1C(OP(=O)(OCCC#N)OCC2OC(n3cnc4c(=O)[nH]c(NC(=O)C(C)C)nc43)C(OC(C)=O)C2OC(C)=O)C(COP(=O)(OCCC#N)OC2C(CO)OC(n3cnc4c(NC(=O)c5ccccc5)ncnc43)C2O[Si](C)(C)C(C)(C)C)OC1n1cnc2c(=O)[nH]c(NC(=O)C(C)C)nc21]

Compound 141 (6.77 g and 5.10 g, 1.0 eq. respectively) and N-benzoyl-5′-O-(4,4-dimethoxytrityl)-2′-O-tert-butyldimethylsilyl-adenosine-3′-(2-cyanoethyl-N,N-diisopropyl)phosphoramidite (X15, 1.25 eq.) were placed in a round-bottom flask and dried under high vacuum overnight. The starting materials were then dissolved in ACN containing 250 mM 4,5-dicyanoimidazole (2.5 eq.) under argon gas atmosphere. The reaction mixture was stirred for 90 min at ambient temperature and was then cooled to 0° C. A solution of tBuOOH in toluene (1 M, 3.0 eq.) was added dropwise and the reaction mixture was stirred at 0° C. for 120 min at 0° C. and for 30 min at ambient temperature. All volatiles were removed in vacuo and the remaining residue was dried for 5 min under high vacuum. It was then dissolved in 80% aqueous acetic acid (540 and 410 mL respectively mL) and stirred for additional 150 min. The solvent was evaporated and the residue was coevaporated trice with methanol. Crude compound 146 was purified by flash chromatography (silica 120 g, A: DCM, B: MeCOH, gradient program from Table Ex-113).

TABLE Ex-113

| Time total [min] | Flow [mL/min] | B [%] |
|---|---|---|
| 0 | 85 | 0 |
| 26.1 | 85 | 15 |
| 27.6 | 85 | 80 |
| 32.8 | 85 | 80 |
| 34.3 | 85 | 20 |
| 38 | 85 | 20 |

Product fractions were analyzed via ESI-MS and NMR. The product was obtained as a white foam in a yield of 60%.

ESI-MS: [M+H]⁺: 1520.484, Calculated 1520.485

³¹P NMR (162 MHz, d₆-DMSO) δ −1.93-(−)2.93 (m, 2P).

Synthesis of Compound 147 with the Following Structure:

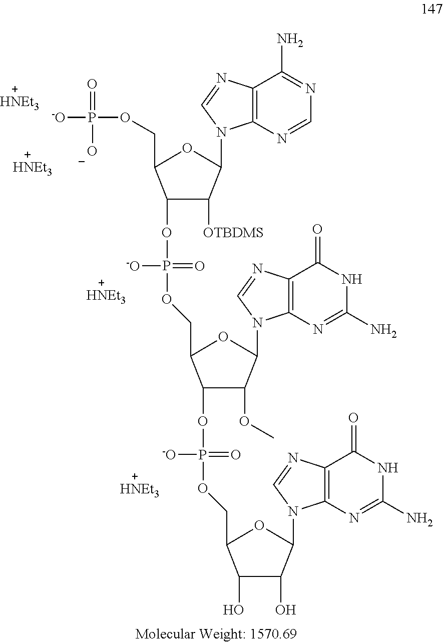

Compound 147 is prepared from compound 146 following the procedure as described for compound 143.

Synthesis of Compound 148 with the Following Structure:

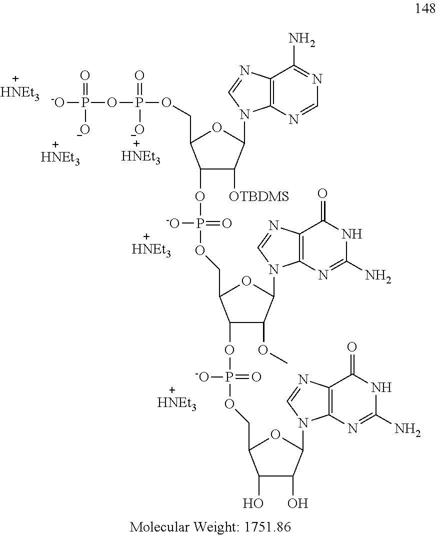

Compound 148 is prepared from compound 147 following the procedure as described for compound 144.

Synthesis of Compound 149 with the Following Structure:

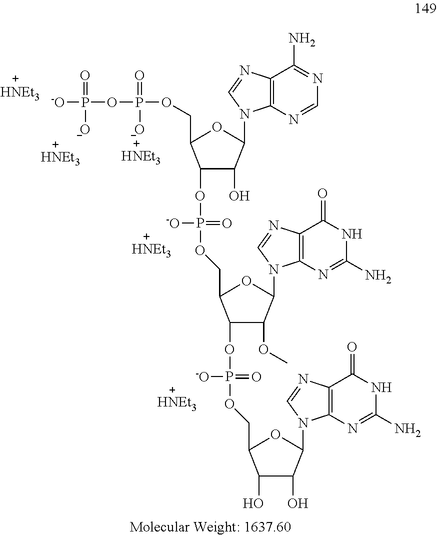

Compound 149 is treated with triethylamine trihydrofluoride and triethylamine or with 1 M TBAF in THE and is stirred until LC-MS indicates complete turnover. All volatiles are removed in vacuo, the product is dissolved in water, pH is adjusted to 7 using concentrated ammonia solution and the crude product is then purified by ion exchange chromatography.

Synthesis of Compound 150 with the Following Structure:

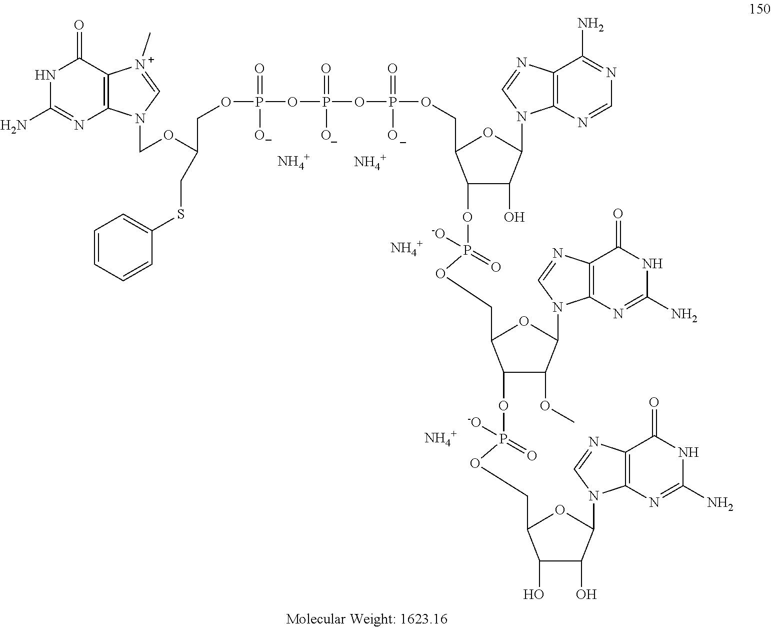

Compound 150 is prepared from compound 149 and compound 102 following the procedure as described for compound 145. Similar compounds can be synthesized in accordance with the above synthesis route when using different m⁷G derivatives as described in the previous synthetic pathways. Exemplary compounds along these lines are shown in FIG. 13, namely i) the “m⁷G-TriEG Cap2-1” compound; ii) the “3′-Ome-N⁷-BiPheMe-Penciclovir Cap2-1” compound and iii) the “m⁷-Ganciclovir Cap2-1” compound, all starting from compound 141.

### Example 2: mRNA Preparation

mRNAs with the commercially available m⁷G(5′)ppp(5′)G cap analogue (from Thermo Fisher Scientific, referred to herein as “mCap”, see also the structure below) were prepared as outlined in the present example. Additionally mRNAs with the commercially available m7G(5′)ppp(5′)(2′OmeA)pG (from TriLink, referred to herein as “CleanCap”) are prepared as outlined in the present example.

The structure of mCap is as follows:

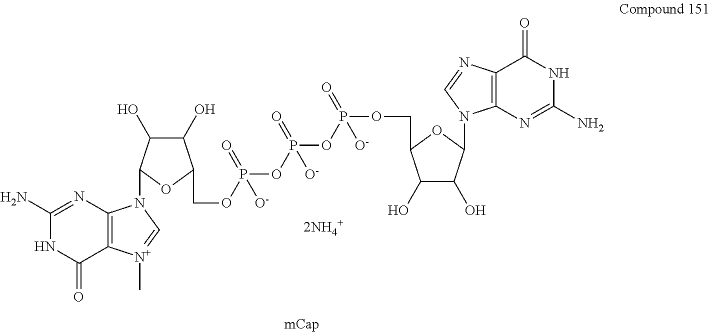 [SMILES: C[n+]1cn(C2OC(COP(=O)([O-])OP(=O)([O-])OP(=O)([O-])OCC3OC(n4cnc5c(=O)[nH]c(N)nc54)C(O)C3O)C(O)C2O)c2nc(N)[nH]c(=O)c21]

The structure of CleanCap is as follows:

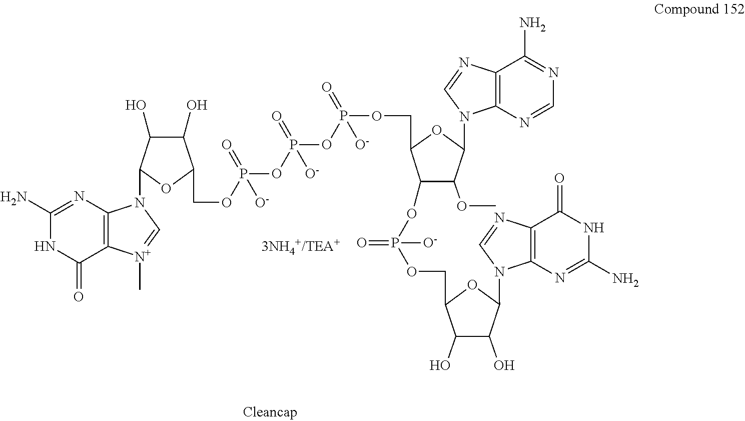 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

Furthermore, mRNAs with the following caps were prepared (see example 1 and Table 114 below): Ganciclovir-phosphate-cap Cap0 (compound 18), Ganciclovir-linked cap Cap0 (compound 19), 3′-OMe-m⁷-Ganciclovir Cap1 (compound 9b), 3′-SPhe-m⁷-Ganciclovir Cap1 (compound 104), 3′-SPhe-m⁷-Ganciclovir Cap0 (compound 103), m⁷-Ganciclovir Cap1 (compound 22), N7-4-ClBn-Ganciclovir Cap1 (compound 109), N7-BiPheMe-Ganciclovir Cap1 (compound 110), m⁷-Penciclovir Cap1 (compound 112), 3′-OMe-N7-4-ClBn-Ganciclovir Cap1 (compound 116), 3′-OMe-N7-BiPheMe-Ganciclovir Cap1 (compound 117), m⁷G-TriEG Cap1 (compound 124), UNA Cap0 (compound 137), m⁷G-TriEG Cap2 (compound 145), m⁷G-TriEG Cap0 (compound 123), Ethylene glycol Cap0 (compound 138), Diethylene glycol cap0 (compound 139) and Diethylene glycol cap1 (compound 140).

TABLE Ex-114
prepared mRNAs

| mRNA ID | Cap analog description | Compound ID | Cap0/Cap1/Cap2 |
|---|---|---|---|
| mRNA (iv)) | Ganciclovir-phosphate | compound 18 | Cap0 |
| mRNA (ii) | Ganciclovir-linked | compound 19 | Cap0 |
| mRNA (v) | 3′-OMe-m⁷-Ganciclovir | Compound 9b | Cap1 |
| mRNA (vi) | 3′-SPhe-m⁷-Ganciclovir | compound 104 | Cap1 |
| mRNA (vii) | 3′-SPhe-m⁷-Ganciclovir | compound 103 | Cap0 |
| mRNA (viii) | m⁷-Ganciclovir | Compound 22 | Cap1 |
| mRNA (ix) | N7-4-ClBn-Ganciclovir | compound 109 | Cap1 |
| mRNA (x) | N7-BiPheMe-Ganciclovir | compound 110 | Cap1 |
| mRNA (xi) | m⁷-Penciclovir | Compound 112 | Cap1 |
| mRNA (xii) | 3′-OMe-N7-4-ClBn- | Compound 116 | Cap1 |
| | Ganciclovir | | |
| mRNA (xiii) | 3′-OMe-N7-BiPheMe- | Compound 117 | Cap1 |
| | Ganciclovir | | |
| mRNA (xiv) | m⁷G-TriEG | Compound 124 | Cap1 |
| mRNA (XV) | m⁷G-TriEG | Compound 123 | Cap0 |
| mRNA (xvi) | CleanCap | Compound 152 | Cap1 |
| mRNA (xvii) | UNA | Compound 137 | Cap0 |
| mRNA (xviii) | m⁷G-TriEG | Compound 145 | Cap2 |
| mRNA (xix) | Ethylene glycol | Compound 138 | Cap0 |
| mRNA (xx) | Diethylene glycol | Compound 139 | Cap0 |
| mRNA (xxi) | Diethylene glycol | Compound 140 | Cap1 |
| mRNA (i) | mCap | Compound 151 | Cap0 |
| mRNA (iii) | | | |
| mRNA (xxii) | | | |

A DNA sequence was introduced into a modified pUC19 derived vector backbone to comprise a 5′-UTR with a ribozyme cleavage site (HSD17B4 5′ UTR), a 3′-UTR (PSMB3 T′UTR), a histone-stem-loop structure and a stretch of adenine nucleotides (A100), at the 3′-terminal end. The obtained plasmid DNA was transformed and propagated in bacteria using common protocols and plasmid DNA was extracted, purified, and enzymatically linearized using a restriction enzyme. The obtained linearized plasmid DNA was used for RNA in vitro transcription as outlined next to obtain the mRNA with the sequence shown in SEQ ID NC: 1 (Cap0). An mRNA with the sequence shown in SEQ ID NO: 2 (Cap0) can be obtained accordingly when using UTP instead of N1-Me-Pseudo-UPT (see below). Further, the obtained linearized plasmid DNA was used for RNA in vitro transcription as outlined next to obtain the mRNA with the sequence shown in SEQ ID NO: 3 (Cap1). An mRNA with the sequence shown in SEQ ID NO: 4 (Cap1) can be obtained accordingly when using UTP instead of N1-Me-Pseudo-UPT (see below). Finally, the obtained linearized plasmid DNA was used for RNA in vitro transcription as outlined next to obtain the mRNA with the sequence shown in SEQ ID NO: 5 (Cap2). An mRNA with the sequence shown in SEQ ID NO: 6 (Cap2) can be obtained accordingly when using UTP instead of N1-Me-Pseudo-UPT (see below).

Linearized plasmid DNA template (50 Ng/mL) was transcribed at 37° C. for 3-5 hours in 80 mM HEPES/KOH, pH 7.5, 24 mM MgCl₂, 2 mM spermidine, 40 mM DTT, 5 U/mL pyrophosphatase (Thermo Fisher Scientific), 200 U/mL RiboLock RNase inhibitor (Thermo Fisher Scientific), 5000 U/mL T7 RNA polymerase (Thermo Fisher Scientific). The modified nucleotide mixture for RNA production with mCap (to arrive at mRNA (i) and to arrive at mRNA (xxii) with a different concentration of mCap used in the reaction to arrive at either mRNA (i) or mRNA (xxii), see description of the transcription conditions below), the Ganciclovir-linked cap (compound 19, to arrive at mRNA (ii)), 3′-OMe-m7-Ganciclovir Cap9 (to arrive at mRNA (v)), 3′-SPhe-m7-Ganciclovir Cap1 (to arrive at mRNA (vi)), 3′-SPhe-m7-Ganciclovir Cap0 (to arrive at mRNA (vii)), m7-Ganciclovir Cap1 (to arrive at mRNA (viii)), N7-4-ClBn-Ganciclovir Cap1 (to arrive at mRNA (ix)), N7-BiPheMe-Ganciclovir Cap1 (to arrive at mRNA (x)), m7-Penciclovir Cap1 (to arrive at mRNA (xi)), 3′-OMe-N7-4-ClBn-Ganciclovir Cap1 (to arrive at mRNA (xii)), 3′-OMe-N7-BiPheMe-Ganciclovir Cap1 (to arrive at mRNA (xiii)), m7G-TriEG Cap1 (to arrive at mRNA (xiv)), m7G-TriEG Cap0 (to arrive at mRNA (xv), CleanCap (to arrive at mRNA (xvi), Ethylene glycol Cap0 (to arrive at mRNA (xix)), Diethylene glycol Cap0 (to arrive at mRNA (xx)), Diethylene glycol Cap1 (to arrive at mRNA (xxi)), and UNA cap0 (to arrive at mRNA (xvii)) was sequence-optimized (herein referred to as sequence-optimized IVT-mix) preferably in accordance with a procedure as described in WO2015/188933, Example 1. In short, the sequence-optimized IVT-mix comprised the four ribonucleoside triphosphates (NTPs) GTP, ATP, CTP and 1-Me-Pseudo-UTP (for SEQ ID NO: 1 (Cap0), SEQ ID NO: 3 (Cap1) and SEQ ID NO: 5 (Cap2)—if SEQ ID NO. 2 (Cap0), SEQ ID NO: 4 (Cap1) or SEQ ID NO: 6 (Cap2) should be obtained, this would be replaced by UTP) in a sequence optimized ratio, wherein the fraction of each of the four ribonucleoside triphosphates in the sequence-optimized IVT-mix corresponded to the fraction of the respective nucleotide in the mRNA molecule to be synthetized, a buffer, a DNA template, and an RNA polymerase. Two further mRNAs, one using mCap (to arrive at mRNA (iii)) and one using the Ganciclovir-phosphate cap (compound 18, to arrive at mRNA (iv)) as cap were produced with equimolar concentrations of ATP, CTP and N1-Me-Pseudo-UTP (for SEQ ID NO: 1, if SEQ ID NO: 2 should be obtained, this would be replaced by UTP) and a reduced amount of GTP.

Transcription to arrive at mRNAs (i) (mCap, sequence-optimized nucleotide mixture), (ii) (Ganciclovir-linked cap, sequence-optimized nucleotide mixture), (vii) (3′-SPhe-m⁷-Ganciclovir Cap0), (xv) (m⁷G-TriEG Cap0), (xix) (Ethylene glycol Cap0) and (xx) (Diethylene glycol Cap0) was carried out at 15.23 mM concentration of the respective cap analogue, 3.81 mM GTP, 3.18 mM ATP, 4.33 mM CTP and 2.13 mM 1-Me-pseudo-UTP. Transcription to arrive at above mentioned mRNAs (v), (vi), (viii)-(xiv), (xvi)-(xviii) and (xxi-xxii) was carried out at 5 mM concentration of the respective cap analogue, 3.81 mM GTP, 3.18 mM ATP, 4.33 mM CTP and 2.13 mM 1-Me-pseudo-UTP (for SEQ ID NO: 1 (Cap0), SEQ ID NO: 3 (Cap1) and SEQ ID NO: 5 (Cap2)—if SEQ ID NO: 2 (Cap0), SEQ ID NO: 4 (Cap1) or SEQ ID NO: 6 (Cap2) should be obtained, this would be replaced by UTP).

Transcription to arrive at mRNAs (iii) (mCap, equimolar concentrations of nucleotides) and (iv) (Ganciclovir-phosphate cap, equimolar concentrations of nucleotides) was carried out at 3 mM concentration of the respective cap analogue, 0.5 mM GTP, 2 mM ATP, 2 mM CTP and 2 mM N1-Me-pseudo-UTP (for SEQ ID NO: 1, if SEQ ID NO: 2 should be obtained, this would be replaced by UTP).

Following RNA in vitro transcription, linear DNA templates were removed by Pulmozyme (Ratiopharm) (2500 U/mL, 3.2 mM CaCl₂), 30 min at 37° C.).

The obtained mRNAs (i) and (ii) and (v), (vi), (viii)-(xvi) and (xviii)-(xxi) were purified using RP-HPLC (PureMessenger®; according to WO2008/077592). mRNA (xxii) was purified using the Monarch RNA cleanup kit (NEB). The obtained mRNAs (iii) and (iv) were purified using Megaclear transcription purification kit from Invitrogen according to the protocol of the manufacturer. mRNA (ii) and further mRNAs as indicated below were used for capping analysis (Example 3), whereas mRNAs (ii) and (iv) and further mRNAs as indicated below were used in in vitro expression experiments (Examples 5 to 8).

Further mRNAs with caps according to the present invention are prepared similarly, namely when carrying out the transcription in the presence of a corresponding cap. The obtained mRNAs are purified using RP-HPLC (PureMessenger®; according to WO2008/077592) or Monarch RNA cleanup kit (mRNA used in Example 8) or Qiagen RNeasy mini kit or Megaclear-kit according to the protocol of the manufacturer.

### Example 3: Determination of the Capping Efficacy when Using Different Cap Analogs

The capping efficacy can be analyzed by an HPLC assay of a 5′ fragment of mRNAs obtained according to the protocol of example 2. Thus, the peaks obtained in such an HPLC assay are indicative of a) correctly capped mRNA, b) capped mRNA lacking a single nucleotide G (which is a typical side product if T7 RNA-polymerase is used), c) uncapped mRNA and d) further products of unknown structure. 5′ fragments for this kind of analysis are typically 15 to 20 nucleotides in length.

In order to provide fragments of a length of 18 nucleotides (assuming that the cap-nucleotide is present, the fragment is 18 nucleotides long; for uncapped mRNAs, the fragment is accordingly 17 nucleotides long) for HPLC analysis, the mRNA (ii) [Ganciclovir-linked cap1 obtained in example 2 was first cleaved at the above-mentioned 5′ ribozyme cleavage site using a ribozyme designed to cleave at the relevant position. The ribozyme reaction contained 150 μM of the respective mRNA, 150 μM of the ribozyme, 50 mM NaCl and 0.625 mM EDTA in a total reaction volume of 120 μL. For the annealing reaction, the mixture was incubated in a PCR cycler for 3 min at 95° C., followed by a cool down ramp of 0.1° C. per second to a final temperature of 25° C., with a final incubation of 10 min at 25° C. After addition of 30 μL of a 200 mM MgCl₂ solution buffered with 250 mM TRIS/HCl (pH 7.5), the reaction mix was incubated at 25° C. for 1 h. The reaction was stopped by addition of 24 μL of a 250 mM EDTA solution. RNA-only and Ribozyme-only controls were prepared per RNA and ribozyme, respectively.

Prior to HPLC analysis, 1446 μL HPLC-grade water and 180 μL 1 M TEAA solution were added to the stopped reaction mix and mixed vigorously. HPLC analysis was performed using a AQUITY PREMIER Oligonucleotide C18 130 Å column (2.1×50 mm, 1.7 μm particle size, Waters) with a column temperature of 65° C. and a flowrate of 0.65 mL/min. Eluent A consisted of 0.1 M TEAA in HPLC grade water, pH 7.0. Eluent B consisted of 0.1 m TEAA, 15% ACN (v/v) in HPLC grade water, pH 7.0. A specific gradient was applied to separate the short RNA fragments (see Table Ex-115). RNA peaks were detected by a UV/VIS spectrophotometer at 260 nm. Peak areas were integrated resulting in the relative fractions of differently capped mRNA.

TABLE Ex-115
HPLC gradient for capping analysis

| Time (min) | Fraction Eluent B (%) |
|---|---|
| 0-3 | 14 |
| 3-5 | 14-19 |
| 5-14 | 19-21 |
| 14-15 | 21 |
| 15-20 | 21-100 |
| 20-23.5 | 100 |
| 23.5-25 | 100-14 |

The identities of the peaks and the obtained relative peak areas are shown in Table Ex-116.

TABLE Ex-116
peak identities and relative peak areas

| | Relative peak area [%] | | | |
|---|---|---|---|---|
| | Correctly | Capped | | |
| | capped | RNA | Uncapped | Unidentified |
| mRNA-sample | RNA | minus G | RNA | peaks |
| Ganciclovir-linked | 62.11 | 15.24 | 18.78 | 3.87 |
| [mRNA (ii)] | | | | |

It is evident from the above result shown in Table Ex-116 that the use of Ganciclovir-linked [mRNA (ii)] cap analog resulted in correctly capped mRNA.

The capping efficacy can be further analyzed by an LC-MS based assay of a 5′ fragment of mRNAs obtained according to the protocol of example 2. Thus, the peaks obtained in such an LC-MS assay are indicative of a) correctly capped mRNA, b) capped mRNA lacking a single nucleotide G (which is a typical side product if T7 RNA-polymerase is used or a side product during the assay using RNaseH), c) uncapped mRNA and d) further products of unknown structure. 5′ fragments for this kind of analysis are typically 10 to 20 nucleotides in length.

In order to provide fragments of a length of 16 nucleotides (assuming that the cap-nucleotide is present, the fragment is 16 nucleotides long, for uncapped mRNAs, the fragment is accordingly 15 nucleotides long) for HPLC analysis, the mRNAs (v) [3′-OMe-m⁷-Ganciclovir Cap1], (vi) [3′-SPhe-m⁷-Ganciclovir Cap1], (viii) [m⁷-Ganciclovir Cap1], (ix) [N7-4-ClBn-Ganciclovir Cap1], (x) [N7-BiPheMe-Ganciclovir Cap1], (xi) [m⁷-Penciclovir Cap1], (xii) [3′-OMe-N7-4-ClBn-Ganciclovir Cap1], (xiii) [3′-OMe-N7-BiPheMe-Ganciclovir Cap1], (xiv) [m⁷G-TriEG Cap1], (xvi) [CleanCap] and (xxi) [Diethylen glycol] obtained in example 2 was first hybridized to a chimeric 3′-biotinylated DNA/2′O-methylated oligonucleotide directing RNaseH to the cleavage site followed by binding to magnetic beads and RNAseH clevage. Subsequently, cleavage fragments were analyzed via LC-MS. mRNAs not explicitly referred to in the present paragraph were analyzed using the ribozyme-assay as described herein above.

RNA peaks were detected by a UVNIS spectrophotometer at 260 nm and identified by a mass spectrometer. Peak areas were integrated resulting in the relative fractions of differently capped mRNA.

The identities of the peaks and the obtained relative peak areas are shown in Table Ex-117.

TABLE Ex-117
peak identities and relative peak areas

| | Relative peak area [%] | | |
|---|---|---|---|
| | capped RNA | | |
| | (Capped + Capped | Uncapped | Unidentified |
| mRNA-sample | minus G) | RNA | peaks |
| 3′-OMe-m⁷-Ganciclovir Cap1 | 87.28 | 0 | 12.72 |
| [mRNA (v)] | | | |
| m⁷-Ganciclovir Cap1 | 91.55 | 4.54 | 3.91 |
| [mRNA (viii)] | | | |
| N7-4-ClBn-Ganciclovir Cap1 | 77.39 | 0 | 22.61 |
| [mRNA (ix)] | | | |
| N7-BiPheMe-Ganciclovir Cap1 | 100 | 0 | 0 |
| [mRNA (x)] | | | |
| m⁷-Penciclovir Cap1 | 94.24 | 3.25 | 2.51 |
| [mRNA (xi)] | | | |
| 3′-OMe-N7-4-ClBn-Ganciclovir Cap1 | 97.62 | 0 | 2.38 |
| [mRNA (xii)] | | | |
| 3′-OMe-N7-BiPheMe-Ganciclovir Cap1 | 100 | 0 | 0 |
| [mRNA (xiii)] | | | |
| m⁷G-TriEG Cap1 | 89.74 | 5.45 | 4.81 |
| [mRNA (xiv)] | | | |
| CleanCap | 90.79 | 1.45 | 7.76 |
| [mRNA (xvi)] | | | |
| 3′-SPhe-m⁷-Ganciclovir Cap1 | 87.85 | 0.78 | 11.37 |
| [mRNA (vi)] | | | |
| mCap | 84.92 | 11.16 | 3.92 |
| [mRNA (i)] | | | |
| UNA Cap0 [mRNA (xvii)] | 59.98 | 35.96 | 4.06 |
| Diethylene glycol Cap1 [mRNA (xxi)] | 93.2 | 0.85 | 5.95 |
| Diethylene glycol Cap0 [mRNA (xx)] | 93.3 | 3.01 | 3.69 |
| Ethylene glycol [mRNA (xix)] | 81.24 | 9.97 | 8.79 |
| mCap [mRNA (iii)] | 83.18 | 10.39 | 6.43 |
| mCap | 65.23 | 31.15 | 3.62 |
| [mRNA (xxii)] | | | |

It is noted that mRNA (xxii) using mCap was prepared at an mCap concentration of 5 mM and thus at a lower concentration compared to mRNAs (i) and (iii), where mCap was used at a concentration of 15.23 mM. This explains the (expected) lower degree of capping of mRNA (xxii) compared to mRNAs (i) and (iii). mRNA (xviii), the Cap2 bearing mRNA (with the m7G-TriEG cap analog) was also analyzed and it was found that correctly capped RNA was obtained. However, the level was slightly lower than for the mRNAs shown in Table Ex-117, which can be explained by the need for optimizing the conditions of the in vitro transcription reaction using a Cap2 analog. Such optimization can easily be carried out in order to arrive at correctly capped RNA (xvii) at a level comparable to the levels shown in Table Ex-117.

It is noted that mRNAs (x) and (xiii) are depicted with a 100% capping efficiency because both carry a biphenyl-methyl-substituent at the N7 such that it was possible to purify the correctly capped fractions from the remaining fractions to arrive at a capping efficiency of up to 100%.

### Example 4: Determination of the Presence of dsRNA

dsRNA can be an undesired side product of in vitro transcription and is mainly resulting from i) a 3-extension of the run-off products annealing to complementary sequences in the body of the run-off transcript in cis (by folding back on the same RNA) or trans (by annealing to a second RNA) to form extended duplexes or to ii) hybridization of an antisense RNA molecule to the run-off transcript. The amount of dsRNA in an RNA preparation can be analyzed inter alia with an ELISA assay using antibodies specific for dsRNA, as described in the following.

9D5 antibody (specific for dsRNA, from absolute antibody) is diluted to 2 μg/mL in PBS and used to coat Nunc MaxiSorp® flat bottom 96-well plates (Thermo Fischer) with 100 μl for 2 h at room temperature. After coating, wells are washed three times using PBS-T (PBS and 0.05% Tween-20). Samples and standards are diluted in 1×TE buffer (AppliChem) and 100 μl are added to each well and incubated over night at 4° C. (approx. 20 h). After incubation, wells are washed three times using PBS-T. K2 antibody (Scicon) is diluted 1:200 in PBST and 100 μl are added to each well and incubated for 2 h at room temperature. Wells are washed three times using PBS-T. Anti-mouse IgM-HRP (Invitrogen) is diluted 1:50 in PBST and 100 μl are added to each well and incubated for 1 h at room temperature Wells are washed three times using PBS-T. Color reagents A and B (R&D systems) are mixed in equal amounts and 100 μl are added to each well and incubated for 9 minutes. Plates are measured in a plate reader at OD450 and OD540. OD540 values are subtracted from OD450 values and used for the determination of absolute amounts of dsRNA with a lower limit for quantification of 0.03 n₉ of dsRNA per μg RNA. dsRNA content was determined for mRNAs (v) to (xvi) and was below limit of quantification.

### Example 5: Luciferase Expression Using mRNAs with Various Cap Analogs

Cells were seeded on 96 well plates (Sarstedt). HDF (human dermal fibroblast) and HeLa were seeded 24 hours before transfection in a compatible complete cell medium (10,000 cells in 200 μl/well). Cells were maintained at 37° C., 5% CO₂. The day of transfection, the complete medium on cells was replaced with serum-free Opti-MEM medium (Gibco). Each mRNA, namely mRNA (ii) [Ganciclovir-linked, compound 19] and mRNA (iv) [Ganciclovir-phosphate cap, compound 18], was complexed with Lipofectamine2000 at a ratio of 1/1.5 (w/v) for 20 minutes in Opti-MEM. Lipocomplexed mRNAs were then added to cells for transfection with 50 ng of mRNA per well in a total volume of 200 μl. 90 minutes post start of transfection, complete supernatant (200 μl/well) of transfection solution was exchanged for 200 μl/well of complete medium. Cells were further maintained at 37° C., 5% CO₂ before harvesting. 24 hours post start of transfection cells were lysed to measure luciferase expression within cells.

First, 100 μl of 1×passive lysis buffer (Promocell) was added to each well. Cells were shaken for 15 minutes at room temperature until there were incubated at −80° C. for at least one hour. After thawing, 20 μl of lysates were used to detect and measure luciferase activity via chemi-luminescence using ATP and D-Luziferin in a Beetlejuice buffer system (p.j.k.). To this end, plates were introduced into a in a plate reader (Tristar 2S Berthold) with injection device for Beetle-juice containing substrate for firefly luciferase. Per well, 50 μl of beetle-juice were added. Raw data containing relative light units were used to plot differences between mRNAs derived from cap analogs.

Expression Analysis of cap0 mRNA:

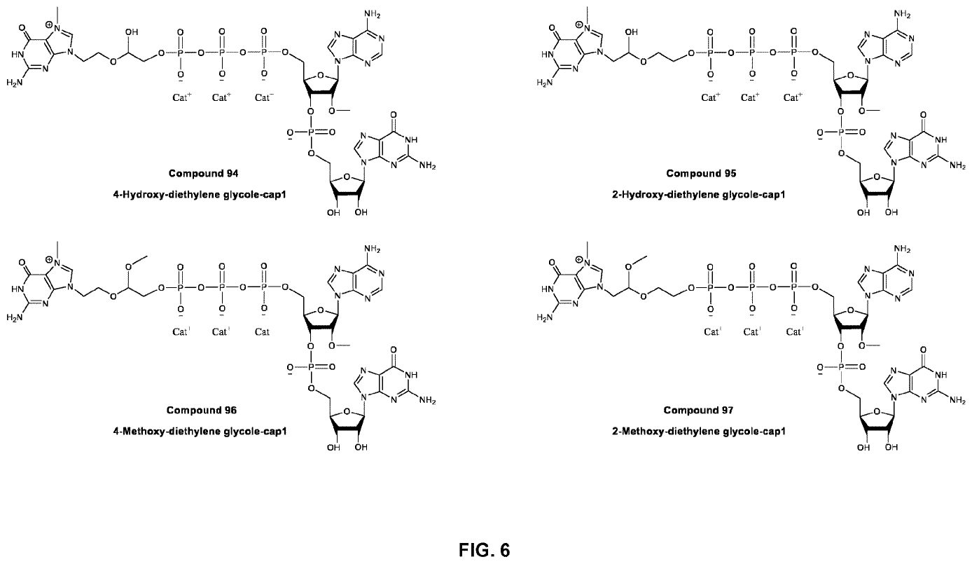

The two tested cap0 mRNAs (ii) and (iv) (see Example 2, wherein mRNA (ii) was prepared using the Ganciclovir-linked cap analog (compound 19) using a sequence optimized nucleotide mixture and mRNA (iv) was prepared using the Ganciclovir-phosphate-linked cap analog (compound 18)) using equimolar nucleotide concentrations showed expression of PpLuc protein after transfection of 50 ng mRNA in HDF (striped bars in FIG. 7) and HeLa cells (unfilled bars in FIG. 7).

### Example 6: Luciferase Expression Using mRNAs with Improved Cap1 Analogs

The experiment was performed to determine a potential difference in the expression when using Cap0 analogs vs. Cap1 analogs of the invention.

HDF cells were seeded and treated as described in Example 5. Each mRNA, see Table Ex-118 and Ex-Table 119, was complexed and transfected as described in Example 5. 24 hours post start of transfection cells were lysed to measure luciferase expression within cells.

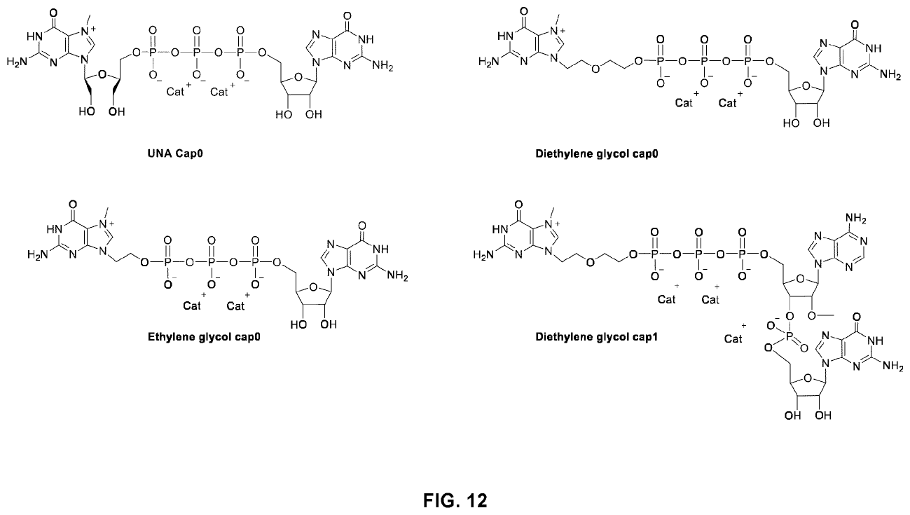

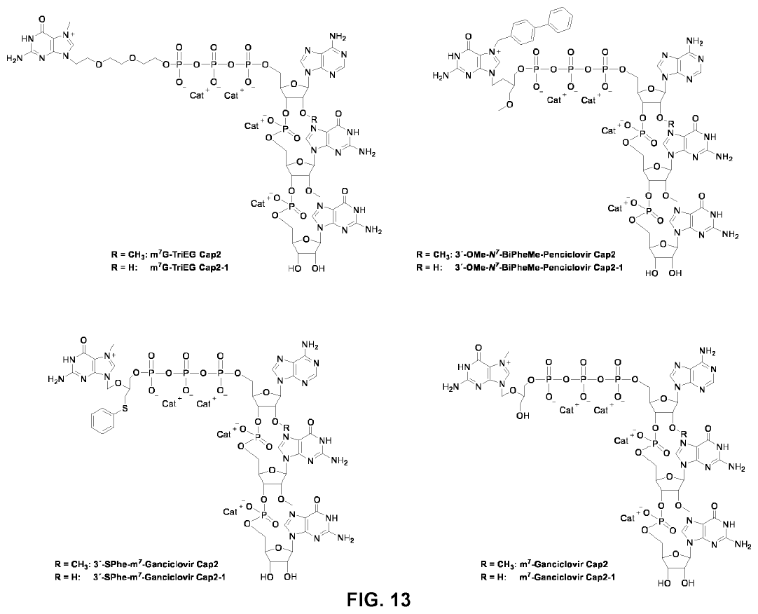

TABLE Ex-118
mRNAs used in Example 6-FIG. 14A

| mRNA ID | Cap analog description | Compound ID | Cap0/Cap1 |
|---|---|---|---|
| mRNA (XV) | m7G-TriEG | Compound 123 | Cap0 |
| mRNA (xiv) | m7G-TriEG | Compound 124 | Cap1 |
| mRNA (vii) | 3′-SPhe-m7-Gancyclovir | Compound 103 | Cap0 |
| mRNA (vi) | 3′-SPhe-m7-Gancyclovir | Compound 104 | Cap1 |

Expression Analysis of Cap0 and Cap1 mRNA:

All tested Cap0 and Cap1 mRNAs (see Table 118) showed expression of PpLuc protein after transfection of 50 ng mRNA in HDF cells (see FIG. 14A). The results obtained with mRNA capped with the inventive Cap1 analogs show a much higher expression compared to the corresponding Cap0 analogs. This dramatic increase in expression was unexpected.

Additionally, the expression of mRNAs capped with the inventive Cap1 analogs was compared to the expression of an mRNA capped with a standard cap1 analog (CleanCap) as shown in Table Ex-119 and FIG. 14B.

TABLE Ex-119
mRNAs used in Example 6-FIG. 14B

| mRNA ID | Cap analog description | Compound ID | Cap0/Cap1 |
|---|---|---|---|
| mRNA (xvi) | CleanCap | Compound 152 | Cap1 |
| mRNA (xiv) | m7G-TriEG | Compound 124 | Cap1 |
| mRNA (vi) | 3′-SPhe-m7-Gancyclovir | Compound 104 | Cap1 |
| mRNA (viii) | m⁷-Ganciclovir linked | Compound 22 | Cap1 |

Expression Analysis of Inventive Cap1 mRNA:

FIG. 14B shows the PpLuc expression of mRNAs capped with the inventive Cap1 analogs compared to a standard Cap1 capped mRNA ((xvi), CleanCap). All mRNAs capped with the inventive Cap1 analogs have an expression that is at least comparable to the expression of mRNA (xvi), wherein mRNAs (xiv) and (vi) even result in a much higher expression. Accordingly, these Cap1 structures are particularly suitable for producing efficient mRNA molecules for mRNA therapy.

### Example 7: Luciferase Expression of (Poly-)Ethylene Glycol Cap Analogs

The experiment was performed to determine a potential effect of the number of ethylene glycol chains, i.e. a potential effect of an increasing ethylene glycol linker, in the inventive cap analogs. HDF cells were seeded and treated as described in Example 5. Each mRNA, see Table Ex-120 and Table Ex-121, was complexed and transfected as described in Example 5. 24 hours post start of transfection cells were lysed to measure luciferase expression within cells.

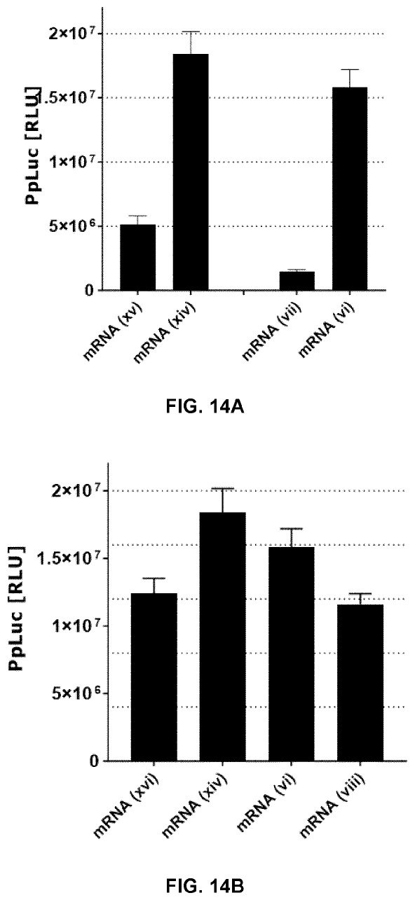

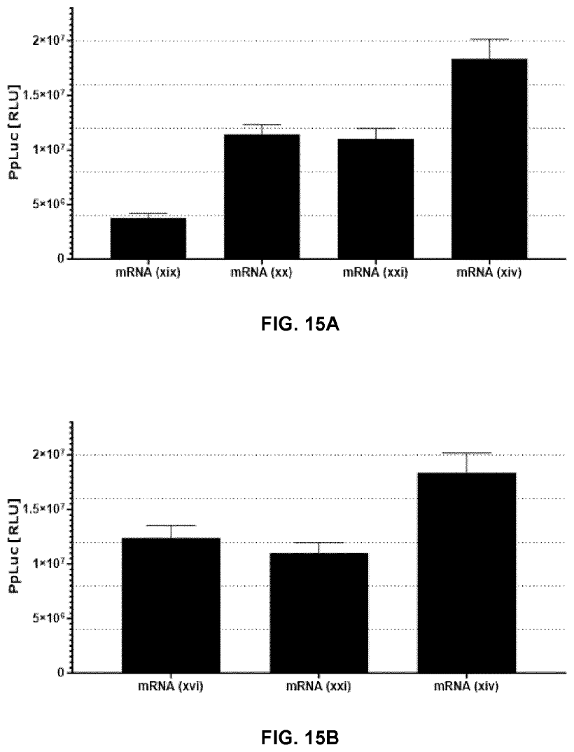

TABLE Ex-120
mRNAs used in Example 7-FIG. 15A

| mRNA ID | Cap analog description | Compound ID | Cap0/Cap1 |
|---|---|---|---|
| mRNA (xix) | Ethylene glycol | Compound 138 | Cap0 |
| mRNA (xx) | Diethylene glycol | Compound 139 | Cap0 |
| mRNA (xxi) | Diethylene glycol | Compound 140 | Cap1 |
| mRNA (xiv) | m7G-TriEG | Compound 124 | Cap1 |

Expression Analysis of mRNA Capped with (Poly) Ethylene Glycol Cap Analogs:

FIG. 15A shows the PpLuc expression of mRNAs capped with different (poly)ethylene glycol cap analogs. Surprisingly, and consistent within either Cap0 or Cap1, the mRNAs with more ethylene glycol units provided for an increased expression. Thus, on Cap0-level, the expression from mRNA (xx) with the “diethylene glycol” cap is increased compared to the mRNA (xix) with the “monoethylene glycol” cap. Further and consistent with this finding, the expression from mRNA (xiv) with the “triethylene glycol” cap is increased compared to the mRNA (xxi) with the “diethylene glycol” cap. This finding is in particular surprising, since the chain length between the nucleobase and the phosphate is with 8 atoms substantial longer than for the natural ribose with 4 atoms.

Additionally, the PpLuc protein expression of mRNA capped with m7G-TriEG Cap1 analog was compared to mRNA capped with Diethylene glycol Cap1 analog and a standard cap1 analog (CleanCap) as shown in Table Ex-121 and FIG. 15B.

TABLE Ex-121
mRNAs used in Example 7-FIG. 15B

| mRNA ID | Cap analog description | Compound ID | Cap0/Cap1 |
|---|---|---|---|
| mRNA (xvi) | CleanCap | Compound 152 | Cap1 |
| mRNA (xxi) | Diethylene glycol | Compound 140 | Cap1 |
| mRNA (xiv) | m7G-TriEG | Compound 124 | Cap1 |

Expression Analysis of mRNA Capped with m7G-TriEG Cap1 Analog:

The results shown in FIG. 15B obtained with mRNA capped with the inventive m7G-TriEG Cap1 analog (xiv) compared to the Diethylene Glycol Cap1 variant (xxi) and the standard mRNA capped with CleanCap (xvi) show a substantially higher expression for mRNA (xiv) with the “triethylene glycol” cap.

### Example 8: Luciferase Expression of UNA Cap Analog Compared to Improved Cap Analogs

The experiment was performed to compare the inventive Cap1 analogs to analogs disclosed in the prior art and thus known in the field, in particular to mCap with the structure shown above and to UNA with the structure shown above, wherein the structure of UNA has two branches in a linear structure on not only a single branch, as is the case in the present invention. UNA is disclosed as compound 36 in WO 2017/066789.

HDF cells were seeded and treated as described in Example 5. Each mRNA, see Table Ex-122, was complexed and transfected as described in Example 5. 24 hours post start of transfection cells were lysed to measure luciferase expression within cells.

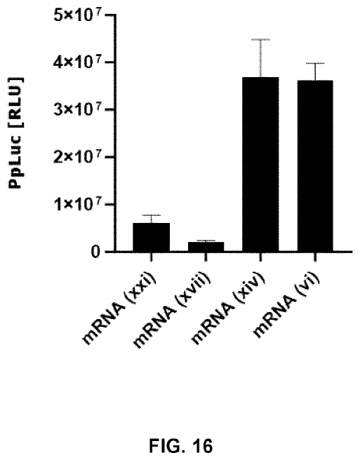

TABLE Ex-122
mRNAs used in Example 8-FIG. 16

| mRNA ID | Cap analog description | Compound ID | Cap0/Cap1 |
|---|---|---|---|
| mRNA (xxii) | mCap | Compound 151 | Cap0 |
| mRNA (xvii) | UNA | Compound 137 | Cap0 |
| mRNA (xiv) | m⁷G-TriEG | Compound 124 | Cap1 |
| mRNA (vi) | 3′-SPhe-m7-Ganciclovir | Compound 104 | Cap1 |

Expression Analysis of mRNA Capped with Inventive Cap Analogs:

FIG. 16 shows the PpLuc expression of the different mRNAs as indicated in Table Ex-122. It can be derived from the expression levels as obtained and shown in FIG. 16 that mRNA (xvii) with the UNA cap results in the lowest expression, and that this expression is substantially lower compared to an mRNA with mCap (mRNA (xxii)), whereas the two mRNAs with cap analogs according to the present invention clearly by far outperform the two afore-mentioned mRNAs. Even when acknowledging that mRNAs with a Cap0 cap ((xxii) and (xvii)) were compared to mRNAs with a Cap1 cap ((xiv) and (vi)), the dramatic difference was unexpected.

### Example 9: In Vivo Study with mRNA Comprising Improved Cap Analogs

The experiment aims at demonstrating improved properties of the inventive cap analogs in LNP formulated mRNA in vivo.

PpLuc intravenous in vivo luciferase expression experiment is conducted in female BALB/C mice of 8 weeks old in groups of 6 animals in two cohorts. Lipid nanoparticle formulations with state of the art LNP formulation include mRNAs described in Table Ex-123 encoding photinus pyralis luciferase. Formulated mRNAs are at a stock concentration of 0.25 g/L and diluted prior injection to a final concentration of 0.05 g/L. 5 μg of the respective mRNAs are injected intravenously with a volume of 100 μL per animal into the tail vein. For luciferase activity determination cohort one is sacrificed 6 h and cohort two 24 h after injection for harvesting of liver and spleen. Organs are lysed prior luciferase measurement. Therefore, 100 mg of the respective organ is lysed with a steel bead in a 2 mL safe lock Eppendorf tube mounted in a tissue lyser for 3 minutes at full speed. 1× passive lysis buffer (800 μL for liver and 500 μL for spleen) is added to each sample and mounted again in a tissue lyser for 6 minutes at full speed. Samples are centrifuged at 13,500 rpm at 4° C. for 10 minutes. Supernatant is transferred into a 96 well plate and stored at −80° C. until luciferase measurement. Lysates are thawed under sterile bench and immediately transferred to ice. PpLuc standards are prepared from a 100 ng/mL aliquot using 1× lysis buffer as diluent by 10-fold dilution down to 0.01 ng/mL. 20 μL each are transferred into a white LIA plate for lysates and PpLuc standards and measured in a TriStar2 S LB 942 plate reader with standard PpLuc protocol comprising injection of 50 μL beetlejuice+D-Luciferin and ATP/well, delay for 0.5 seconds and measurement for 2 seconds. Raw data are normalized to individual organ weights.

TABLE Ex-123
mRNAs of Example 9

| | | | | Cap0/ |
|---|---|---|---|---|
| Column | mRNA ID | Cap analog description | Compound ID | Cap1 |
| 1 | mRNA (i) | mCap | Compound 151 | Cap0 |
| 2 | mRNA (xvi) | CleanCap | Compound 152 | Cap1 |
| 3 | mRNA (xiv) | m⁷G-TriEG | Compound 124 | Cap1 |
| 4 | mRNA (xxi) | Diethylene glycol | Compound 140 | Cap1 |
| 5 | mRNA (vi) | 3′-SPhe-m⁷-Gancyclovir | Compound 104 | Cap1 |
| 6 | mRNA (viii) | m7-Ganciclovir | Compound 22 | Cap1 |
| 7 | mRNA (v) | 3′-OMe-m⁷-Ganciclovir | Compound 9b | Cap1 |
| 8 | mRNA (xii) | 3′-OMe-N7-4-ClBn- | Compound 116 | Cap1 |
| | | Ganciclovir | | |
| 9 | mRNA (xiii) | 3′-OMe-N7-BiPheMe- | Compound 117 | Cap1 |
| | | Ganciclovir | | |
| 10 | mRNA (xi) | m⁷-Penciclovir | Compound 112 | Cap1 |

1. A compound of formula (I): 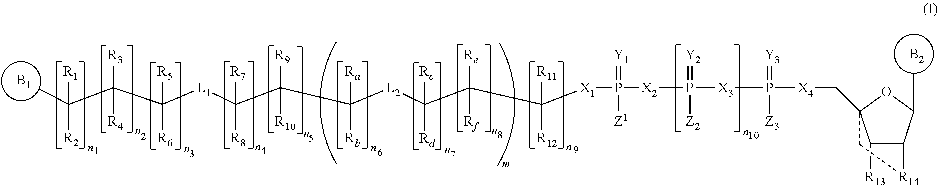
or a salt, stereoisomer, tautomer, or deuterated version thereof, wherein
R₁₃ is 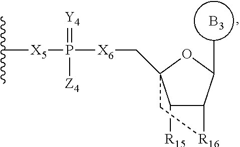
wherein
R₁₅ is OH or 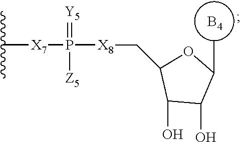
ring B₁ is guanine, a modified guanine or a guanine analog;
each of ring B₂ through ring B₄ is independently a nucleobase, a modified nucleobase or a nucleobase analog;
n₁ and n₉ are independently selected from an integer ranging from 1 to 10;
each of R₁ and R₂ is independently H, OH, SH, NH₂ or halogen, wherein, if n₁ is greater than 1, R₁ may be same or different in each repeating unit, and R₂ may be same or different in each repeating unit;
each of R₁₁ and R₁₂ is independently H, OH, SH, NH₂ or halogen, wherein, if n₉ is greater than 1, R₁₁ may be same or different in each repeating unit, and R₁₂ may be same or different in each repeating unit;
each of n₃, n₄, n₆, and n₇ is independently selected from an integer ranging from 0 to 5; wherein, if m is greater than 1, n₆ may be same or different in each repeating unit, and n₇ may be same or different in each repeating unit;
each of R₅ and R₆, if present, is independently H, OH, SH, NH₂ or halogen, wherein, if n₃ is greater than 1, R₅ may be same or different in each repeating unit, and R₆ may be same or different in each repeating unit;
each of R₇ and R₈, if present, is independently H, OH, SH, NH₂ or halogen, wherein, if n₄ is greater than 1, R₇ may be same or different in each repeating unit, and R₈ may be same or different in each repeating unit;
each of Rₐ and R_{b}, if present, is independently H, OH, SH, NH₂ or halogen, wherein, if n₆ is greater than 1, Rₐ may be same or different in each repeating unit, and R_{b} may be same or different in each repeating unit;
each of R_{c} and R_{d}, if present, is independently H, OH, SH, NH₂ or halogen, wherein, if n₇ is greater than 1, R_{d} may be same or different in each repeating unit, and R_{d} may be same or different in each repeating unit;
n₂, n₅ and n₈ are independently selected from 0 or 1; wherein, if m is greater than 1, n₈ may be same or different in each repeating unit;
one of R₃ and R₄, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of R₃ and R₄, if present, is selected from the group consisting of H, OH, SH, NH₂ and halogen;
one of R₉ and R₁₀, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of R₉ and R₁₀, if present, is selected from the group consisting of H, OH, SH, NH₂ and halogen;
one of Rₑ and R_{f}, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of Rₑ and R_{f}, if present, is selected from the group consisting of H, OH, SH, NH₂ and halogen;
m is selected from an integer ranging from 0 to 10, with the proviso that (i) if m is 0, one of n₂ and n₅ is 1 and the other one of n₂ and n₅ is 0, (ii) if m is 1, (a) n₂, n₅ and n₈ are 0 or (b) one of n₂, n₅ and n₈ is 1 and the other two of n₂, n₅ and n₈ are 0, and (iii) if m is an integer ranging from 2 to 10, (a) n₂, n₅ and each n₈ are 0, (b) one of n₂ and n₅ is 1 and the other one of n₂ and n₅ as well as each n₈ are 0, or (c) one n₈ is 1 and each other n₈ as well as n₂ and n₅ are 0;
n₁₀ is selected from 0, 1 or 2;
L₁ is selected from the group consisting of CH₂, O, S, SO, SO₂, NR^{L}, CH(OH), CH(SH) and CH(halogen), with the proviso that, if m is 1 and n₂, n₅ and n₈ are 0, L₁ is selected from the group consisting of O, S, SO, SO₂ and NR^{L};
L₂ is selected from the group consisting of O, S, SO, SO₂ and NH; wherein, if m is greater than 1, L₂ may be same or different in each repeating unit;
each of X₁ through X₈ is independently O, S, NH or CH₂, wherein, if n₁₀ is 2, X₃ may be same or different in each repeating unit;
each of Y₁ through Y₅ is independently O, S or Se, wherein, if n₁₀ is 2, Y₂ may be same or different in each repeating unit;
each of Z₁ through Z₅ is independently OH, SH, SeH or BH₃, wherein, if n₁₀ is 2, Z₂ may be same or different in each repeating unit;
R₁₄ is (i) selected from the group consisting of H, OH, OC₁-C₃-alkyl, and Opropargyl, wherein the dashed methylene bridge between R₁₄ and the 4′ C is absent, or (ii) O, wherein the dashed methylene bridge between R₁₄ being O and the 4′ C is present;
R₁₆ is (i) selected from the group consisting of H, OH, OC₁-C₃-alkyl, and Opropargyl, wherein the dashed methylene bridge between R₁ and the 4′ C is absent, or (ii) O, wherein the dashed methylene bridge between R₁₅ being O and the 4′ C is present;
R^{L} is (i) H, if one of n₂, n₅ and n₈ is 1, or (ii) selected from the group consisting of H, C₁-C₃-alkyl, C₁-C₃-alkyl-O—R^{O}, C₁-C₃-alkyl-S—R^{S}, C₁-C₃-alkyl-NR^{N1}R^{N2}, C₁-C₃-alkyl-R^{P} and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized, and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, if n₂, n₅ and n₈ are 0;
R^{O}, R^{S}, R^{N1} and R^{N2} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{D} and R^{E} are each independently selected from the group consisting of C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{N3} and R^{N4} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl- O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, with the proviso that R^{N3} and R^{N4} are not both H; and
R^{P} is 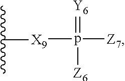
wherein
X₉ is O, S, NH or CH₂;
Y₆ is O, S or Se; and
each of Z₆ and Z₇ is independently OH, SH, SeH or BH₃.

2. The compound according to claim 1, wherein m is selected from 0 or 1.

3. The compound according to claim 1 or 2, wherein m is 0, one of n₂ and n₅ is 1 and the other one of n₂ and n₅ is 0.

4. The compound according to claim 1 or 2, wherein m is 1 and one of n₂, n₅ and n₈ is 1 and the other two of n₂, n₅ and n₈ are 0.

5. The compound according to any one of claims 1 to 4, wherein n₂ is 1.

6. The compound according to any one of claims 1 to 4, wherein n₅ is 1.

7. The compound according to claim 1 or 4, wherein n₅ is 1.

8. The compound according to any one of the preceding claims, wherein each of n₁ and n₉ is independently selected from 1 or 2, preferably wherein n₁ and n₉ are 1.

9. The compound according to any one of the preceding claims, wherein each of n₃, n₄, and, if present, each of n₆ and n₇ is independently selected from 0, 1, or 2, preferably from 0 or 1.

10. The compound according to claim 9, wherein n₃, n₄, and if present, n₆ and n₇ are 0.

11. The compound according to claim 9, wherein n₃, n₄, and if present, n₆ and n₇ are 1.

12. The compound according to any one of the preceding claims, wherein each of R₁, R₂, R₅, R₆, R₇, R₈, Rₐ, R_{b}, R_{c}, R_{d}, R₁₁, and R₁₂, if present, is the same in each repeating unit.

13. The compound according to any one of the preceding claims, wherein, if present, one of R₁ and R₂, one of R₅ and R₆, one of R₇ and R₈, one of Rₐ and R_{b}, one of R_{c} and R_{d}, and one of R₁₁ and R₁₂ is H.

14. The compound according to any one of the preceding claims, wherein each of R₁, R₂, R₅, R₆, R₇, R₈, Rₐ, R_{b}, R_{c}, R_{d}, R₁₁, and R₁₂, if present, is H.

15. The compound according to any one of the preceding claims, wherein
if n₂ is 1, one of R₃ and R₄ is selected from the group consisting of C₁-alkyl-O—R^{O}, C₁-alkyl-S—R^{S}, C₁-alkyl-S—S—R^{S}, C₁-alkyl-R^{P}, O—R_{c}, R^{P}, and 5- or 6-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl; and the other one of R₃ and R₄ is H;
if n₅ is 1, one of R₉ and R₁₀ is selected from the group consisting of C₁-alkyl-O—R^{O}, C₁-alkyl-S—R^{S}, C₁-alkyl-S—S—R^{S}, C₁-alkyl-R^{P}, O—R^{O}, R^{P}, and 5- or 6-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl; and the other one of R₉ and R₁₀ is H; and
if n₅ is 1, one of Rₑ and R_{f} is selected from the group consisting of C₁-alkyl-O—R^{O}, C₁-alkyl-S—R^{S}, C₁-alkyl-S—S—R^{S}, C₁-alkyl-R^{P}, O—R^{O}, R^{P}, and 5- or 6-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl; and the other one of R₃ and R₄ is H.

16. The compound according to any one of the preceding claims, wherein
if n₂ is 1, one of R₃ and R₄ is selected from the group consisting of C₁-alkyl-O—R^{O}, C₁-alkyl-S—R^{S}, C₁-alkyl-S—S—R^{S}, C₁-alkyl-R^{P}, O—R^{D}, R^{P}; and the other one of R₃ and R₄ is H;
if n₅ is 1, one of R₉ and R₁₀ is selected from the group consisting of C₁-alkyl-O—R^{O}, C₁-alkyl-S—R^{S}, C₁-alkyl-S—S—R^{S}, C₁-alkyl-R^{P}, O—R^{O}, R^{P}; and the other one of R₉ and R₁₀ is H; and
if n₈ is 1, one of Rₑ and R_{f} is selected from the group consisting of C₁-alkyl-O—R^{O}, C₁-alkyl-S—R^{S}, C₁-alkyl-S—S—R^{S}, C₁-alkyl-R^{P}, O—R^{D}, R^{P}; and the other one of R₃ and R₄ is H.

17. The compound according to any one of claims 1 to 16, wherein
if n₂ is 1, one of R₃ and R₄ is C₁-alkyl-O—R^{O}, and the other one of R₃ and R₄ is H;
if n₅ is 1, one of R₉ and R₁₀ is C₁-alkyl-O—R^{O}, and the other one of R₉ and R₁₀ is H; and
if n₅ is 1, one of Rₑ and R_{f} is C₁-alkyl-O—R^{O}, and the other one of Rₑ and R_{f} is H.

18. The compound according to any one of claims 1 to 16, wherein
if n₂ is 1, one of R₃ and R₄ is C₁-alkyl-S—R^{S} or C₁-alkyl-S—S—R^{S}, preferably C₁-alkyl-S—R^{S}, and the other one of R₃ and R₄ is H;
if n₅ is 1, one of R₉ and R₁₀ is C₁-alkyl-S—R^{S} or C₁-alkyl-S—S—R^{S}, preferably C₁-alkyl-S—R^{S}, and the other one of R₉ and R₁₀ is H; and
if n₄ is 1, one of Rₑ and R_{f} is C₁-alkyl-S—R^{S} or C₁-alkyl-S—S—R^{S}, preferably C₁-alkyl-S—R^{S}, and the other one of Rₑ and R_{f} is H.

19. The compound according to any one of claims 1 to 16, wherein
if n₂ is 1, one of R₃ and R₄ is C₁-alkyl-R^{P} or R^{P} and the other one of R₃ and R₄ is H;
if n₅ is 1, one of R₉ and R₁₀ is C₁-alkyl-R^{P} or R^{P} and the other one of R₉ and R₁₀ is H; and
if n₄ is 1, one of Rₑ and R_{f} is C₁-alkyl-R^{P} or R^{P} and the other one of Rₑ and R_{f} is H;
wherein R^{P} is preferably 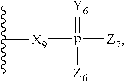
wherein
X₉ is O; Y₆ is O or S; and each of Z₆ and Z₇ is OH.

20. The compound according to any one of the preceding claims, wherein
R^{O}, R^{S}, R^{N1} and R^{N2}, if present, are each independently selected from the group consisting of H, C₁-C₄-alkyl, and 5- or 6-membered saturated, partially or fully unsaturated, or aromatic carbocyclyl, carbocyclyl-C₁-C₂-alkyl, heterocyclyl, or heterocyclyl-C₁-C₂-alkyl, wherein the aforementioned heterocyclic rings comprise one or two, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one substituent R^{X}, wherein R^{X} is halogen, CN, OH, SH, NH₂, or C₁-C₂-alkyl; and
R^{D} and R^{E}, if present, are each independently selected from the group consisting of C₁-C₄-alkyl, and 5- or 6-membered saturated, partially or fully unsaturated, or aromatic carbocyclyl, carbocyclyl-C₁-C₂-alkyl, heterocyclyl, or heterocyclyl-C₁-C₂-alkyl, wherein the aforementioned heterocyclic rings comprise one or two, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one substituent R^{X}, wherein R^{X} is halogen, CN, OH, SH, NH₂, or C₁-C₂-alkyl.

21. The compound according to any one of the preceding claims, wherein
R^{O}, R^{S}, R^{N1} and R^{N2}, if present, are each independently selected from the group consisting of H, C₁-C₃-alkyl, C₁-C₂-cyanoalkyl, and 6-membered saturated, partially or fully unsaturated, or aromatic carbocyclyl, carbocyclyl-C₁-alkyl, heterocyclyl, or heterocyclyl-C₁-alkyl, wherein the aforementioned heterocyclic rings comprise one or two heteroatoms selected from O, N, or S, wherein said N- and/or S-atoms are independently oxidized or non-oxidized;
and wherein each substitutable carbon or heteroatom in the aforementioned groups is independently unsubstituted or substituted with one substituent R^{X}, wherein R^{X} is halogen; and
R^{D} and R^{E}, if present, are each independently C₁-C₂-alkyl, preferably CH₃.

22. The compound according to claim 1, wherein m is an integer ranging from 2 to 10 and n₂, n₅ and each n₈ are 0.

23. The compound according to claim 1, wherein m is 1 and n₂, n₅ and n₈ are 0.

24. The compound according to claim 22 or 23, wherein each of n₁, n₃, n₄, n₆, nm and n₉ is 1, and wherein each of R₁, R₂, R₅, R₆, R₇, R₈, Rₐ, R_{b}, R_{c}, R_{d}, R₁₁ and R₁₂ is H.

25. The compound according to any one of the preceding claims, wherein L₁ is selected from the group consisting of CH₂, O, S, SO, SO₂, NH, CH(OH), CH(SH) and CH(halogen), with the proviso that, if m is 1 and n₂, n₅ and n₈ are O, L₁ is selected from the group consisting of O, S, SO, SO₂ and NH.

26. The compound according to any one of the preceding claims, wherein L₁ is selected from the group consisting of CH₂, O, CH(OH), CH(SH) and CH(halogen), preferably consisting of CH₂ and O, with the proviso that, if m is 1 and n₂, n₅ and n₈ are O, L₁ is O or S, preferably O.

27. The compound according to any one of the preceding claims, wherein L₂, if present, is O.

28. The compound according to any one of the preceding claims, wherein
R₁₄ is selected from the group consisting of H, OH, OC₁-C₃-alkyl, and Opropargyl, wherein the dashed methylene bridge between R₁₄ and the 4′ C is absent; and
R₁₆ is selected from the group consisting of H, OH, OC₁-C₃-alkyl, and Opropargyl, wherein the dashed methylene bridge between R₁₄ and the 4′ C is absent.

29. The compound according to any one of the preceding claims, wherein B₂ is selected from the group consisting of guanine, a modified guanine, a guanine analog, adenine, a modified adenine, and an adenine analog.

30. The compound according to any one of the preceding claims, wherein B₃ is guanine, a modified guanine or a guanine analog.

31. The compound according to any one claims 1 to 30, wherein R₁₃ is 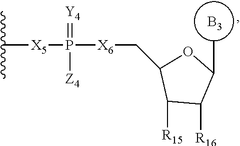
wherein R₁₅ and R₁₆ are each OH.

32. The compound according to claim 31, wherein R₁₄ is H or OC₁-C₃-alkyl, preferably OCH₃, wherein the dashed methylene bridge between R₁₄ and the 4′ C is absent.

33. The compound according to any one of claim 1 to 30, wherein R₁₃ is 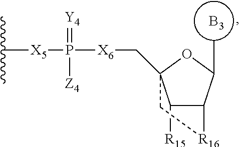
wherein R₁₅ is

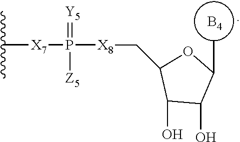

34. The compound according to claim 33, wherein
R₁₄ is H or OC₁-C₃-alkyl, preferably OCH₃, wherein the dashed methylene bridge between R₁₄ and the 4′ C is absent; and/or
R₁₆ is H or OC₁-C₃-alkyl, preferably OCH₃, wherein the dashed methylene bridge between R₁₆ and the 4′ C is absent.

35. The compound according to claim 33, wherein
R₁₄ is OCH₃, wherein the dashed methylene bridge between R₁₄ and the 4′ C is absent; and
R₁₆ is OCH₃, wherein the dashed methylene bridge between R₁₆ and the 4′ C is absent.

36. The compound according to claim 33, wherein
R₁₄ is OH, wherein the dashed methylene bridge between R₁₄ and the 4′ C is absent; and
R₁₆ is OCH₃, wherein the dashed methylene bridge between R₁₆ and the 4′ C is absent.

37. The compound according to any one of the preceding claims, wherein ring B₁ is a modified guanine, preferably selected from the group consisting of N⁷-methylguanine, N⁷-4-chloro-benzyl-guanine, N⁷-biphenyl-methylene-guanine, N⁷-naphtyl-methylene-guanine and N⁷-3,5-dimethyl-benzyl-guanine.

38. The compound according to any one of the preceding claims, wherein ring B₁ is N⁷-methylguanine.

39. The compound according to any one of the preceding claims, wherein n₁₀ is 1.

40. The compound according to any one of the preceding claims, wherein X₁ is CH₂ or O, and each of X₂ through X₈ is O.

41. The compound according to any one of claims 1 to 40, wherein X₁ is O.

42. The compound according to any one of claims 1 to 40, wherein X₁ is CH₂.

43. The compound according to any one of the preceding claims, wherein each of Y₁ through Y₅ is O; or one of Y₁ through Y₅, preferably Y₁, is S and each of the remaining ones of Y₁ through Y₅ is O.

44. The compound according to any one of the preceding claims, wherein each of Z₁ through Z₅ is OH.

45. A cap analog comprising a 5′ terminal acyclonucleoside, wherein the acyclonucleoside comprises a linear unbranched or linear single-branched structure instead of a ribose, wherein the cap analog is preferably a Cap1 analog or a Cap2 analog or a Cap2-1 analog, wherein the 5′ terminal acyclonucleoside is optionally deuterated, and wherein the linear unbranched or linear single-branched structure has the structure of formula (II): 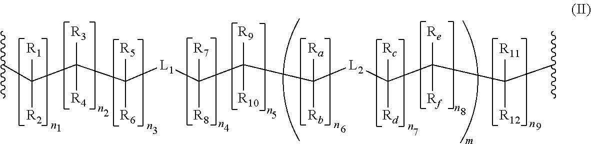
wherein
n₁ and n₉ are independently selected from an integer ranging from 1 to 10;
each of R₁ and R₂ is independently H, OH, SH, NH₂ or halogen, wherein, if n₁ is greater than 1, R₁ may be same or different in each repeating unit, and R₂ may be same or different in each repeating unit;
each of R₁₁ and R₁₂ is independently H, OH, SH, NH₂ or halogen, wherein, if n₉ is greater than 1, R₁₁ may be same or different in each repeating unit, and R₁₂ may be same or different in each repeating unit;
each of n₃, n₄, n₆, and n₇ is independently selected from an integer ranging from 0 to 5; wherein, if m is greater than 1, ne may be same or different in each repeating unit, and n₇ may be same or different in each repeating unit;
each of R₅ and R₆, if present, is independently H, OH, SH, NH₂ or halogen, wherein, if n₃ is greater than 1, R₅ may be same or different in each repeating unit, and R₅ may be same or different in each repeating unit;
each of R₇ and R₈, if present, is independently H, OH, SH, NH₂ or halogen, wherein, if n₄ is greater than 1, R₇ may be same or different in each repeating unit, and R₈ may be same or different in each repeating unit;
each of Rₐ and R_{b}, if present, is independently H, OH, SH, NH₂ or halogen, wherein, if n₆ is greater than 1, Rₐ may be same or different in each repeating unit, and R_{b} may be same or different in each repeating unit;
each of R_{c} and R_{d}, if present, is independently H, OH, SH, NH₂ or halogen, wherein, if n₇ is greater than 1, R_{c} may be same or different in each repeating unit, and R_{d} may be same or different in each repeating unit;
n₂, n₅ and n₈ are independently selected from 0 or 1; wherein, if m is greater than 1, n₈ may be same or different in each repeating unit;
one of R₃ and R₄, if present, is selected from the group consisting of C₁-C₄-alkyl, C₁₀C₄-alkenyl, C₁₀C₄-alkinyl, C₁₀C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of R₃ and R₄, if present, is selected from the group consisting of H, OH, SH, NH₂ and halogen;
one of R₉ and R₁₀, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of R₉ and R₁₀, if present, is selected from the group consisting of H, OH, SH, NH₂ and halogen;
one of Rₑ and R_{f}, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of Rₑ and R_{f}, if present, is selected from the group consisting of H, OH, SH, NH₂ and halogen;
m is selected from an integer ranging from 0 to 10, with the proviso that (i) if m is 0, one of n₂ and n₅ is 1 and the other one of n₂ and n₅ is 0, (ii) if m is 1, (a) n₂, n₅ and n₈ are 0 or (b) one of n₂, n₅ and n₈ is 1 and the other two of n₂, n₅ and n₈ are 0; and (iii) if m is an integer ranging from 2 to 10, (a) n₂, n₅ and each no are 0, (b) one of n₂ and n₅ is 1 and the other one of n₂ and n₅ as well as each n₈ are 0, or (c) one n₈ is 1 and each other n₈ as well as n₂ and n₅ are 0;
L₁ is selected from the group consisting of CH₂, O, S, SO, SO₂, NR^{L}, CH(OH), CH(SH) and CH(halogen), with the proviso that, if m is 1 and n₂, n₅ and n₈ are 0, L₁ is selected from the group consisting of O, S, SO, SO₂ and NR^{L};
L₂ is selected from the group consisting of O, S, SO, SO₂ and NH; wherein, if m is greater than 1, L₂ may be same or different in each repeating unit;
R^{L} is (i) H, if one of n₂, n₅ and n₈ is 1, or (ii) selected from the group consisting of H, C₁-C₃-alkyl, C₁-C₃-alkyl-O—R^{O}, C₁-C₃-alkyl-S—R^{S}, C₁-C₃-alkyl-NR^{N1}R^{N2}, C₁-C₃-alkyl-R^{P} and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized, and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, if n₂, n₅ and n₈ are 0,
R^{O}, R^{S}, R^{N1} and R^{N2} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{D} and R^{E} are each independently selected from the group consisting of C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{N3} and R^{N4} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl- O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, with the proviso that R^{N3} and R^{N4} are not both H;
R^{P} is 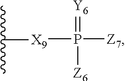
, wherein
X₉ is O, S, NH or CH₂;
Y₆ is O, S or Se; and
each of Z₆ and Z₇ is independently OH, SH, SeH or BH₃;
and wherein the linear unbranched or linear single-branched structure is optionally deuterated.

46. The cap analog according to claim 45, wherein m is selected from 0 and 1.

47. The cap analog according to claim 45, wherein the acyclonucleoside comprises a linear unbranched structure of formula (II), wherein m is selected from an integer ranging from 1 to 10, preferably 1, n₂, n₅ and each n₈ are 0, and L₁ is selected from the group consisting of O, S, SO, SO₂ and NR^{L}, preferably L₁ is O.

48. The cap analog according to claim 45, wherein the acyclonucleoside comprises a linear single-branched structure of formula (II), wherein
(i) m is 0, one of n₂ and n₅ is 1, and the other one of n₂ and n₅ is 0; (ii) m is 1, one of n₂, n₅ and n₈ is 1, and the other two of n₂, n₅ and n₈ are 0; (iii) m is an integer ranging from 2 to 10, one of n₂ and n₅ is 1 and the other one of n₂ and n₅ as well as each n₈ are 0, or (iv) m is an integer ranging from 2 to 10, one n₈ is 1 and each other n₈ as well as n₂ and n₅ are 0; and
L₁ is selected from the group consisting of CH₂, O, S, SO, SO₂, NR^{L}, CH(OH), CH(SH) and CH(halogen), preferably L₁ is CH₂ or 0.

49. The cap analog according to claim 48, wherein the acyclonucleoside comprises a linear single-branched structure of formula (II), wherein m is 0, one of n₂ and n₅ is 1, and the other one of n₂ and n₅ is 0.

50. The cap analog according to any one of claims 45 to 49, wherein
each of n₁ and n₉ is independently selected from 1 or 2, preferably n, is 1 and n₉ is 1;
each of n₃, n₄, and, if present, each of n₆ and n₇, is independently selected from 0 or 1.

51. The cap analog according to any one of claims 45 to 50, wherein
one of R₃ and R₄ if present, one of R₉ and R₁₀ if present, or one of Rₑ and R_{f} if present, is selected from the group consisting of C₁-alkyl-O—R^{O}, C₁-alkyl-S—R^{S}, C₁-alkyl-S—S—R^{S}, C₁-alkyl-R^{P};
wherein
R^{O} and R^{S} are each independently selected from the group consisting of H, C₁-C₄-alkyl, C₁-C₂-cyanoalkyl, and 5- or 6-membered saturated, partially or fully unsaturated, or aromatic carbocyclyl, carbocyclyl-C₁-alkyl, heterocyclyl, or heterocyclyl-C₁-alkyl, wherein the aforementioned heterocyclic rings comprise one or two, same or different heteroatoms selected from O, N, or S, wherein said N- and/or S-atoms are independently oxidized or non-oxidized; and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₂-alkyl, preferably halogen;
R^{P} is 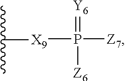
wherein
X₉ is O; Y₆ is O or S; and each of Z₆ and Z₇ is OH; and
the other one of R₃ and R₄ if present, R₉ and R₁₀ if present, Rₑ and R_{f} if present, is H; and
wherein preferably each of R₁, R₂, R₅, R₆, R₇, R₈, Rₐ, R_{b}, R_{c}, R_{d}, R₁₁, and R₁₂, if present, is H.

52. The cap analog according to any one of claims 45 to 51, wherein the acyclonucleoside comprises as the nucleobase guanine, a modified guanine or a guanine analog.

53. An RNA molecule comprising at least three nucleotides and comprising a 5′ end of formula (III) 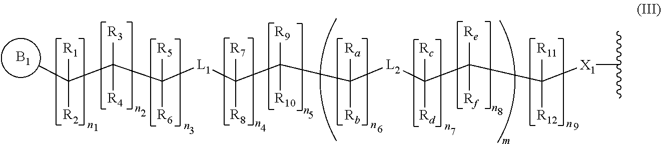
wherein
ring B₁ is guanine, a modified guanine or a guanine analog;
n₁ and n₉ are independently selected from an integer ranging from 1 to 10;
each of R₁ and R₂ is independently H, OH, SH, NH₂ or halogen, wherein, if n₁ is greater than 1, R₁ may be same or different in each repeating unit, and R₂ may be same or different in each repeating unit;
each of R₁₁ and R₁₂ is independently H, OH, SH, NH₂ or halogen, wherein, if n₉ is greater than 1, R₁₁ may be same or different in each repeating unit, and R₁₂ may be same or different in each repeating unit;
each of n₃, n₄, n₆, and n₇ is independently selected from an integer ranging from 0 to 5; wherein, if m is greater than 1, n₆ may be same or different in each repeating unit, and n₇ may be same or different in each repeating unit;
each of R₅ and R₆, if present, is independently H, OH, SH, NH₂ or halogen, wherein, if n₃ is greater than 1, R₅ may be same or different in each repeating unit, and R₆ may be same or different in each repeating unit;
each of R₇ and R₈, if present, is independently H, OH, SH, NH₂ or halogen, wherein, if n₄ is greater than 1, R₇ may be same or different in each repeating unit, and R₈ may be same or different in each repeating unit;
each of Rₐ and R_{b}, if present, is independently H, OH, SH, NH₂ or halogen, wherein, if no is greater than 1, Rₐ may be same or different in each repeating unit, and R_{b} may be same or different in each repeating unit;
each of R_{c} and R_{d}, if present, is independently H, OH, SH, NH₂ or halogen, wherein, if n₇ is greater than 1, R_{c} may be same or different in each repeating unit, and R_{d} may be same or different in each repeating unit;
n₂, n₅ and n₈ are independently selected from 0 or 1; wherein, if m is greater than 1, n₈ may be same or different in each repeating unit;
one of R₃ and R₄, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of R₃ and R₄, if present, is selected from the group consisting of H, OH, SH, NH₂ and halogen;
one of R₉ and R₁₀, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of R₉ and R₁₀, if present, is selected from the group consisting of H, OH, SH, NH₂ and halogen;
one of Rₑ and R_{f}, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of Rₑ and R_{f}, if present, is selected from the group consisting of H, OH, SH, NH₂ and halogen;
m is selected from an integer ranging from 0 to 10, with the proviso that (i) if m is 0, one of n₂ and n₅ is 1 and the other one of n₂ and n₅ is 0, (ii) if m is 1, (a) n₂, n₅ and n₈ are 0 or (b) one of n₂, n₅ and n₈ is 1 and the other two of n₂, n₅ and n₈ are 0, and (iii) if m is an integer ranging from 2 to 10, (a) n₂, n₅ and each n₈ are 0, (b) one of n₂ and n₅ is 1 and the other one of n₂ and n₅ as well as each n₈ are 0, or (c) one n₅ is 1 and each other n₈ as well as n₂ and n₅ are 0;
L₁ is selected from the group consisting of CH₂, O, S, SO, SO₂, NR^{L}, CH(OH), CH(SH) and CH(halogen), with the proviso that, if m is 1 and n₂, n₅ and n₈ are 0, L₁ is selected from the group consisting of O, S, SO, SO₂ and NR^{L};
L₂ is selected from the group consisting of O, S, SO, SO₂ and NH, wherein, if m is greater than 1, L₂ may be same or different in each repeating unit;
X₁ is O, S, NH or CH₂;
R^{L} is (i) H, if one of n₂, n₅ and n₈ is 1, or (ii) selected from the group consisting of H, C₁-C₃-alkyl, C₁-C₃-alkyl-O—R^{O}, C₁-C₃-alkyl-S—R^{S}, C₁-C₃-alkyl-NR^{N1}R^{N2}, C₁-C₃-alkyl-R^{P} and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized, and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, if n₂, n₅ and n₈ are 0;
R^{O}, R^{S}, R^{N1} and R^{N2} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{D} and R^{E} are each independently selected from the group consisting of C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{N3} and R^{N4} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl- O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, with the proviso that R^{N3} and R^{N4} are not both H;
R^{P} is 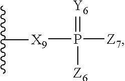
wherein
X₉ is O, S, NH or CH₂;
Y₆ is O, S or Se, and
each of Z₆ and Z₇ is independently OH, SH, SeH or BH₃;
and wherein the 5′ end is optionally deuterated.

54. The RNA molecule according to claim 53, wherein m is selected from 0 or 1.

55. The RNA molecule according to claim 53, wherein m is selected from an integer ranging from 1 to 10, preferably 1, n₂, n₅ and each n₈ are 0, and L₁ is selected from the group consisting of O, S, SO, SO₂ and NR^{L}, preferably L₁ is 0.

56. The RNA molecule according to claim 53, wherein
(i) m is 0, one of n₂ and n₅ is 1, and the other one of n₂ and n₅ is 0; (ii) m is 1, one of n₂, n₅ and n₈ is 1, and the other two of n₂, n₅ and n₈ are 0, (iii) m is an integer ranging from 2 to 10, one of n₂ and n₅ is 1 and the other one of n₂ and n₅ as well as each n₈ are 0, or (iv) m is an integer ranging from 2 to 10, one n₅ is 1 and each other n₈ as well as n₂ and n₅ are 0; and
L₁ is selected from the group consisting of CH₂, O, S, SO, SO₂, NR^{L}, CH(OH), CH(SH) and CH(halogen), preferably L₁ is CH₂ or 0.

57. The RNA molecule according to claim 56, wherein m is 0, one of n₂ and n₅ is 1, and the other one of n₂ and n₅ is 0.

58. The RNA molecule according to any one of claims 53 to 57, wherein ring B₁ is a modified guanine, preferably N⁷-methylguanine.

59. The RNA molecule according to any one of claims 53 to 58, wherein
each of n₁ and no is independently selected from 1 or 2, preferably n₁ is 1 and no is 1;
each of n₃, n₄, and, if present, each of n₆ and n₇, is independently selected from 0 or 1.

60. The RNA molecule according to any one of claims 53 to 59, wherein
one of R₃ and R₄ if present, one of R₉ and R₁₀ if present, or one of Rₑ and R_{f} if present, is selected from the group consisting of C₁-alkyl-O—R^{O}, C₁-alkyl-S—R^{S}, C₁-alkyl-S—S—R^{S}, C₁-alkyl-R^{P};
wherein
R^{O} and R^{S} are each independently selected from the group consisting of H, C₁-C₄-alkyl, C₁-C₂-cyanoalkyl, and 5- or 6-membered saturated, partially or fully unsaturated, or aromatic carbocyclyl, carbocyclyl-C₁-alkyl, heterocyclyl, or heterocyclyl-C₁-alkyl, wherein the aforementioned heterocyclic rings comprise one or two, same or different heteroatoms selected from O, N, or S, wherein said N- and/or S-atoms are independently oxidized or non-oxidized; and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₂-alkyl, preferably halogen;
R^{P} is 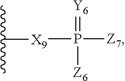
wherein
X₉ is O; Y₆ is O or S; and each of Z₆ and Z₇ is OH; and
the other one of R₃ and R₄ if present, R₉ and R₁₀ if present, Rₑ and R_{f} if present, is H; and
wherein preferably each of R₁, R₂, R₅, R₆, R₇, R₈, Rₐ, R_{b}, R_{c}, R_{d}, R₁₁, and R₁₂, if present, is H.

61. The RNA molecule according to any one of claims 53 to 60, wherein the RNA molecule comprises a 5′ end of formula (I): 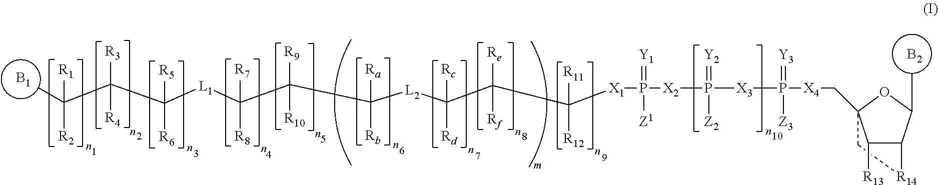
wherein
R₁₃ is 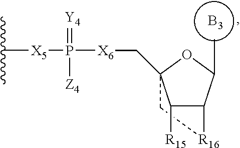
wherein
R₁₅ is 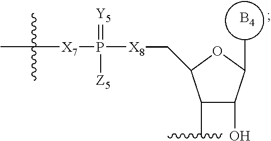
and wherein
n₁₀ is selected from 0, 1 or 2;
each of X₂ through X₃ is independently O, S, NH or CH₂, wherein, if n₁₀ is 2, X₃ may be same or different in each repeating unit;
each of Y₁ through Y₅ is independently O, S or Se, wherein, if n₁₀ is 2, Y₂ may be same or different in each repeating unit;
each of Z₁ through Z₅ is independently OH, SH, SeH or BH₃, wherein, if n₁₀ is 2, Z₂ may be same or different in each repeating unit;
R₁₄ is (i) selected from the group consisting of H, OH, OC₁-C₃-alkyl, and Opropargyl, wherein the dashed methylene bridge between R₁₄ and the 4′ C is absent, or (ii) O, wherein the dashed methylene bridge between R₁₄ being O and the 4′ C is present;
R₁₆ is (i) selected from the group consisting of H, OH, OC₁-C₃-alkyl, and Opropargyl, wherein the dashed methylene bridge between R₁₆ and the 4′ C is absent, or (ii) O, wherein the dashed methylene bridge between R₁₆ being O and the 4′ C is present;
each of ring B₂ through ring B₄ is independently a nucleobase, a modified nucleobase, or a nucleobase analog.

62. The RNA molecule according to claim 61,
wherein R₁₄ is OC₁-C₃-alkyl, preferably wherein R₁₄ is OCH₃, wherein the dashed methylene bridge between R₁₄ and the 4′ C is absent; and/or
wherein R₁₆ is OC₁-C₃-alkyl, preferably wherein R₁₆ is OCH₃, wherein the dashed methylene bridge between R₁₆ and the 4′ C is absent.

63. The RNA molecular according to claim 61 or 62, wherein
n₁₀ is 1;
each of X₂ through X₈ is O;
each of Y₁ through Y₅ is O; or one of Y₁ through Y₅, preferably Y₁, is S and each of the remaining ones of
Y₁ through Y₅ is O;
each of Z₁ through Z₅ is OH; and
each of ring B₂ through ring B₄ is a nucleobase.

64. An RNA molecule comprising at least three nucleotides and comprising a 5′ terminal acyclonucleoside, wherein the acyclonucleoside comprises a linear unbranched or linear single-branched structure instead of a ribose, wherein the 5′ terminal acyclonucleoside is optionally deuterated, and wherein the linear unbranched or linear single-branched structure has the structure of formula (II): 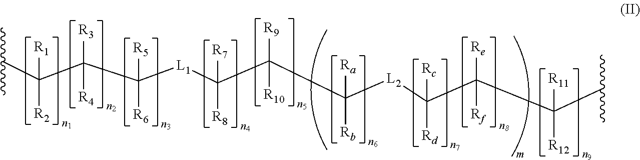
wherein
n₁ and ne are independently selected from an integer ranging from 1 to 10;
each of R₁ and R₂ is independently H, OH, SH, NH₂ or halogen, wherein, if n₁ is greater than 1, R₁ may be same or different in each repeating unit, and R₂ may be same or different in each repeating unit;
each of R₁₁ and R₁₂ is independently H, OH, SH, NH₂ or halogen, wherein, if n₆ is greater than 1, R₁₁ may be same or different in each repeating unit, and R₁₂ may be same or different in each repeating unit;
each of n₃, n₄, n₆, and n₇ is independently selected from an integer ranging from 0 to 5; wherein, if m is greater than 1, n₆ may be same or different in each repeating unit, and n₇ may be same or different in each repeating unit;
each of R₅ and R₆, if present, is independently H, OH, SH, NH₂ or halogen, wherein, if n₃ is greater than 1, R₅ may be same or different in each repeating unit, and R₅ may be same or different in each repeating unit;
each of R₇ and R₈, if present, is independently H, OH, SH, NH₂ or halogen, wherein, if n₄ is greater than 1, R₇ may be same or different in each repeating unit, and R₈ may be same or different in each repeating unit;
each of Rₐ and R_{b}, if present, is independently H, OH, SH, NH₂ or halogen, wherein, if n₆ is greater than 1, Rₐ may be same or different in each repeating unit, and R_{b} may be same or different in each repeating unit;
each of R_{c} and R_{d}, if present, is independently H, OH, SH, NH₂ or halogen, wherein, if n₇ is greater than 1, R_{c} may be same or different in each repeating unit, and R_{d} may be same or different in each repeating unit;
n₂, n₅ and n₈ are independently selected from 0 or 1; wherein, if m is greater than 1, ne may be same or different in each repeating unit;
one of R₃ and R₄, if present, is selected from the group consisting of C₁-C₄-alkyl, C₁₀C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{O}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of R₃ and R₄, if present, is selected from the group consisting of H, OH, SH, NH₂ and halogen;
one of R₉ and R₁₀, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of R₉ and R₁₀, if present, is selected from the group consisting of H, OH, SH, NH₂ and halogen;
one of Rₑ and R_{f}, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of Rₑ and R_{f}, if present, is selected from the group consisting of H, OH, SH, NH₂ and halogen;
m is selected from an integer ranging from 0 to 10, with the proviso that (i) if m is 0, one of n₂ and n₅ is 1 and the other one of n₂ and n₅ is 0, (ii) if m is 1, (a) n₂, n₅ and n₈ are 0 or (b) one of n₂, n₅ and n₃ is 1 and the other two of n₂, n₅ and n₈ are 0; and (iii) if m is an integer ranging from 2 to 10, (a) n₂, n₅ and each n₈ are 0, (b) one of n₂ and n₅ is 1 and the other one of n₂ and n₅ as well as each n₈ are 0, or (c) one n₅ is 1 and each other n₈ as well as n₂ and n₅ are 0;
L₁ is selected from the group consisting of CH₂, O, S, SO, SO₂, NR^{L}, CH(OH), CH(SH) and CH(halogen), with the proviso that, if m is 1 and n₂, n₅ and n₈ are 0, L₁ is selected from the group consisting of O, S, SO, SO₂ and NR^{L};
L₂ is selected from the group consisting of O, S, SO, SO₂ and NH, wherein, if m is greater than 1, L₂ may be same or different in each repeating unit;
R^{L} is (i) H, if one of n₂, n₅ and n₈ is 1, or (ii) selected from the group consisting of H, C₁-C₃-alkyl, C₁-C₃-alkyl-O—R^{O}, C₁-C₃-alkyl-S—R^{S}, C₁-C₃-alkyl-NR^{N1}N², C₁-C₃-alkyl-R^{P} and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized, and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, if n₂, n₅ and n₈ are 0;
R^{O}, R^{S}, R^{N1} and R^{N2} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{D} and R^{E} are each independently selected from the group consisting of C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{N3} and R^{N4} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl- O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, with the proviso that R^{N3} and R^{N4} are not both H;
R^{P} is 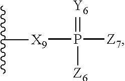
wherein
X₉ is O, S, NH or CH₂;
Y₆ is O, S or Se; and
each of Z₆ and Z₇ is independently OH, SH, SeH or BH₃;
and wherein the linear unbranched or linear single-branched structure is optionally deuterated.

65. The RNA molecule according to claim 64, wherein m is selected from 0 or 1.

66. The RNA molecule according to claim 64, wherein the acyclonucleoside comprises a linear unbranched structure of formula (II), wherein m is is selected from an integer ranging from 1 to 10, preferably 1, n₂, n₅ and each n₈ are 0, and L₁ is selected from the group consisting of O, S, SO, SO₂ and NR^{L}, preferably L₁ is O.

67. The RNA molecule according to claim 64, wherein the acyclonucleoside comprises a linear single-branched structure of formula (II), wherein
(i) m is 0, one of n₂ and n₅ is 1, and the other one of n₂ and n₅ is 0; (ii) m is 1, one of n₂, n₅ and n₈ is 1, and the other two of n₂, n₅ and n₈ are 0; (iii) m is an integer ranging from 2 to 10, one of n₂ and n₅ is 1 and the other one of n₂ and n₅ as well as each n₈ are 0, or (iv) m is an integer ranging from 2 to 10, one n₅ is 1 and each other n₈ as well as n₂ and n₅ are 0; and
L₁ is selected from the group consisting of CH₂, O, S, SO, SO₂, NR^{L}, CH(OH), CH(SH) and CH(halogen), preferably L₁ is CH₂ or O.

68. The RNA molecule according to claim 67, wherein the acyclonucleoside comprises a linear single-branched structure of formula (II), wherein m is 0, one of n₂ and n₅ is 1, and the other one of n₂ and n₅ is 0.

69. The RNA molecule according to any one of claims 64 to 68, wherein
each of n₁ and n₉ is independently selected from 1 or 2, preferably n₁ is 1 and n₅ is 1;
each of n₃, n₄, and, if present, each of n₆ and n₇, is independently selected from 0 or 1.

70. The RNA molecule according to any one of claims 64 to 69, wherein
one of R₃ and R₄ if present, one of R₉ and R₁₀ if present, or one of Rₑ and R_{f} if present, is selected from the group consisting of C₁-alkyl-O—R^{O}, C₁-alkyl-S—R^{S}, C₁-alkyl-S—S—R^{S}, C₁-alkyl-R^{P};
wherein
R^{O} and R^{S} are each independently selected from the group consisting of H, C₁-C₄-alkyl, C₁-C₂-cyanoalkyl, and 5- or 6-membered saturated, partially or fully unsaturated, or aromatic carbocyclyl, carbocyclyl-C₁-alkyl, heterocyclyl, or heterocyclyl-C₁-alkyl, wherein the aforementioned heterocyclic rings comprise one or two, same or different heteroatoms selected from O, N, or S, wherein said N- and/or S-atoms are independently oxidized or non-oxidized; and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₂-alkyl, preferably halogen;
R^{P} is 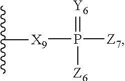
wherein
X₉ is O; Y₅ is O or S; and each of Z₆ and Z₇ is OH; and
the other one of R₃ and R₄ if present, R₉ and R₁₀ if present, Rₑ and R_{f} if present, is H; and
wherein preferably each of R₁, R₂, R₅, Rₑ, R₇, R₈, Rₐ, R_{b}, R_{c}, R_{d}, R₁₁, and R₁₂, if present, is H.

71. The RNA molecule according to any one of claims 64 to 70, wherein the acyclonucleoside comprises as the nucleobase guanine, a modified guanine or a guanine analog.

72. An RNA molecule whose 5′ end comprises a compound according to any one of claims 1 to 44.

73. An in vitro method for synthesizing an RNA molecule, the method comprising reacting nucleotides, (i) the compound according to any one of claims 1 to 44 or (ii) the cap analog according to any one of claims 45 to 52, and a DNA template in the presence of a DNA-dependent RNA polymerase under conditions suitable for the transcription of the DNA template into an RNA molecule by the DNA-dependent RNA polymerase.

74. An RNA molecule obtained by the in vitro method according to claim 73.

75. The RNA molecule according to any one of claims 53 to 72 and 74, wherein the RNA molecule comprises at least one chemical modification.

76. The RNA molecule according to claim 75, wherein the at least one chemical modification is selected from the group consisting of a base modification, a sugar modification and a backbone modification.

77. The RNA molecule according to claim 75 or 76, wherein the at least one chemical modification is a base modification, wherein the base modification is preferably selected from the group consisting of pseudouracil (psi or ψ), N1-methylpseudouracil (N1Mpsi or N1M1ψ), 1-ethylpseudouracil, 2-thiouracil (s2U), 4-thiouracil, 5-methylcytosine, 5-methyluracil, 5-methoxyuracil, and any combination thereof, more preferably is N1-methylpseudouracil.

78. The RNA molecule according to any one of claims 53 to 72 and 74 to 77, wherein the RNA molecule is a coding RNA comprising at least one coding sequence, preferably wherein the coding RNA is an mRNA.

79. The RNA molecule according to any one of claims 53 to 72 and 74 to 78, wherein the RNA molecule is a therapeutic mRNA.

80. A composition comprising the RNA molecule according to any one of claims 53 to 72 and 74 to 79.

81. The composition according to claim 80, wherein the composition is a pharmaceutical composition, optionally comprising the RNA molecule formulated in a lipid-based carrier.

82. A kit comprising (i) the compound according to any one of claims 1 to 44 or (ii) the cap analog according to any one of claims 45 to 52, and a DNA-dependent RNA polymerase.

83. The kit according to claim 82, wherein the kit further comprises nucleotides.

84. The kit according to claim 82 or 83, wherein the kit further comprises a ribonuclease inhibitor.

85. The kit according to any one of claims 82 to 84, wherein the kit further comprises a buffer.

86. Use of (i) the compound according to any one of claims 1 to 44 or (ii) the cap analog according to any one of claims 45 to 52 in an in vitro transcription reaction for producing a capped RNA molecule.

87. The use according to claim 86, wherein the capped RNA molecule is the RNA molecule according to any one of claims 53 to 72 and 74 to 79.

88. A process for preparing a compound of formula (I): 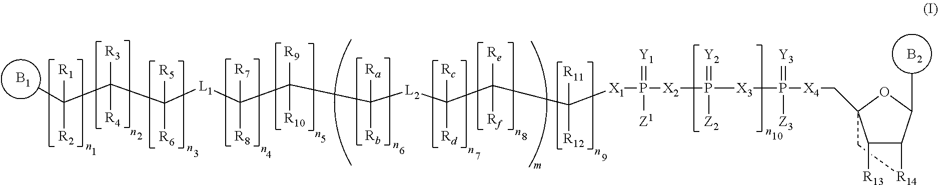
or a salt, stereoisomer, tautomer, or deuterated version thereof, wherein
R₁₃ is 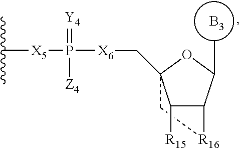
wherein
R₁₅ is OH or 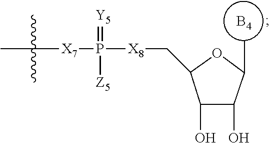
ring B₁ is guanine, a modified guanine or a guanine analog;
n₁ and n₉ are independently selected from an integer ranging from 1 to 10;
each of R₁ and R₂ is independently H, OH, SH, NH₂ or halogen, wherein, if n₁ is greater than 1, R₁ may be same or different in each repeating unit, and R₂ may be same or different in each repeating unit;
each of R₁₁ and R₁₂ is independently H, OH, SH, NH₂ or halogen, wherein, if n₉ is greater than 1, R₁₁ may be same or different in each repeating unit, and R₁₂ may be same or different in each repeating unit;
each of n₃, n₄, n₆, and n₇ is independently selected from an integer ranging from 0 to 5; wherein, if m is greater than 1, n₆ may be same or different in each repeating unit, and n₇ may be same or different in each repeating unit;
each of R₅ and R₆, if present, is independently H, OH, SH, NH₂ or halogen, wherein, if n₃ is greater than 1, R₅ may be same or different in each repeating unit, and R₆ may be same or different in each repeating unit;
each of R₇ and Rₐ, if present, is independently H, OH, SH, NH₂ or halogen, wherein, if n₄ is greater than 1, R₇ may be same or different in each repeating unit, and Rₐ may be same or different in each repeating unit;
each of Rₐ and R_{b}, if present, is independently H, OH, SH, NH₂ or halogen, wherein, if n₆ is greater than 1, Rₐ may be same or different in each repeating unit, and R_{b} may be same or different in each repeating unit;
each of R_{c} and R_{d}, if present, is independently H, OH, SH, NH₂ or halogen, wherein, if n₇ is greater than 1, R_{d} may be same or different in each repeating unit, and R_{d} may be same or different in each repeating unit;
n₂, n₅ and n₈ are independently selected from 0 or 1; wherein, if m is greater than 1, n₈ may be same or different in each repeating unit;
one of R₃ and R₄, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of R₃ and R₄, if present, is selected from the group consisting of H, OH, SH, NH₂ and halogen;
one of R₉ and R₁₀, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of R₉ and R₁₀, if present, is selected from the group consisting of H, OH, SH, NH₂ and halogen;
one of Rₑ and R_{f}, if present, is selected from the group consisting of C₁-C₄-alkyl, C₁-C₄-alkenyl, C₁-C₄-alkinyl, C₁-C₄-haloalkyl, C₁-C₃-alkyl-O—R^{O}, C₁-C₃-alkyl-S—R^{S}, C₁-C₃-alkyl-S—S—R^{S}, C₁-C₃-alkyl-NR^{N1}R^{N2}, C₁-C₃-alkyl-R^{P}, O—R^{D}, S—R^{E}, NR^{N3}R^{N4}, R^{P}, and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, C₁-C₄-alkyl, C₁-C₄-alkenyl, or C₁-C₄-alkinyl, or two R^{X} attached to the same atom form an oxo group;
and the other one of Rₑ and R_{f}, if present, is selected from the group consisting of H, OH, SH, NH₂ and halogen;
m is selected from an integer ranging from 0 to 10, with the proviso that (i) if m is 0, one of n₂ and n₅ is 1 and the other one of n₂ and n₅ is 0, (ii) if m is 1, (a) n₂, n₅ and n₈ are 0 or (b) one of n₂, n₅ and n₈ is 1 and the other two of n₂, n₅ and n₈ are 0, and (iii) if m is an integer ranging from 2 to 10, (a) n₂, n₅ and each n₈ are 0, (b) one of n₂ and n₅ is 1 and the other one of n₂ and n₅ as well as each n₈ are 0, or (c) one n₅ is 1 and each other n₈ as well as n₂ and n₅ are 0;
n₁₀ is selected from 0, 1 or 2;
L₁ is selected from the group consisting of CH₂, O, S, SO, SO₂, NR^{L}, CH(OH), CH(SH) and CH(halogen), with the proviso that, if m is 1 and n₂, n₅ and n₈ are 0, L₁ is selected from the group consisting of O, S, SO, SO₂ and NR^{L};
L₂ is selected from the group consisting of O, S, SO, SO₂ and NH; wherein, if m is greater than 1, L₂ may be same or different in each repeating unit;
each of X₁ through X₃ is independently O, S, NH or CH₂, wherein, if n₁₀ is 2, X₃ may be same or different in each repeating unit;
each of Y₁ through Y₅ is independently 0, S or Se, wherein, if n₁₀ is 2, Y₂ may be same or different in each repeating unit;
each of Z₁ through Z₅ is independently OH, SH, SeH or BH₃, wherein, if n₁₀ is 2, Z₂ may be same or different in each repeating unit;
R₁₄ is (i) selected from the group consisting of H, OH, OC₁-C₃-alkyl, and Opropargyl, wherein the dashed methylene bridge between R₁₄ and the 4′ C is absent, or (ii) O, wherein the dashed methylene bridge between R₁₄ being O and the 4′ C is present;
R₁₆ is (i) selected from the group consisting of H, OH, OC₁-C₃-alkyl, and Opropargyl, wherein the dashed methylene bridge between R₁₆ and the 4′ C is absent, or (ii) O, wherein the dashed methylene bridge between R₁₆ being O and the 4′ C is present;
R^{L} is (i) H, if one of n₂, n₅ and n₈ is 1, or (ii) selected from the group consisting of H, C₁-C₃-alkyl, C₁-C₃-alkyl-O—R^{O}, C₁-C₃-alkyl-S—R^{S}, C₁-C₃-alkyl-NR^{N1}R^{N2}, C₁-C₃-alkyl-R^{P} and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized, and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, if n₂, n₅ and n₈ are 0;
R^{O}, R^{S}, R^{N1} and R^{N2} are each independently selected from the group consisting of H, C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{D} and R^{E} are each independently selected from the group consisting of C₁-C₄-alkyl, C₁-C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl-O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group;
R^{N3} and R^{N4} are each independently selected from the group consisting of H, C₁-C₄-alkyl, C₁₋C₄-alkenyl, C₁-C₄-alkinyl, C₁-C₄alkyl-O—C₁-C₄alkyl, C₁-C₄alkyl-O—C₁-C₄alkyl-O—C₁-C₄alkyl, C₁-C₄alkyl-O—C₁-C₄alkyl- O—C₁-C₄alkyl-O—C₁-C₄alkyl, C₁-C₄alkyl-S—C₁-C₄alkyl, C₁-C₄alkyl-S—C₁-C₄alkyl-S—C₁-C₄alkyl, C₁-C₄alkyl-S—C₁-C₄alkyl-S—C₁-C₄alkyl-S—C₁-C₄alkyl, C₁-C₄alkyl-NH—C₁-C₄alkyl, C₁-C₄alkyl-NH—C₁-C₄alkyl-NH—C₁-C₄alkyl, C₁-C₄alkyl-NH—C₁-C₄alkyl-NH—C₁-C₄alkyl-NH—C₁-C₄alkyl and 3- to 10-membered saturated, partially or fully unsaturated, or aromatic carbocyclyl, carbocyclyl-C₁-C₃-alkyl, heterocyclyl, or heterocyclyl-C₁-C₃-alkyl, wherein the aforementioned heterocyclic rings comprise one or more, same or different heteroatoms selected from O, N, or S, wherein said N- and/or S-atoms are independently oxidized or non-oxidized;
and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₄-alkyl, or two R^{X} attached to the same atom form an oxo group, with the proviso that R^{N3} and R^{N4} are not both H,
R^{P} is 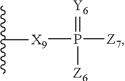
wherein
X₉ is O, S, NH or CH₂;
Y₆ is O, S or Se, and
each of Z₆ and Z₇ is independently OH, SH, SeH or BH₃; and
each of ring B₂ through ring B₄ is independently a nucleobase, a modified nucleobase or a nucleobase analog;
wherein the process comprises
reacting a compound of formula (IV) 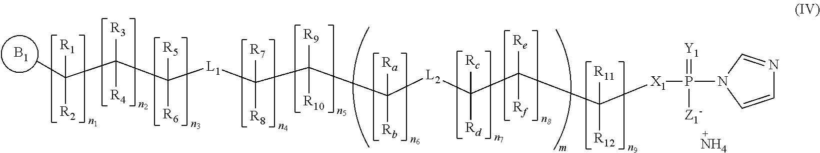
wherein B₁, R₁, R₂, R₃, R₄, R₅, R₆, R₇, R₈, R₉, R₁₀, R₁₁, R₁₂, Rₐ, R_{b}, R_{c}, R_{d}, Rₑ, R_{f}, n₁, n₂, n₃, n₄, n₅, n₆, n₇, n₈, n₉, m, L₁, L₂, X₁, Y₁, Z₁ are as defined above for formula (I),
with a compound of formula (V) 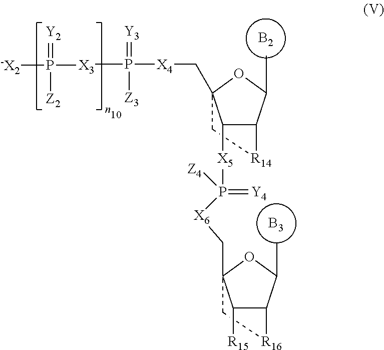
wherein X₂, Y₂, Z₂, X₃, Y₃, Z₃, X₄, Y₄, Z₄, X₅, X₆, R₁₄, R₁₅, R₁₆, B₂, and B₃ are as defined above for formula (I).

89. The process according to claim 88, wherein the reaction is performed in the presence of a metal chloride, preferably zinc chloride, manganese chloride or magnesium chloride, more preferably magnesium chloride.

90. The process according to claim 88 or 89, wherein the reaction is performed in an aqueous solution and/or an organic solvent, preferably a mixture of water and acetonitrile or a mixture of water and N-methylmorpholine.

91. The process according to any one of claims 88 to 90, wherein the product is purified by desalting via reverse-phase HPLC.

92. The process according to any one of claims 88 to 91, wherein the process further comprises preparing the compound of formula (IV) comprising reacting a compound of formula (VI) 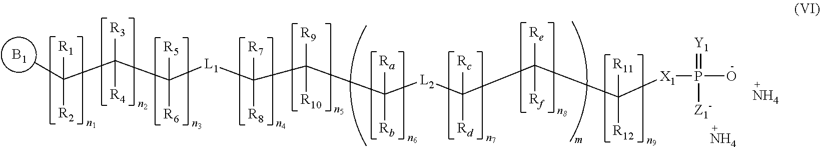
wherein B₁, R₁, R₂, R₃, R₄, R₅, R₆, R₇, R₈, R₉, R₁₀, R₁₁, R₁₂, Rₐ, R_{b}, R_{c}, R_{d}, Rₑ, R_{f}, n₁, n₂, n₃, n₄, n₅, n₆, n₇, n₈, n₉, m, L₁, L₂, X₁, Y₁, Z₁ are as defined above for formula (IV);
with carbonyldiimidazole.

93. The process according to claim 92, wherein the reaction of a compound of formula (VI) with carbonyldiimidazole is performed in DMSO.

94. The process according to claim 92 or 93, wherein excess carbonyldiimidazole is quenched with water.

95. The process according to any one of claims 88 to 94, wherein in the compound of formula (I) m is selected from 0 or 1.

96. The process according to any one of claims 88 to 94, wherein in the compound of formula (I)
m is selected from an integer ranging from 1 to 10, preferably 1, n₂, n₅ and each n₈ are 0, and L₁ is selected from the group consisting of O, S, SO, SO₂ and NR^{L}, preferably L₁ is O.

97. The process according to any one of claims 88 to 94, wherein in the compound of formula (I)
(i) m is 0, one of n₂ and n₅ is 1, and the other one of n₂ and n₅ is 0; (ii) m is 1, one of n₂, n₅ and n₈ is 1, and the other two of n₂, n₅ and n₈ are 0; (iii) m is an integer ranging from 2 to 10, one of n₂ and n₅ is 1 and the other one of n₂ and n₅ as well as each n₈ are 0, or (iv) m is an integer ranging from 2 to 10, one n₅ is 1 and each other n₈ as well as n₂ and n₅ are 0; and
L₁ is selected from the group consisting of CH₂, O, S, SO, SO₂, NR^{L}, CH(OH), CH(SH) and CH(halogen), preferably L₁ is CH₂ or 0.

98. The process according to claim 97, wherein in the compound of formula (I)
m is 0, one of n₂ and n₅ is 1, and the other one of n₂ and n₅ is 0.

99. The process according to any one of claims 88 to 98, wherein in the compound of formula (I)
n₁₀ is 1;
each of n₁ and n₉ is independently selected from 1 or 2, preferably n₁ is 1 and n₉ is 1;
each of n₃, n₄, and, if present, each of n₆ and n₇, is independently selected from 0 or 1;
R₁₄ is H or OC₁-C₃-alkyl, wherein the dashed methylene bridge between R₆ and the 4′ C is absent, preferably wherein R₁₄ is OCH₃;
R₁₆ is H, OH, or OC₁-C₃-alkyl, wherein the dashed methylene bridge between Rₐ and the 4′ C is absent, preferably wherein R₁₆ is OH;
R₁₅ is OH;
X₁ is CH₂ or O, and each of X₂ through X₆ is O;
each of Y₁ through Y₄ is O; and
each of Z₁ through Z₄ is OH.

100. The process according to any one of claims 88 to 99, wherein in the compound of formula (I)
one of R₃ and R₄ if present, one of R₉ and R₁₀ if present, or one of Rₑ and R_{f} if present, is selected from the group consisting of C₁-alkyl-O—R^{O}, C₁-alkyl-S—R^{S}, C₁-alkyl-S—S—R^{S}, C₁-alkyl-R^{P}, wherein
R^{O} and R^{S} are each independently selected from the group consisting of H, C₁-C₄-alkyl, C₁-C₂-cyanoalkyl, and 5- or 6-membered saturated, partially or fully unsaturated, or aromatic carbocyclyl, carbocyclyl-C₁-alkyl, heterocyclyl, or heterocyclyl-C₁-alkyl, wherein the aforementioned heterocyclic rings comprise one or two, same or different heteroatoms selected from O, N, or S, wherein said N- and/or S-atoms are independently oxidized or non-oxidized; and wherein each substitutable carbon or heteroatom in the aforementioned groups is independently unsubstituted or substituted with one or more, same or different substituents R^{X}, wherein R^{X} is halogen, CN, OH, SH, NH₂, or C₁-C₂-alkyl, preferably halogen;
R^{P} is 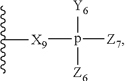
wherein
X₉ is O; Y₆ is O or S; and each of Z₆ and Z₇ is OH; and
the other one of R₃ and R₄ if present, R₉ and R₁₀ if present, Rₑ and R_{f} if present, is H; and
wherein preferably each of R₁, R₂, R₅, R₆, R₇, R₈, Rₐ, R_{b}, R_{c}, R_{d}, R₁₁, and R₁₂, if present, is H.

101. The process according to any one of claims 88 to 94, wherein the compound of formula (I) is the compound according to any one of claims 1 to 44.
